(19) United States
(12) Patent Application Publication
(10) Pub. No.: US 2004/0142999 A1
(43) Pub. Date: Jul. 22, 2004

(54) Novel compounds and compositions as cathepsin inhibitors

(75) Inventors: Michael Graupe, Pacifica, CA (US); Jiayao Li, Foster City, CA (US); John Link, San Francisco, CA (US); Sheila Zipfel, Mountain View, CA (US); Andreas Paul Timm, Bridgewater, NJ (US); David John Aldous, Gillette, NJ (US); Sukathini Thurairatnam, Bedminster, NJ (US)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignees: Aventis Pharmaceuticals Inc., Bridgewater, NJ (US); Axys Pharmaceuticals, Inc., South San Francisco, CA

(21) Appl. No.: 10/719,080

(22) Filed: Nov. 21, 2003

Related U.S. Application Data
(63) Continuation of application No. PCT/US02/17411, filed on Jun. 3, 2002.
(60) Provisional application No. 60/295,301, filed on Jun. 1, 2001.

(51) Int. Cl. A61K 31/40

(52) U.S. Cl. 514/408; 514/616; 548/571; 564/152; 514/563

(57) ABSTRACT

The present invention relates to novel selective cathepsin S inhibitors, the pharmaceutically acceptable salts and N-oxides thereof, their uses as therapeutic agents and the methods of their making.

### CROSS-REFERENCE TO RELATED APPLICATIONS

This application is a continuation of PCT/US02/17411, filed Jun. 3, 2002, which claims priority from U.S. Provisional Application No. 60/295,301 filed on Jun. 1, 2001; all of these applications incorporated herein by reference.

### BACKGROUND OF THE INVENTION

This Application relates to compounds and compositions for treating diseases associated with cysteine protease activity, particularly diseases associated with activity of cathepsin S.

Cysteine proteases represent a class of peptidases characterized by the presence of a cysteine residue in the catalytic site of the enzyme. Cysteine proteases are associated with the normal degradation and processing of proteins. The aberrant activity of cysteine proteases, e.g., as a result of increase expression or enhanced activation, however, may have pathological consequences. In this regard, certain cysteine proteases are associated with a number of disease states, including arthritis, muscular dystrophy, inflammation, tumor invasion, glomerulonephritis, malaria, periodontal disease, metachromatic leukodystrophy and others. An increase in cathepsin S activity contributes to the pathology and/or symptomatology of a number of diseases. Accordingly, molecules that inhibit the activity of cathepsin S protease are useful as therapeutic agents in the treatment of such diseases.

### SUMMARY OF THE INVENTION

This Application relates to compounds of Formula I:

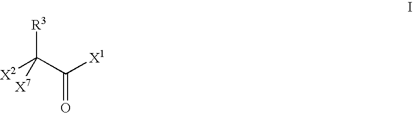

in which:

X¹ is —NHC(R¹)(R²)X³ or —NHX⁴;

X² is hydrogen, fluoro, —OH, —OR⁴, —NHR¹⁵ or —NR¹⁷R¹⁸ and X⁷ is hydrogen or X² and X⁷ both represent fluoro;

X³ is cyano, —C(R⁷)(R⁸)R¹⁶, —C(R⁶)(OR⁶)₂, —CH₂C(O)R¹⁶, —CH═CHS(O)₂R⁵, —C(O)CF₂C(O)NR⁵R⁵, —C(O)C(O)N⁵R⁶, —C(O)C(O)OR⁵, —C(O)CH₂OR⁵, —C(O)CH₂N(R⁶)SO₂R⁵ or —C(O)C(O)R⁵; wherein R⁵ is hydrogen, (C₁₋₄)alkyl, (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl; R⁶ is hydrogen, hydroxy or (C₁₋₆)alkyl; or where X³ contains an —NR⁵R⁶ group, R⁵ and R⁶ together with the nitrogen atom to which they are both attached, form hetero(C₃₋₁₀)cycloalkyl, hetero(C₅₋₁₀)aryl or hetero(C₈₋₁₀)bicycloaryl; R⁷ is hydrogen or (C₁₋₄)alkyl and R⁸ is hydroxy or R⁷ and R⁸ together form oxo; R¹⁶ is hydrogen, —X⁴, —CF₃, —CF₂CF₂R⁹ or —N(R⁶)OR⁶; R⁹ is hydrogen, halo, (C₁₋₄)alkyl, (C₅₋₁₀)aryl(C₀₋₆)alkyl or (C₅₋₁₀)heteroaryl(C₀₋₆)alkyl, with the proviso that when X³ is cyano, then X² is hydrogen, fluoro, —OH, —OR⁴ or —NR¹⁷R¹⁸ and X⁷ is hydrogen or X² and X⁷ both represent fluoro;

X⁴ comprises a heteromonocyclic ring containing 4 to 7 ring member atoms or a fused heterobicyclic ring system containing 8 to 14 ring member atoms and any carbocyclic ketone, iminoketone or thioketone derivative thereof, with the proviso that when —X⁴ is other than a heteromonocyclic ring containing 5 ring member atoms, wherein no more than two of the ring member atoms comprising the ring are heteroatoms, then X² is fluoro, —OH, —OR⁴, —NHR¹⁵ or —NR¹⁷R¹⁸ and X⁷ is hydrogen or X² and X⁷ both represent fluoro;

wherein within R⁵, X³ or X⁴ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(R¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵ is a bond or (C₁₋₆)alkylene; R¹² at each occurrence independently is hydrogen, (C₁₋₆)alkyl or halo-substituted(C₁₋₆)alkyl; R¹³ is (C₁₋₆)alkyl or halo-substituted(C₁₋₆)alkyl; and R¹⁴ is (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl;

R¹ is hydrogen or (C₁₋₆)alkyl and R² is selected from a group consisting of hydrogen, cyano, —X⁵NR¹²R¹², —X⁵NR¹²C(O)R¹², —X⁵NR¹²C(O)OR¹², —R¹², —X⁵NR¹²C(O)NR¹²R¹², —X⁵NR¹²C(NR¹² )NR¹²R¹², —X⁵OR¹², —X⁵SR¹², —X⁵C(O)OR¹², —X⁵ C(O)R¹², —X⁵OC(O)R¹², —X⁵C(O)NR¹² R¹², —X⁵S(O)₂NR¹²R¹², —X⁵NR¹²S(O)₂R¹², —X⁵P(O)(OR¹²)OR¹², —X⁵ OP(O)(OR¹²)OR₁₂, —X⁵NR¹²C(O)R¹³, —X⁵S(O)R¹³, —X⁵S(O)₂R¹³, —R¹⁴, —X⁵OR¹⁴, —X⁵SR¹⁴, —X⁵ S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵, R¹², R¹³ and R¹⁴ are as defined above; or R¹ and R² taken together with the carbon atom to which both R¹ and R² are attached form (C₃₋₈)cycloalkylene or (C₃₋₈)heterocycloalkylene; wherein within said R² any heteroaryl, aryl, cycloalkyl, heterocycloalkyl, cycloalkylene or heterocycloalkylene is unsubstituted or substituted with 1 to 3 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C (O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵S(O)₂R¹³ and —X⁵C(O)R¹³, wherein X⁵, R¹² and R¹³ are as defined above;

R³ is (C₁₋₆)alkyl or —C(R⁶)(R⁶)X⁶, wherein R⁶ is hydrogen or (C₁₋₆)alkyl and X⁶ is selected from —X⁵NR¹²R¹², —X⁵NR¹²C(O)R¹², —X⁵NR¹²C(O)OR¹², ⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵C(O)R¹³, —X⁵NR¹²C(O)R¹³, —X⁵NR¹²C(O)R¹³, —X⁵S(O)R¹³, —X⁵S(O)₂R¹³, —R¹⁴, —X⁵OR¹⁴, —X⁵SR¹⁴, —X⁵S(O)R¹⁴, —X⁵S(O)₂R¹⁴, —X⁵C(O)R¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹²R¹² and —X⁵NR¹²C(NR¹²)NR¹⁴R¹² wherein X⁵, R¹², R¹³ and R¹⁴ are as defined above;

R⁴ is selected from —X⁸NR¹²R¹², —X⁸NR¹²C(O)R¹², —X⁸NR¹²C(O)OR¹², —X⁸NR¹²C(O)NR¹²R¹², —X⁸NR¹²C(NR¹²)NR¹²R¹², —X⁸OR¹², —X⁵C(O)OR¹², —X⁵C(O)R¹², —X⁸OC(O)R¹², —X⁵C(O)NR¹²R¹², —X⁸S(O)₂NR¹²R¹², —X⁸NR¹²S(O)₂R¹², —X⁸P(O)(OR¹²)OR¹², —X⁸OP(O)(OR¹²)OR¹², —X⁵C(O)R¹³, —X⁸NR¹²C(O)R¹³, —X⁸S(O)R¹³, —X⁸S(O)₂R¹³, —R¹⁴, —X⁸OR¹⁴, —X⁸SR¹⁴, —X⁸S(O)R¹⁴, —X⁸S(O)₂R¹⁴, —X⁵C(O)R¹⁴, —X⁵C(O)OR¹⁴, —X⁸OC(O)R¹⁴, —X⁸NR¹⁴R¹², —X⁸NR¹²C(O)R¹⁴, —X⁸NR¹²C(O)OR¹⁴, —X⁵C(O)NR¹⁴R¹², —X⁸S(O)₂NR¹⁴R¹², —X⁸NR¹²S(O)₂R¹⁴, —X⁸NR¹²C(O)NR¹⁴R¹² and —X⁸NR¹²C(NR¹²)NR¹⁴R¹² wherein X⁸ is (C₁₋₆)alkylene and X⁵, R¹², R¹³ and R¹⁴ are as defined above, with the proviso that when X³ is cyano and X² is —OR⁴, where R⁴ is defined as —R¹⁴, then R¹⁴ is (C₃₋₁₀)cycloalkyl(C₁₋₆)alkyl, hetero(C₃₋₁₀)cycloalkyl(C₁₋₃)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl;

R¹⁵ is (C₆₋₁₀)aryl, hetero(C₅₋₁₀)aryl, (C₉₋₁₀)bicycloaryl or hetero(C₈₋₁₀)bicycloaryl;

R¹⁷ is (C₁₋₆)alkyl, (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl, with the proviso that when X³ is cyano, then R¹⁷ is (C₁₋₆)alkyl, (C₃₋₁₀)cycloalkyl(C₁₋₆)alkyl, hetero(C₃₋₁₀)cycloalkyl(C₁₋₆)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl;

R¹⁸ is hydrogen, (C₁₋₆)alkyl, (C₃₋₁₀)cycloalkyl(C₀₋₆)alkyl, hetero(C₃₋₁₀)cycloalkyl(C₀₋₆)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl, with the proviso that when X³ is cyano, then R¹⁸ is (C₁₋₆)alkyl, (C₃₋₁₀)cycloalkyl(C₁₋₆)alkyl, hetero(C₃₋₁₀)cycloalkyl(C₁₋₆)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl; and

wherein within R³, R⁴, R¹⁵, R¹⁷ and R¹⁸ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵C(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹²; and within R³ and R⁴ any aliphatic moiety is unsubstituted or substituted further by 1 to 5 radicals independently selected from cyano, halo, nitro, —NR¹²R¹², —NR¹²C(O)R¹², —NR¹²C(O)OR¹², —NR¹²C(O)NR¹²R¹², —NR¹²C(NR¹²)NR¹²R¹², —OR¹², —SR¹², —C(O)OR¹², —OC(O)R¹², —C(O)NR¹²R¹², —S(O)₂NR¹²R¹², —NR¹²S(O)₂R¹², —P(O)(OR¹²)OR¹², —OP(O)(OR)¹², —NR¹²C(O)R¹³, —S(O)R¹³ and —S(O)₂R¹³; wherein X⁵, R¹², R¹³ and R¹⁴ are as described above, with the proviso that when X³ is cyano and X² is —OR⁴, where R⁴ is defined as —R¹⁴, or —NHR¹⁸, then any aromatic ring system present within R¹⁴ or R¹⁸ is not substituted further by halo, (C₃₋₁₀)cycloalkyl, hetero(C₃₋₁₀)cycloalkyl, (C₆₋₁₀)aryl, hetero(C₅₋₁₀)aryl, (C₉₋₁₀)bicycloaryl or hetero(C₈₋₁₀)bicycloaryl; with the proviso that only one bicyclic ring structure is present within R³, R⁴ or R¹⁵; and the N-oxide derivatives, prodrug derivatives, protected derivatives, individual isomers and mixtures of isomers thereof; and the pharmaceutically acceptable salts and solvates of such compounds and the N-oxide derivatives, prodrug derivatives, protected derivatives, individual isomers and mixtures of isomers thereof.

A second aspect of the invention is a pharmaceutical composition which contains a compound of Formula I or their N-oxide derivatives, individual isomers or mixture of isomers thereof, or pharmaceutically acceptable salts thereof, in admixture with one or more suitable excipients.

A third aspect of the invention is a method for treating a disease in an animal in which inhibition of cathepsin S can prevent, inhibit or ameliorate the pathology and/or symptomatology of the disease, which method comprises administering to the animal a therapeutically effective amount of compound of Formula I or a N-oxide derivative, individual isomer or mixture of isomers thereof; or a pharmaceutically acceptable salt thereof.

A fourth aspect of the invention is the processes for preparing compounds of Formula I and the N-oxide derivatives, prodrug derivatives, protected derivatives, individual isomers and mixtures of isomers thereof; and the pharmaceutically acceptable salts thereof.

### DETAILED DESCRIPTION OF THE INVENTION

Definitions:

Unless otherwise stated, the following terms used in the specification and claims are defined for the purposes of this Application and have the following meanings.

“Alicyclic” means a moiety characterized by arrangement of the carbon atoms in closed non-aromatic ring structures having properties resembling those of aliphatics and may be saturated or partially unsaturated with two or more double or triple bonds.

“Aliphatic” means a moiety characterized by a straight or branched chain arrangement of the constituent carbon atoms and may be saturated or partially unsaturated with two or more double or triple bonds.

“Alkyl” represented by itself means a straight or branched, saturated or unsaturated, aliphatic radical having the number of carbon atoms indicated (e.g., (C₁₋₆)alkyl includes methyl, ethyl, propyl, isopropyl, butyl, sec-butyl, isobutyl, tert-butyl, vinyl, allyl, 1-propenyl, isopropenyl, 1-butenyl, 2-butenyl, 3-butenyl, 2-methylallyl, ethynyl, 1-propynyl, 2-propynyl, and the like). Alkyl represented along with another radical (e.g., as in arylalkyl) means a straight or branched, saturated or unsaturated aliphatic divalent radical having the number of atoms indicated or when no atoms are indicated means a bond (e.g., (C₆₋₁₀)aryl(C₀₋₃)alkyl includes phenyl, benzyl, phenethyl, 1-phenylethyl 3-phenylpropyl, and the like).

“Alkylene”, unless indicated otherwise, means a straight or branched, saturated or unsaturated, aliphatic, divalent radical having the number of carbon atoms indicated (e.g., (C₁₋₆)alkylene includes methylene(—CH₂—), ethylene(—CH₂CH₂—), trimethylene(—CH₂CH₂CH₂—), tetramethylene(—CH₂CH₂CH₂CH₂—) 2-butenylene(—CH₂CH═CHCH₂—), 2-methyltetramethylene(—CH₂CH(CH₃)CH₂CH₂—), pentamethylene(—CH₂CH₂CH₂CH₂CH₂—), and the like).

“Alkylidene” means a straight or branched saturated or unsaturated, aliphatic, divalent radical having the number of carbon atoms indicated (e.g. (C₁₋₆)alkylidene includes methylidene (═CH₂), ethylidene(═CHCH₃), isopropylidene(═C(CH₃)₂), propylidene(═CHCH₂CH₃), allylidene(═CH⁻CH═CH₂), and the like).

“Amino” means the radical —NH₂. Unless indicated otherwise, the compounds of the invention containing amino moieties include protected derivatives thereof. Suitable protecting groups for amino moieties include acetyl, tert-butoxycarbonyl, benzyloxycarbonyl, and the like.

“Animal” includes humans, non-human mammals (e.g., dogs, cats, rabbits, cattle, horses, sheep, goats, swine, deer, and the like) and non-mammals (e.g., birds, and the like).

“Aromatic” means a moiety wherein the constituent atoms make up an unsaturated ring system, all atoms in the ring system are sp² hybridized and the total number of pi electrons is equal to 4n+2.

“Aryl” means a monocyclic or fused bicyclic ring assembly containing the total number of ring carbon atoms indicated, wherein each ring is comprised of 6 ring carbon atoms and is aromatic or when fused with a second ring forms an aromatic ring assembly. For example, optionally substituted (C₆₋₁₀)aryl as used in this Application includes, but is not limited to, biphenyl-2-yl, 2-bromophenyl, 2-bromocarbonylphenyl, 2-bromo-5-fluorophenyl, 4-tert-butylphenyl, 4-carbamoylphenyl, 4-carboxy-2-nitrophenyl, 2-chlorophenyl, 4-chlorophenyl, 3-chlorocarbonylphenyl, 4-chlorocarbonylphenyl, 2-chloro-4-fluorophenyl, 2-chloro-6-fluorophenyl, 4-chloro-2-nitrophenyl, 6-chloro-2-nitrophenyl, 2,6-dibromophenyl, 2,3-dichlorophenyl, 2,5-dichlorophenyl, 3,4-dichlorophenyl, 2-difluoromethoxyphenyl, 3,5-dimethylphenyl, 2-ethoxycarbonylphenyl, 2-fluorophenyl, 2-iodophenyl, 4-isopropylphenyl, 2-methoxyphenyl, 4-methoxyphenyl, 2-methylphenyl, 3-methylphenyl, 4-methylphenyl, 5-methyl-2-nitrophenyl, 4-methylsulfonylphenyl, naphth-2-yl, 2-nitrophenyl, 3-nitrophenyl, 4-nitrophenyl, 2,3,4,5,6-pentafluorophenyl, phenyl, 2-trifluoromethoxyphenyl, 3-trifluoromethoxyphenyl, 4-trifluoromethoxyphenyl, 2-trifluoromethylphenyl, 3-trifluoromethylphenyl, 4-trifluoromethylphenyl, 2-trifluoromethylsulfanylphenyl, 4-trifluoromethylsulfanylphenyl, and the like. Optionally substituted (C₆₋₁₀)aryl as used in this Application includes 3-acetylphenyl, 3-tert-butoxycarbonylaminomethylphenyl, biphenyl-4-yl, 3-hydroxyphenyl, 4-hydroxyphenyl, 3-methoxyphenyl, naphth-2-yl, 3-phenoxyphenyl, phenyl, and the like.

“Bicycloaryl” means a bicyclic ring assembly containing the number of ring carbon atoms indicated, wherein the rings are linked by a single bond or fused and at least one of the rings comprising the assembly is aromatic, and any carbocyclic ketone, thioketone or iminoketone derivative thereof (e.g., (C₉₋₁₀)bicycloaryl includes cyclohexylphenyl, 1,2-dihydronaphthyl, 2,4-dioxo-1,2,3,4-tetrahydronaphthyl, indanyl, indenyl, 1,2,3,4-tetrahydronaphthyl, and the like).

“Carbamoyl” means the radical —C(O)NH₂. Unless indicated otherwise, the compounds of the invention containing carbamoyl moieties include protected derivatives thereof. Suitable protecting groups for carbamoyl moieties include acetyl, tert-butoxycarbonyl, benzyloxycarbonyl, and the like and both the unprotected and protected derivatives fall within the scope of the invention.

“Carbocyclic ketone derivative” means a derivative containing the moiety —C(O)—.

“Carboxy” means the radical —C(O)OH. Unless indicated otherwise, the compounds of the invention containing carboxy moieties include protected derivatives thereof. Suitable protecting groups for carboxy moieties include benzyl, tert-butyl, and the like.

“Cycloalkyl” means a saturated or partially unsaturated, monocyclic, fused bicyclic or bridged polycyclic ring assembly containing the number of ring carbon atoms indicated, and any carbocyclic ketone, thioketone or iminoketone derivative thereof (e.g., (C₃₋₁₀)cycloalkyl includes cyclopropyl, cyclobutyl, cyclopentyl, cyclohexyl, cyclohexenyl, 2,5-cyclohexadienyl, bicyclo[2.2.2]octyl, adamantan-1-yl, decahydronaphthyl, oxocyclohexyl, dioxocyclohexyl, thiocyclohexyl, 2-oxobicyclo[2.2.1]hept-1-yl, and the like).

“Cycloalkylene” means a divalent saturated or partially unsaturated, monocyclic ring or bridged polycyclic ring assembly containing the number of ring carbon atoms indicated, and any carbocyclic ketone, thioketone or iminoketone derivative thereof. For example, the instance wherein “R¹ and R² together with the carbon atom to which both R¹ and R² are attached form (C₃₋₈)cycloalkylene” includes, but is not limited to, the following:

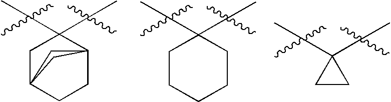

“Disease” specifically includes any unhealthy condition of an animal or part thereof and includes an unhealthy condition that may be caused by, or incident to, medical or veterinary therapy applied to that animal, i.e., the “side effects” of such therapy.

“Halo” means fluoro, chloro, bromo or iodo.

“Halo-substituted alkyl”, as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like (e.g. halo-substituted (C₁₋₃)alkyl includes chloromethyl, dichloromethyl, difluoromethyl, trifluoromethyl, 2,2,2-trifluoroethyl, perfluoroethyl, 2,2,2-trifluoro-1,1-dichloroethyl, and the like).

“Heteroatom moiety” includes —N═, —NR—, —O—, —S— or —S(O)₂—, wherein R is hydrogen, (C₁₋₆)alkyl or a protecting group.

“Heterocycloalkylene” means cycloalkylene, as defined in this Application, provided that one or more of the ring member carbon atoms indicated, is replaced by heteroatom moiety selected from —N═, —NR—, —O—, —S— or —S(O)₂—, wherein R is hydrogen or (C₁₋₆)alkyl. For example, the instance wherein R¹ and R² together with the carbon atom to which both R¹ and R² are attached form hetero(C₃₋₈)cycloalkyl” includes, but is not limited to, the following:

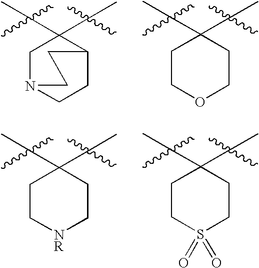

in which R is hydrogen, (C₁₋₆)alkyl, or a protecting group.

“Heteroaryl” means aryl, as defined in this Application, provided that one or more of the ring carbon atoms indicated are replaced by a heteroatom moiety selected from —N═, —NR—, —O— or —S—, wherein R is hydrogen, (C₁₋₆)alkyl, a protecting group or represents the free valence which serves as the point of attachment to a ring nitrogen, and each ring is comprised of 5 or 6 ring atoms. For example, optionally substituted hetero(C₅₋₁₀)aryl as used in this Application includes, but is not limited to, 4-amino-2-hydroxypyrimidin-5-yl, benzothiazol-2-yl, 1H-benzoimidazol-2-yl, 2-bromopyrid-5-yl, 5-bromopyrid-2-yl, 4-carbamoylthiazol-2-yl, 3-carboxypyrid-4-yl, 5-carboxy-2,6-dimethylpyrid-3-yl, 3,5-dimethylisoxazol-4-yl, 5-ethoxy-2,6-dimethylpyrid-3-yl, 5-fluoro-6-hydroxypyrimidin-4-yl, fur-2-yl, fur-3-yl, 5-hydroxy-4,6-dimethylpyrid-3-yl, 8-hydroxy-5,7-dimethylquinolin-2-yl, 5-hydroxymethylisoxazol-3-yl, 3-hydroxy-6-methylpyrid-2-yl, 3-hydroxypyrid-2-yl, 1H-imidazol-2-yl, 1H-imidazol-4-yl, 1H-indol-3-yl, isothiazol-4-yl, isoxazol-4-yl, 2-methylfur-3-yl, 5-methylfur-2-yl, 1-methyl-1H-imidazol-2-yl, 5-methyl-3H-imidazol-4-yl, 5-methylisoxazol-3-yl, 5-methyl-2H-pyrazol-3-yl, 3-methylpyrid-2-yl, 4-methylpyrid-2-yl, 5-methylpyrid-2-yl, 6-methylpyrid-2-yl, 2-methylpyrid-3-yl, 2-methylthiazol-4-yl, 5-nitropyrid-2-yl, 2H-pyrazol-3-yl, 3H-pyrazol-4-yl, pyridazin-3-yl, pyrid-2-yl, pyrid-3-yl, pyrid-4-yl, 5-pyrid-3-yl-2H-[1,2,4]triazol-3-yl, pyrimidin-4-yl, pyrimidin-5-yl, 1H-pyrrol-3-yl, quinolin-2-yl, 1H-tetrazol-5-yl, thiazol-2-yl, thiazol-5-yl, thien-2-yl, thien-3-yl, 2H-[1,2,4]triazol-3-yl, 3H-[1,2,3]triazol-4-yl, 5-trifluoromethylpyrid-2-yl, and the like. Suitable protecting groups include tert-butoxycarbonyl, benzyloxycarbonyl, benzyl, 4-methoxybenzyl, 2-nitrobenzyl, and the like. Optionally substituted hetero(C₅₋₁₀)aryl as used in this Application to define R⁴ includes benzofur-2-yl, fur-2-yl, fur-3-yl, pyrid-3-yl, pyrid-4-yl, quinol-2-yl, quinol-3-yl, thien-2-yl, thien-3-yl, and the like.

“Heterobicycloaryl” means bicycloaryl, as defined in this Application, provided that one or more of the ring carbon atoms indicated are replaced by a heteroatom moiety selected from —N═, —NR—, —O— or —S—, wherein R is hydrogen, (C₁₋₆)alkyl, a protecting group or represents the free valence which serves as the point of attachment to a ring nitrogen, and any carbocyclic ketone, thioketone or iminoketone derivative thereof. For example, optionally substituted hetero(C₈₋₁₀)bicycloaryl as used in this Application includes, but is not limited to, 2-amino-4-oxo-3,4-dihydropteridin-6-yl, and the like. In general, the term heterobicycloaryl as used in this Application includes, for example, benzo[1,3]dioxol-5-yl, 3,4-dihydro-2H-[1,8]naphthyridinyl, 3,4-dihydro-2H-quinolinyl, 2,4-dioxo-3,4-dihydro-2H-quinazolinyl, 1,2,3,4,5,6-hexahydro[2,2′]bipyridinylyl, 3-oxo-2,3-dihydrobenzo[1,4]oxazinyl, 5,6,7,8-tetrahydroquinolinyl, and the like.

“Heterocycloalkyl” means cycloalkyl, as defined in this Application, provided that one or more of the ring carbon atoms indicated are replaced by a heteroatom moiety selected from —N═, —NR—, —O— or —S—, wherein R is hydrogen, (C₁₋₆)alkyl, a protecting group or represents the free valence which serves as the point of attachment to a ring nitrogen, and any carbocyclic ketone, thioketone or iminoketone derivative thereof (e.g., the term hetero(C₅₋₁₀)cycloalkyl includes imidazolidinyl, morpholinyl, piperazinyl, piperidyl, pyrrolidinyl, pyrrolinyl, quinuclidinyl, and the like). Suitable protecting groups include tert-butoxycarbonyl, benzyloxycarbonyl, benzyl, 4-methoxybenzyl, 2-nitrobenzyl, and the like. Both the unprotected and protected derivatives fall within the scope of the invention.

“Heteromonocyclic ring” means a saturated or partially unsaturated, monocyclic ring assembly containing the number of ring carbon atoms indicated, as defined in this Application, provided that one or more of the ring carbon atoms indicated are replaced by one or more heteroatoms selected from —N═, —NY³—, —O— or —S—, wherein Y³ is hydrogen, alkyl, aryl, arylalkyl, —C(═O)—R¹⁴, —C(═O)—OR¹⁴ or —SO₂R¹⁴.

“Heterobicyclic ring” means a saturated or partially unsaturated fused bicyclic or bridged polycyclic ring assembly containing the number of ring carbon atoms indicated, as defined in this Application, provided that one or more of the ring carbon atoms indicated are replaced by one or more heteroatoms selected from —N═, —NY³—, —O— or —S—, wherein Y³ is hydrogen, alkyl, aryl, arylalkyl, —C(═O)—R¹⁴, —C(═O)—OR or —SO₂R¹⁴.

“Hydroxy” means the radical —OH. Unless indicated otherwise, the compounds of the invention containing hydroxy radicals include protected derivatives thereof. Suitable protecting groups for hydroxy moieties include benzyl and the like.

“Iminoketone derivative” means a derivative containing the moiety —C(NR)—, wherein R is hydrogen or (C₁₋₆)alkyl.

“Isomers” mean compounds of Formula I having identical molecular formulae but differ in the nature or sequence of bonding of their atoms or in the arrangement of their atoms in space. Isomers that differ in the arrangement of their atoms in space are termed “stereoisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center has two enantiomeric forms of opposite chirality is termed a “racemic mixture”. A compound that has more than one chiral center has 2ⁿ⁻¹ enantiomeric pairs, where n is the number of chiral centers. Compounds with more than one chiral center may exist as ether an individual diastereomers or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992). It is understood that the names and illustration used in this Application to describe compounds of Formula I are meant to be encompassed all possible stereoisomers. Thus, for example, the name N-[1-(1-benzothiazol-2-yl-methanoyl)-propyl]-2-hydroxy-3-phenylmethanesulfonyl-propionamide is meant to include (S)—N-[1-(1-benzothiazol-2-yl-methanoyl)-propyl]-2-hydroxy-3-phenylmethanesulfonyl-propionamide, (R)—N-[1-(1-benzothiazol-2-yl-methanoyl)-propyl]-2-hydroxy-3-phenylmethanesulfonyl-propionamide, (R)—N—[(S)-1-(1-benzothiazol-2-yl-methanoyl)-propyl]-2-hydroxy-3-phenylmethanesulfonyl-propionamide, (S)—N—[(R)-1-(1-benzothiazol-2-yl-methanoyl)-propyl]-2-hydroxy-3-phenylmethanesulfonyl-propionamide, (R)—N—[(R)-1-(1-benzothiazol-2-yl-methanoyl)-propyl]-2-hydroxy-3-phenylmethanesulfonyl-propionamide, N—[(S)-1-(1-benzothiazol-2-yl-inethanoyl)-propyl]-2-hydroxy-3-phenylmethanesulfonyl-propionamide, N—[(R)-1-(1-benzothiazol-2-yl-methanoyl)-propyl]-2-hydroxy-3-phenylmethanesulfonyl-propionamide, (S)—N—[(S)-1-(1-benzothiazol-2-yl-methanoyl)-propyl]-2-hydroxy-3-phenylmethanesulfonyl-propionamide and any mixture, racemic or otherwise, thereof

“Ketone derivative” means a derivative containing the moiety —C(O)—. For example, in this Application X³ can be 2-acetoxy-azetidin-3-yl. The “carbocyclic ketone derivative” of this example of X³ would be 2-acetoxy-4-oxo-azetidin-3-yl (see Table 3, C32).

“Nitro” means the radical —NO₂.

“Optional” or “optionally” means that the subsequently described event or circumstance may or may not occur, and that the description includes instances where the event or circumstance occurs and instances in which it does not. For example, the phrase “wherein within R³ and R⁴ any alicyclic or aromatic ring system may be substituted further by 1-5 radicals . . .” means that R³ and R⁴ may or may not be substituted in order to fall within the scope of the invention.

“Oxoalkyl” means alkyl, as defined above, wherein one of the number of carbon atoms indicated is replaced by an oxygen group (—O—), e.g., oxo(C₂₋₆)alkyl includes methoxymethyl, etc.

“N-oxide derivatives” means derivatives of compounds of Formula I in which nitrogens are in an oxidized state (i.e., O—N) and which possess the desired pharmacological activity.

“Pathology” of a disease means the essential nature, causes and development of the disease as well as the structural and functional changes that result from the disease processes.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of Formula I which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartatic acid, citric acid, benzoic acid, o-(4-hydroxybenzoyl)benzoic acid, cinnamic acid, madelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Prodrug” means a compound which is convertible in vivo by metabolic means (e.g. by hydrolysis) to a compound of Formula I. For example an ester of a compound of Formula I containing a hydroxy group may be convertible by hydrolysis in vivo to the parent molecule. Alternatively an ester of a compound of Formula I containing a carboxy group may be convertible by hydrolysis in vivo to the parent molecule. Suitable esters of compounds of Formula I containing a hydroxy group, are for example acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methanesulphonates, ethanesulphonates, benzenesulphonates, p-toluenesulphonates, cyclohexylsulphamates and quinates. Suitable esters of compounds of Formula I containing a carboxy group, are for example those described by F. J. Leinweber, Drug Metab. Res., 1987, 18, page 379. An especially useful class of esters of compounds of Formula I containing a hydroxy group, may be formed from acid moieties selected from those described by Bundgaard et al., J. Med. Chem., 1989, 32, page 2503-2507, and include substituted (aminomethyl)-benzoates, for example, dialkylamino-methylbenzoates in which the two alkyl groups may be joined together and/or interrupted by an oxygen atom or by an optionally substituted nitrogen atom, e.g. an alkylated nitrogen atom, more especially (morpholino-methyl)benzoates, e.g. 3- or 4-(morpholinomethyl)-benzoates, and (4-alkylpiperazin-1-yl)benzoates, e.g. 3- or 4-(4-alkylpiperazin-1-yl)benzoates.

“Protected derivatives” means derivatives of compounds of Formula I in which a reactive site or sites are blocked with protecting groups. Protected derivatives of compounds of Formula I are useful in the preparation of compounds of Formula I or in themselves may be active cathepsin S inhibitors. A comprehensive list of suitable protecting groups can be found in T. W. Greene, Protecting Groups in Organic Synthesis, 3rd edition, John Wiley & Sons, Inc. 1999.

“Therapeutically effective amount” means that amount which, when administered to an animal for treating a disease, is sufficient to effect such treatment for the disease.

“Thioketone derivative” means a derivative containing the moiety —C(S)—.

“Treatment” or “treating” means any administration of a compound of the present invention and includes:

(1) preventing the disease from occurring in an animal which may be predisposed to the disease but does not yet experience or display the pathology or symptomatology of the disease,

(2) inhibiting the disease in an animal that is experiencing or displaying the pathology or symptomatology of the diseased (i.e., arresting further development of the pathology and/or symptomatology), or

(3) ameliorating the disease in an animal that is experiencing or displaying the pathology or symptomatology of the diseased (i.e., reversing the pathology and/or symptomatology).

Nomenclature:

The compounds of Formula I and the intermediates and starting materials used in their preparation are named in accordance with IUPAC rules of nomenclature in which the characteristic groups have decreasing priority for citation as the principle group as follows: acids, esters, amides, etc. Alternatively, the compounds are named by AutoNom 4.0 (Beilstein Information Systems, Inc.). For example, a compound of Formula I wherein X² is hydroxy, R³ is phenylmethanesulfonylmethyl and X¹ is —NHC(R¹)(R²)X³ (in which R¹ is hydrogen, R² is ethyl and X³ is 1-benzothiazol-2-yl-methanocyl); that is, a compound having the following structure:

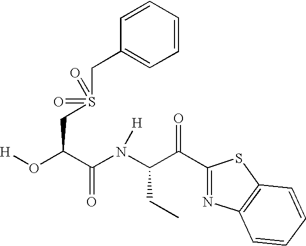

is named (R)—N—[(S)-1-(1-benzothiazol-2-yl-methanoyl)-propyl]-2-hydroxy-3-phenylmethanesulfonyl-propionamide;

### PRESENTLY PREFERRED EMBODIMENTS

While the scope of the invention is set forth in the Summary of the Invention, certain aspects of the invention are preferred. For example, preferred is a compound of Formula I:

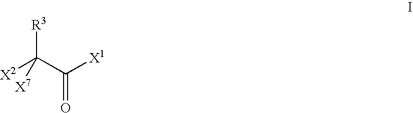

in which:

X¹ is —NHC(R¹)(R²)X³ or —NHCH(R¹⁹)C(O)R²⁰,

X² is hydrogen, fluoro, —OH, —OR⁴, NHR¹⁵ or —NR¹⁷R¹⁸ and X⁷ is hydrogen or X²and X⁷ both represent fluoro;

X³ is cyano, —C(R⁷)(R⁶)R¹⁶, —C(R⁶)(OR⁶)₂, —CH₂C(O)R¹⁶, —CH═CHS(O)₂R⁵, —C(O)CF₂C(O)NR⁵R⁵, —C(O)C(O)NR⁵R⁶, —C(O)C(O)OR⁵, —C(O)CH₂OR⁵, —C(O)CH₂N(R⁶)SO₂R⁵ or —C(O)C(O)R⁵; wherein R⁵ is hydrogen, (C₁₋₄)alkyl, (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl; R⁶ is hydrogen, hydroxy or (C₁₋₆)allyl; or where X³ contains an —NR⁵R⁶ group, R⁵ and R⁶ together with the nitrogen atom to which they are both attached, form hetero(C₃₋₁₀)cycloalkyl, hetero(C₅₋₁₀)aryl or hetero(C₈₋₁₀)bicycloaryl; R⁷ is hydrogen or (C₁₋₄)alkyl and R⁸ is hydroxy or R⁷ and R⁸ together form oxo; R¹⁶ is hydrogen, —X⁴, —CF₃, —CF₂CF₂R⁹ or —N(R⁶)OR⁶; R⁹ is hydrogen, halo, (C₁₋₄)alkyl, (C₅₋₁₀)aryl(C₀₋₆)alkyl or (C₅₋₁₀)heteroaryl(C₀₋₆)alkyl, with the proviso that when X³ is cyano, then X² is hydrogen, fluoro, —OH, —OR⁴ or —NR¹⁷R¹⁸ and X⁷ is hydrogen or X² and X⁷ both represent fluoro;

X⁴ comprises a heteromonocyclic ring containing 4 to 7 ring member atoms or a fused heterobicyclic ring system containing 8 to 14 ring member atoms and any carbocyclic ketone, iminoketone or thioketone derivative thereof, with the proviso that when —X⁴ is other than a heteromonocyclic ring containing 5 ring member atoms, wherein no more than two of the ring member atoms comprising the ring are heteroatoms, then X² is fluoro, —OH, —OR⁴, —NHR¹⁵ or —NR¹⁷R¹⁸ and X⁷ is hydrogen or X² and X⁷ both represent fluoro;

wherein within R⁵, X³ or X⁴ any alicyclic or aromatic ring system is unsubstituted or substituted further by 1 to 5 radicals independently selected from (C ₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹²,—X⁵S(O)₂NR¹²R¹², —X NR¹²S(O)₂R¹², —X⁵ P(O)(OR¹²)OR¹², —X⁵OP(O)(OR¹²)OR¹², —X⁵NR¹²C(O)R¹³, —X⁵S(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹³C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵ is a bond or (C₁₋₆)alkylene; R¹² at each occurrence independently is hydrogen, (C₁₋₆)alkyl or halo-substituted(C₁₋₆)alkyl; R¹³ is (C₁₋₆)alkyl or halo-substituted(C₁₋₆)alkyl; and R¹⁴ is (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl;

R¹ is hydrogen or (C₁₋₆)alkyl and R² is selected from a group consisting of hydrogen, cyano, —X⁵NR¹² R¹², —X⁵NR¹²C(O)R¹², —X⁵NR¹²C(O)OR¹², —R¹³, —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵S(O)₂R¹³, —R¹⁴, X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)R₂R¹⁴, —X⁵NR¹²C(O)NR¹⁴R¹² and —X⁵NR¹²C(NR¹²)NR¹⁴R¹², wherein X⁵, R¹², R¹³ and R¹⁴ are as defined above; or R¹ and R² taken together with the carbon atom to which both R¹ and R² are attached form (C₃₋₈)cycloalkylene or (C₃₋₈)heterocycloalkylene; wherein within said R² any heteroaryl, aryl, cycloalkyl, heterocycloalkyl, cycloalkylene or heterocycloalkylene is unsubstituted or substituted with 1 to 3 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR²C(O)R¹², —X⁵NR¹³C(O)OR¹², —X^{NR}¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵S(O)₂R¹³ and —X⁵C(O)R¹³, wherein X⁵, R¹² and R¹³ are as defined above;

R³ is (C₁₋₆)alkyl or —C(R⁶)(R⁶)X⁶, wherein R⁶ is hydrogen or (C₁₋₆)alkyl and X⁶ is selected from —X⁵NR¹²R¹², —X¹²NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵C(O)R¹³, —X⁵NR¹²C(O)R¹³, —X⁵S(O)R¹³, —X⁵S(O)₂R¹³, —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹⁴R¹² and —X⁵NR¹³C(NR¹²)NR¹⁴R¹² wherein X⁵, R¹², R¹³ and R¹⁴ are as defined above;

R⁴ is selected from —X⁸NR¹²R¹², —⁸NR¹²C(O)R¹², —X^{NR}¹²C(O)OR¹², —X⁸NR¹²C(O)NR¹²R¹², —X⁸NR¹²C(NR¹²)NR¹²R¹², —X⁸SR¹², —X⁵C(O)OR¹², —X⁵C(O)R¹², —X⁸OC(O)R¹², —X⁵C(O)NR¹²R¹², —X⁸S(O)₂NR¹²R¹², —X⁸NR¹²S(O)₂R¹², —X⁸P(O)(OR¹²)OR¹², —X⁸OP(O)(OR¹²)OR¹², —X⁵C(O)R¹³, —X⁸NR¹²C(O)R¹³, —X⁸S(O)R¹³, —X⁸S(O)₂R¹³, —R¹⁴, —X⁸OR¹⁴, —X⁸SR¹⁴, —X⁸S(O)R¹⁴, —X⁸S(O)₂R¹⁴, —X⁵C(O)R¹⁴, —X⁵C(O)OR¹⁴, —X⁸ OC(O)R¹⁴, —X⁸NR¹⁴R¹², —X⁸NR¹²C(O)R¹⁴, —X⁸NR¹²C(O)OR¹⁴, —X⁵C(O)NR¹⁴R¹², —X⁸S(O)₂NR¹⁴R¹², —X⁸NR¹²S(O)₂R¹⁴, —X⁸NR¹²C(O)NR¹⁴R¹² and —X⁸NR¹²C(NR¹²)NR¹⁴R¹² wherein X⁸ is (C₁₋₆)alkylene and X⁵, R¹², R¹³ and R¹⁴ are as defined above, with the proviso that when X³ is cyano and X² is —OR⁴, where R⁴ is defined as -R¹⁴, then R¹⁴ is (C₃₋₁₀)cycloalkyl(C₁₋₆)alkyl, hetero(C₃₋₁₀)cycloalkyl(C₁₋₃)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl;

R¹⁵ is (C₆₋₁₀)aryl, hetero(C₅₋₁₀)aryl, (C₉₋₁₀)bicycloaryl or hetero(C₈₋₁₀)bicycloaryl;

R¹⁷ is (C₁₋₆)alkyl, (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl, with the proviso that when X³ is cyano, then R¹⁷ is (C₁₋₆)alkyl, (C₃₋₁₀)cycloalkyl(C₁₋₆)alkyl, hetero(C₃₋₁₀)cycloalkyl(C₁₋₆)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl;

R¹⁸ is hydrogen, (C₁₋₆)alkyl, (C₃₋₁₀)cycloalkyl(C₀₋₆)alkyl, hetero(C₃₋₁₀)cycloalkyl(C₀₋₆)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl, with the proviso that when X³ is cyano, then R¹⁸ is (C₁₋₆)alkyl, (C₃₋₁₀)cycloalkyl(C₀₋₆)alkyl, hetero(C₃₋₁₀)cycloalkyl(C₁₋₆)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl; and

R¹⁹ and R²⁰ together with the atoms to which R¹⁹ and R²⁰ are attached form (C₄₋₈)heterocycloalkylene, wherein no more than one of the ring member atoms comprising the ring is a heteroatom selected from —NR²¹— or —O—, wherein the ring is unsubstituted or substituted with R², wherein R² is as defined above, and R²¹ is hydrogen, —C(O)OR¹², —C(O)R¹², —C(O)NR¹²R¹², —S(O)₂NR¹²R¹², —S(O)R¹³ and —S(O)₂R¹³, —S(O)R¹⁴, —S(O)₂R¹⁴, —C(O)R¹⁴, —C(O)OR¹⁴, —C(O)NR¹²R¹² and —S(O)₂NR¹⁴R¹², wherein R¹², R¹³ and R¹⁴ are as defined above;

wherein within R³, R⁴, R¹⁵, R¹⁷ and R¹⁸ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵N¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵PO(O)(OR¹²)OR¹², —X⁵NR¹²C(O)R¹³, —X⁵S(O)R¹³, —X⁵C(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹²; and within R³ and R⁴ any aliphatic moiety is unsubstituted or substituted further by 1 to 5 radicals independently selected from cyano, halo, nitro, —NR¹²R¹², —NR¹²C(O)R¹², —NR¹²C(O)OR¹², —NR¹²C(O)NR¹²R¹², —NR¹²C(NR¹²)NR¹²R¹², —OR¹², —SR¹², —C(O)OR¹², —C(O)R¹², —OC(O)R¹², —C(O)NR¹²R¹², —S(O)₂NR¹²R¹², —NR¹²S(O)₂R¹², —P(O)(OR¹²)OR¹², —OP(O)(OR¹²)OR¹², —NR¹²C(O)R¹³, —S(O)R¹³ and —S(O)₂R¹³; wherein X⁵, R¹², R¹³ and R¹⁴ are as described above, with the proviso that when X³ is cyano and X² is —OR⁴, where R⁴ is defined as —R¹⁴, or —NHR¹⁸, then any aromatic ring system present within R¹⁴ or R¹⁸ is not substituted further by halo, (C₃₋₁₀)cycloalkyl, hetero(C₃₋₁₀)cycloalkyl, (C₆₋₁₀)aryl, hetero(C₅₋₁₀)aryl, (C₉₋₁₀)bicycloaryl or hetero(C₈₋₁₀)bicycloaryl; with the proviso that only one bicyclic ring structure is present within R³, R⁴ or R¹⁵; and the N-oxide derivatives, prodrug derivatives, protected derivatives, individual isomers and mixtures of isomers thereof; and the pharmaceutically acceptable salts and solvates of such compounds and the N-oxide derivatives, prodrug derivatives, protected derivatives, individual isomers and mixtures of isomers thereof.

Preferred is a compound of Formula I:

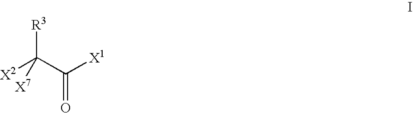

in which:

X¹ is —NHC(R¹)(R²)X³ or —NHCH(R¹⁹)C(O)R²⁰;

X² is hydrogen, fluoro, —OH, —OR⁴, —NHR¹⁵ or —NR¹⁷R¹⁸ and X⁷ is hydrogen or X² and X⁷ both represent fluoro;

X³ is —C(R⁷)(R⁸)R¹⁶, —C(R⁶)(OR⁶)₂, —CH₂C(O)R¹⁶, —CH═CHS(O)₂R⁵, —C(O)CF₂C(O)NR⁵R⁵, —C(O)C(O)NR⁵R⁶, —C(O)C(O)OR⁵, —C(O)CH₂OR⁵, —C(O)CH₂N(R⁶)SO₂R⁵ or —C(O)C(O)R⁵; wherein R⁵ is hydrogen, (C₁₋₄)alkyl, (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl; R⁶ is hydrogen, hydroxy or (C₁₋₆)alkyl; or where X³ contains an —NR⁵R⁶ group, R⁵ and R⁶ together with the nitrogen atom to which they are both attached, form hetero(C₃₋₁₀)cycloalkyl, hetero(C₅₋₁₀)aryl or hetero(C₈₋₁₀)bicycloaryl; R⁷ is hydrogen or (C₁₋₄)alkyl and R⁸ is hydroxy or R⁷ and R⁸ together form oxo; R¹⁶ is hydrogen, —X⁴, —CF₃, —CF₂CF₂R⁹ or —N(R⁶)OR⁶; R⁹ is hydrogen, halo, (C₁₋₄)alkyl, (C₅₋₁₀)aryl(C₀₋₆)alkyl or (C₅₋₁₀)heteroaryl(C₀₋₆)alkyl;

X⁴ comprises a heteromonocyclic ring containing 4 to 7 ring member atoms or a fused heterobicyclic ring system containing 8 to 14 ring member atoms and any carbocyclic ketone, iminoketone or thioketone derivative thereof, with the proviso that when —X⁴ is other than a heteromonocyclic ring containing 5 ring member atoms, wherein no more than two of the ring member atoms comprising the ring are heteroatoms, then X² is fluoro, —OH, —OR⁴, —NHR¹⁵ or —NR¹⁷R¹⁸ and X⁷ is hydrogen or X² and X⁷ both represent fluoro;

wherein within R⁵, X³ or X⁴ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X^{NR}¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵ is a bond or (C₁₋₆)alkylene; R¹² at each occurrence independently is hydrogen, (C₁₋₆)alkyl or halo-substituted(C₁₋₆)alkyl; R¹³ is (C₁₋₆)alkyl or halo-substituted(C₁₋₆)alkyl; and R¹⁴ is (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl;

R¹ is hydrogen or (C₁₋₆)alkyl and R² is selected from a group consisting of hydrogen, cyano, —X⁵NR¹²R¹², —X⁵NR¹²C(O)R¹², —X⁵NR¹²C(O)OR¹², —X⁵R¹², —X⁵NR¹²C(O)NR¹²R¹², —X^{NR}¹²C(NR¹²)NR¹²R¹², —X⁵OR¹², —X⁵SR¹², —X⁵C(O)OR¹², —X⁵C(O)R¹²R¹², —X⁵OC(O)R¹², —X⁵C(O)NR¹²R¹², —X⁵S(O)₂NR¹²R¹², —X⁵NR¹²S(O)₂R¹², —X⁵P(O)(OR)¹²)OR¹², —X⁵OP(O)(OR¹²)OR¹², X⁵NR¹²C(O)R¹³, —X⁵S(O)R¹³, —X⁵S(O)₂R¹³, —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X^{NR}¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵, R¹², R¹³ and R¹⁴ are as defined above; or R¹ and R² taken together with the carbon atom to which both R¹ and R² are attached form (C₃₋₈)cycloalkylene or (C₃₋₈)heterocycloalkylene; wherein within said R² any heteroaryl, aryl, cycloalkyl, heterocycloalkyl, cycloalkylene or heterocycloalkylene is unsubstituted or substituted with 1 to 3 radicals independently selected from (C₋₁₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵S(O)₂R¹³ and —X⁵C(O)R¹³, wherein X⁵, R¹² and R¹³ are as defined above;

R³ is —C(R⁶)(R⁶)X⁶, wherein R⁶ is hydrogen or (C₁₋₆)alkyl and X⁶ is selected from —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵C(O)R¹³, —X⁵NR¹²C(O)R¹³, —X⁵S(O)R¹³, —X⁵S(O)₂R¹³, R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴, R¹², —X⁵NR¹²S(O)₂R¹⁴, —X⁵NR¹²C(O)NR¹⁴R¹² and —X⁵NR¹²C(NR¹²)NR¹⁴R¹² wherein X⁵, R¹², R¹³ and R¹⁴ are as defined above;

R⁴ is selected from —X⁸NR¹²R¹², —X⁸NR¹²C(O)R¹², —X⁸NR¹²C(O)OR¹², —X⁸NR¹²C(O)NR¹²R¹², —X⁸NR¹²C(NR¹²)NR¹², R¹², —X⁸OR¹², —X⁵C(O)OR¹², —X⁵C(O)R¹², —X⁸OC(O)R¹², —X⁵C(O)NR¹², —X⁸S(O)₂NR¹²R¹², —X⁸NR¹²S(O)₂R¹², —X⁸P(O)(OR¹²)OR¹², —X⁸OP(O)(OR¹²)OR¹², —X⁵C(O)R¹³, —X⁸NR¹²C(O)R¹³, —X⁸S(O)R¹³, —X⁸S(O)₂R¹³, —R¹⁴, —X⁸OR¹⁴, —X⁸SR¹⁴, —X⁸S(O)R¹⁴, —X⁵S(O)₂R¹⁴, —X⁵C(O)R¹⁴, —X⁵C(O)OR¹⁴, —X⁸OC(O)R¹⁴, —X⁸NR¹⁴R¹², —X⁸NR¹²C(O)R¹⁴, —X⁸NR¹²C(O)OR¹⁴, —X⁵C(O)NR¹⁴R¹², —X⁸S(O)₂NR¹⁴R¹², —X⁸NR¹²S(O)₂R¹⁴, —X⁸NR¹²C(O)NR¹⁴R¹² and —X⁸NR¹²C(NR¹²C(NR¹²)NR¹⁴R¹² wherein X⁸ is (C₁₋₆)alkylene and X⁵, R¹², R¹³ and R¹⁴ are as defined above;

R¹⁵ is (C₆₋₁₀)aryl, hetero(C₅₋₁₀)aryl, (C₉₋₁₀)bicycloaryl or hetero(C₈₋₁₀)bicycloaryl;

R¹⁷ is hydrogen, (C₁₋₆)alkyl, (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl;

R¹⁸ is (C₁₋₆)alkyl, (C₃₋₁₀)cycloalkyl(C₀₋₆)alkyl, hetero(C₃₋₁₀)cycloalkyl(C₀₋₆)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl; and

R¹⁹ and R²⁰ together with the atoms to which R¹⁹ and R²⁰ are attached form (C₄₋₈)heterocycloalkylene, wherein no more than one of the ring member atoms comprising the ring is a heteroatom selected from —NR²¹— or —O—, wherein the ring is unsubstituted or substituted with R¹, wherein R¹ is as defined above, and R²¹ is hydrogen, —C(O)OR¹², —C(O)R¹², —C(O)NR¹²R¹², —S(O)₂NR¹²R¹², —S(O)R¹³ and —S(O)₂R¹³, —S(O)R¹⁴, —S(O)₂R¹⁴, —C(O)R¹⁴, —C(O)OR¹⁴, —C(O)NR¹²R¹² and —S(O)₂NR¹⁴R¹², wherein R¹², R¹³ and R¹⁴ are defined above;

wherein within R³, R⁴, R¹⁵, R¹⁷ and R¹⁸ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵C(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹²; and within R³ and R⁴ any aliphatic moiety is unsubstituted or substituted further by 1 to 5 radicals independently selected from cyano, halo, nitro, —NR¹²R¹², —NR¹²C(O)R¹², —NR¹²C(O)OR¹², —NR¹²C(O)NR¹²R¹⁴. —NR¹²C(NR¹²)NR¹²R¹², —OR¹², —SR¹², —C(O)OR¹², —C(O)R¹², —OC(O)R¹², —C(O)NR¹²R¹², —S(O)₂NR¹²R¹², —NR¹²S(O)₂R¹², —P(O)(OR¹²)OR², —OP(O)(OR¹²)OR¹², —NR¹²C(O)R¹³, —S(O)R¹³ and —S(O)₂R¹³; wherein X⁵, R¹², R¹³ and R¹⁴ are as described above; with the proviso that only one bicyclic ring structure is present within R³, R⁴ or R¹⁵; and the N-oxide derivatives, prodrug derivatives, protected derivatives, individual isomers and mixtures of isomers thereof; and the pharmaceutically acceptable salts and solvates of such compounds and the N-oxide derivatives, prodrug derivatives, protected derivatives, individual isomers and mixtures of isomers thereof.

Preferred is a compound of Formula I:

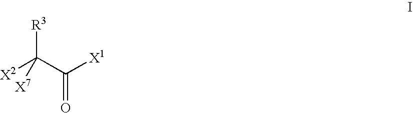

in which:

X¹ is —NHC(R¹)(R²)X³ or —NHCH(R¹⁹)C(O)R²⁰;

X² is hydrogen, fluoro, —OH, —OR⁴ or —NR¹⁷R¹⁸ and X⁷ is hydrogen or X² and X⁷ both represent fluoro;

X³ is cyano;

wherein within X³ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³ and —X⁵S(O)₂R¹³ and/or 1 radical selected from —R¹⁴, —X⁵OR¹⁴, —⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹³C(O)R¹⁴, —X⁵NR¹³C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X¹ is a bond or (C₁₋₆)alkylene; R¹² at each occurrence independently is hydrogen, (C₁₋₆)alkyl or halo-substituted(C₁₋₆)alkyl; R¹³ is (C₁₋₆)alkyl or halo-substituted(C₁₋₆)alkyl; and R¹⁴ is (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl;

R¹ is hydrogen or (C₁₋₆)alkyl and R² is selected from a group consisting of hydrogen, cyano, —X⁵NR¹²R¹², —X⁵NR¹²C(O)R¹², —X⁵NR¹²C(O)OR¹², —X⁵R¹², —X⁵NR¹²C(O)NR¹²C(O)NR¹²R¹², —X⁵NR¹²C(NR¹²)NR¹²R¹², —X⁵OR¹², —X^{SR}¹², —X⁵C(O)OR¹², —X⁵OC(O)R¹², —X⁵C(O)NR¹²R¹², —X⁵S(O)₂NR¹²R¹², —X⁵NR¹²S(O)₂R¹², —X⁵P(O)OR¹²)OR¹², —X⁵OP(O)(OR¹²)OR¹², —X⁵NR¹²C(O)R¹³, —X⁵S(O)R¹³, —X⁵S(O)R¹³, —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹², and —X^{NR}¹²C(NR¹²)NR¹⁴R¹², wherein X⁵, R¹², R¹³ and R¹⁴ are as defined above; or R¹ and R² taken together with the carbon atom to which both R¹ and R² are attached form (C₃₋₈)cycloalkylene or (C₃₋₈)heterocycloalkylene; wherein within said R² any heteroaryl, aryl, cycloalkyl, heterocycloalkyl, cycloalkylene or heterocycloalkylene is unsubstituted or substituted with 1 to 3 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵S(O)₂R¹³ and —X⁵C(O)R¹³, wherein X⁵, R¹² and R¹³ are as defined above;

R³ is —C(R⁶)(R⁶)X⁶, wherein R⁶ is hydrogen or (C₁₋₆)alkyl and X⁶ is selected from —X⁵NR¹²R¹², —X⁵NR¹²C(O)R¹², —X⁵NR¹²C(O)OR¹², —X⁵NR¹²OC(NR¹²R¹², 13 X⁵NR¹²C(NR¹²)NR¹²R^{12, —X}⁵OR¹², —X⁵SR¹², —X⁵C(O)OR¹², —X⁵C(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵C(O)R¹³, —X⁵NR¹²C(O)R¹³, —XS(O)R¹³, —X⁵S(O)₂R¹³, —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹² wherein X⁵, R¹², R¹³ and R¹⁴ are as defined above;

R⁴ is selected from —X⁸NR¹²R¹², —X⁸NR¹²C(O)R¹², —X⁸NR¹²C(O)OR¹², —X⁸NR¹²C(O)NR¹²R¹², —X⁸NR¹²C(NR¹²)NR¹²R¹², —X⁸OR¹², —X⁸SR¹², —X⁵C(O)OR¹², —X⁵C(O)R¹², —X⁸OC(O)R¹², —X⁵C(O)NR¹²R¹², —X⁸S(O)₂NR¹²R¹², —X⁸NR¹²S(O)₂R¹², —X⁸P(O)(OR¹²)OR¹², —X⁸OP(O)(OR¹²)OR¹², —X⁵C(O)R¹³, —X⁸NR¹²C(O)R¹³, —X⁸S(O)R¹³, —X⁸S(O)₂R¹³, —R¹⁴, —X⁸OR¹⁴, —X⁸SR¹⁴, —X⁸S(O)R¹⁴, —X⁸S(O)₂R¹⁴, —X⁵C(O)R¹⁴, —X⁵C(O)OR¹⁴, —X⁸OC(O)R¹⁴, —X⁸NR¹⁴R¹², —X⁸NR¹²C(O)R¹⁴, —X⁸NR¹²C(O)OR¹⁴, —X⁵C(O)NR¹⁴R¹², —X⁸S(O)₂NR¹⁴R¹², —X^{NR}¹²S(O)₂R¹⁴, —X⁸NR¹²C(O)NR¹⁴R¹² and —X⁸NR¹²C(NR¹²)NR¹⁴R¹² wherein X⁸ is (C₁₋₆)alkylene and X⁵, R¹², R¹³ and R¹⁴ are as defined above, with the proviso that when X³ is cyano and X² is —OR⁴, where R⁴ is defined as —R¹⁴, then R¹⁴ is (C₃₋₁₀)cycloalkyl(C₁₋₆)alkyl, hetero(C₃₋₁₀)cycloalkyl(C₁₋₃)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl;

R¹⁵ is (C₆₋₁₀)aryl, hetero(C₅₋₁₀)aryl, (C₉₋₁₀)bicycloaryl or hetero(C₈₋₁₀)bicycloaryl;

R¹⁷ is (C₁₋₆)alkyl, (C₃₋₁₀)cycloalkyl(C₁₋₆)alkyl, hetero(C₃₋₁₀)cycloalkyl(C₁₋₆)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl;

R¹⁸ is (C₁₋₆)alkyl, (C₃₋₁₀)cycloalkyl(C₁₋₆)alkyl, hetero(C₃₋₁₀)cycloalkyl(C₁₋₆)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl; and

R¹⁹ and R²⁰ together with the atoms to which R¹⁹ and R²⁰ are attached form (C₄₋₈)heterocycloalkylene, wherein no more than one of the ring member atoms comprising the ring is a heteroatom selected from —NR²¹— or —O—, wherein the ring is unsubstituted or substituted with R¹, wherein R¹ is as defined above, and R²¹ is hydrogen, —C(O)OR¹², —C(O)R¹², —C(O)NR¹²R¹², —S(O)₂NR¹²R¹², —S(O)R¹³ and —S(O)₂R¹³, —S(O)R¹⁴, —S(O)₂R¹⁴, —C(O)R¹⁴, —C(O)OR¹⁴, —C(O)NR¹²R¹² and —S(O)₂NR¹⁴R¹², wherein R¹², R¹³ and R¹⁴ are as defined above;

wherein within R³, R⁴, R¹⁵, R¹⁷ and R¹⁸ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X^{NR}¹³C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹³R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵C(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹²; and within R³ and R⁴ any aliphatic moiety is unsubstituted or substituted further by 1 to 5 radicals independently selected from cyano, halo, nitro, —NR¹²R¹², —NR¹²C(O)R¹², —NR¹²C(O)OR¹², —NR¹²C(O)NR¹²R¹², —NR¹²C(NR¹²)NR¹²R¹², —OR¹², —SR¹², —C(O)OR¹², —C(O)R¹², —OC(O)R¹², —C(O)NR¹²R¹², —S(O)₂NR¹²R¹², —NR¹² S(O)₂R¹², —P(O)(OR¹²)OR¹², —OP(O)(OR¹²)OR¹², —NR¹²C(O)R¹³, —S(O)R¹³ and —S(O)₂R¹³; wherein X⁵, R¹², R¹³ and R¹⁴ are as described above, with the proviso that when X² is —OR⁴, where R⁴ is defined as —R¹⁴, or —NHR¹⁸, then any aromatic ring system present within R¹⁴ or R¹⁸ is not substituted further by halo, (C₃₋₁₀)cycloalkyl, hetero(C₃₋₁₀)cycloalkyl, (C₆₋₁₀)aryl, hetero(C₅₋₁₀)aryl, (C₉₋₁₀)bicycloaryl or hetero(C₈₋₁₀)bicycloaryl; with the proviso that only one bicyclic ring structure is present within R³, R⁴ or R¹⁵; and the N-oxide derivatives, prodrug derivatives, protected derivatives, individual isomers and mixtures of isomers thereof; and the pharmaceutically acceptable salts and solvates of such compounds and the N-oxide derivatives, prodrug derivatives, protected derivatives, individual isomers and mixtures of isomers thereof.

Preferred is a compound of Formula I:

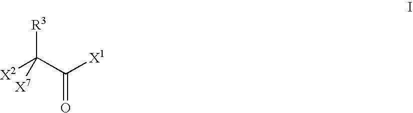

in which:

X¹ is —NHC(R¹)(R²)X³ or —NHCH(R¹⁹)C(O)R²⁰;

X² is —OH, —OC(O)NR¹²R¹² or —OC(O)R¹⁴, wherein R¹² and R¹⁴ are as defined below;

X³ is cyano, —C(R⁷)(R⁸)R¹⁶, —C(R⁶)(OR⁶)₂, —CH₂C(O)R¹⁶, —CH═CHS(O)₂R⁵, —C(O)CF₂C(O)NR⁵R⁵, —C(O)C(O)NR⁵R⁶, —C(O)C(O)OR⁵, —C(O)CH₂OR⁵, —C(O)CH₂N(R⁶)SO₂R⁵ or —C(O)C(O)R⁵; wherein R⁵ is hydrogen, (C₁₋₄)alkyl, (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl; R⁶ is hydrogen, hydroxy or (C₁₋₆)alkyl; or where X³ contains an —NR⁵R⁶ group, R⁵ and R⁶ together with the nitrogen atom to which they are both attached, form hetero(C₃₋₁₀)cycloalkyl, hetero(C₅₋₁₀)aryl or hetero(C₈₋₁₀)bicycloaryl; R⁷ is hydrogen or (C₁₋₄)alkyl and R⁸ is hydroxy or R⁷ and R⁸ together form oxo; R¹⁶ is hydrogen, —X⁴, —CF₃, —CF₂CF₂R⁹ or —N(R⁶)OR⁶; R⁹ is hydrogen, halo, (C₁₋₄)alkyl, (C₅₋₁₀aryl(C₀₋₆)alkyl or (C₅₋₁₀)heteroaryl(C₀₋₆)alkyl;

X⁴ comprises a heteromonocyclic ring containing 4 to 7 ring member atoms or a fused heterobicyclic ring system containing 8 to 14 ring member atoms and any carbocyclic ketone, iminoketone or thioketone derivative thereof;

wherein within R⁵, X³ or X⁴ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹² R², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹² S(O)₂R¹², —X⁵P(O)(OR¹²)OR¹², —X⁵OP(O)(OR¹²)OR¹², —X⁵NR¹²C(O)R¹³, —X⁵S(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and X⁵NR¹²C(NR¹²)NR¹⁴R¹², wherein X⁵ is a bond or (C₁₋₆)alkylene; R¹² at each occurrence independently is hydrogen, (C₁₋₆)alkyl or halo-substituted(C₁₋₆)alkyl; R¹³ is (C₁₋₆)alkyl or halo-substituted(C₁₋₆)alkyl; and R¹⁴ is (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl;

R¹ is hydrogen or (C₁₋₆)alkyl and R² is selected from a group consisting of hydrogen, cyano, —X⁵NR¹²R¹², —X⁵NR¹²C(O)R¹², —X⁵NR¹²C(O)OR¹², —X⁵R¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵S(O)₂R¹³, —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵, R¹², R¹³ and R¹⁴ are as defined above; or R¹ and R² taken together with the carbon atom to which both R¹ and R² are attached form (C₃₋₈)cycloalkylene or (C₃₋₈)heterocycloalkylene; wherein within said R² any heteroaryl, aryl, cycloalkyl, heterocycloalkyl, cycloalkylene or heterocycloalkylene is unsubstituted or substituted with 1 to 3 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(NR¹²)NR¹²R¹², —X⁵OR¹², —X⁵SR¹², —X⁵C(O)OR¹², —X⁵C(O)R¹², —X⁵C(O)R¹², —X⁵C(O)NR¹², R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵S(O)₂R¹³ and —X⁵C(O)R¹³, wherein X⁵, R¹² and R¹³ are as defined above;

R³ is —C(R⁶)(R⁶)X⁶, wherein R⁶ is hydrogen or (C₁₋₆)alkyl and X⁶ is selected from —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵ P(O)(OR¹²)OR¹², —X⁵OP(O)(OR¹²)OR¹², —X⁵C(O)R¹³, —X⁵NR¹²C(O)R¹³, —X⁵S(O)R¹³, —X⁵S(O)₂R¹³, —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹² wherein X⁵, R¹², R¹³ and R¹⁴ are as defined above; and

R¹⁹ and R²⁰ together with the atoms to which R¹⁹ and R²⁰ are attached form (C₄₋₈)heterocycloalkylene, wherein no more than one of the ring member atoms comprising the ring is a heteroatom selected from —NR²¹— or —O—, wherein and the ring is unsubstituted or substituted with R¹, wherein R¹ is as defined above, and R²¹ is hydrogen, —C(O)OR¹², —C(O)R¹², —C(O)NR¹²R¹², —S(O)₂NR¹²R¹², —S(O)R¹³ and —S(O)₂R¹³, —S(O)R¹⁴, —S(O)₂R¹⁴, —C(O)R¹⁴, —C(O)OR¹⁴, —C(O)NR¹²R¹² and —S(O)₂NR¹⁴R¹², wherein R¹², R¹³ and R¹⁴ are as defined above;

wherein within R³, R⁴, R¹⁵, R¹⁷ and R¹⁸ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵C(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹²; and within R³ and R⁴ any aliphatic moiety is unsubstituted or substituted further by 1 to 5 radicals independently selected from cyano, halo, nitro, —NR¹²R¹², —NR¹²C(O)R¹², —NR¹²C(O)OR¹², —NR¹²C(O)NR¹²R¹², —NR¹²C(NR¹²)NR¹²R¹², —OR¹², —SR¹², —C(O)OR¹², —C(O)R¹², —OC(O)R¹², —C(O)NR¹²R¹², —S(O)₂NR¹²R¹², —NR¹²S(O)₂R¹², —P(O)(OR¹²)OR¹², —OP(O)(OR¹²)OR¹², —NR¹²C(O)R¹³, —S(O)R¹³ and —S(O)₂R¹³; wherein X⁵, R¹², R¹³ and R¹⁴ are as described above; with the proviso that only one bicyclic ring structure is present within R³, R⁴ or R¹⁵; and the N-oxide derivatives, prodrug derivatives, protected derivatives, individual isomers and mixtures of isomers thereof, and the pharmaceutically acceptable salts and solvates of such compounds and the N-oxide derivatives, prodrug derivatives, protected derivatives, individual isomers and mixtures of isomers thereof.

Preferred is a compound of Formula I:

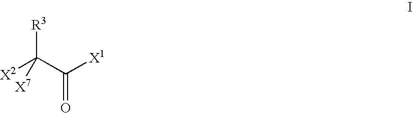

in which:

X¹ is —NHC(R¹)(R²) C(O)C(O)NR⁵R⁶, wherein R⁵ is hydrogen, (C₁₋₄)alkyl, (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl and R⁶ is hydrogen, hydroxy or (C₁₋₆)alkyl or R⁵ and R⁶ together with the nitrogen atom to which they are both attached form hetero(C₃₋₁₀)cycloalkyl, hetero(C₅₋₁₀)aryl or hetero(C₈₋₁₀)bicycloaryl;

X² is hydrogen;

wherein within X¹ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵R¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵ is a bond or (C₁₋₆)alkylene; R¹² at each occurrence independently is hydrogen, (C₁₋₆)alkyl or halo-substituted(C₁₋₆)alkyl; R¹³ is (C₁₋₆)alkyl or halo-substituted(C₁₋₆)alkyl; and R¹⁴ is (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl;

s R¹ is hydrogen and R² is (C₁₋₆)alkyl; and

R³ is —CH₂X⁶, wherein X⁶ is —X⁵NR¹²S(O)₂R¹² or —X⁵S(O)₂R¹⁴ wherein X⁵, R¹² and R¹⁴ are as defined above;

wherein within R³ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹³R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X^{NR}⁵S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵C(O)R¹³ and —X⁵S(O)₂R¹³ and within R³ any aliphatic moiety is unsubstituted or substituted further by 1 to 5 radicals independently selected from cyano, halo, nitro, —NR¹²R¹², —NR¹²C(O)R¹², —NR¹²C(O)OR¹², —NR¹²C(O)NR¹²R¹², —NR¹²C(NR¹²)NR¹²R¹², —OR¹², —SR¹², —C(O)OR¹², —C(O)R¹², —OC(O)R¹², —C(O)NR¹²R¹², —S(O)₂NR¹²R¹², —NR¹²S(O)₂R¹², —P(O)(OR¹²)OR¹², —OP(O)(OR¹²)OR¹², —NR¹²C(O)R¹³, —S(O)R¹³ and —S(O)₂R¹³; wherein X⁵, R¹², R¹⁴ are as described above; with the proviso that only one bicyclic ring structure is present within R³; and the N-oxide derivatives, prodrug derivatives, protected derivatives, individual isomers and mixtures of isomers thereof; and the pharmaceutically acceptable salts and solvates of such compounds and the N-oxide derivatives, prodrug derivatives, protected derivatives, individual isomers and mixtures of isomers thereof.

Preferred are compounds of the invention in which X¹ is —NHC(R¹)(R²)X³ or —NHCH(R¹⁹)C(O)R²⁰, wherein R¹ is hydrogen or (C₁₋₆)alkyl and R² is hydrogen, (C₁₋₆)alkyl, —X⁵OR¹², —X⁵S(O)R¹³, —X⁵OR¹⁴, (C₆₋₁₀)aryl(C₀₋₆)alkyl or hetero(C₅₋₁₀)aryl(C₀₋₆)alkyl or R¹ and R² taken together with the carbon atom to which both R¹ and R² are attached form (C₃₋₆)cycloalkylene or (C₃₋₆)heterocycloalkylene, wherein within said R² any heteroaryl, aryl, cycloalkylene or heterocycloalkylene is unsubstituted or substituted with (C₁₋₆)alkyl or hydroxy, particularly wherein X³ is cyano, —C(O)R¹⁶, —C(R⁶)(OR⁶)₂, —CH═CHS(O)₂R⁵, —CH₂C(O)R¹⁶, —C(O)CF₂C(O)NR⁵R⁵, —C(O)C(O)NR⁵R⁶, —C(O)C(O)OR⁵, —C(O)CH₂OR⁵, —C(O)CH₂N(R⁶)SO₂R⁵ or —C(O)C(O)R⁵, wherein R⁵, R⁶ and R¹⁶ are as described above, and R¹⁹ and R²⁰ together with the atoms to which R¹⁹ and R²⁰ are attached form (C₄₋₈)heterocycloalkylene, wherein no more than one of the ring member atoms comprising the ring is a heteroatom selected from —NR²¹— or —O—, particularly wherein the ring is unsubstituted or substituted with (C₁₋₆)alkyl or —X⁵C(O)OR¹² and R²¹ is hydrogen, (C₁₋₆)alkyl, —X⁵C(O)R¹², —X⁵C(O)OR¹², —R¹⁴, —X⁵C(O)R¹⁴ or —C(O)OR¹⁴.

Particularly preferred are compounds of the invention in which X³ is cyano, —C(O)X⁴, —C(O)H, —C(O)N(CH₃)OCH₃, —CH(OCH₃)₂, —C(O)CF₃, —C(O)CF₂CF₃, —CH₂C(O)R¹⁶, (E)-2-benzenesulfonyl-vinyl, 2-dimethylcarbamoyl-2,2-difluoro-acetyl, 2-oxo-2-pyrrolidin-1-yl-acetyl, 2-morpholin-4-yl-2-oxo-acetyl, 2-oxo-2-piperazin-1-yl-acetyl, 2-(4-methanesulfonyl-piperazin-1-yl)-2-oxo-acetyl, 2-(1,1-dioxo-1□⁶-thiomorpholin-4-yl)-2-oxo-acetyl, dimethylaminooxalyl, tetrahydro-pyran-4-ylaminooxalyl, 2-morpholin-4-yl-ethylaminooxalyl, cyclopentyl-ethyl-aminooxalyl, pyridin-3-ylaminooxalyl, phenylaminooxalyl, 1-benzoyl-piperidin-4-ylaminooxalyl, 1-benzylcarbamoyl-methanoyl, 1-benzyloxy(oxalyl), 2-benzyloxy-acetyl, 2-benzenesulfonylamino-ethanoyl, 2-oxo-2-phenyl-ethanoyl, 3H-oxazole-2-carbonyl, 5-trifluoromethyl-oxazole-2-carbonyl, 3-trifluoromethyl-[1,2,4]oxadiazole-5-carbonyl, 2,2,3,3,3-pentafluoro-propionyl, hydroxyaminooxalyl, oxalyl, 2-(1,3-dihydro-isoindol-2-yl)-2-oxo-acetyl, benzothiazol-2-ylaminooxalyl, 2-oxo-ethyl, 2-oxazol-2-yl-2-oxo-ethyl or 2-benzooxazol-2-yl-2-oxo-ethyl, particularly wherein X⁴ is 1H-benzoimidazol-2-yl, pyrimidin-2-yl, benzooxazol-2-yl, benzothiazol-2-yl, pyridazin-3-yl, 3-phenyl-[1,2,4]oxadiazol-5-yl or 3-ethyl-[1,2,4]oxadiazol-5-yl; and R¹⁹ and R²⁰ together with the atoms to which R¹⁹ and R²⁰ are attached form 1-benzoyl-3-oxo-piperidin-4-yl, 1-benzoyl-3-oxo-azepan-4-yl, 2-methyl-4-oxo-tetrahydro-furan-3-yl, 2-ethyl-4-oxo-tetrahydro-furan-3-yl, 4-oxo-1-(1-phenyl-methanoyl)-pyrrolidin-3-yl or (S)-2-acetoxy-4-oxo-azetidin-3-yl.

Most particularly preferred are compounds of the invention in which X³ is —C(O)X⁴, in particular 1H-benzoimidazol-2-ylcarbonyl, pyrimidin-2-ylcarbonyl, benzooxazol-2-ylcarbonyl, benzothiazol-2-ylcarbonyl, pyridazin-3-ylcarbonyl, 3-phenyl-[1,2,4]oxadiazol-5-ylcarbonyl or 3-ethyl-[1,2,4]oxadiazol-5-ylcarbonyl, or —C(O)C(O)NR⁵R⁶, in particular 2-oxo-2-pyrrolidin-1-yl-acetyl, 2-morpholin-4-yl-2-oxo-acetyl, 2-oxo-2-piperazin-1-yl-acetyl, 2-(4-methanesulfonyl-piperazin-1-yl)-2-oxo-acetyl, 2-(1,1-dioxo-1□⁶-thiomorpholin-4-yl)-2-oxo-acetyl, dimethylaminooxalyl, tetrahydro-pyran-4-ylaminooxalyl, 2-morpholin-4-yl-ethylaminooxalyl, cyclopentyl-ethyl-aminooxalyl, pyridin-3-ylaminooxalyl, phenylaminooxalyl or 1-benzoyl-piperidin-4-ylaminooxalyl.

Preferred are compounds of the invention in which X² is —OH or —OC(O)NR¹²R¹², particularly wherein each R¹² independently represent hydrogen or (C₁₋₆)alkyl, wherein said alkyl is unsubstituted or substituted with hydroxy or methoxy, or X² is —OC(O)NHR¹⁴, wherein R¹⁴ is (C₃₋₁₀)cycloalkyl(C₀₋₆)alkyl or hetero(C₃₋₁₀)cycloalkyl(C₁₋₃)alkyl, or X² is —OC(O)R¹⁴, wherein R¹⁴ is —NR²²R²³ and R²² and R²³ together with the nitrogen atom to which both R²² and R²³ attached form a hetero(C₄₋₆)cycloalkyl ring, which ring may be unsubstituted or substituted with hydroxy, particularly in which X² is selected from —OH, dimethylcarbamoyloxy, morpholin-4-ylcarbonyloxy, piperidin-1-yl-carbonyloxy, pyrrolidin-1-yl-carbonyloxy, pyrimidin-2-ylamino, tetrahydro-pyran-4-ylamino, 1-methyl-piperidin-4-ylamino, N-(2-methoxyethyl)-N-(tetrahydro-pyran-4-yl)amino, isopropylamino and cyclohexylamino, 4-tert-butoxycarbonylpiperazin-1-ylcarbonyloxy, N-benzyl-carbamoyloxy, pyrrolidin-1-yl-carbonyloxy, N,N-dimethyl-carbamoyloxy, piperidin-1-yl-carbonyloxy, 4-methanesulfonyl-piperazin-1-yl-carbonyloxy, 4-ethoxycarbonylpiperazin-1-ylcarbonyloxy, N-cyclohexyl-carbamoyloxy, N-phenyl-carbamoyloxy, N-(5,6,7,8-tetrahydro-naphthalen-1-yl)-carbamoyloxy, N-butyl-N-methyl-carbamoyloxy, N-pyridin-3-yl-carbamoyloxy, N-isopropyl-carbamoyloxy, N-pyridin-4-yl-carbamoyloxy, N-cyanomethyl-N-methyl-carbamoyloxy, N,-bis-(2-methoxy-ethyl)-carbamoyloxy, N-phenethyl-carbamoyloxy, piperazine-carbonyloxy, N-naphthalen-2-yl-carbamoyloxy, 4-benzyl-piperazine-1-carbamoyloxy, 4-(1-furan-2-yl-carbonyl)-piperazine-1-carbamoyloxy, thiomorpholin-4-yl-carbonyloxy, 1,1-dioxo-1λ⁶-thiomorpholin-4-yl)-carbonyloxy, bis-(2-methoxy-ethyl)-carbamoyloxy, morpholin-4-ylcarbonyloxy, 2-methoxyethylcarbamoyloxy, diethylcarbamoyloxy, pyrrolidin-1-ylcarbonyloxy, 2-hydroxyethylcarbamoyloxy, tetrahydro-furan-2-ylmethylcarbamoyloxy, cyclopropylcarbamoyloxy, tert-butylcarbamoyloxy, 3-hydroxy-pyrrolidin-1-yl-carbonyloxy and carbamoyloxy, more particularly morpholin-4-ylcarbonyloxy, 2-methoxyethylcarbamoyloxy, diethylcarbamoyloxy, pyrrolidin-1-ylcarbonyloxy, 2-hydroxyethylcarbamoyloxy, tetrahydro-furan-2-ylmethylcarbamoyloxy, cyclopropylcarbamoyloxy, tert-butylcarbarnoyloxy, 3-hydroxy-pyrrolidin-1-yl-carbonyloxy and carbamoyloxy.

Preferred are compounds of the invention in which X² is —NHR¹⁵, wherein R¹⁵ is (C₆₋₁₀)aryl, hetero(C₅₋₁₀)aryl, (C₉₋₁₀)bicycloaryl or hetero(C₈₋₁₀)bicycloaryl, or —NR¹⁷R¹⁸, wherein R¹⁷ is hetero(C₃₋₁₀)cycloalkyl and R¹⁸ is hydrogen or R¹⁷ and R¹⁸ independently are (C₆₋₁₀)aryl(C₁₋₆)alkyl or hetero(C₅₋₁₀)aryl(C₁₋₆)alkyl, wherein within R¹⁵, R¹⁷ and R¹⁸ any alicyclic or aromatic ring system is unsubstituted or substituted further by 1 to 5 radicals independently selected from (C₁₋₆)alkyl, cyano, halo, nitro, halo-substituted(C₁₋₄)alkyl, —X⁵OR¹², —X⁵C(O)OR¹², —X⁵C(O)R¹³, —X⁵C(O)NR¹²R¹², —X⁵NR¹²S(O)₂R¹² and/or 1 radical selected from —R¹⁴, —X⁵OR¹⁴ and —X⁵C(O)NR¹⁴R¹², in particular in which X² is selected from 5-nitrothiazol-2-ylamino, 2-nitrophenylamino, pyrimidin-2-ylamino, tetrahydro-pyran-4-ylamino, N-(2-methoxyethyl)-N-(tetrahydro-pyran-4-yl)amino, 1-methyl-piperidin-4-ylamino, isopropylamino, di(thien-2-ylmethyl)amino or di(benzyl)amino.

Preferred are compounds of the invention in which X² is —OR⁴ wherein R⁴ is 4-methoxy-phenyl, 4′-hydroxymethyl-phenyl, methoxymethyl, phenyl-methanoyl, 1-(4-phenoxy-phenyl)-methanoyl, 3-biphenyl, 4-biphenyl, 1-biphenyl-4-yl-methanoyl, naphthalen-2-yl-methanoyl, benzo[1,3]dioxol-5-yl-methanoyl, (4-methanesulfonylamino-phenyl)-methanoyl, benzo[b]thien-2-yl-methanoyl, 4′-chloro-4-biphenyl, 4-hydroxy-phenyl-methanoyl, 3-chloro-benzo[b]thien-2-yl-methanoyl, thien-2-yl-methanoyl, thien-3-yl-methanoyl, 3-chloro-thien-2-yl-methanoyl, 5-methyl-thien-2-yl-methanoyl, 4-methoxy-phenyl methanoyl, 4-trifluoromethoxy-phenyl methanoyl, 4-chloro-phenyl-methanoyl, 3-bromo-phenyl, cyclohexylmethyl, 3,4-dimethoxy-phenyl-methanoyl, 3,4-difluorophenyl-methanoyl, 3-fluoro, 4-methoxy-phenyl-methanoyl, 4-fluorophenyl-methanoyl, 4-trifluoromethyl-phenyl-methanoyl, 4-formyl-phenyl-formyl, 3-formyl-phenyl-formyl, 4-methyl-pentanoyl, tetrahydro-pyran-4-ylmethyl 2-morpholin-4-yl-2-oxo-ethyl.

Most particularly preferred are compounds of the invention in which X² is selected from —OH, dimethylcarbamoyloxy, morpholin-4-ylcarbonyloxy, piperidin-1-yl-carbonyloxy, pyrrolidin-1-yl-carbonyloxy, pyrimidin-2-ylamino, tetrahydro-pyran-4-ylamino, 1-methyl-piperidin-4-ylamino, N-(2-methoxyethyl)-N-(tetrahydro-pyran-4-yl)amino, isopropylamino and cyclohexylamino.

Preferred are compounds of the invention in which R¹ is hydrogen or (C₁₋₆)alkyl and R² is hydrogen, —X⁵OR¹², —X⁵R¹², (C₅₋₁₀)heteroaryl(C₀₋₆)alkyl, (C₅₋₁₀)aryl(C₀₋₆)alkyl, (C₅₋₁₀)cycloalkyl(C₀₋₆)alkyl, (C₅₋₁₀)heterocycloalkyl(C₀₋₆)alkyl or (C₁₋₆)alkyl; or R¹ and R² taken together with the carbon atom to which both R¹ and R² are attached form (C₃₋₈)cycloalkylene or (C₃₋₈)heterocycloalkylene; wherein within said R² any heteroaryl, aryl, cycloalkyl, heterocycloalkyl, cycloalkylene or heterocycloalkylene is optionally substituted with 1 to 3 radicals independently selected from (C₁₋₆)alkyl and hydroxy; particularly in which R¹ is hydrogen or methyl and R² is hydrogen, methoxymethyl, (C₁₋₆)alkyl, phenethyl, thien-2-yl or 5-methyl-furan-2-yl or R¹ and R² taken together with the carbon atom to which both R¹ and R² are attached form cyclopropylene, tetrahydro-pyran4-ylene or methyl-piperidin-4-ylene.

Preferred are compounds of the invention in which R³ is —CH₂X⁶; wherein X⁶ is is selected from —X⁵SR¹², —X⁵C(O)NR¹²R¹², —X⁵S(O)₂R¹³, —X⁵C(O)R¹³, —X⁵OR¹², —X⁵SR¹⁴, —X⁵R¹⁴, —X⁵S(O)₂R¹⁴, —X⁵C(O)R¹⁴, —X⁵C(O)NR¹⁴R¹², wherein X⁵, R¹², R¹³ and R¹⁴ are as defined above; particularly wherein R³ is thiophene-2-sulfonyl-methyl, 3-chloro-2-fluoro-phenyl-methane-sulfonyl-methyl, benzene-sulfonyl-methyl, phenyl-methane-sulfonyl-methyl, 2-(I1,1-difluoro-methoxy)-phenyl-methane-sulfonyl-methyl, 2-benzene-sulfonyl-ethyl, 2-(pyridine-2-sulfonyl)-ethyl, 2-(pyridine-4-sulfonyl)-ethyl, 2-phenyl-methanesulfonyl-ethyl, oxy-pyridin-2-yl-methane-sulfonyl-methyl, prop-2-ene-1-sulfonyl-methyl, 4-methoxy-phenyl-methane-sulfonyl-methyl, p-tolyl-methane-sulfonyl-methyl, 4-chloro-phenyl-methane-sulfonyl-methyl, o-tolyl-methane-sulfonyl-methyl, 3,5-dimethyl-phenyl-methane-sulfonyl-methyl, 4-trifluoro-methyl-phenyl-methane-sulfonyl-methyl, 4-trifluoro-methoxy-phenyl-methane-sulfonyl-methyl, 2-bromo-phenyl-methane-sulfonyl-methyl, pyridin-2-yl-methane-sulfonyl-methyl, pyridin-3-yl-methane-sulfonyl-methyl, pyridin-4-yl-methane-sulfonyl-methyl, naphthalen-2-yl-methane-sulfonyl-methyl, 3-methyl-phenyl-methane-sulfonyl-methyl, 3-trifluoro-methyl-phenyl-methane-sulfonyl-methyl, 3-trifluoro-methoxy-phenyl-methane-sulfonyl-methyl, 4-fluoro-2-trifluoromethoxy-phenyl-methane-sulfonylmethyl, 2-fluoro-6-trifluoromethyl-phenylmethanesulfonylmethyl, 3-chloro-phenylmethanesulfonylmethyl, 2-fluoro-phenylmrethanesulfonylmethyl, 2-trifluoro-phenylmethanesulfonylmethyl, 2-cyano-phenylmethanesulfonylmethyl, 4-tert-butyl-phenylmethanesulfonylmethyl, 2-fluoro-3-methyl-phenyl-methane-sulfonyl-methyl, 3-fluoro-phenylmethanesulfonylmethyl, 4-fluoro-phenylmethane-sulfonylmethyl, 2-chloro-phenylmethanesulfonylmethyl, 2,5-difluoro-phenylmethane-sulfonylmethyl, 2,6-difluoro-phenylmethanesulfonylmethyl, 2,5-dichloro-phenyl-methane-sulfonylmethyl, 3,4-dichloro-phenylmethanesulfonylmethyl, 2-(1,1-difluoro-methoxy)-phenyl-methanesulfonylmethyl, 2-cyano-phenyl-methane-sulfonyl-methyl, 3-cyano-phenylmethanesulfonylmethyl, 2-trifluoro-methoxy-phenyl-methane-sulfonylmethyl, 2,3-difluoro-phenylmethanesulfonylmethyl, 2,5-difluoro-phenyl-methanesulfonylmethyl, biphenyl-2-ylmethanesulfonylmethyl, cyclohexylmethyl, 3-fluoro-phenyl-methanesulfonylmethyl, 3,4-difluoro-phenyl-methanesulfonylmethyl, 2,4-difluoro-phenylmethanesulfonylmethyl, 2,4,6-trifluoro-phenylmethanesulfonylmethyl, 2,4,5-trifluoro-phenylmethanesulfonylmethyl, 2,3,4-trifluoro-phenylmethanesulfonylmethyl, 2,3,5-trifluoro-phenyl-methane-sulfonylmethyl, 2,5,6-trifluoro-phenylmethanesulfonylmethyl, 2-chloro-5-trifluoro-methylphenylmethanesulfonylmethyl, 2-methyl-propane-1-sulfonyl, 2-fluoro-3-trifluoro-methylphenylmethanesulfonylmethyl, 2-fluoro-4-trifluoro-methylphenylmethanesulfonylmethyl, 2-fluoro-5-trifluoro-methyl-phenyl-methane-sulfonyl-methyl, 4-fluoro-3-trifluoro-methylphenylmethanesulfonylmethyl, 2-methoxy-phenyl-methanesulfonylmethyl, 3,5-bis-trifluoromethyl-phenylmethanesulfonylmethyl, 4-difluoromethoxy-phenylmethanesulfonylmethyl, 2-difluoro-methoxy-phenyl-methanesulfonylmethyl, 3-difluoromethoxy-phenylmethanesulfonylmethyl, 2,6-dichloro-phenylmethanesulfonylmethyl, biphenyl-4-ylmethanesulfonylmethyl, 3,5-dimethyl-isoxazol-4-ylmethanesulfonylmethyl, 5-chloro-thien-2-yl-methane-sulfonylmethyl, 2-[4-(1,1-difluoro-methoxy)-benzenesulfonyl]-ethyl, 2-[2-(1,1-difluoro-methoxy)-benzenesulfonyl]-ethyl, 2-[3-(1,1-difluoro-methoxy)-benzenesulfonyl]-ethyl, 2-(4-trifluoromethoxy-benzenesulfonyl)-ethyl, 2-(3-trifluoromethoxy-benzenesulfonyl)-ethyl, 2-(2-trifluoro-methoxy-benzene-sulfonyl)-ethyl, (cyanomethyl-methyl-carbamoyl)-methyl, biphenyl-3-ylmethyl, 2-oxo-2-pyrrolidin-1-yl-ethyl, 2-benzenesulfonyl-ethyl, isobutylsulfanylmethyl, 2-phenylsulfanyl-ethyl, cyclohexylmethanesulfonylmethyl, 2-cyclohexyl-ethanesulfonyl, benzyl, naphthalen-2-yl, benzylsulfanylmethyl, 2-trifluoromethyl-benzylsulfanylmethyl, phenylsulfanyl-ethyl, cyclopropyl-methanesulfonylmethyl, 5-bromo-thien-2-ylmethyl, 3-phenyl-propyl, 2,2-difluoro-3-phenyl-propyl, 3,4,5-trimethoxy-phenylmethanesulfonylmethyl, 2,2-difluoro-3-thien-2-yl-propyl, cyclohexylethyl, cyclohexylmethyl, tert-butylmethyl, 1-methylcyclohexylmethyl, 1-methylcyclopentylmethyl, 2,2-difluoro-3-phenylpropyl, 2,2-dimethyl-3-phenylpropyl, 1-benzylcyclopropylmethyl, —X⁵S(O)₂R¹³ and —X⁵S(O)₂R¹⁴, wherein R¹³ is alkyl and R¹⁴ is phenyl which phenyl is unsubstituted or substituted.

Preferred are compounds of the invention in which R³ is cyclohexylethyl, cyclohexylmethyl, tert-butylmethyl, 1-methylcyclohexylmethyl, 1-methylcyclopentylmethyl, 2,2-difluoro-3-phenylpropyl, 2,2-dimethyl-3-phenylpropyl, 1-benzylcyclopropylmethyl, —X⁵S(O)₂R¹³ or —X⁵S(O)₂R¹⁴, wherein R¹³ is alkyl and R¹⁴ is phenyl which phenyl is unsubstituted or substituted.

The following tables are intended to provide guidance to better carry out the present invention. However, they do not limit the scope of the invention. People of ordinary skill may selectively make particular compounds by joining O*, HN* or H* of one of the fragments (A1 to A62) shown in Table 1 to the methine carbon atom (*CH*) of one of the fragments (B 1 to B93) shown in Table 2, and joining the methine carbon atom (*CH* or *CF*) of one of the fragments (B1 to B93) shown in Table 2 to the acyl carbon atom (C*) of one of the fragments (C1 to C91) depicted in Table 3.

TABLE 1

| | |
|---|---|
| A1 | 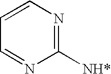 |
| A2 | 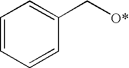 |
| A3 | 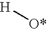 |
| A4 | 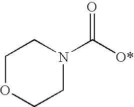 |
| A5 | 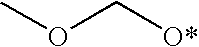 |
| A6 | 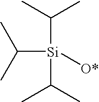 |
| A7 | 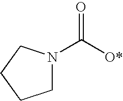 |
| A8 | 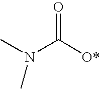 |
| A9 | 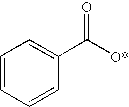 |
| A10 | 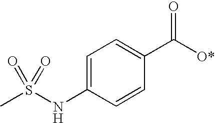 |
| A11 | 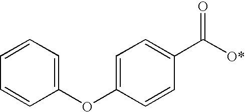 |
| A12 | 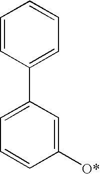 |
| A13 | 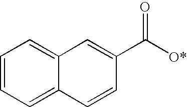 |
| A14 | 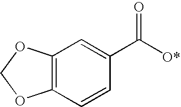 |
| A15 | 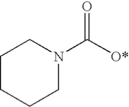 |
| A16 | 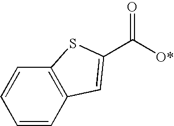 |
| A17 | 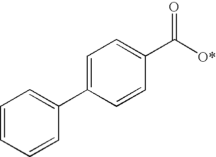 |
| A18 | 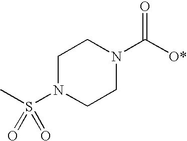 |
| A19 | 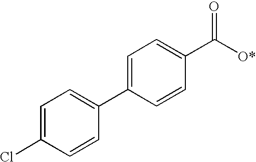 |
| A20 | 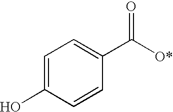 |
| A21 | 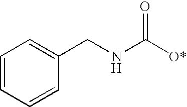 |
| A22 | 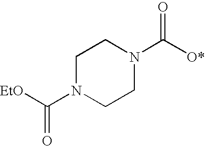 |
| A23 | 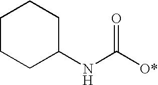 |
| A24 | 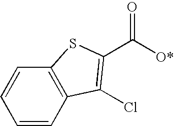 |
| A25 | 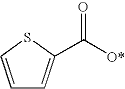 |
| A26 | 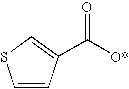 |
| A27 | 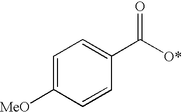 |
| A28 | 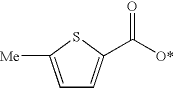 |
| A29 | 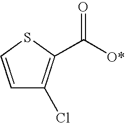 |
| A30 | 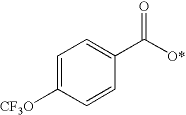 |
| A31 | 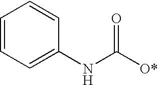 |
| A32 | 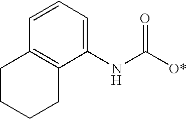 |
| A33 | 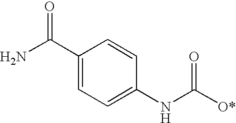 |
| A34 | 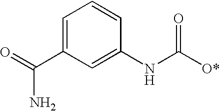 |
| A35 | 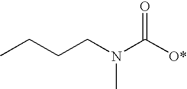 |
| A36 | 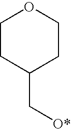 |
| A37 | 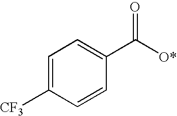 |
| A38 | 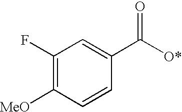 |
| A39 | 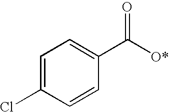 |
| A40 | 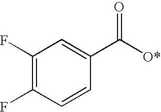 |
| A41 | 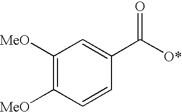 |
| A42 | 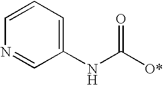 |
| A43 | 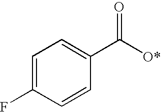 |
| A44 | 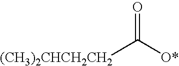 |
| A45 | 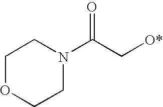 |
| A46 | 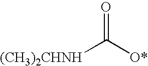 |
| A47 | 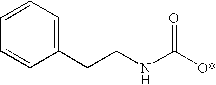 |
| A48 | 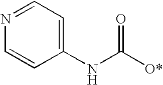 |
| A49 | 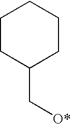 |
| A50 | 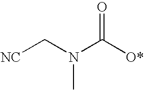 |
| A51 | 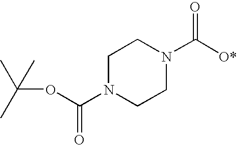 |
| A52 | 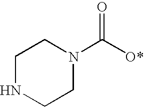 |
| A53 | 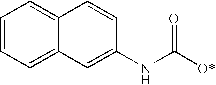 |
| A54 | 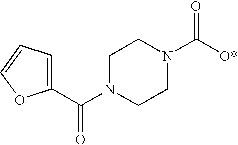 |
| A55 | 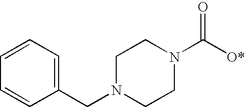 |
| A56 | 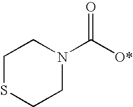 |
| A57 | 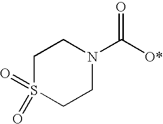 |
| A58 | 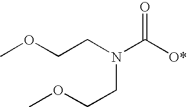 |
| A59 | 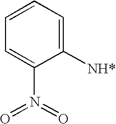 |
| A60 | 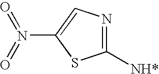 |
| A61 | H* |
| A62 | F* |

TABLE 2

| | |
|---|---|
| B1 | 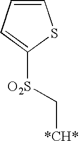 |
| B2 | 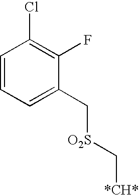 |
| B3 | 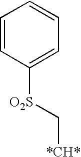 |
| B4 | 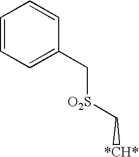 |
| B5 | 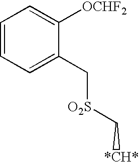 |
| B6 | 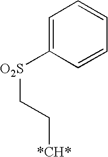 |
| B7 | 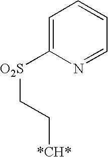 |
| B8 | 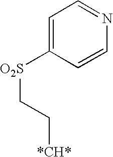 |
| B9 | 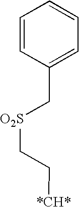 |
| B10 | 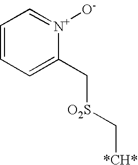 |
| B11 | 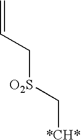 |
| B12 | 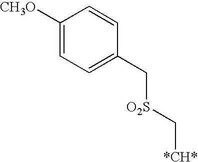 |
| B13 | 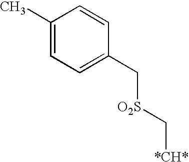 |
| B14 | 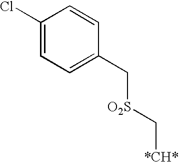 |
| B15 | 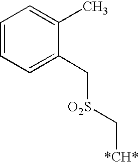 |
| B16 | 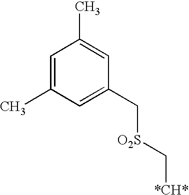 |
| B17 | 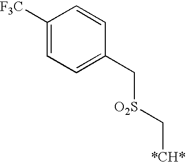 |
| B18 | 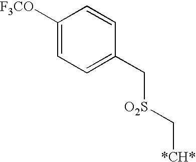 |
| B19 | 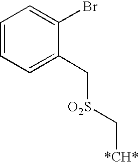 |
| B20 | 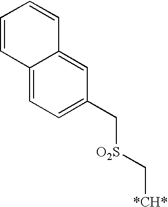 |
| B21 | 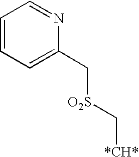 |
| B22 | 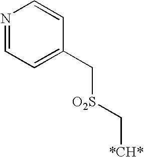 |
| B23 | 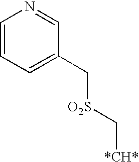 |
| B24 | 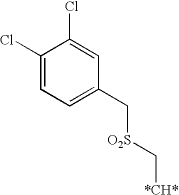 |
| B25 | 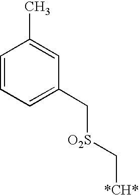 |
| B26 | 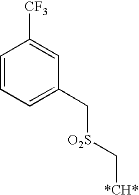 |
| B27 | 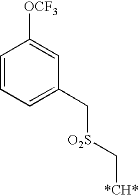 |
| B28 | 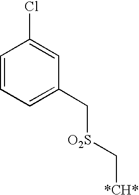 |
| B29 | 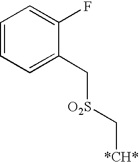 |
| B30 | 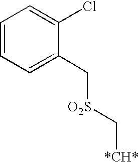 |
| B31 | 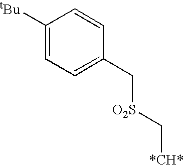 |
| B32 | 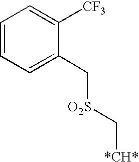 |
| B33 | 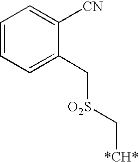 |
| B34 | 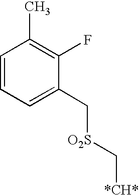 |
| B35 | 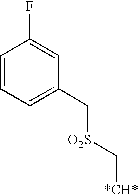 |
| B36 | 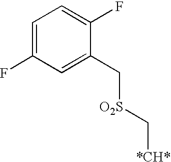 |
| B37 | 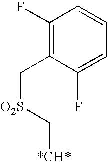 |
| B38 | 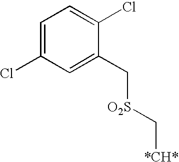 |
| B39 | 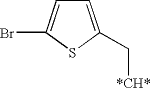 |
| B40 | 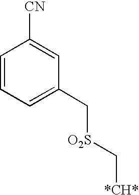 |
| B41 | 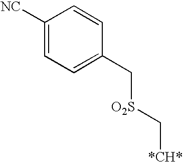 |
| B42 | 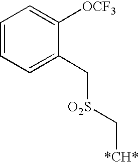 |
| B43 | 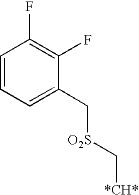 |
| B44 | 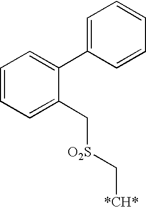 |
| B45 | 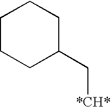 |
| B46 | 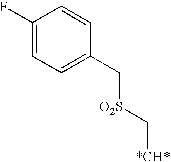 |
| B47 | 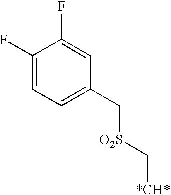 |
| B48 | 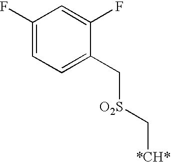 |
| B49 | 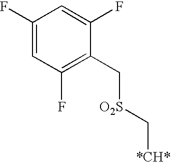 |
| B50 | 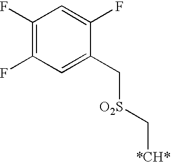 |
| B51 | 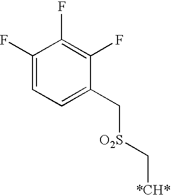 |
| B52 | 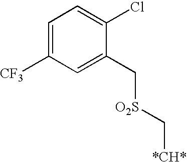 |
| B53 | 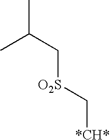 |
| B54 | 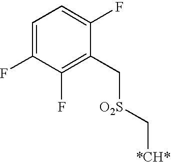 |
| B55 | 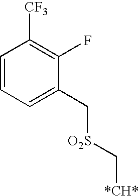 |
| B56 | 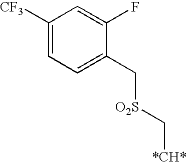 |
| B57 | 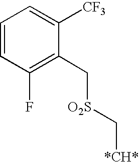 |
| B58 | 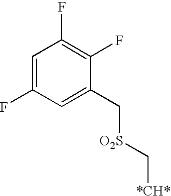 |
| B59 | 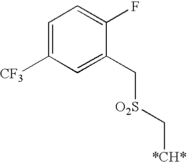 |
| B60 | 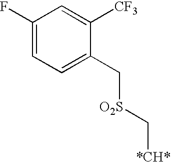 |
| B61 | 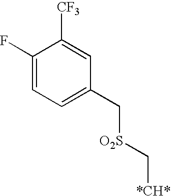 |
| B62 | 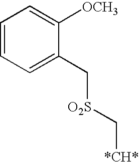 |
| B63 | 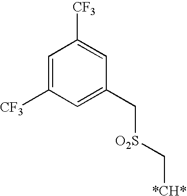 |
| B64 | 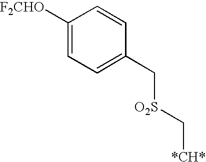 |
| B65 | 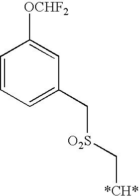 |
| B66 | 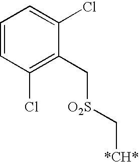 |
| B67 | 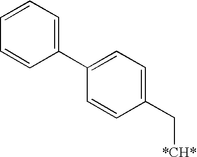 |
| B68 | 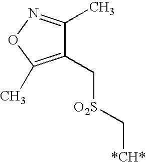 |
| B69 | 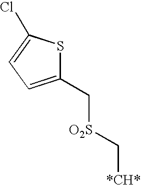 |
| B70 | 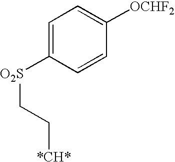 |
| B71 | 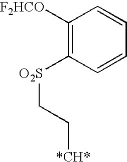 |
| B72 | 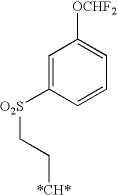 |
| B73 | 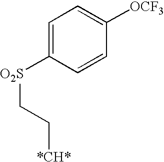 |
| B74 | 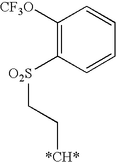 |
| B75 | 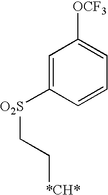 |
| B76 | 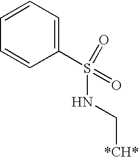 |
| B77 | 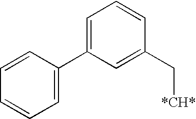 |
| B78 | 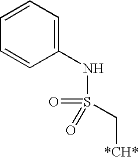 |
| B79 | 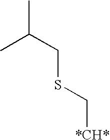 |
| B80 | 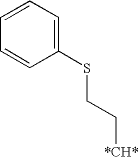 |
| B81 | 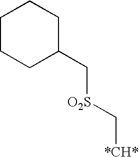 |
| B82 | 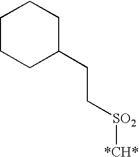 |
| B83 | 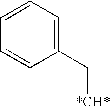 |
| B84 | 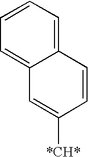 |
| B85 | 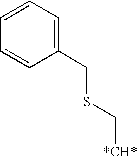 |
| B86 | 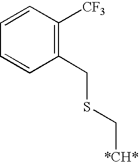 |
| B87 | 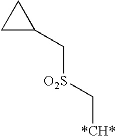 |
| B88 | 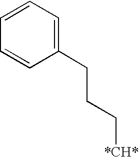 |
| B89 | 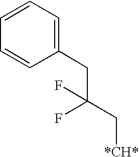 |
| B90 | 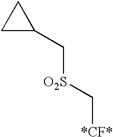 |
| B91 | 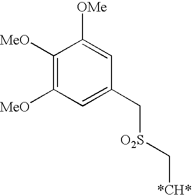 |
| B92 | 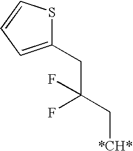 |
| B93 | 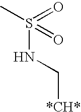 |

TABLE 3

| | |
|---|---|
| C1 | 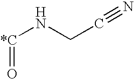 |
| C2 | 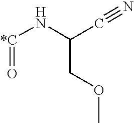 |
| C3 | 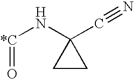 |
| C4 | 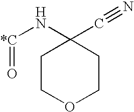 |
| C5 | 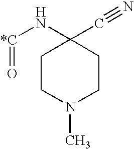 |
| C6 | 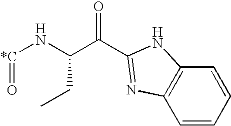 |
| C7 | 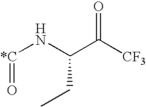 |
| C8 | 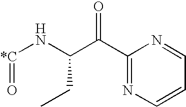 |
| C9 | 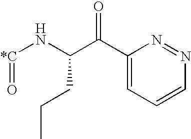 |
| C10 | 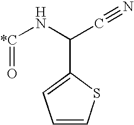 |
| C11 | 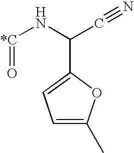 |
| C12 | 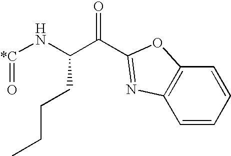 |
| C13 | 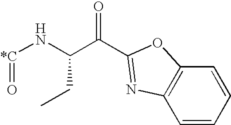 |
| C14 | 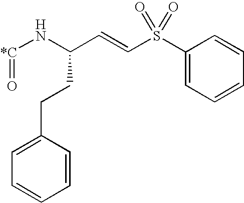 |
| C15 | 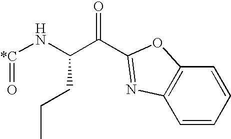 |
| C16 | 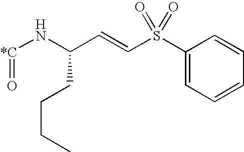 |
| C17 | 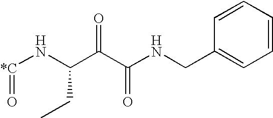 |
| C18 | 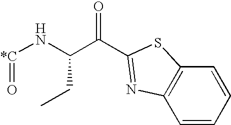 |
| C19 | 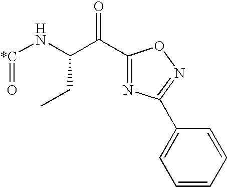 |
| C20 | 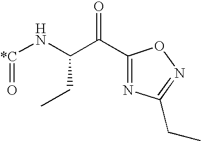 |
| C21 | 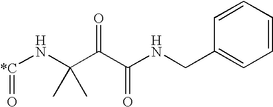 |
| C22 | 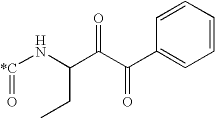 |
| C23 | 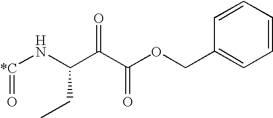 |
| C24 | 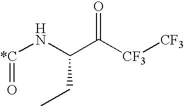 |
| C25 | 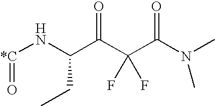 |
| C26 | 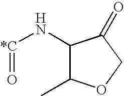 |
| C27 | 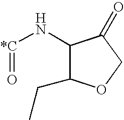 |
| C28 | 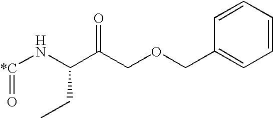 |
| C29 | 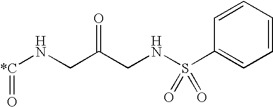 |
| C30 | 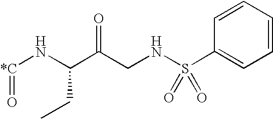 |
| C31 | 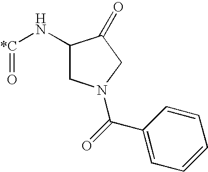 |
| C32 | 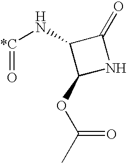 |
| C33 | 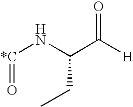 |
| C34 | 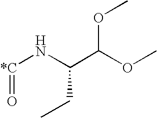 |
| C35 | 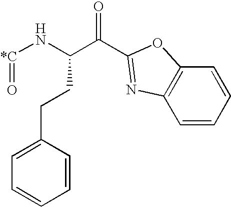 |
| C36 | 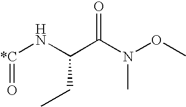 |
| C37 | 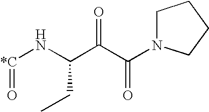 |
| C38 | 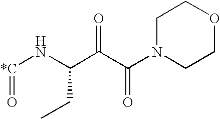 |
| C39 | 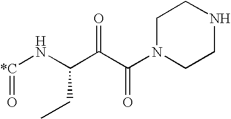 |
| C40 | 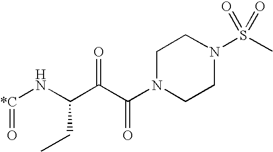 |
| C41 | 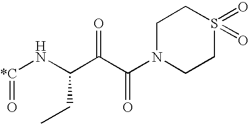 |
| C42 | 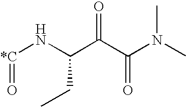 |
| C43 | 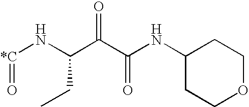 |
| C44 | 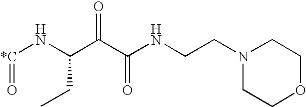 |
| C45 | 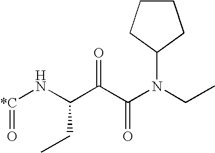 |
| C46 | 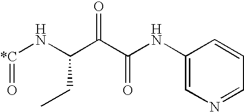 |
| C47 | 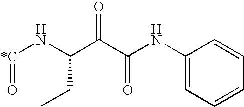 |
| C48 | 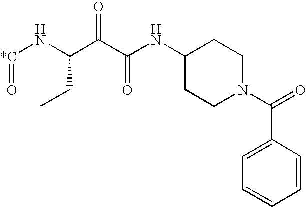 |
| C49 | 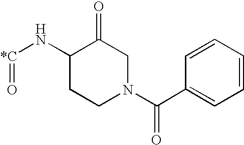 |
| C50 | 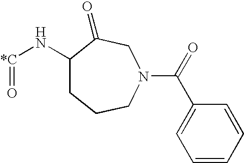 |
| C51 | 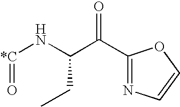 |
| C52 | 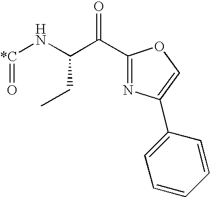 |
| C53 | 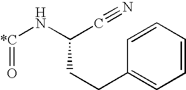 |
| C54 | 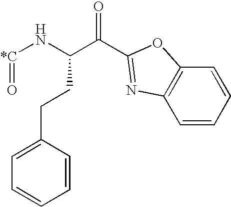 |
| C55 | 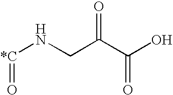 |
| C56 | 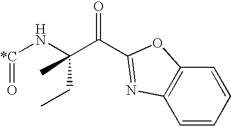 |
| C57 | 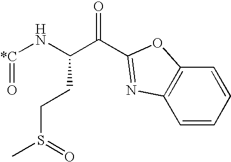 |
| C58 | 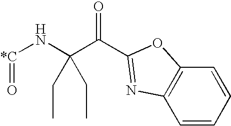 |
| C59 | 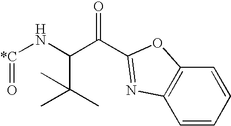 |
| C60 | 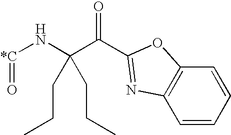 |
| C61 | 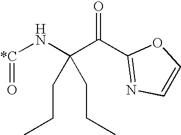 |
| C62 | 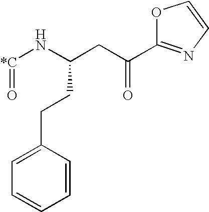 |
| C63 | 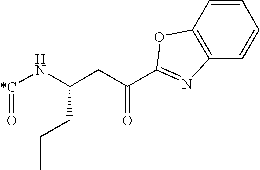 |
| C64 | 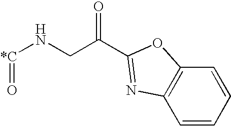 |
| C65 | 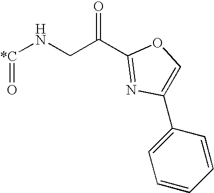 |
| C66 | 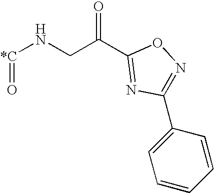 |
| C67 | 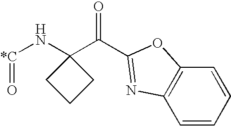 |
| C68 | 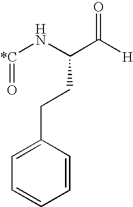 |
| C69 | 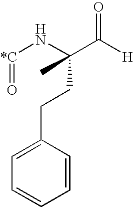 |
| C70 | 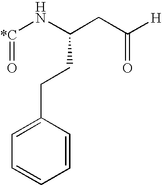 |
| C71 | 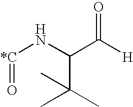 |
| C72 | 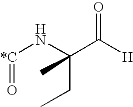 |
| C73 | 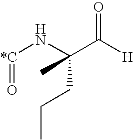 |
| C74 | 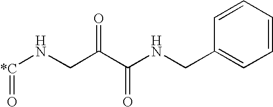 |
| C75 | 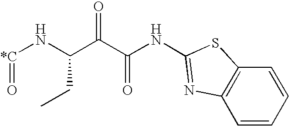 |
| C76 | 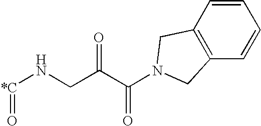 |
| C77 | 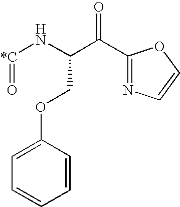 |
| C78 | 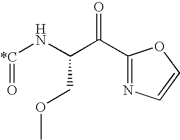 |
| C79 | 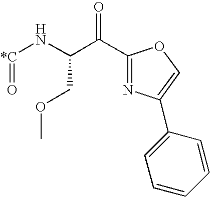 |
| C80 | 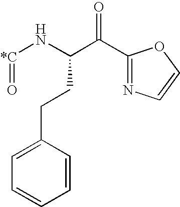 |
| C81 | 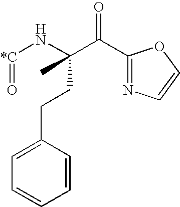 |
| C82 | 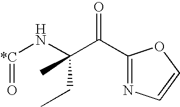 |
| C83 | 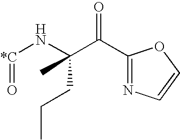 |
| C84 | 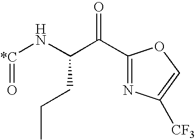 |
| C85 | 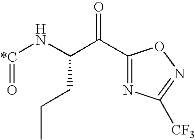 |
| C86 | 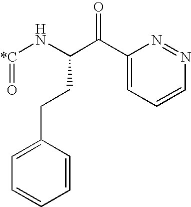 |
| C87 | 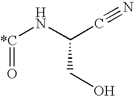 |
| C88 | 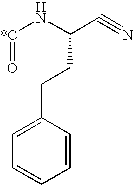 |
| C89 | 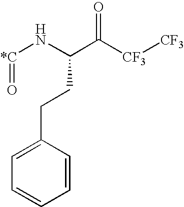 |
| C90 | 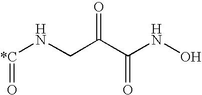 |
| C91 | 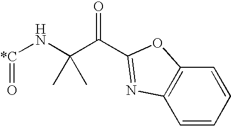 |

For convenience, compounds of the present invention may be referenced to by their “A”, “B”, and “C” fragment combinations. Thus, for example, the compound referenced as A7-B4-C13 is the product of the combination of group A7 in Table 1 and B4 in Table 2 and C13 in Table 3, namely pyrrolidine-1-carboxylic acid (R)-1-[(S)-1-(1-benzooxazol-2-yl-methanoyl)-propylcarbamoyl]-2-phenylmethanesulfonyl-ethyl ester:

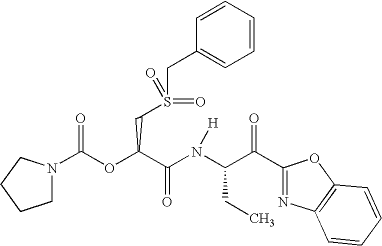

Further preferred compounds of Formula I are provided in the following:

(R)—N-cyanomethyl-2-hydroxy-3-phenylmethanesulfonyl-propionamide;

(R)—N-(1-cyano-1-thiophen-2-yl-methyl)-2-hydroxy-3-phenylmethanesulfonyl-propionamide;

(R)—N-(1-cyano-1-thiophen-2-yl-methyl)-3-[2-(1,1-difluoro-methoxy)-phenylmethanesulfonyl]-2-hydroxy-propionamide;

(R)—N-cyanomethyl-3-[2-(1,1-difluoro-methoxy)-phenylmethanesulfonyl]-2-hydroxy-propionamide;

morpholine-4-carboxylic acid (R)-1-(cyanomethyl-carbamoyl)-2-phenylmethanesulfonyl-ethyl ester;

morpholine-4-carboxylic acid (R)-1-(cyanomethyl-carbamoyl)-2-[2-(1,1-difluoro-methoxy)-phenylmethanesulfonyl]-ethyl ester;

(R)-(2-methoxy-ethyl)-carbamic acid 1-(cyanomethyl-carbamoyl)-2-phenylmethanesulfonyl-ethyl ester;

(S)-diethyl-carbamic acid 1-(cyanomethyl-carbamoyl)-2-cyclohexyl-ethyl ester;

(S)-pyrrolidine-1-carboxylic acid 1-(cyanomethyl-carbamoyl)-2-cyclohexyl-ethyl ester;

(S)-morpholine-4-carboxylic acid 1-(cyanomethyl-carbamoyl)-2-cyclohexyl-ethyl ester;

(S)-4-Ethyl-piperazine-1-carboxylic acid 1-(cyanomethyl-carbamoyl)-2-cyclohexyl-ethyl ester;

(S)-2-hydroxymethyl-pyrrolidine-1-carboxylic acid (S)-1-(cyanomethyl-carbamoyl)-2-cyclohexyl-ethyl ester;

(S)-(2,2,2-Trifluoro-ethyl)-carbamic acid 1-(cyanomethyl-carbamoyl)-2-cyclohexyl-ethyl ester;

(S)-(2-hydroxyethyl)-carbamic acid 1-(cyanomethyl-carbamoyl)-2-cyclohexyl-ethyl ester;

(Tetrahydrofuran-2-ylmethyl)-carbamic acid (S)-1-(cyanomethyl-carbamoyl)-2-cyclohexyl-ethyl ester;

(S)-Azetidine-1-carboxylic acid 1-(cyanomethyl-carbamoyl)-2-cyclohexyl-ethyl ester;

(S)-cyclopropyl-carbamic acid 1-(cyanomethyl-carbamoyl)-2-cyclohexyl-ethyl ester;

(S)-piperidine-1-carboxylic acid 1-(cyanomethyl-carbamoyl)-2-cyclohexyl-ethyl ester;

(S)-(2-methoxy-ethyl)-carbamic acid 1-(cyanomethyl-carbamoyl)-2-cyclohexyl-ethyl ester;

(R)-3-hydroxy-pyrrolidine-1-carboxylic acid (S)-1-(cyanomethyl-carbamoyl)-2-cyclohexyl-ethyl ester;

(S)-3-hydroxy-pyrrolidine-1-carboxylic acid (S)-1-(cyanomethyl-carbamoyl)-2-cyclohexyl-ethyl ester;

(S)-morpholine-4-carboxylic acid 1-(cyanomethyl-carbamoyl)-3-cyclohexyl-propyl ester;

morpholine-4-carboxylic acid (R)-1-[(S)-1-(1-benzooxazol-2-yl-methanoyl)-propylcarbamoyl]-2-phenylmethanesulfonyl-ethyl ester;

morpholine-4-carboxylic acid (R)-1-[(S)-1-(1-benzooxazol-2-yl-methanoyl)-propylcarbamoyl]-2-[2-(1,1-difluoro-methoxy)-phenylmethanesulfonyl]-ethyl ester;

morpholine-4-carboxylic acid (R)-1-[(S)-1-(1-benzothiazol-2-yl-methanoyl)-propylcarbamoyl]-2-[2-(1,1-difluoro-methoxy)-phenylmethanesulfonyl]-ethyl ester;

pyrrolidine-1-carboxylic acid (R)-1-[(S)-1-(1-benzooxazol-2-yl-methanoyl)-propylcarbamoyl]-2-phenylmethanesulfonyl-ethyl ester;

dimethyl-carbamic acid (R)-1-[(S)-1-(1-benzooxazol-2-yl-methanoyl)-propylcarbamoyl]-2-phenylmethanesulfonyl-ethyl ester;

morpholine-4-carboxylic acid (R)-1-[(S)-1-(1-benzylcarbamoyl-methanoyl)-propylcarbamoyl]-2-phenylmethanesulfonyl-ethyl ester;

morpholine-4-carboxylic acid (S)-1-[(S)-1-(oxazolo[4,5-b]pyridine-2-carbonyl)-propylcarbamoyl]-2-phenylmethanesulfonyl-ethyl ester;

morpholine-4-carboxylic acid (S)-1-[(S)-1-(5-ethyl-[1,3,4]oxadiazole-2-carbonyl)-propylcarbamoyl]-2-phenylmethanesulfonyl-ethyl ester;

(S)-2-{(R)-3-[2-(1,1-difluoro-methoxy)-phenylmethanesulfonyl]-2-hydroxy-propanoylamino}-N-methoxy-N-methyl-butyramide;

(R)-3-[2-(1,1-difluoro-methoxy)-phenylmethanesulfonyl]-N—((S)-1-formyl-propyl)-2-hydroxy-propionamide;

(R)—N—[(S)-1-(1-benzooxazol-2-yl-methanoyl)-propyl]-2-hydroxy-3-phenyl-methanesulfonyl-propionamide;

(S)-3-{3-[2-(1,1-difluoro-methoxy)-phenylmethanesulfonyl]-propanoylamino}-2-oxo-pentanoic acid benzylamide;

N—[(S)-1-(1-benzooxazol-2-yl-methanoyl)-propyl]-3-[2-(1,1-difluoro-methoxy)-phenylmethanesulfonyl]-propionamide;

N—[(S)-1-(1-benzooxazol-2-yl-methanoyl)-3-phenyl-propyl]-3-p-tolylmethanesulfonyl-propionamide;

3-(2-difluoromethoxy-phenylmethanesulfonyl)-N-(1-ethyl-2,3-dioxo-3-pyrrolidin-1-yl-propyl)-propionamide;

3-(2-difluoromethoxy-phenylmethanesulfonyl)-N-(1-ethyl-3-morpholin-4-yl-2,3-dioxo-propyl)-propionamide;

3-(2-difluoromethoxy-phenylmethanesulfonyl)-N-(1-ethyl-2,3-dioxo-3-piperazin-1-yl-propyl)-propionamide;

3-(2-difluoromethoxy-phenylmethanesulfonyl)-N-[3-(1,1-dioxo-116-thiomorpholin-4-yl)-1-ethyl-2,3-dioxo-propyl]-propionamide;

3-(2-difluoromethoxy-phenylmethanesulfonyl)-N-[1-ethyl-3-(4-methyl-sulfonyl-piperazin-1-yl)-2,3-dioxo-propyl]-propionamide;

3-[3-(2-difluoromethoxy-phenylmethanesulfonyl)-propionylamino]-2-oxo-pentanoic acid dimethylamide;

3-[3-(2-difluoromethoxy-phenylmethanesulfonyl)-propionylamino]-2-oxo-pentanoic acid cyclopentyl-ethyl-amide;

3-[3-(2-difluoromethoxy-phenylmethanesulfonyl)-propionylamino]-2-oxo-pentanoic acid phenylamide;

3-[3-(2-difluoromethoxy-phenylmethanesulfonyl)-propionylamino]-2-oxo-pentanoic acid pyridin-3-ylamide;

3-[3-(2-difluoromethoxy-phenylmethanesulfonyl)-propionylamino]-2-oxo-pentanoic acid (tetrahydro-pyran-4-yl)-amide;

3-[3-(2-difluoromethoxy-phenylmethanesulfonyl)-propionylamino]-2-oxo-pentanoic acid (1-benzoyl-piperidin-4-yl)-amide;

3-[3-(2-difluoromethoxy-phenylmethanesulfonyl)-propionylamino]-2-oxo-pentanoic acid (2-morpholin-4-yl-ethyl)-amide;

(R)—N—[(S)-1-(1-benzooxazol-2-yl-methanoyl)-propyl]-2-(2-nitro-phenylamino)-3-phenylmethanesulfonyl-propionamide;

N-[1-(benzooxazole-2-carbonyl)-propyl]-3-phenylmethanesulfonyl-2-(pyrimidin-2-ylamino)-propionamide.

(R)—N—[(S)-1-(1-benzooxazol-2-yl-methanoyl)-butyl]-2-(5-nitro-thiazol-2-ylamino)-3-phenylmethanesulfonyl-propionamide;

(2S) (4,4-difluoro-2-hydroxy-5-phenyl-pentanoic acid (1(S)-cyano-3-phenyl-propyl)-amide;

N-(1(S)-cyano-3-phenyl-propyl)-2-(S)-(2-morpholin-4-yl-2-oxo-ethoxy)-4-phenyl-butyramide;

N-(1-(S)-cyano-3-phenyl-propyl)-2-(S)-fluoro-4-phenyl-butyramide;

N-(1-(S)-cyano-3-phenyl-propyl)-2,2-difluoro-4-phenyl-butyramide;

N-(1-(S)-cyano-3-phenyl-propyl)-2-(S)-hydroxy-4-phenyl-butyramide;

N-(1-(S)-cyano-3-phenyl-propyl)-2-(R)-hydroxy-4-phenyl-butyramide;

N-(1-(S)-cyano-3-phenyl-propyl)-2-(R)-methoxy-4-phenyl-butyramide;

2,2-difluoro-5-phenyl-pentanoic acid (1-cyano-cyclopropyl)-amide;

N-(1-(S)-cyano-3-phenyl-propyl)-4-phenyl-butyramide;

2,2-difluoro-5-phenyl-pentanoic acid ((S)-1-cyano-3-phenyl-propyl)-amide;

N-(4-cyano-1-ethyl-piperidin-4-yl)-3-cyclohexyl-propionamide;

N-(4-cyano-1-ethyl-piperidin-4-yl)-3-(2-difluoromethoxy-phenylmethanesulfonyl)-propionamide;

(S)-tert-butyl-carbamic acid 1-(cyanomethyl-carbamoyl)-2-cyclohexyl-ethyl ester;

(R)-carbamic acid 1-(cyanomethyl-carbamoyl)-2-(2-difluoromethoxy-phenylmethanesulfonyl)-ethyl ester;

(S)-carbamic acid 1-(cyanomethyl-carbamoyl)-2-cyclohexyl-ethyl ester;

(R)-morpholine-4-carboxylic acid 1-(1-cyano-cyclopropylcarbamoyl)-2-phenylmethanesulfonyl-ethyl ester;

(R)-morpholine-4-carboxylic acid 1-(4-cyano-tetrahydro-pyran-4-ylcarbamoyl)-2-phenylmethanesulfonyl-ethyl ester;

3-cyclohexyl-2-hydroxy-N-[1-(oxazolo[4,5-b]pyridine-2-carbonyl)-propyl]-propionamide;

(R)—N-[1-(benzothiazole-2-carbonyl)-butyl]-2-isopropylamino-3-phenylmethanesulfonyl-propionamide;

(R)—N-[1-(benzothiazole-2-carbonyl)-butyl]-3-phenylmethanesulfonyl-2-(tetrahydro-pyran-4-ylamino)-propionamide;

(R)—N-[1-(benzothiazole-2-carbonyl)-butyl]-2-dibenzylamino-3-phenylmethanesulfonyl-propionamide;

(R)—N-[1-(benzothiazole-2-carbonyl)-butyl]-2-dimethylamino-3-phenylmethanesulfonyl-propionamide;

(R)—N—[(S)-1-(benzoxazole-2-carbonyl)-butyl]-3-phenylmethanesulfonyl-2-(tetrahydro-pyran-4-ylamino)-propionamide;

(R)—N—[(S)-1-(benzoxazole-2-carbonyl)-butyl]-2-(1-methyl-piperidin-4-ylamino)-3-phenylmethanesulfonyl-propionamide;

(R)—N—[(S)-1-(benzoxazole-2-carbonyl)-butyl]-2-(bis-thiophen-2-ylmethyl-amino)-3-phenylmethanesulfonyl-propionamide;

(R)—N—[(S)-1-(benzoxazole-2-carbonyl)-butyl]-2-dibenzylamino-3-phenylmethanesulfonyl-propionamide;

(S)—N—[(S)-1-(benzoxazole-2-carbonyl)-butyl]-2-(tetrahydro-pyran-4-ylamino)-3-thiophen-2-yl-propionamide;

(S)—N—[(S)-1-(benzoxazole-2-carbonyl)-butyl]-2-isopropylamino-3-thiophen-2-yl-propionamide;

(R)—N-[1-(benzothiazole-2-carbonyl)-butyl]-3-phenylmethanesulfonyl-2-(tetrahydro-pyran-4-ylamino)-propionamide;

(R)—N—[(S)-1-(benzoxazole-2-carbonyl)-butyl]-3-phenylmethanesulfonyl-2-(tetrahydro-pyran-4-ylamino)-propionamide;

(R)—N—[(S)-1-(benzoxazole-2-carbonyl)-butyl]-2-isopropylamino-3-phenylmethanesulfonyl-propionamide;

(R)—N—[(S)-1-(benzoxazole-2-carbonyl)-butyl]-2-[(2-methoxy-ethyl)-(tetrahydro-pyran-4-yl)-amino]-3-phenylmethanesulfonyl-propionamide;

(R)—N—[(S)-1-(benzoxazole-2-carbonyl)-butyl]-2-cyclohexylamino-3-phenylmethanesulfonyl-propionamide;

(R)—N—[(S)-1-(benzoxazole-2-carbonyl)-butyl]-2-dimethylamino-3-phenylmethanesulfonyl-propionamide;

(1S)—N-[1-(benzooxazole-2-carbonyl)-butyl]-2-(S)-fluoro-4-phenyl-butyramide;

2,2-difluoro-5-phenyl-pentanoic acid [(S)-1-(benzoxazole-2-carbonyl)-butyl]-amide;

morpholine-4-carboxylic acid (S)-1-[(S)-1-(benzooxazole-2-carbonyl)-propylcarbamoyl]-2-cyclohexyl-ethyl ester;

morpholine-4-carboxylic acid (S)-2-cyclohexyl-1[(S)-1-(oxazolo[4,5-b]pyridine-2-carbonyl)-propylcarbamoyl]-ethyl ester;

morpholine-4-carboxylic acid (S)-2-cyclohexyl-1-[(S)-1-(5-ethyl-[1,3,4]oxadiazole-2-carbonyl)-propylcarbamoyl]-ethyl ester;

morpholine-4-carboxylic acid (S)-2-cyclohexyl-1-[(S)-1-(5-phenyl-[1,3,4]oxadiazole-2-carbonyl)-propylcarbamoyl]-ethyl ester;

morpholine-4-carboxylic acid (S)-1-[(S)-1-(benzooxazole-2-carbonyl)-propylcarbamoyl]-3-cyclohexyl-propyl ester;

4-[4,4-dimethyl-2-(morpholine-4-carbonyloxy)-pentanoylamino]-3-oxo-azepane-1-carboxylic acid benzyl ester;

(R)—N—[(S)-1-(benzoxazole-2-carbonyl)-butyl]-3-cyclopropylmethanesulfonyl-2-(tetrahydro-pyran-4-ylamino)-propionamide;

(R)—N-[1-(benzoxazole-2-carbonyl)-butyl]-2-cyclohexylamino-3-cyclopropylmethanesulfonyl-propionamide;

(R)—N-[1-(benzoxazole-2-carbonyl)-butyl]-2-cycloheptylamino-3-cyclopropylmethanesulfonyl-propionamide;

(R)-3-phenylmethanesulfonyl-N—[(S)-3-phenyl-1-(thiazole-2-carbonyl)-propyl]-2-(tetrahydro-pyran-4-ylamino)-propionamide;

(R)—N—[(S)-1-(benzoxazole-2-carbonyl)-3-phenyl-propyl]-3-cyclopropylmethanesulfonyl-2-(tetrahydro-pyran-4-ylamino)-propionamide;

(R)-3-cyclopropylmethanesulfonyl-N-[1-(5-ethyl-1,2,4-oxadiazole-3-carbonyl)-propyl]-2-(tetrahydro-pyran-4-ylamino)-propionamide;

(R)-3-phenylmethanesulfonyl-N-[1-(3-phenyl-1,2,4-oxadiazole-5-carbonyl)-propyl]-2-(tetrahydro-pyran-4-ylamino)-propionamide;

(R)—N-[1-(3-cyclopropyl-1,2,4-oxadiazole-5-carbonyl)-propyl]-3-phenylmethanesulfonyl-2-(tetrahydro-pyran-4-ylamino)-propionamide;

{(R)-1-[1-(benzothiazol-2-yl-hydroxy-methyl)-butylcarbamoyl]-2-phenylmethanesulfonyl-ethyl}-carbamic acid tert-butyl ester;

{(R)-1-[(S)-1-(benzoxazol-2-yl-hydroxy-methyl)-butylcarbamoyl]-2-phenylmethanesulfonyl-ethyl}-carbamic acid tert-butyl ester;

{(S)-1-[(S)-1-(benzoxazol-2-yl-hydroxy-methyl)-butylcarbamoyl]-2-thiophen-2-yl-ethyl}-carbamic acid tert-butyl ester;

{(R)-1-[1-(benzothiazol-2-yl-hydroxy-methyl)-butylcarbamoyl]-2-phenylmethanesulfonyl-ethyl}-carbamic acid tert-butyl ester;

{(R)-1-[(S)-1-(benzoxazol-2-yl-hydroxy-methyl)-butylcarbamoyl]-2-phenylmethanesulfonyl-ethyl}-carbamic acid tert-butyl ester;

{(R)-1-[(S)-1-(benzoxazol-2-yl-hydroxy-methyl)-butylcarbamoyl]-2-cyclopropylmethanesulfonyl-ethyl}-carbamic acid tert-butyl ester;

(R)-1-{1-[hydroxy-(3-phenyl-1,2,4-oxadiazol-5-yl)-methyl]-propylcarbamoyl}-2-phenylmethanesulfonyl-ethyl)-carbamic acid tert-butyl ester;

((R)-2-cyclopropylmethanesulfonyl-1-{(S)-1-[(5-ethyl-1,2,4-oxadiazol-3-yl)-hydroxy-methyl]-propylcarbamoyl}-ethyl)-carbamic acid tert-butyl ester;

{(R)-1-[1-(benzoxazol-2-yl-hydroxy-methyl)-butylcarbamoyl]-2-phenylmethanesulfonyl-ethyl}-carbamic acid tert-butyl ester;

{(R)-1-[(S)-1-(benzoxazol-2-yl-hydroxy-methyl)-3-phenyl-propylcarbamoyl]-2-cyclopropylmethanesulfonyl-ethyl}-carbamic acid tert-butyl ester;

{(R)-1-[(S)-1-(hydroxy-thiazol-2-yl-methyl)-3-phenyl-propylcarbamoyl]-2-phenylmethanesulfonyl-ethyl}-carbamic acid tert-butyl ester;

{(R)-1-[(S)-1-(benzoxazol-2-yl-hydroxy-methyl)-butylcarbamoyl]-2-cyclopropylmethanesulfonyl-ethyl}-carbamic acid tert-butyl ester;

(R)-1-{1-[hydroxy-(3-phenyl-1,2,4-oxadiazol-5-yl)-methyl]-propylcarbamoyl}-2-phenylmethanesulfonyl-ethyl}-carbamic acid tert-butyl ester;

((R)-2-cyclopropylmethanesulfonyl-1-{(S)-1-[(5-ethyl-1,2,4-oxadiazol-3-yl)-hydroxy-methyl]-propylcarbamoyl}-ethyl)-carbamic acid tert-butyl ester;

{(R)-1-[1-(benzoxazol-2-yl-hydroxy-methyl)-butylcarbamoyl]-2-phenylmethanesulfonyl-ethyl}-carbamic acid tert-butyl ester;

{(R)-1-[(S)-1-(benzoxazol-2-yl-hydroxy-methyl)-3-phenyl-propylcarbamoyl]-2-cyclopropylmethanesulfonyl-ethyl}-carbamic acid tert-butyl ester;

{(R)-1-[(S)-1-(hydroxy-thiazol-2-yl-methyl)-3-phenyl-propylcarbamoyl]-2-phenylmethanesulfonyl-ethyl}-carbamic acid tert-butyl ester;

(R)-2-phenylmethanesulfonyl-1-{(S)-1-[(3-cyclopropyl-1,2,4-oxadiazol-5-yl)-hydroxy-methyl]-propylcarbamoyl}-ethyl)-carbamic acid tert-butyl ester;

(R)—N-[1-(Benzoxazole-2-carbonyl)-butyl]-2-[cyclopropylmethyl-(tetrahydro-pyran-4-ylmethyl)-amino]-3-phenylmethanesulfonyl-propionamide;

(R)—N-[1-(benzothiazol-2-yl-hydroxy-methyl)-butyl]-2-dibenzylamino-3-phenylmethanesulfonyl-propionamide;

(R)—N-[1-(benzothiazol-2-yl-hydroxy-methyl)-butyl]-3-phenylmethanesulfonyl-2-(tetrahydro-pyran-4-ylamino)-propionamide;

(R)—N-[1-(benzothiazol-2-yl-hydroxy-methyl)-butyl]-2-isopropylamino-3-phenylmethanesulfonyl-propionamide;

(R)—N-[1-(benzothiazol-2-yl-hydroxy-methyl)-butyl]-2-dimethylamino-3-phenylmethanesulfonyl-propionamide;

(R)—N—[(S)-1-(benzoxazol-2-yl-hydroxy-methyl)-butyl]-3-phenylmethanesulfonyl-2-(tetrahydro-pyran-4-ylamino)-propionamide;

(R)—N—[(S)-1-(benzoxazol-2-yl-hydroxy-methyl)-butyl]-2-(1-methyl-piperidin-4-ylamino)-3-phenylmethanesulfonyl-propionamide;

(R)—N—[(S)-1-(benzoxazol-2-yl-hydroxy-methyl)-butyl]-2-(bis-thiophen-2-ylmethyl-amino)-3-phenylmethanesulfonyl-propionamide;

(R)—N—[(S)-1-(benzoxazol-2-yl-hydroxy-methyl)-butyl]-2-dibenzylamino-3-phenylmethanesulfonyl-propionamide;

(S)—N—[(S)-1-(benzoxazol-2-yl-hydroxy-methyl)-butyl]-2-(tetrahydro-pyran-4-ylamino)-3-thiophen-2-yl-propionamide;

(S)—N—[(S)-1-(benzoxazol-2-yl-hydroxy-methyl)-butyl]-2-isopropylamino-3-thiophen-2-yl-propionamide;

(R)—N—[(S)-1-(benzoxazol-2-yl-hydroxy-methyl)-butyl]-2-isopropylamino-3-phenylmethanesulfonyl-propionamide;

(R)—N-[1-(benzothiazol-2-yl-hydroxy-methyl)-butyl]-3-phenylmethanesulfonyl-2-(tetrahydro-pyran-4-ylamino)-propionamide;

R)—N—[(S)-1-(benzoxazol-2-yl-hydroxy-methyl)-butyl]-3-phenylmethanesulfonyl-2-(tetrahydro-pyran-4-ylamino)-propionamide;

(R)—N—[(S)-1-(benzoxazol-2-yl-hydroxy-methyl)-butyl]-3-phenylmethanesulfonyl-2-(tetrahydro-pyran-4-ylamino)-propionamide;

(R)—N—[(S)-1-(benzoxazol-2-yl-hydroxy-methyl)-butyl]-2-[(2-methoxy-ethyl)-(tetrahydro-pyran-4-yl)-amino]-3-phenylmethanesulfonyl-propionamide;

(R)—N—[(S)-1-(benzoxazol-2-yl-hydroxy-methyl)-butyl]-2-cyclohexylamino-3-phenylmethanesulfonyl-propionamide;

(R)—N—[(S)-1-(benzoxazol-2-yl-hydroxy-methyl)-butyl]-2-dimethylamino-3-phenylmethanesulfonyl-propionamide;

N-cyanomethyl-3-cyclohexyl-propionamide;

N-cyanomethyl-3-(2-difluoromethoxy-phenylmethanesulfonyl)-propionamide;

3-(3-cyclohexyl-propionylamino)-2-oxo-5-phenyl-pentanoic acid thiazol-2-ylamide;

3-cyclohexyl-N-(1-formyl-3-phenyl-propyl)-propionamide;

3-(2-difluoromethoxy-phenylmethanesulfonyl)-N—[(S)-1-(5-ethyl-[1,3,4]oxadiazole-2-carbonyl)-propyl]-propionamide;

N—[(S)-1-(benzooxazole-2-carbonyl)-propyl]-2-(2-cyano-phenylamino)-3-cyclohexyl-propionamide;

N-Cyanomethyl-3-cyclohexyl-2-(4-methoxy-phenoxy)-propionamide;

2-benzyloxy-N-cyanomethyl-3-cyclohexyl-propionamide;

(R)—N—[(S)-1-(1-benzooxazol-2-yl-methanoyl)-butyl]-2-benzyloxy-3-phenylmethanesulfonyl-propionamide;

(R)—N—[(S)-1-(1-benzooxazol-2-yl-methanoyl)-propyl]-2-methoxymethoxy-3-phenylmethanesulfonyl-propionamide;

(S)—N—[(S)-1-(1-benzooxazol-2-yl-methanoyl)-butyl]-2-hydroxy-3-phenyl-propionamide;

(R)—N—[(S)-1-(1-benzooxazol-2-yl-methanoyl)-propyl]-3-phenylmethanesulfonyl-2-triisopropylsilanyloxy-propionamide;

(R)—N—[(S)-1-(1-benzothiazol-2-yl-methanoyl)-propyl]-2-hydroxy-3-phenylmethanesulfonyl-propionamide;

(R)-2-hydroxy-3-phenylmethanesulfonyl-N—[(S)-1-(1-pyridazin-3-yl-methanoyl)-butyl]-propionamide;

(S)-3-((R)-2-hydroxy-3-phenylmethanesulfonyl-propanoylamino)-2-oxo-pentanoic acid benzylamide;

(R)—N—[(S)-1-(1-benzooxazol-2-yl-methanoyl)-propyl]-3-[2-(1,1-difluoro-methoxy)-phenylmethanesulfonyl]-2-hydroxy-propionamide;

(R)—N—[(S)-1-(1-benzothiazol-2-yl-methanoyl)-propyl]-3-[2-(1,1-difluoro-methoxy)-phenylmethanesulfonyl]-2-hydroxy-propionamide;

(2R,5S)-2-[2-(1,1-difluoro-methoxy)-phenylmethanesulfonylmethyl]-6-ethoxy-5-ethyl-morpholin-3-one;and their corresponding N-oxides, and their prodrugs, and their protected derivatives, individual isomers and mixtures of isomers thereof; and the pharmaceutically acceptable salts and solvates (e.g. hydrates) of such compounds and their N-oxides and their prodrugs, and their protected derivatives, individual isomers and mixtures of isomers thereof.

Pharmacology and Utility:

The compounds of the invention are selective inhibitors of cathepsin S and, as such, are useful for treating diseases in which cathepsin S activity contributes to the pathology and/or symptomatology of the disease. For example, the compounds of the invention may be useful in treating autoimmune disorders, including, but not limited to, juvenile onset diabetes, multiple sclerosis, pemphigus vulgaris, Graves' disease, myasthenia gravis, systemic lupus erythemotasus, rheumatoid arthritis and Hashimoto's thyroiditis, allergic disorders, including, but not limited to, asthma, and allogeneic immune responses, including, but not limited to, organ transplants or tissue grafts.

Cathepsin S also is implicated in disorders involving excessive elastolysis, such as chronic obstructive pulmonary disease (e.g., emphysema), bronchiolitis, excessive airway elastolysis in asthma and bronchitis, pneumonities and cardiovascular disease such as plaque rupture and atheroma. Cathepsin S is implicated in fibril formation and, therefore, inhibitors of cathepsins S are of use in treatment of systemic amyloidosis.

The cysteine protease inhibitory activities of the compounds of the invention can be determined by methods known to those of ordinary skill in the art. Suitable in vitro assays for measuring protease activity and the inhibition thereof by test compounds are known. Typically, the assay measures protease induced hydrolysis of a peptide based substrate. Details of assays for measuring protease inhibitory activity are set forth in ENZYME ASSAY EXAMPLES, infra.

Administration and Pharmaceutical Compositions:

In general, compounds of Formula I will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. For example, therapeutically effective amounts of a compound of Formula I may range from about 1 microgram per kilogram body weight (□g/kg) per day to about 60 milligram per kilogram body weight (mg/kg) per day, typically from about 1 □g/kg/day to about 20 mg/kg/day. Therefore, a therapeutically effective amount for a 80 kg human patient may range from about 80 □g/day to about 4.8 g/day, typically from about 80 □g/day to about 1.6 g/day. In general, one of ordinary skill in the art, acting in reliance upon personal knowledge and the disclosure of this Application, will be able to ascertain a therapeutically effective amount of a compound of Formula I for treating a given disease.

The compounds of Formula I can be administered as pharmaceutical compositions by one of the following routes: oral, systemic (e.g., transdermal, intranasal or by suppository) or parenteral (e.g., intramuscular, intravenous or subcutaneous). Compositions can take the form of tablets, pills, capsules, semisolids, powders, sustained release formulations, solutions, suspensions, elixirs, aerosols, or any other appropriate composition and are comprised of, in general, a compound of Formula I in combination with at least one pharmaceutically acceptable excipient. Acceptable excipients are non-toxic, aid administration, and do not adversely affect the therapeutic benefit of the active ingredient. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from water, ethanol, glycerol, propylene glycol and various oils, including those of petroleum, animal, vegetable or synthetic origin (e.g., peanut oil, soybean oil, mineral oil, sesame oil, and the like). Preferred liquid carriers, particularly for injectable solutions, include water, saline, aqueous dextrose and glycols.

The amount of a compound of Formula I in the composition may vary widely depending upon the type of formulation, size of a unit dosage, kind of excipients and other factors known to those of skill in the art of pharmaceutical sciences. In general, a composition of a compound of Formula I for treating a given disease will comprise from 0.01%w to 10%w, preferably 0.3%w to 1%w, of active ingredient with the remainder being the excipient or excipients. Preferably the pharmaceutical composition is administered in a single unit dosage form for continuous treatment or in a single unit dosage form ad libitum when relief of symptoms is specifically required. Representative pharmaceutical formulations containing a compound of Formula I are described in Example 15, infra.

Chemistry:

Processes for Making Compounds of Formula I:

Compounds of the invention may be prepared by the application or adaptation of known methods, by which is meant methods used heretofore or described in the literature, for example those described by R. C. Larock in Comprehensive Organic Transformations, VCH publishers, 1989.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1991.

Compounds of Formula I, where X¹ is —NHC(R¹)(R²)X³, can be prepared by proceeding as in the following Reaction Scheme 1:

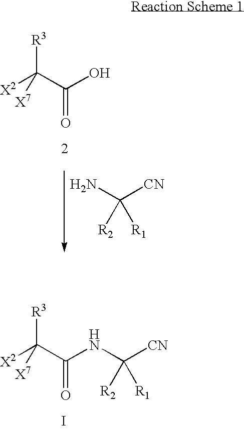

in which each X², X³, X⁷, R¹, R² and R³ are as defined for Formula I in the Summary of the Invention.

Compounds of Formula I can be prepared by condensing an acid of Formula II with an amino compound of formula NH₂CR¹R²X³. The condensation reaction can be effected with an appropriate coupling agent (e.g., benzotriazol-1-yloxytrispyrrolidinophosphonium hexafluorophosphate (PyBOP®), tetra-methyluroniumhexafluorophosphate (HATU), 1-(3-dimethylaminopropyl)-3-ethylcarbodiimide hydrochloride (EDCI), O-benzotriazol-1-yl-N,N,N′,N′-tetramethyluronium hexafluorophosphate (HBTU), 1,3-dicyclohexylcarbodiimide (DCC), N-cyclohexylcarbodiimide, N′-methylpolystyrene, or the like) and optionally an appropriate catalyst (e.g., 1-hydroxybenzotriazole (HOBt), 1-hydroxy-7-azabenzotriazole (HOAt), O-(7-azabenzotrizol-1-yl)-1,1,3,3, or the like) and non-nucleophilic base (e.g., triethylamine, N-methylmorpholine, and the like, or any suitable combination thereof) at ambient temperature and requires 5 to 10 hours to complete.

An oxidation step, if required, can be carried out with an oxidizing agent (e.g., Oxone®, metachloroperbenzoic acid or the like) in a suitable solvent (e.g., methanol, water, or the like, or any suitable combination thereof) at ambient temperature and requires 16 to 24 hours to complete. Detailed descriptions for the synthesis of a compound of Formula I by the processes in Reaction Scheme 1 are set forth in the Examples 1 to 10, infra.

Compounds of Formula I, where X¹ is —NHX⁴, can be prepared by proceeding as in the following Reaction Scheme 2:

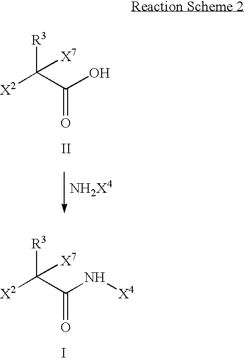

in which each X², X⁴, X⁷ and R³ are as defined for Formula I in the Summary of the Invention.

Compounds of Formula I can be prepared by condensing an acid of Formula II with an amino compound of formula NH₂X⁴. The condensation reaction can be effected with an appropriate coupling agent (e.g., benzotriazol-1-yloxytrispyrrolidinophosphonium hexafluorophosphate (PyBOP®), O-(7-azabenzotrizol-1-yl)-1,1,3,3,tetra-methyluroniumhexafluorophosphate (HATU), 1-(3-dimethylaminopropyl)-3-ethylcarbodiimide hydrochloride (EDCI), O-benzotriazol-1-yl-N,N,N′,N′-tetramethyluronium hexafluorophosphate (HBTU), 1,3-dicyclohexylcarbodiimide (DCC), N-cyclohexylcarbodiimide, N′-methylpolystyrene, or the like) and optionally an appropriate catalyst (e.g., 1-hydroxybenzotriazole (HOBt), 1-hydroxy-7-azabenzotriazole (HOAt), or the like) and non-nucleophilic base (e.g., triethylamine, N-methylmorpholine, and the like, or any suitable combination thereof) at ambient temperature and requires 5 to 10 hours to complete.

An oxidation step, if required, can be carried out with an oxidizing agent (e.g., Oxone®, metachloroperbenzoic acid or the like) in a suitable solvent (e.g., methanol, water, or the like, or any suitable combination thereof) at ambient temperature and requires 16 to 24 hours to complete.

Compounds of Formula I in which X² is —OR⁴, can be prepared by reacting a compound of Formula 3 with a compound of Formula R⁴L according to the following reaction scheme:

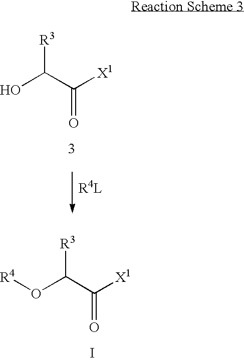

in which L is a leaving group and X¹, R³ and R⁴ are as defined in the Summary of the Invention. A detailed description for the synthesis of a compound of Formula I by the process described above is set forth in Example 4, infra.

Compounds of Formula I, in which X² is —NHR¹⁵, can be prepared by reacting a compound of Formula 4 with a compound of Formula R¹⁵L according to the following reaction scheme:

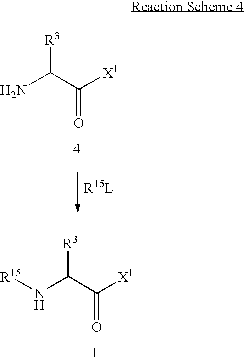

in which L is a leaving group and X¹, R³ and R¹⁵ are as defined in the Summary of the Invention. A detailed description for the synthesis of a compound of Formula I by the process described above is set forth in [update], infra.

Additional Processes for Preparing Compounds of Formula I:

A compound of Formula I can be prepared as a pharmaceutically acceptable acid addition salt by reacting the free base form of the compound with a pharmaceutically acceptable inorganic or organic acid. Alternatively, a pharmaceutically acceptable base addition salt of a compound of Formula I can be prepared by reacting the free acid form of the compound with a pharmaceutically acceptable inorganic or organic base. Inorganic and organic acids and bases suitable for the preparation of the pharmaceutically acceptable salts of compounds of Formula I are set forth in the definitions section of this Application. Alternatively, the salt forms of the compounds of Formula I can be prepared using salts of the starting materials or intermediates.

The free acid or free base forms of the compounds of Formula I can be prepared from the corresponding base addition salt or acid addition salt form. For example, a compound of Formula I in an acid addition salt form can be converted to the corresponding free base by treating with a suitable base (e.g., ammonium hydroxide solution, sodium hydroxide, and the like). A compound of Formula I in a base addition salt form can be converted to the corresponding free acid by treating with a suitable acid (e.g., hydrochloric acid, etc).

The N-oxides of compounds of Formula I can be prepared by methods known to those of ordinary skill in the art. For example, N-oxides can be prepared by treating an unoxidized form of the compound of Formula I with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds of Formula I can be prepared from the N-oxide of an appropriate starting material.

Compounds of Formula I in unoxidized form can be prepared from N-oxides of compounds of Formula I by treating with a reducing agent (e.g., sulfur, sulfur dioxide, triphenyl phosphine, lithium borohydride, sodium borohydride, phosphorus trichloride, tribromide, or the like) in an suitable inert organic solvent (e.g., acetonitrile, ethanol, aqueous dioxane, or the like) at 0 to 80° C.

Prodrug derivatives of the compounds of Formula I can be prepared by methods known to those of ordinary skill in the art (e.g., for further details see Saulnier et al.(1994), Bioorganic and Medicinal Chemistry Letters, Vol. 4, p. 1985). For example, appropriate prodrugs can be prepared by reacting a non-derivatized compound of Formula I with a suitable carbamylating agent (e.g., 1,1-acyloxyalkylcarbonochloridate, para-nitrophenyl carbonate, or the like).

Protected derivatives of the compounds of Formula I can be made by means known to those of ordinary skill in the art. A detailed description of the techniques applicable to the creation of protecting groups and their removal can be found in T. W. Greene, Protecting Groups in Organic Synthesis, 3^{rd} edition, John Wiley & Sons, Inc. 1999. Compounds of the present invention may be conveniently prepared, or formed during the process of the invention, as solvates (e.g. hydrates). Hydrates of compounds of the present invention may be conveniently prepared by recrystallisation from an aqueous/organic solvent mixture, using organic solvents such as dioxin, tetrahydrofuran or methanol. Compounds of Formula I can be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diasteromeric derivatives of compounds of Formula I, dissociable complexes are preferred (e.g., crystalline diastereoisomeric salts). Diastereomers have distinct physical properties (e.g., melting points, boiling points, solubilities, reactivity, etc.) and can be readily separated by taking advantage of these dissimilarities. The diastereomers can be separated by chromatography or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet, Samuel H. Wilen, Enantiomers, Racemates and Resolutions, John Wiley & Sons, Inc. (1981).

In summary, the compounds of Formula I are made by a process which comprises:

(A) reacting a compound of Formula II:

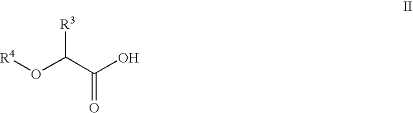

with a compound of the formula NH₂CR¹R²X³, in which X³, R¹, R², R³ and R⁴ are as defined in the Summary of the Invention for Formula I; or

(B) reacting a compound of Formula II with a compound of the formula NH₂X⁴, in which X⁴, R³ and R⁴ are as defined in the Summary of the Invention for Formula I; or

(C) reacting a compound of Formula 3:

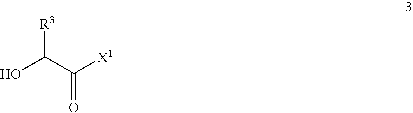

with a compound of formula R⁴L, in which X¹, R³ and R⁴ are as defined in the Summary of the Invention and L is a leaving group; or

(D) reacting a compound of Formula 4:

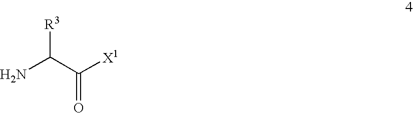

with a compound of formula R¹⁵L, in which X¹, R³ and R⁴ are as defined in the Summary of the Invention and L is a leaving group; and

(E) optionally converting a compound of Formula I into a pharmaceutically acceptable salt;

(F) optionally converting a salt form of a compound of Formula I to non-salt form;

(G) optionally converting an unoxidized form of a compound of Formula I into a pharmaceutically acceptable N-oxide;

(H) optionally converting an N-oxide form of a compound of Formula I its unoxidized form;

(I) optionally resolving an individual isomer of a compound of Formula I from a mixture of isomers;

(J) optionally converting a non-derivatized compound of Formula I into a pharmaceutically prodrug derivative; and

(K) optionally converting a prodrug derivative of a compound of Formula I to its non-derivatized form.

### EXAMPLES

The present invention is further exemplified, but not limited by, the following examples that illustrate the preparation of compounds of Formula I and II (Examples) and intermediates (References) according to the invention.

### LC/MS-Procedures

LC/MS (Method A):

Mass Spectrometer (MS)—LCT Time-of-Flight (Micromass UK Ltd) Serial No. KA014

Ionization Mode: Electrospray (Positive Ion)

Scan: Tof MS (Full Scan m/z 100-1200, sum for 0.4 s @ 50 us/scan) Centroid Mode

Liquid Chromatograph (LC): Hewlett Packard HP1100 Series Binary Pump (Serial #US80301343) & Degasser (serial #JP73008973)

Mobile Phase:

A=Water+0.05% TFA (trifluoroacetic acid) buffer

B=Acetonitrile+0.05% TFA buffer

Gradient: 5% B to 100% B in 5 minutes

Column: Hypersil BDS C-18, 3 u, 4.6 mm×50 mm Reverse Phase

Injection volume: 5 uL

Flow rate: 1 ml/min to column & to UV detector, flow split after UV detector

such that 0.75 ml/min to ELS detector and 0.25 ml/min to mass spectrometer.

Auxiliary Detectors: (i) Hewlett Packard Model HP1100 Series UV detector (serial #JP73704703) wavelength=220 nm

(ii) Sedere (France) Model SEDEX 75 Evaporative Light Scattering (ELS) detector (serial #9970002A)

temperature=46 deg C., Nitrogen pressure=4 bar

Autosampler/Injector: Gilson Model 215 Liquid Handler with Model 819 injection valve (serial #259E8280)

LC/MS (Method B):

Same as method A, but with a different gradient: 5% B to 90% B in 3 minutes, 90% B to 100% B in 2 min

LC/MS (Method C):

Mass Spectrometer (MS)—LCT Time-of-Flight (Micromass UK Ltd) Serial No. KA014

Ionization Mode: Electrospray (Positive Ion)

Scan: Tof MS (Full Scan m/z 100-1200, sum for 0.4 s @50 us/scan) Centroid Mode

Liquid Chromatograph (LC): Hewlett Packard HP1100 Series Binary Pump (Serial #US80301343) & Degasser (serial #JP73008973)

Mobile Phase:

A=Water+0.1% formic acid buffer

B=Acetonitrile+0.1% formic acid buffer

Gradient: 5% B to 90% B in 3 minutes, 90% B to 100% B in 2 min

Column: Phenomenex Synergi C-18, 2 u, 4. mm×20 mm Reverse Phase Injection volume: 5 uL

Flow rate: 1 ml/min to column & to UV detector, flow split after UV detector such that 0.75 ml/min to ELS detector and 0.25 ml/min to mass spectrometer.

Auxiliary Detectors: (i) Hewlett Packard Model HP1100 Series UV detector (serial #JP73704703) wavelength=220 nm

(ii) Sedere (France) Model SEDEX 75 Evaporative Light Scattering (ELS) detector (serial #9970002A)

temperature=46 deg C., Nitrogen pressure=4 bar

Autosampler/Injector: Gilson Model 215 Liquid Handler with Model 819 injection valve (serial #259E8280)

### Reference Example 1

### (a) (R)-3-[2-(1,1-Difluoro-methoxy)-phenylmethanesulfonyl]-2-hydroxy-propionic acid

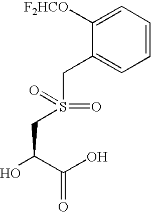

A solution of (R)-2-tert-Butoxycarbonylamino-3-[2-(1,1-difluoro-methoxy)-phenylmethanesulfonyl]-propionic acid (5.19 g) in CH₂Cl₂ (20 mL), was treated with trifluoroacetic acid (20 mL) at room temperature. After two hours, the reaction mixture was concentrated under reduced pressure. The white solid obtained was dissolved in 1M H₂SO₄ (100 mL) and dioxane (30 mL). The solution was cooled to 0° C., NaNO₂(1.95 g in 50 mL of water) was added with stirring for 1 hour. The reaction mixture was stirred overnight at ambient temperature. The product was then concentrated and extracted with ethyl acetate, dried with anhydrous MgSO₄, filtered, concentrated and recrystallized from ethyl acetate to yield (R)-3-[2-(1,1-difluoro-methoxy)-phenylmethanesulfonyl]-2-hydroxy-propionic acid (2.36 g).

### (b) (R)-2-hydroxy-3-phenylmethanesulfonyl-propionic acid

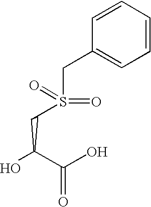

By proceeding in a manner similar to Reference Example 1(a) above but using (R)-2-tert-butoxycarbonylamino-3-[phenylmethanesulfonyl]-propionic acid there was prepared (R)-2-hydroxy-3-[phenylmethanesulfonyl]-propionic acid.

### Reference Example 2

### (R)-2-Amino-N-methoxy-N-methyl-butyramide

To a solution of [(R)-1-(methoxy-methyl-carbamoyl)-propyl]-carbamic acid tert-butyl ester (4.92 g, 20 mmol) in CH₂Cl₂ (20 ml) was added TFA (10 mL) at room temperature. After stirring for 2 hours, the reaction mixture was concentrated to dryness under reduced pressure to produce (R)-2-amino-N-methoxy-N-methyl-butyramide TFA salt (5.4 g).

### Reference Example 3

### (R)-3-[2-(1,1-Difluoro-methoxy)-phenylmethanesulfonyl]-2-triisopropylsilanyloxy-propionic acid

(R)-3-[2-(1,1-difluoro-methoxy)-phenylmethanesulfonyl]-2-hydroxy-propionic acid (7.0 g, 22.58 mmol), in CH₂Cl₂ (50 mL) was reacted with 2,6-lutidine (12.09 g, 112.9 mmol) and triisopropylsilyl-trifluoro-methanesulfonate (20.75 g, 67.74 mmol) at −78° C. for one hour. The reaction mixture was allowed to warm to room temperature before being quenched by the addition of saturated ammonium chloride solution. The product was extracted with ethyl acetate, the solvent was removed under reduced pressure and the oil residue was then dissolved in EtOH:THF:H₂O (3:1:1, 60 mL). Solid K₂CO₃ (24 g) was added at room temperature and the mixture was stirred for one hour, filtered, extracted with ethyl acetate, dried with anhydrous MgSO₄, filtered and concentrated to yield (R)-3-[2-(1,1-Difluoro-methoxy)-phenylmethanesulfonyl]-2-triisopropylsilanyloxy-propionic acid (8.58 g).

Following as in reference 3 provided the following intermediate: (R)-3-Phenylmethanesulfonyl-2-triisopropylsilanyloxy-propionic acid

### Reference Example 4

### 3-[2-(1,1-Difluoro-methoxy)-phenylmethanesulfonyl]-propionic acid

A mixture of [2-(1,1-difluoro-methoxy)-phenyl]-methanethiol (190 mg, 1.0 mmol), acrylic acid (69 □L, 1.0 mmol), diisopropylethylamine (44.0 □L, 1.1 mmol) and 0.5 mL dimethylformamide was stirred at 45° C. for 4 hours. Diethyl ether (5 mL) and 1N HCl (2 mL) was added. The layers were separated and the organic layer was washed with 1N HCl (2 mL), dried over MgSO₄ and concentrated. The resulting oil was dissolved in methanol (5 mL), treated with an aqueous solution (5 mL) of Oxone® (921 mg, 1.5 mmol), and stirred for 1 hour. Methanol was removed under reduced pressure and 20 mL water was added. The mixture was extracted with two 60 mL portions of ethyl acetate, dried over MgSO₄ and concentrated to give 3-[2-(1,1-difluoro-methoxy)-phenylmethanesulfonyl]-propionic acid (160 mg; 0.54 mmol, 54% yield).

### Reference Example 5

### 3-Benzylsulfanyl-2-(2-nitro-phenylamino)-propionic acid

S-benzylcysteine (1.06 g, 5.0 mmol), 2-fluoronitrobenzene (1.05 mL, 10.0 mmol), potassium carbonate (1.38 g, 10.0 mmol) and dimethylformamide (3 mL) were combined and stirred at 100° C. for 4 hours. The mixture was diluted with 40 mL water and washed with two 15 mL portions of diethyl ether. The aqueous layer was acidified to pH 4 with 6N HCl and extracted with two 30 mL portions of ethyl acetate. The ethyl acetate layer was dried over MgSO₄, and concentrated. Diethyl ether was added and then decanted to give 3-benzalsulfanyl-2-(2-nitro-phenylamino)-propionic acid (541 mg, 1.63 mmol, 33% yield).

### Reference Example 6

### (R)-3-Benzylsulfanyl-2-(5-nitro-thiazol-2-ylamino)-propionic acid

S-benzylcysteine (0.845 g, 4 mmol) and bis(trimethylsilyl)acetamide (3 mL, 16 mmol) were stirred at 75° C. for 1 hour. 2-Bromo-5-nitrothiazole (837 mg, 4 mmol) and toluene (8 mL) was added and the mixture was stirred at 100° C. for 1 day. Toluene was removed under reduced pressure. The residue was stirred in 5 mL dioxane and 5 mL 1N HCl for 30 minutes. Dioxane was removed under reduced pressure and the mixture was basified with saturated NaHCO₃ and washed with 50 mL ethyl acetate. The aqueous layer was acidified with 6N HCl and extracted with two 25 mL portions of ethyl acetate, dried over MgSO₄, concentrated and chromatographed using a gradient of 5-10% methanol in methylene chloride to yield (R)-3-benzylsulfanyl-2-(5-nitro-thiazol-2-ylamino)-propionic acid (42.7 mg, 0.123 mmol, 3% yield).

### Reference Example 7

### (2S)-4,4-Difluoro-2-hydroxy-5-phenyl-pentanoic acid

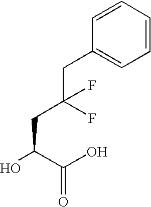

To a suspension of (S)-2-Amino-4,4-difluoro-5-phenyl-pentanoic acid (1.0 mmol, 230 mg) in water (3 mL) was added 2M sulfuric acid dropwise until the solid dissolved (ca 3 mL). A solution of sodium nitrite (1.5 eq., 1.5 mmol, 104 mg) in 1 ml of water was then added dropwise. The mixture was stirred at room temperature for 21 hours then extracted twice with ether (30 ml). The organic layers were dried over MgSO₄ and then concentrated in vacuum to afford (2S)-4,4-difluoro-2-hydroxy-5-phenyl-pentanoic acid (90 mg, 39%) as a white solid. ¹H NMR (CDCl₃) 7.3 (m, 5H), 5.6 (b, 1H), 4.61 (dd, J=8.5, 2.9 Hz, 1H), 3.3 (t, J=16.8 Hz 2H), 2.45 (m, 1H), 2.2 (m, 2H).

### Reference Example 8

### 2-(S)-(2-Morpholin-4-yl-2-oxo-ethoxy)-4-phenyl-butyric acid

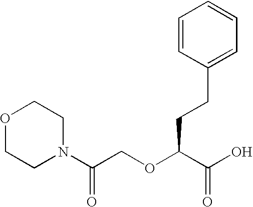

Step (i): To a cooled (0° C.) solution of ethyl (2R) 2-hydroxy-4-phenylbutyrate (1.81 g, 8.71 mmol), 4-nitro-benzoic acid (1.1 eq., 9.56 mmol, 1.598 g) and triphenyl phosphine (1.1 eq., 9.5 mmol, 2.50 g) in dry THF (80 mL) under nitrogen was added slowly diethyl azodicarboxylate (1.1 eq., 9.56 mmol, 1.67 g). The mixture was stirred at 0° C. for 2.5 hours and then concentrated in vacuum. The residue was triturated with a mixture of ethyl acetate and heptane (1:3, 150 mL) and the resulting solids were filtered off. The filtrate was concentrated in vacuum and purified over 110 g silica gel, eluting with a mixture of ethyl acetate and heptane (1:4, v/v) to afford 4-nitro-benzoic acid (S)-1-ethoxycarbonyl-3-phenyl-propyl ester (3.4 g, 98%).

Step (ii): To a cooled (0° C.) solution of 4-nitro-benzoic acid (S)-1-ethoxycarbonyl-3-phenyl-propyl ester (2.04 g, 5.83 mmol) in MeOH (30 mL) was added potassium carbonate (1.5 eq., 8.75 mmol, 1.21 g). The mixture was stirred at 0° C. for 5 minutes then at room temperature for 1.5 hours and concentrated in vacuum. The residue was partitioned between water (40 mL) and ethyl acetate (40 mL). The organic layer was dried over MgSO₄ and then concentrated in vacuum. The residue was purified over 35 g silica gel, eluted with dichloromethane to afford methyl-(2S)-2-hydroxy-4-phenyl-butyrate as a colorless oil (933 mg, 82%).

Step (iii): To a solution of methyl-(2S)-2-hydroxy-4-phenyl-butyrate (300 mg, 1.54 mmol) in dry DMF (3 mL) under nitrogen was added sodium hydride (60%, 1.5 eq., 2.32 mmol, 92.7 mg). After 5 min, 4-(2-chloroacetyl) morpholine (1.1 eq., 1.69 mmol, 277 mg) was added and the mixture was stirred at room temperature for 24 hours, then diluted with water (60 mL) and then neutralized with 1N HCl. The aqueous solution was extracted twice with ethyl acetate (40 mL). The organic layer was washed with water (50 mL), dried over MgSO₄ and then concentrated in vacuum. The residue was purified over 35 g silica gel, eluting with ethyl acetate then with 5% MeOH in ethyl acetate to afford (S)-2-(2-morpholin-4-yl-2-oxo-ethoxy)-4-phenyl-butyric acid methyl ester (117 mg, 24%).

Step (iv): To a solution of (S)-2-(2-morpholin-4-yl-2-oxo-ethoxy)-4-phenyl-butyric acid methyl ester (117 mg, 0.36 mmol) in MeOH:H₂O (2:1 vol, 3 mL) was added lithium hydroxide hydrate (2.0 eq., 0.73 mmol, 30.5 mg). The mixture was stirred at room temperature for 5 hours, then diluted with water (30 mL) and then extracted with ether (30 mL). The aqueous layer was acidified with 1N HCl and extracted twice with ether (30 mL). The acidic extracts were dried over MgSO₄ and then concentrated in vacuum to afford (S)-2-(2-Morpholin-4-yl-2-oxo-ethoxy)-4-phenyl-butyric acid (85.5 mg, 77%) as a colorless oil. ¹H NMR (CDCl₃) 10.5 (b, 1H), 7.2 (m, 5H), 4.55 (d, J=15.2 Hz, 1H), 4.14 (d, J=15.2 Hz, 1H), 3.9 (dd, J=7.6, 4.2 Hz, 1H), 4.6 (m, 6H), 3.4 (m, 2H), 2.8 (m, 2H), 2.3 (m, 1H), 2.15 (m, 1H). LC/MS 96% (M+1) 308.

### Reference Example 9

### (2S)-2-Fluoro-4-phenyl-butyric acid

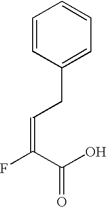

Step (i): To a cooled (0° C.) solution of methyl-(2R)-2-hydroxy-4-phenyl-butyrate (1.00 g, 4.80 mmol) in dry dichloromethane (3 mL) was added DAST (3.0 eq., 14.4 mmol, 2.32 g). The mixture was stirred at room temperature for 18 hours then diluted with dichloromethane (20 mL) and carefully quenched with saturated sodium bicarbonate (150 mL). The aqueous layer was extracted with dichloromethane (30 mL) and the organic layers were dried over MgSO₄ and then concentrated in vacuo. The residue was purified over 90 g silica gel, eluting with a mixture of dichloromethane and heptane (1:2 then 1:1, v/v) to afford methyl-2S-fluoro-4-phenyl-butyrate as a light yellow oil (578 mg, 57%).

Step (ii): To a solution of methyl-2S-fluoro-4-phenyl-butyrate (577 mg, 2.74 mmol) in a mixture of MeOH:H2O (2:1 vol, 6 mL) was added lithium hydroxide monohydrate (1.5 eq., 4.11 mmol, 173 mg). The mixture was stirred at room temperature for 5 hours and then concentrated in vacuum. The residue was diluted with water (30 mL) and extracted with ether (20 mL). The aqueous layer was acidified with HCl and extracted with ether (30 mL). The acidic extract was dried over MgSO₄ and then concentrated in vacuum to afford 2(S)-fluoro-4-phenyl-butyric acid as a yellow oil (486 mg, 97%). ¹H NMR (CDCl₃) 7.5 (b, 1H), 7.3 (m, 5H), 4.95 (ddd, J=48.9, 6.9, 5.4 Hz, 1H), 2.85 (m, 2H), 2.25 (m, 2H). MS (CI) M+1 183.

### Reference Example 10

### 2(R)-Methoxy-4-phenyl-butyric acid

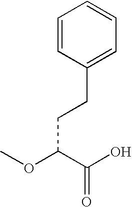

Step 1: To a solution of ethyl-(2R)-2-hydroxy-4-phenyl-butyrate (500 mg, 2.40 mmol) in dry DMF (4 mL) under nitrogen was added sodium hydride (60%, 2.0 eq., 4.80 mmol, 192 mg) followed by methyl iodide (3.0 eq., 7.20 mmol, 1.02 g). The mixture was stirred at room temperature for 22 hours, then diluted with NH₄Cl (100 mL) and extracted with ethyl acetate (50 mL). The organic layer was dried over MgSO4 and then concentrated in vacuum. The residue was purified over 35 g silica gel, eluting with ethyl acetate and heptane (1:3, v/v) to afford (R)-2-methoxy-4-phenyl-butyric acid ethyl ester(480 mg, 90%).

Step 2: To a solution of (R)-2-methoxy-4-phenyl-butyric acid ethyl ester (480 mg, 2.8 mmol) in MeOH:H₂O (2:1 vol, 9 mL) was added lithium hydroxide hydrate (2.0 eq., 4.32 mmol, 181 mg). The mixture was stirred at room temperature for 2.5 hours, then diluted with water (20 mL) and then extracted with ether (20 mL). The aqueous layer was acidified with 1N HCl and then extracted twice with ether (30 mL). The combined extracts were dried over MgSO4 and then concentrated in vacuum to afford 2(R)-methoxy-4-phenyl-butyric acid (426 mg, quant.) as a colorless solid. ¹H NMR (CDCl₃) 7.25 (m, 5H), 3.8 (dd, J=6.8, 5.2 Hz, 1H), 3.48 (s, 3H), 2.78 (t, J=7.3 Hz, 2H), 2.1 (m, 2H). MS (CI) M 194.

Following as in reference 10 but using benzyl bromide in step 2 provided the following intermediate:

### 2(R)-Benzyloxy-4-phenyl-butyric acid

### REFERENCE EXAMPLE 11

### (a) (R)-2-Amino-N-[1-(benzothiazol-2-yl-hydroxy-methyl)-butyl-3-phenylmethanesulfonyl-propionamide

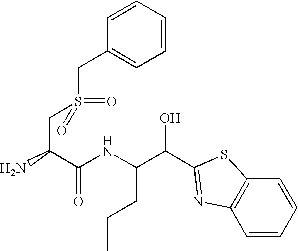

A solution of {(R)-1-[1-(benzothiazol-2-yl-hydroxy-methyl)-butylcarbamoyl]-2-phenylmethanesulfonyl-ethyl}-carbamic acid tert-butyl ester {888 mg, 1.58 mmol, Example 27(a)} in dichloromethane (5 mL) was treated with trifluoroacetic acid (5 mL). The mixture was stirred at room temperature for one hour and then evaporated. The residue was dissolved in dichloromethane (20 mL) and this solution was treated with Silicycle Triamine (4.3 g, 16 mmol). The mixture was stirred at room temperature for two hours and then filtered. The filtrate was evaporated to give the title compound (692 mg, 94%). LC/MS m/z=562 (M+H).

### (b) (S)-2-Amino-N-[(S)-1-(benzoxazol-2-yl-hydroxy-methyl)-butyl]-3-thiophen-2-yl-propionamide

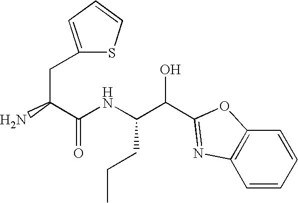

By proceeding in a manner similar to Reference Example 11(a) above but using {(S)-1-[(S)-1-(benzoxazol-2-yl-hydroxy-methyl)-butylcarbamoyl]-2-thiophen-2-yl-ethyl}-carbamic acid tert-butyl ester {790 mg, 1.67 mmol, Example 27(c)} and subjecting the reaction product to flash chromatography on silica eluting with a mixture of ethyl acetate and methanol (9:1, v/v) here was prepared (S)-2-amino-N-[(S)-1-(benzoxazol-2-yl-hydroxy-methyl)-butyl]-3-thiophen-2-yl-propionamide (415 mg, 66%). LC/MS m/z=374 (M+H).

### (c) (R)-2-Amino-N-[(S)-1-(benzoxazol-2-yl-hydroxy-methyl)-butyl]-3-phenylmethanesulfonyl-propionamide

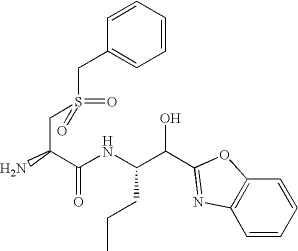

By proceeding in a manner similar to Reference Example 11(a) above but using {(R)-1-[(S)-1-(Benzoxazol-2-yl-hydroxy-methyl)-butylcarbamoyl]-2-phenylmethanesulfonyl-ethyl}-carbamic acid tert-butyl ester {908 mg, 1.66 mmol, Example 27(b)} there was prepared (R)-2-amino-N-[(S)-1-(benzoxazol-2-yl-hydroxy-methyl)-butyl]-3-phenylmethanesulfonyl-propionamide (726 mg, 98%). LC/MS m/z=446 (M+H).

### (d) (R)-2-Amino-N-[1-(benzothiazol-2-yl-hydroxy-methyl)-butyl]-3-phenylmethanesulfonyl-propionamide

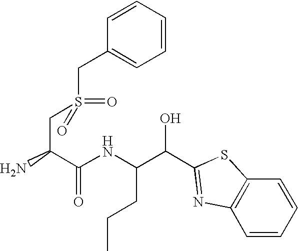

By proceeding in a manner similar to Reference Example 11(a) above but using {(R)-1-[1-(Benzothiazol-2-yl-hydroxy-methyl)-butylcarbamoyl]-2-phenylmethanesulfonyl-ethyl}-carbamic acid tert-butyl ester {0.63 mmol, Example 27(d)} there was prepared (R)-2-Amino-N-[1-(benzothiazol-2-yl-hydroxy-methyl)-butyl]-3-phenylmethanesulfonyl-propionamide (212 mg, 73%). LC/MS m/z=462 (M+H).

### (e) (R)-2-Amino-N-[(S)-1-(benzoxazol-2-yl-hydroxy-methyl)-butyl]-3-phenylmethanesulfonyl-propionamide

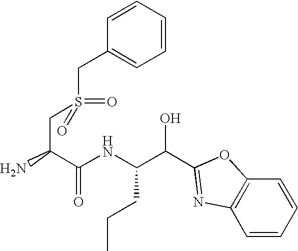

By proceeding in a manner similar to Reference Example 11(a) above but using {(R)-1-[(S)-1-(Benzoxazol-2-yl-hydroxy-methyl)-butylcarbamoyl]-2-phenylmethanesulfonyl-ethyl}-carbamic acid tert-butyl ester {1.7 mmol, Example 27(e)} there was prepared (R)-2-amino-N-[(S)-1-(benzoxazol-2-yl-hydroxy-methyl)-butyl]-3-phenylmethanesulfonyl-propionamide (726 mg, 98%). LC/MS m/z=446 (M+H).

### (f) (R)-2-Amino-N-[(S)-1-(benzoxazol-2-yl-hydroxy-methyl)-butyl]-3-cyclopropylmethanesulfonyl-propionamide

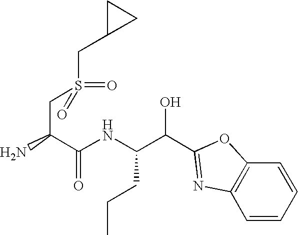

By proceeding in a manner similar to Reference Example 11(a) above but using {(R)-1-[(S)-1-(benzoxazol-2-yl-hydroxy-methyl)-butylcarbamoyl]-2-cyclopropylmethanesulfonyl-ethyl)}-carbamic acid tert-butyl ester {450 mg, 0.88 mmol, Example 27(f)} there was prepared (R)-2-amino-N-[(S)-1-(benzoxazol-2-yl-hydroxy-methyl)-butyl-3-cyclopropylmethanesulfonyl-propionamide (360 mg, 0.879 mmol, 100%).

LC/MS m/z410(M+H)

### (g) (R)-2-Amino-N-{1-[hydroxy-(3-phenyl-1,2,4-oxadiazol-5-yl)-methyl]-propyl}-3-phenylmethanesulfonyl-propionamide

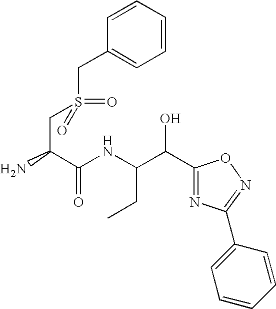

By proceeding in a manner similar to Reference Example 11(a) above but using (R)-1-{1-[Hydroxy-(3-phenyl-1,2,4-oxadiazol-5-yl)-methyl]-propylcarbamoyl}-2-phenylmethanesulfonyl-ethyl)-carbamic acid tert-butyl ester {Example 27(g)} there was prepared (R)-2-amino-N-{1-[hydroxy-(3-phenyl-1,2,4-oxadiazol-5-yl)-methyl]-propyl}-3-phenylmethanesulfonyl-propionamide. LC/MS m/z=481 (M+Na), 459(M+H)

### (h) (R)-2-Amino-3-cyclopropylmethanesulfonyl-N-{(S)-1-[(5-ethyl-1,2,4-oxadiazol-3-yl)-hydroxy-methyl]-propyl}-propionamide

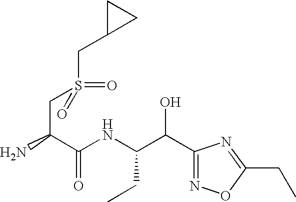

By proceeding in a manner similar to Reference Example 11(a) above but using ((R)-2-cyclopropylmethanesulfonyl-1-{(S)-1-[(5-ethyl-1,2,4-oxadiazol-3-yl)-hydroxy-methyl]-propylcarbamoyl}-ethyl)-carbamic acid tert-butyl ester {Example 27(i)} there was prepared (R)-2-amino-3-cyclopropylmethanesulfonyl-N-{(S)-1-[(5-ethyl-1,24-oxadiazol-3-yl)-hydroxy-methyl]-propyl}-propionamide. LC/MS m/z=375(M+H)

### (i) (R)-2-Amino-N-[1-(benzoxazol-2-yl-hydroxy-methyl)-butyl]-3-phenylmethanesulfonyl-propionamide

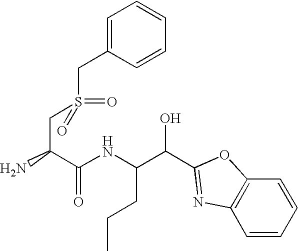

By proceeding in a manner similar to Reference Example 11(a) above but using {(R)-1-[1-(Benzoxazol-2-yl-hydroxy-methyl)-butylcarbamoyl]-2-phenylmethanesulfonyl-ethyl}-carbamic acid tert-butyl ester {Example 27(j)} there was prepared (R)-2-Amino-N-[1-(benzoxazol-2-yl-hydroxy-methyl)-butyl]-3-phenylmethanesulfonyl-propionamide. LC/MS m/z=446(M+H)

### (j) (R)-2-Amino-N-[(S)-1-(benzoxazol-2-yl-hydroxy-methyl)-3-phenyl-propyl]-3-cyclopropylmethanesulfonyl-propionamide

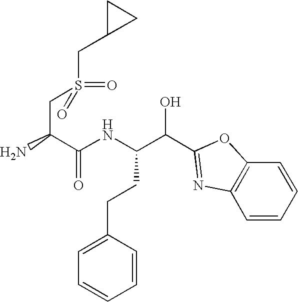

By proceeding in a manner similar to Reference Example 11(a) above but using {(R)-1-[(S)-1-(benzoxazol-2-yl-hydroxy-methyl)-3-phenyl-propylcarbamoyl]-2-cyclopropylmethanesulfonyl-ethyl}-carbamic acid tert-butyl ester {Example 27(k)} there was prepared (R)-2-amino-N-[(S)-1-(benzoxazol-2-yl-hydroxy-methyl)-3-phenyl-propyl]-3-cyclopropylmethanesulfonyl-propionamide. LC/MS m/z=472(M+H)

### (k) (R)-2-Amino-N-[(S)-1-(hydroxy-thiazol-2-yl-methyl)-3-phenyl-propyl]-3-phenylmethanesulfonyl-propionamide

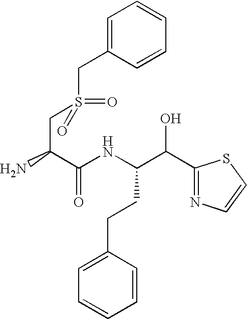

By proceeding in a manner similar to Reference Example 11(a) above but using {(R)-1-[(S)-1-(Hydroxy-thiazol-2-yl-methyl)-3-phenyl-propylcarbamoyl]-2-phenylmethanesulfonyl-ethyl}-carbamic acid tert-butyl ester {Example 27(l)} there was prepared (R)-2-amino-N-[(S)-1-(hydroxy-thiazol-2-yl-methyl)-3-phenyl-propyl]-3-phenylmethanesulfonyl-propionamide.

### (l) (R)-2-Amino-3-phenylmethanesulfonyl-N-{(S)-1-[(3-cyclopropyl-1,2,4-oxadiazol-5-yl)-hydroxy-methyl]-propyl}-propionamide

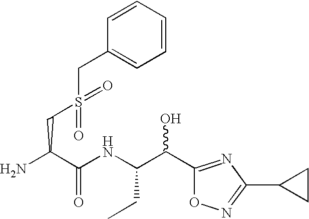

By proceeding in a manner similar to Reference Example 11(a) above but using ((R)-2-phenylmethanesulfonyl-1-{(S)-1-[(3-cyclopropyl-1,2,4-oxadiazol-5-yl)-hydroxy-methyl]-propylcarbamoyl}-ethyl)-carbamic acid tert-butyl ester {Example 27(s)} there was prepared (R)-2-amino-3-phenylmethanesulfonyl-N-{(S)-1-[(3-cyclopropyl-1,2,4-oxadiazol-5-yl)-hydroxy-methyl]-propyl}-propionamide.

### (m) 2-amino-1-(5-ethyl-[1,3,4]oxadiazol-2-yl-butan-1-ol

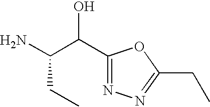

By proceeding in a manner similar to Reference Example 11(a) above but using {1-[(5-ethyl-[1,3,4]oxadiazol-2-yl)-hydroxy-methyl]-propyl}-carbamic acid tert-butyl ester (Reference Example 16) there was prepared 2-amino-1-(5-ethyl-[1,3,4]oxadiazol-2-yl-butan-1-ol.

### REFERENCE EXAMPLE 12

### [(S)-1-(Hydroxy-thiazol-2-yl-methyl)-3-phenyl-propyl]-carbamic acid tert-butyl ester

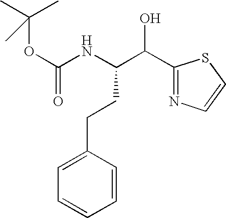

n-Butyllithium (4.2 ml, 10.5 mmol, 2.5M solution in hexanes) was mixed with 16 ml diethylether and the resulting solution cooled to −78° C. 2-Bromothiazole (1.64 g, 10 mmol) was dissolved in a mixture of 2 ml diethylether and 1 ml THF. This solution was added dropwise to the n-butyllithium solution. The resulting reaction mixture was stirred for 15 min. A solution of [(S)-1-(Methoxy-methyl-carbamoyl)-3-phenyl-propyl]-carbamic acid tert-butyl ester (1.4 g, 4.3 mmol) in 20 ml THF was added dropwise to the reaction mixture. Stirring was continued for one hour and the reaction mixture quenched by addition of 50 ml water. After warming to room temperature the phases were separated and the aqueous phase extracted with ethyl acetate. The combined organic phases were washed with brine and dried with magnesium sulfate. The solvents were evaporated under vacuum to give 1.4 g [(S)-3-Phenyl-1-(thiazole-2-carbonyl)-propyl]-carbamic acid tert-butyl ester as a brown solid. [(S)-3-Phenyl-1-(thiazole-2-carbonyl)-propyl]-carbamic acid tert-butyl ester (1.41 g, 4.1 mmol) was dissolved in 50 ml ethanol and the solution cooled to 0° C. Sodium borohydride (155 mg, 4.1 mmol) was added and the reaction mixture stirred for 90 minutes. Water was added and the aqueous phase acidified by addition of 1M hydrochloric acid. The aqueous phase was extracted with ethyl acetate. The combined organic phases were washed with brine and dried with magnesium sulfate. The solvent was evaporated under reduced pressure. (1.32, 3.8 mmol, 88%). LC/MS m/z=271 (M+H-isobutene), 249(M+H-boc)

### REFERENCE EXAMPLE 13

### (S)-2-Amino-4-phenyl-1-thiazol-2-yl-butan-1-ol

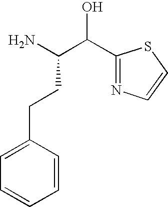

[(S)-1-(Hydroxy-thiazol-2-yl-methyl)-3-phenyl-propyl]-carbamic acid tert-butyl ester (1.32 g, 3.8 mmol, Reference Example 12) was dissolved in 10 ml dichloromethane. Trifluoroacetic acid was added and the resulting reaction mixture stirred for two hours. The solvents were evaporated under reduced pressure and saturated sodium bicarbonate solution was added. The solution was extracted with ethyl acetate. The combined organic phases were washed with brine and dried with magnesium sulfate. The solvent was evaporated and the crude product purified via flash chromatography (eluted with ethyl acetate followed by 10% methanol in ethyl acetate) to give (S)-2-amino-4-phenyl-1-thiazol-2-yl-butan-1-ol (466 mg, 1.87 mmol, 49%). LC/MS m/z=249(M+H).

### REFERENCE EXAMPLE 14

### (S)-2-Amino-1-(3-cyclopropyl-1,2,4-oxadiazol-5-yl)-butan-1-ol

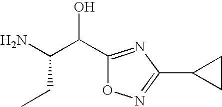

A solution of boc-3S-amino-2-hydroxypentanoic acid (2.00 g, 8.57 mmol) and 1.20 equivalents of cyclopropanecarboxamidoxime (1.03 g, 10.29 mmol) in 20 mL of dichloromethane was stirred at 0° C. as 1.25 equivalents of N-cyclohexylcarbodiimide-N′-methyl polystyrene (1.70 mmol/g, 6.30 g, 10.72 mmol) was added in portions and the reaction mixture stirred under nitrogen for three hours while warming to 15° C. The reaction mixture was filtered and the resin washed with dichloromethane. Evaporate under vacuum to dryness. [LC/MS m/z=338 (M+H+Na)] The residue is dissolved in 20 mL of tetrahydrofuran and heated in a microwave reactor at 160° C. for three minutes. Evaporate under vacuum to dryness. [LC/MS m/z=320 (M+H+Na)] The residue is dissolved in 50 mL of dichloromethane and stirred at room temperature as a 50 mL solution of 50% trifluoroacetic acid in dichloromethane was added dropwise. After three hours the reaction was evaporated under vacuum to dryness and dissolved in 50 mL of dichloromethane again. Three equivalents of Silicycle triamine-3 was added and the mixture stirred at room temperature overnight. The mixture was filtered and washed with dichloromethane. Evaporate under vacuum to give (S)-2-Amino-1-(3-cyclopropyl-1,2,4-oxadiazol-5-yl)-butan-1-ol 1.04 g (61% overall). [LC/MS m/z=198 (M+H)]

### REFERENCE EXAMPLE 15

### Ethyl-1,3,4-oxadiazole

A mixture of the formic hydrazide (60 g, 1 mole), triethylorthopropionate (176.26 g, 1 mole) and p-toluenesulfonic acid (250 mg) was heated at 120° C. for 12 hours. The ethanol was removed under vacuum and the residue was distilled under vacuum to yield 24 g of ethyl-1,3,4-oxadiazole. H¹ NMR (DMSO-□): 9.34 (1H, s), 2.86 (2H, q), 1.25(3H, t).

### REFERENCE EXAMPLE 16

### {1-[(5-Ethyl-[1,3,4]oxadiazol-2-yl)-hydroxy-methyl]-propyl}-carbamic acid tert-butyl ester

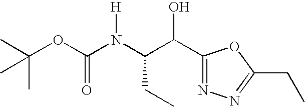

To a stirred solution of the ethyl-1,3,4-oxadiazole (4.66 g, 48 mmol, Reference Example 15) in THF (50 ml) was added n-BuLi (1.6M solution in 30 ml of hexane) drop-wise under N₂ at −78° C. After 1 hour, MgBr.Et₂O (12.38 g, 48 mmol) was added and the reaction mixture was allowed to warm to −45° C. for 1 hour before being treated with 2-Boc-Nlu-aldehyde (3.2 g, 24 mmol) in THF (20 ml). The reaction mixture was stirred for 1 hour, quenched with saturated NH₄Cl, and extracted with ethyl acetate. The organic layer was washed with brine, dried with MgSO₄ and concentrated. The residue was purified by silica gel column chromatography to yield {1-[(5-ethyl-[1,3,4]oxadiazol-2-yl)-hydroxy-methyl]-propyl}-carbamic acid tert-butyl ester (2.13 g). ¹ NMR (DMSO-□): 6.65, 6.52(1H, d, d, J=9.2 Hz, J=9.2 Hz, NH, diastereomer), 6.14, 5.95(1H, d, d, J=5.6 Hz, J=5.6 Hz, OH, diastereomer), 4.758, 4.467(1H, m, diastereomer), 3.7-3.55(1H, m), 2.8(2H, q), 1.33(12H, t), 1.25-1.21(2H, m), 0.82(3H, m). MS: 284.1 (M-1), 286 (M+1), 308(M+Na).

### REFERENCE EXAMPLE 17

### (a) (S)-2-Amino-1-benzooxazol-2-yl-butan-1-ol

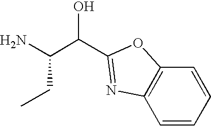

Step 1. Benzoxazole (600 mg, 5 mmol) in 20 ml THF was cooled to −5° C. and isopropyl magnesium chloride (2M in THF, 2.5 ml, 5 mmol ) was added. After stirring for 1 hour at −5° C., (S)-(1-formyl-propyl)-carbamic acid tert-butyl ester {561 mg, 3 mmol, Reference Example 18(a)}, prepared as in reference 15, in 10 ml THF was added. The reaction was allowed to warm to room temperature with stirring for 2 hours. The reaction was quenched with saturated ammonium chloride solution, excess THF solvent removed. The residue was extracted with EtOAc, washed with brine, dried with anhydrous MgSO₄, filtered and concentrated. The crude residue was purified by chromatograph to yield 688 mg product (75%); LC-MS: 305.2 (M-1), 307.0 (M+1).

Step 2. (S)-[1-(Benzooxazol-2-yl-hydroxy-methyl)-propyl]-carbamic acid tert-butyl ester (275 mg, 0.89 mmol) and MeCl₂ (5 ml) were mixed and TFA (1 ml) was added at room temperature. After stirring for 1 hour, the solvent and excess TFA were removed under vacuum to produce 260 mg of (S)-2-amino-1-benzooxazol-2-yl-butan-1-ol TFA salt.

### (b) (S)-2-Amino-1-benzothiazol-2-yl-butan-1-ol

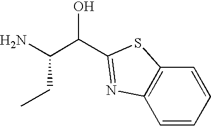

By proceeding in a similar manner to Example 17(a) but using benzothiazole in Step 1 there was prepared (S)-2-amino-1-benzothiazol-2-yl-butan-1-ol TFA salt.

### (c) (S)-2-amino-1-benzooxazol-2-yl-pentan-1-ol

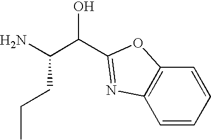

By proceeding in a similar manner to Example 17(a) but using (S)-(1-formyl-butyl)-carbamic acid tert-butyl ester {561 mg, 3 mmol, Reference Example 18(b)} in Step 1 there was prepared (S)-2-amino-1-benzooxazol-2-yl-pentan-1-ol.

### (d) 2-amino-1-benzothiazol-2-yl-pentan-1-ol

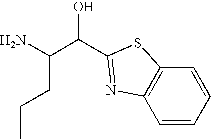

By proceeding in a similar manner to Example 17(a) but using benzothiazole and (S)-(1-formyl-butyl)-carbamic acid tert-butyl ester {561 mg, 3 mmol, Reference Example 18(b)} in Step 1 there was prepared 2-amino-1-benzothiazol-2-yl-pentan-1-ol.

### REFERENCE EXAMPLE 18

### (a) (S)-(1-Formyl-propyl)-carbamic acid tert-butyl ester

(S)-(+)-2-amino-1-butanol (50 g, 561 mmol) in 200 ml of water and 200 ml dioxane was cooled to 0° C. and mixed with NaOH (26.9 g, 673 mmol) and di-t-butyl-dicarbonate (146.96 g, 673 mmol). After the addition, the reaction was allowed to warm to room temperature. The reaction mixture was stirred for 2 hours. After removing the dioxane, the residue was extracted with EtOAc, then washed with brine and dried with anhydrous MgSO₄, filtered and concentrated. Without further purification, the crude product (120 g) was used for next step reaction.

A solution of oxylyl chloride (40.39 g, 265 mmol) in 700 ml of MeCl₂ was stirred and cooled to −60° C. Dimethylsulfoxide (51.7 g, 663 mmol) in 100 ml of MeCl₂ was added drop wise. After 10 minutes a solution of (S)-2-boc-amino-1-butanol (50 g, 265 mmol) in 100 ml of MeCl₂ was added drop wise at −70° C. The reaction mixture was allowed to warm to −40° C. for 10 minutes and then cooled to −70° C. again. A solution of triethylamine (74.9 g, 742 mmol) in 100 ml of MeCl₂ was added. The reaction mixture was allowed to warm to room temperature over 2 hours. 100 mls of saturated sodium dihydrogen phosphate was added, and then the organic layer was washed with brine and dried over MgSO₄. The solvent was removed to yield 45 g of (S)-(1-formyl-propyl)-carbamic acid tert-butyl ester; H¹ NMR (DMSO-□): 9.4(1H, s), 7.29(1H, br.), 3.72(1H, m), 1.69(2H, m), 1.4-1.2(9H, s), 0.86(3H, t).

### (b) By proceeding in a similar manner to Reference Example 18(a) but using (S)-(+)-2-amino-1-pentanol there was prepared (S)-(1-formyl-butyl)-carbamic acid tert-butyl ester.

### REFERENCE EXAMPLE 19

### (S)-3-Amino-2-hydroxy-pentanoic acid benzylamide

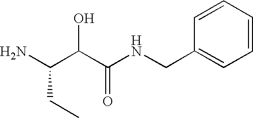

Step 1. (1S)-(2-Cyano-1-ethyl-2-hydroxyethyl)carbamic acid tert-butyl ester (10 g, 46.7 mmol) was dissolved in 1,4-dioxane (100 mL). Anisole (5 mL) was added and then concentrated HCl (100mL). The mixture was heated under reflux for 24 hours. The mixture was evaporated to dryness under vacuum and re-dissolved in 100 mL water. The solution was washed with ether and then neutralized with saturated aqueous NaHCO₃. Di-tert-butyl dicarbonate (10 g, 46 mmol) was added with 1,4-dioxane (200 mL), and the mixture was stirred at ambient temperature for 24 hours. The dioxane was removed under vacuum and the remaining aqueous solution was washed with ether. The solution was acidified with 1N HCl and extracted with ethyl acetate. The combined organic layers were washed with brine, dried with magnesium sulfate and evaporated to yield 3-tert-Butoxycarbonylamino-2-hydroxy-pentanoic acid (4.5 g) as yellowish oil.

Step 2. 3-tert-Butoxycarbonylamino-2-hydroxy-pentanoic acid (300 mg, 1.29 mmol) was combined with EDC (400 mg, 2.1 mmol) and HOBt (400 mg, 2.6 mmol). A solution of benzylamine (0.22 mL) and 4-methylmorpholine (0.5 mL) in dichloromethyl (4 mL) was added in one portion. The mixture was stirred at ambient temperature for 2 hours. After dilution with ethyl acetate (150 mL), the solution was washed with 1 N aqueous HCl, water, saturated aqueous NaHCO₃ solution and brine. The resultant mixture was dried with magnesium sulfate and evaporated under vacuum to yield (S)-3-amino-2-hydroxy-pentanoic acid benzylamide (380 mg) as a white solid.

Step 3. (S)-3-Amino-2-hydroxy-pentanoic acid benzylamide was dissolved in a mixture of TFA/dichloromethyl (1:1; 6 mL), stirred for 1 hour and evaporated to dryness. (3S)-3-Amino-2-hydroxy-pentanoic acid benzylamide was obtained as the TFA salt and used without further purification.

### REFERENCE EXAMPLE 20

### (S)-2-Amino-1-oxazolo[4,5-b]pyridin-2-yl-butan-1-ol

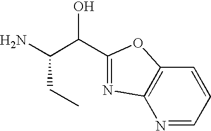

Step 1. A mixture of 2-amino-3-hydroxy pyridine (25 g, 227 mmol), triethylorthoformate (75 ml) and p-toluenesulfonic acid (61 mg) was heated at 140° C. for 8 hours. Excess triethylorthoformate was removed under vacuum. The product was crystallized from ethyl acetate to yield 22.5 g of pyridyloxazole; H¹ NMR (DMSO-□): 9.26 (1H, s), 8.78 (1H, d), 8.45 (1H, d), 7.7(1H, dd); MS: 120.8 (M+1).

Step 2. Pyridyloxazole (600 mg, 5 mmol) in 30 ml THF was cooled to 0° C. before the addition of isopropanyl magnesium chloride (2M in THF, 2.5 ml, 5 mmol). After stirring for 1 hour at 0° C., (S)-(1-formyl-propyl)-carbamic acid tert-butyl ester (573 mg, 3 mmol, Reference Example 18) in 20 ml THF was added. The ice bath was removed and the reaction allowed to warm to room temperature. The reaction mixture was stirred for 2 hours and quenched with saturated ammonium chloride solution. Excess THF was removed and the residue was extracted with EtOAc, washed with brine, dried with anhydrous MgSO₄, filtered and concentrated. The crude residue was purified by chromatography to yield [1-(hydroxy-oxazolo[4,5-b]pyridin-2-yl-methyl)-propyl}-carbamic acid tert-butyl ester (383 mg) H¹ NMR (DMSO-□): 8.42(1H, m), 8.18(1H, m), 7.3(1H, m), 6.8, 6.6(1H, dd, d, OH, diastereomeric), 6.3, 6.02(1H, d, d, NH, diastereomeric), 4.82, 4.5(1H, m, m, diastereomeric), 1.8-1.3(2H, m), 1.2, 1.05(9H, s,s, diastereomeric), 0.89(3H, m); MS: 306.2(M-1), 308.6(M+1).

Step 3. To a stirred solution of the [1-(hydroxy-oxazolo[4,5-b]pyridin-2-yl-methyl)-propyl]-carbamic acid tert-butyl ester (12 g, 100 mmol) in THF (300 ml) was added n-BuLi (1.6M solution in 62.5 ml of hexane) drop wise under N₂ at −78° C. After 1 hour, MgBr.Et₂O (25.8 g, 100 mmol) was added and the reaction mixture was allowed to warm to −45° C. for 1 hour before being treated with 2-boc-amino-butyl-aldehyde (11.46 g, 60 mmol) in THF (50 ml). The reaction mixture was stirred for 1 hour, quenched with saturated NH₄Cl, and extracted with ethyl acetate. The organic layer was washed with brine, dried with MgSO₄ and concentrated. The residue was purified by silica gel column chromatography to yield 2-boc-amino-1-(5-pyridyloxazole-2-yl)-1-butanol (14.1 g).

Step 4. 2-Boc-amino-1-(5-pyridyloxazole-2-yl)-1-butanol (311 mg, 1 mmol) and MeCl₂ (5 ml) were mixed and TFA (1 ml) was added at room temperature. After stirring for 1 hour, the solvent and excess TFA were removed under vacuum to produce 355 mg of 2-amino-1-oxazolo[4,5-b]pyridin-2-yl-butan-1-ol TFA salt.

### REFERENCE EXAMPLE 21

### (S)-2-Amino-1-(3-phenyl-[1,2,4]oxadiazol-5-yl)-butan-1-ol

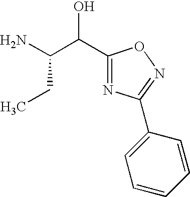

3-tert-Butoxycarbonylamino-2-hydroxy-pentanoic acid (500 mg, 2.14 mmol) was combined with EDC (600 mg, 3.14 mmol), HOBt (600 mg, 3.92 mmol), and N-hydroxy-benzamidine (292 mg, 2.14 mmol). Dichloromethyl (10 mL) was added and then 4-methylmorpholine (1 mL). The mixture was stirred at ambient temperature for 16 hours. After dilution with ethyl acetate (200 mL), the solution was washed with water (30 mL), saturated aqueous NaHCO₃ solution and brine, dried with MgSO₄ and evaporated under vacuum. The crude product was dissolved in pyridine (10 mL) and heated at 80° C. for 15 hours. The pyridine was evaporated under vacuum and the residue was purified by flash chromatography on silica gel (eluent: ethyl acetate) to yield 290 mg (0.83 mmol). The oxadiazole (145 mg, 0.41 mmol) was dissolved in CH₂Cl₂ (4 mL) and TFA (4 mL) was added. After stirring for 1 hour, the mixture was evaporated to dryness to yield (S)-2-Amino-1-(3-phenyl-[1,2,4]oxadiazol-5-yl)-butan-1-ol.

### REFERENCE EXAMPLE 22

### (R)-2-tert-butoxycarbonylamino-3-cyclopropylmethanesulfonyl-propionic acid

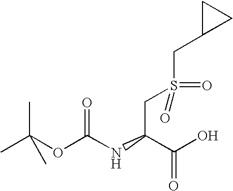

Step 1. Sodium hydroxide (2.16 g, 54 mmol) was dissolved in 27 ml water and the solution added to a suspension of (R)-2-tert-butoxycarbonylamino-3-mercapto-propionic acid (8.2 g, 37 mmol) in 54 ml methanol. After a clear solution had formed bromomethyl-cyclopropane (5 g, 37 mmol) was added and the resulting reaction mixture stirred for three days. Methanol was removed under reduced pressure. The residue was treated with 200 ml 1 M hydrochloric acid and then extracted three times with 200 ml of dichloromethane. The combined organic phases were washed with brine and dried with magnesium sulfate. The solvent was evaporated under reduced pressure to give 2-tert-butoxycarbonylamino-3-cyclopropylmethylsulfanyl-propionic acid (7.94 g).

Step 2. Sodium hydroxide (2.32 g, 58 mmol) was dissolved in 75 ml water. 2-tert-butoxycarbonylamino-3-cyclopropylmethylsulfanyl-propionic acid (7.94 g, 29 mmol) was added. A solution of Oxone™ in 100 ml water was added slowly. The pH was adjusted to 3 by addition of sodium bicarbonate and the reaction mixture stirred for 30 minutes. It was extracted three times with 200 ml ethyl acetate. The combined organic phases were washed with 100 ml brine and dried with magnesium sulfate. The solvent was removed to yield (R)-2-tert-butoxycarbonylamino-3-cyclopropylmethanesulfonyl-propionic acid (4.64 g, 15 mmol, 31%).

### REFERENCE EXAMPLE 23

### (S)-2-Amino-1-(5-ethyl-1,2,4-oxadiazol-3-yl)-butan-1-ol trifluoro-acetic acid salt

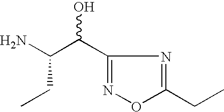

Step 1. A solution of (2-Cyano-1-ethyl-2-hydroxy-ethyl)-carbamic acid tert-butyl ester (1, 9.53 g, 44 mmol) in methanol (80 ml) was cooled to 0° C. and treated successively with hydroxylamine hydrochloride (3.05, 44 mmol) in methanol (80 ml) and 25% sodium methoxide solution in methanol (10.2 ml). Stirred at 0° C. for 5 min., cold bath removed and the reaction mixture stirred at room temperature for 5 hr. Methanol evaporated off under reduced pressure, crude partitioned between ethyl acetate and water. Organic layer separated, dried (MgSO4) and evaporated under reduced pressure to give yellow oil. Purified by mplc eluting with a mixture of ethyl acetate—heptane to give {(S)-1-[Hydroxy-(N!-hydroxycarbamimidoyl)-methyl]-propyl}-carbamic acid tert-butyl ester as white solid (3.5 g).

MS: M(H⁺) 248.

Step 2. A mixture of {(S)-1-[Hydroxy-(N!-hydroxycarbamimidoyl)-methyl]-propyl}-carbamic acid tert-butyl ester (525 mg, 2.16 mmol), propionic anhydride (0.3 ml, 2.37 mmol) in dioxane (5 ml) was heated at 150° C. in a microwave (Smith Creator, S00219) for 35 min. Crude evaporated under reduced pressure and purified by flash column chromatography to give {(S)-1-[(5-Ethyl-1,2,4-oxadiazol-3-yl)-hydroxy-methyl]-propyl}-carbamic acid tert-butyl ester as yellow solid (0.8 g, 67%).

H¹ NMR (CDCl₃): 4.88-4.80 (2H, m), 4.01-3.84 (1H, 2 broad m), 3.64-3.45 (1H, 2 bs), 2.95-2.86 (2H, dq, J=4.2 Hz, 7.6 Hz), 1.73-1.62 (1H, m), 1.6-1.32 (13H, m), 1.02-0.94 (3H, q J=7.5 Hz). MS: 304(M+1)

Step 3. {(S)-1-[(5-Ethyl-1,2,4-oxadiazol-3-yl)-hydroxy-methyl]-propyl}-carbamic acid tert-butyl ester (214 mg, 0.75 mmol) in dichloromethane (3 ml)) was treated with trifluoro acetic acid at room temperature for 3 h. Solvent evaporated under reduced pressure to give (S)-2-Amino-1-(5-ethyl-1,2,4-oxadiazol-3-yl)-butan-1-ol trifluoro-acetic acid salt as brown oil (0.3 g). H¹ NMR (CDCl₃): 7.9-7.4(3H, 2 bs), 5.07 & 5.24 (1H, 2×d, J=3.5 Hz & 5.5 Hz), 3.8-3.6 (1H, 2 bs), 2.96-2.87 (2H, dq, J=2.4 Hz, 7.5 Hz), 1.8-1.4 (2H, m), 1.40-1.34 (3H, dt, J=1.4 Hz, 7.5 Hz), 1.06-0.98 (3H, dt, J=7.5 Hz, 10.5 Hz).

MS: 186(M+1)

### EXAMPLE 1

### (a) (R)-N-cyanomethyl-2-hydroxy-3-phenylmethanesulfonyl-propionamide, (Compound 4)

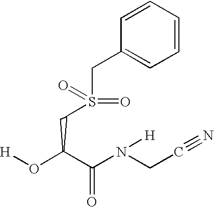

DMF (5 mL) was added to a mixture of 2-hydroxy-3-phenylmethanesulfonyl-propionic acid [200 mg, 0.82 mmol, Reference Example 1(b)], EDC (300 mg, 1.57 mmol), HOBt (300 mg, 1.96 mmol) and aminoacetonitrile hydrochloride (200 mg, 2.1 mmol). 4-Methylmorpholine (0.5 mL) was added and the mixture was stirred at ambient temperature for 2 hours. The mixture was diluted with ethyl acetate (200 mL), washed with 1N HCl, brine, saturated aqueous NaHCO₃ solution, and brine, dried with MgSO₄ and evaporated under vacuum. (R)-N-cyanomethyl-2-hydroxy-3-phenylmethanesulfonyl-propionamide was crystallized from ethyl acetate/hexane to yield 154 mg (0.55 mmol); ¹H NMR: (DMSO) 8.89-8.77 (m, 1H), 7.46-7.37 (m, 5H), 6.71-6.62 (m, 1H), 4.60-4.45 (m, 3H), 4.17-4.08 (m, 2H), 3.39-3.28 (m, 2H). MS: (M⁺+1) 283.

### (b) (R)-N-(1-cyano-1-thiophen-2-yl-methyl)-2-hydroxy-3-phenylmethanesulfonyl-propionamide, (Compound 7)

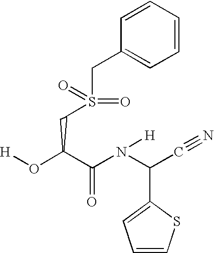

By proceeding in a manner similar to Example 1 (a) above but using (R)-2-hydroxy-3-phenylmethanesulfonyl-propionic acid [Reference Example 1(b)] and DL-α-amino-2-thiopheneacetic acid there was prepared (R)-N-(1-cyano-1-thiophen-2-yl-methyl)-2-hydroxy-3-phenylmethanesulfonyl-propionamide. ¹H NMR (DMSO): 9.55(d, J=6.5 Hz, 1H), 7.58(d, J=5.21 Hz, 1H), 7.42-7.39(m, 5H), 7.23(m, 1H), 7.05(dd, J=3.51 Hz, J=5.21 Hz, 1H), 6.58(dd, J=3.45 Hz, J=6.66 Hz, 1H), 6.41(s, 1H), 4.59-4.50(m, 3H), 3.29(s, 2H); MS: 362.6(M⁻¹), 365(M⁺¹).

### (c) (R)-N-(1-cyano-1-thiophen-2-yl-methyl)-3-[2-(1,1-difluoro-methoxy)-phenylmethanesulfonyl]-2-hydroxy-propionamide, (Compound 8)

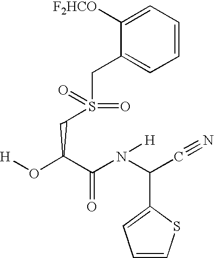

By proceeding in a manner similar to Example 1(a) above but using (R)-3-[2-(1,1-difluoro-methoxy)-phenylmethanesulfonyl]-2-hydroxy-propionic acid [Reference Example 1(a)] and DL-α-amino-2-thiopheneacetic acid there was prepared (R)-N-(1-cyano-1-thiophen-2-yl-methyl)-3-[2-(1,1-difluoro-methoxy)-phenylmethanesulfonyl]-2-hydroxy-propionamide. ¹HNMR (CD₃Cl): δ7.6-7.2(m, 7H), 7.01(t, J=73.6 Hz, 1H), 6.62(s, 1H), 6.21(d, J=8.15, 1H), 4.71-4.67(m, 1H), 4.46(s, 2H), 3.68(s, 2H), 3.22-3.18(m, 1H); MS: 428.6(M−1), 453(M+23).

### (d) (R)-N-cyanomethyl-3-[2-(1,1-difluoro-methoxy)-phenylmethanesulfonyl]-2-hydroxy-propionamide, (Compound 17)

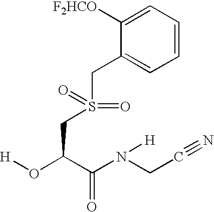

By proceeding in a manner similar to Example 1(a) above but using (R)-3-[2-(1,1-difluoro-methoxy)-phenylmethanesulfonyl]-2-hydroxy-propionic acid [Reference Example 1(a)] there was prepared (R)-N-cyanomethyl-3-[2-(1,1-difluoro-methoxy)-phenylmethanesulfonyl]-2-hydroxy-propionamide. ¹HNMR (DMSO): 8.81(t, J=5.67 Hz, 1H), 7.55-7.4(m, 2H), 7.35-7.2(m, 2H), 7.13(t, J=73.68 Hz, 1H), 6.62(d, J=6.67 Hz, 1H), 4.58(s, 2H), 4.52-4.45(m, 1H), 4.12(d, J=5.94 Hz, 2H), 3.45-3.4(m, 2H). MS: 347.4(M−1), 371(M+23).

### EXAMPLE 2

### Morpholine-4-carboxylic acid (R)-1-(cyanomethyl-carbamoyl)-2-phenylmethanesulfonyl-ethyl ester, (Compound 6);

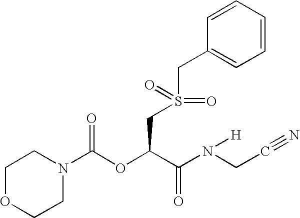

Phosgene solution (0.77 mL, 1.93M in toluene) was added to CH₂Cl₂ (5 mL) and cooled to 0° C. under nitrogen. Quinoline (0.12 mL, 1.0 mmol) was added followed by (R)-N-cyanomethyl-2-hydroxy-3-phenylmethanesulfonyl-propionamide [100mg, 0.354 mmol, Example 1(a)]. The mixture was stirred at ambient temperature for 3 hours. Morpholine (1 mmol) was added and stirring was continued for 3 hours. The mixture was diluted with ethyl acetate (200 mL) and washed sequentially with 1N HCl, brine, saturated aqueous NaHCO₃ solution and brine. The product was dried with MgSO₄ and evaporated under vacuum and crystallized from an ethyl acetate/hexane solution to yield morpholine-4-carboxylic acid (R)-1-(cyanomethyl-carbamoyl)-2-phenylmethanesulfonyl-ethyl ester. (85 mg; 0.215 mmol); ¹H NMR: (DMSO) 8.99-8.88 (m, 1H), 7.46-7.37 (m, 5H), 5.42-5.32 (m, 1H), 4.60-4.45 (m, 2H), 4.20-4.13 (m, 2H), 3.70-3.28 (m, 10H). MS: (M⁺+1) 396.

### EXAMPLE 3

### (a) Morpholine-4-carboxylic acid (R)-1-(cyanomethyl-carbamoyl)-2-[2-(1,1-difluoro-methoxy)-phenylmethanesulfonyl]-ethyl ester, (Compound 31)

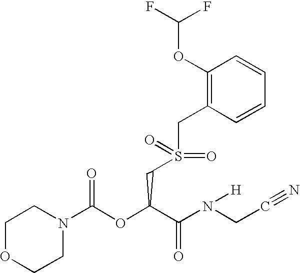

(R)-N-cyanomethyl-3-[2-(1,1-difluoro-methoxy)-phenylmethanesulfonyl]-2-hydroxy-propionamide [100 mg, 0.287 mmol, Example 1(d)], was dissolved in CH₂Cl₂ (2 mL). Pyridine (32.4 μL, 0.4 mmol) and then trichloromethylchloroformate (36.2 μL, 0.3 mmol) were added. The mixture was stirred at ambient temperature for 3 hours. Morpholine (0.5 mL) was added and stirring was continued for 3 hours. The mixture was diluted with ethyl acetate (200 mL), washed with 1N HCl, brine, saturated aqueous NaHCO₃ solution and brine. The product was dried with MgSO₄, evaporated under vacuum and crystallized from a solution of ethyl acetate/hexane to yield moroholine-4-carboxylic acid (R)-1-(cyanomethyl-carbamoyl)-2-[2-(1,1-difluoro-methoxy)-phenylmethanesulfonyl]-ethyl ester (60 mg; 0.130 mmol); ¹H NMR: (DMSO) δ8.95 (t, J=5.2 Hz, 1H), 7.51-7.44 (m, 2H), 7.32-7.22 (m, 2H), 7.14 (t, J_{H,F}=73 Hz, 1H), 5.39-5.35 (m, 1H), 4.67-4.53 (m, 2H), 4.19-4.15 (m, 2H), 3.83-3.28 (m, 10H); MS: (M⁺+1) 462.

### (b) (R)-(2-Methoxy-ethyl)-carbamic acid 1-(cyanomethyl-carbamoyl)-2-phenylmethanesulfonyl-ethyl ester

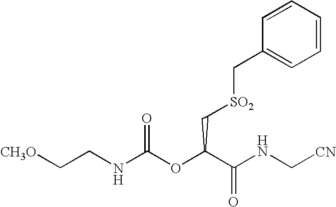

By proceeding in a manner similar to Example 3(a) above but using (R)-N-cyanomethyl-2-hydroxy-3-phenylmethanesulfonyl-propionamide [Example 1(a)] and 2-methoxyethylamine there was prepared (R)-(2-Methoxy-ethyl)-carbamic acid 1-(cyanomethyl-carbamoyl)-2-phenylmethanesulfonyl-ethyl ester. ¹H NMR: (DMSO) 8.91 (t, J=5.6 Hz, 1H), 7.64 (t, J=5.6 Hz, 1H), 7.40-7.32 (m, 5H), 5.30-5.25 (m, 1H), 4.59-4.50 (m, 2H), 4.17-4.13 (m 2H), 3.58 (dd, J=9.2 Hz, J=14.8 Hz, 1H), 3.43 (d, 14.8 Hz, 1H), 3.33 (s, 3H), 3.38-3.12 (m, 4H). MS: (M+H)⁺ 384.

### (c) (S)-Diethyl-carbamic acid 1-(cyanomethyl-carbamoyl)-2-cyclohexyl-ethyl ester

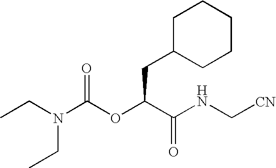

By proceeding in a manner similar to Example 3(a) above but using (R)-N-cyanomethyl-3-cyclohexyl-2-hydroxy-propionamide and diethylamine there was prepared (S)-Diethyl-carbamic acid 1-(cyanomethyl-carbamoyl)-2-cyclohexyl-ethyl ester. ¹H NMR: (DMSO) 8.62 (t, J=5.6 Hz, 1H), 4.87-4.82 (m, 1H), 4.12 (d, J=5.6, 2H), 3.42-3.10 (m, 4H), 1.72-0.82 (m, 19H). MS: (M+H)⁺ 310.

### (d) (S)-Pyrrolidine-1-carboxylic acid 1-(cyanomethyl-carbamoyl)-2-cyclohexyl-ethyl ester

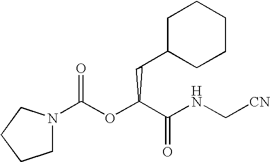

By proceeding in a manner similar to Example 3(a) above but using (R)-N-cyanomethyl-3-cyclohexyl-2-hydroxy-propionamide and pyrrolidine there was prepared (S)-Pyrrolidine-1-carboxylic acid 1-(cyanomethyl-carbamoyl)-2-cyclohexyl-ethyl ester. ¹H NMR: (DMSO) 8.59 (t, J=4.8 Hz, 1H), 4.86-4.81 (m, 1H), 4.11 (d, J=4.8, 2H), 3.48-3.19 (m, 4H), 1.87-0.82 (m, 17H). MS: (M+H)⁺ 308.

### (e) (S)-Morpholine-4-carboxylic acid 1-(cyanomethyl-carbamoyl)-2-cyclohexyl-ethyl ester

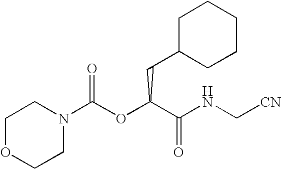

By proceeding in a manner similar to Example 3(a) above but using (R)-N-cyanomethyl-3-cyclohexyl-2-hydroxy-propionamide and morpholine there was prepared (S)-Morpholine-4-carboxylic acid 1-(cyanomethyl-carbamoyl)-2-cyclohexyl-ethyl ester. ¹H NMR: (DMSO) 8.66 (t, J=5.2 Hz, 1H), 4.88-4.83 (m, 1H), 4.13 (d, J=4.8, 2H), 3.60-3.26 (m, 8H), 1.71-0.82 (m, 13H). MS: (M+H)⁺ 324.

### (f) (S)-4-Ethyl-piperazine-1-carboxylic acid 1-(cyanomethyl-carbamoyl)-2-cyclohexyl-ethyl ester

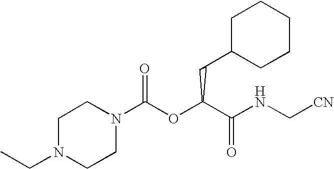

By proceeding in a manner similar to Example 3(a) above but using (R)-N-cyanomethyl-3-cyclohexyl-2-hydroxy-propionamide and 4-ethylpiperazine there was prepared (S)-4-Ethyl-piperazine-1-carboxylic acid 1-(cyanomethyl-carbamoyl)-2-cyclohexyl-ethyl ester. LC-MS: elution time=2.08 min. 349.2(M−1), 351.3(M+1). (MS: API 150EX. LC: HP Agilent 1100 Series. Column: Phenomenex, 5 u ODS3 100 A 100×3 mm.; Flow Rate: 2 ml/min. Two solvent gradient: Solvent A, 99% water, 1% acetonitrile, 0.1% AcOH. Solvent B, 99% actonitrile, 1% water, 0.1% AcOH. Gradient from 100% A, 0% B to 0% A, 100% B from t=0 to t=6 min. Then gradient back to 100% A, 0% B from t=7 to t=15 min.).

### (g) (S)-2-Hydroxymethyl-pyrrolidine-1-carboxylic acid (S)-1-(cyanomethyl-carbamoyl)-2-cyclohexyl-ethyl ester

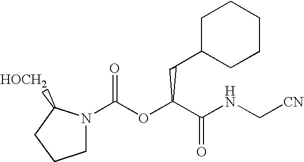

By proceeding in a manner similar to Example 3(a) above but using (R)-N-cyanomethyl-3-cyclohexyl-2-hydroxy-propionamide and (S)-2-hydroxymethyl-pyrrolidine there was prepared (S)-2-Hydroxymethyl-pyrrolidine-1-carboxylic acid (S)-1-(cyanomethyl-carbamoyl)-2-cyclohexyl-ethyl ester. LC-MS: elution time=3.73 min. 336.5(M−1), 338.2(M+1). (MS: API 150EX. LC: HP Agilent 1100 Series. Column: Phenomenex, 5 u ODS3 100 A 100×3 mm.; Flow Rate: 2 ml/min. Two solvent gradient: Solvent A, 99% water, 1% acetonitrile, 0.1% AcOH. Solvent B, 99% actonitrile, 1% water, 0.1% AcOH. Gradient from 100% A, 0% B to 0% A, 100% B from t=0 to t=6 min. Then gradient back to 100% A, 0% B from t=7 to t=15 min.)

### (h) (S)-(2,2,2-Trifluoro-ethyl)-carbamic acid 1-(cyanomethyl-carbamoyl)-2-cyclohexyl-ethyl ester

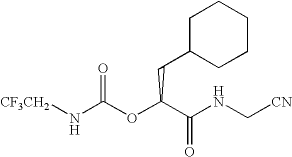

By proceeding in a manner similar to Example 3(a) above but using (R)-N-cyanomethyl-3-cyclohexyl-2-hydroxy-propionamide and 2,2,2-trifluoroethylamine there was prepared (S) (2,2,2-Trifluoro-ethyl)-carbamic acid 1-(cyanomethyl-carbamoyl)-2-cyclohexyl-ethyl ester. LC-MS: elution time=4.07 min. 334.1(M−1), 336.2(M+1). (MS: API 150EX. LC: HP Agilent 1100 Series. Column: Phenomenex, 5 u ODS3 100 A 100×3 mm.; Flow Rate: 2 ml/min. Two solvent gradient: Solvent A, 99% water, 1% acetonitrile, 0.1 % AcOH. Solvent B, 99% actonitrile, 1% water, 0.1% AcOH. Gradient from 100% A, 0% B to 0% A, 100% B from t=0 to t=6 min. Then gradient back to 100% A, 0% B from t=7 to t=15 min.)

### (i) (S)-(2-Hydroxyethyl)-carbamic acid 1-(cyanomethyl-carbamoyl)-2-cyclohexyl-ethyl ester

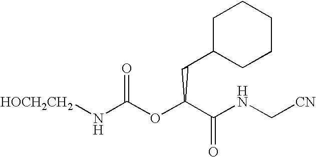

By proceeding in a manner similar to Example 3(a) above but using (R)-N-cyanomethyl-3-cyclohexyl-2-hydroxy-propionamnide and 2-hydroxyethylamine there was prepared (S)-(2-Hydroxyethyl)-carbamic acid 1-(cyanomethyl-carbamoyl)-2-cyclohexyl-ethyl ester. ¹H NMR: (DMSO) 8.65 (t, J=5.2 Hz, 1H), 7.16 (t, J=5.2 Hz, 1H), 4.85-4.80 (m, 1H), 4.62 (t, J=5.6 Hz, 1H), 4.12 (d, J=5.6 Hz, 2H), 3.45-3.33 (m, 2H), 3.12-2.96 (m, 2H), 1.74-0.80 (m, 13H). MS: (M+H)⁺ 298.

### (j) (Tetrahydrofuran-2-ylmethyl)-carbamic acid (S)-1-(cyanomethyl-carbamoyl)-2-cyclohexyl-ethyl ester

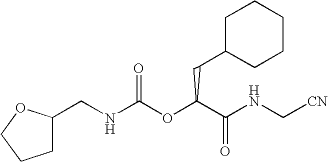

By proceeding in a manner similar to Example 3(a) above but using (R)-N-cyanomethyl-3-cyclohexyl-2-hydroxy-propionamide and tetrahydrofurfurylamine there was prepared (tetrahydrofuran-2-ylmethyl)-carbamic acid (S)-1-(cyanomethyl-carbamoyl)-2-cyclohexyl-ethyl ester as a 1:1 mixture of diastereomers, ¹H NMR: (DMSO) 8.66 (t, J=5.2 Hz, 1H), 7.28 (t, J=5.2 Hz, 1H), 4.86-4.81 (m, 1H), 4.12 (d, J=5.2 Hz, 2H), 3.83-3.54 (m, 3H), 3.09-2.92 (m, 2H), 1.89-0.80 (m, 17H). MS: (M+H)⁺ 338.

### (k) (S)-Azetidine-1-carboxylic acid 1-(cyanomethyl-carbamoyl)-2-cyclohexyl-ethyl ester

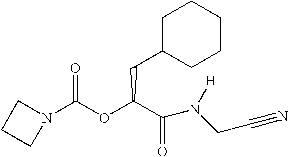

By proceeding in a mariner similar to Example 3(a) above but using (R)-N-cyanomethyl-3-cyclohexyl-2-hydroxy-propionamide and azetidine there was prepared (S)-azetidine-1-carboxylic acid 1-(cyanomethyl-carbamoyl)-2-cyclohexyl-ethyl ester. ¹H NMR: (DMSO) 8.59 (t, J=5.2 Hz, 1H), 4.82-4.77 (m, 1H), 4.11 (d, J=5.2 Hz, 2H), 4.13-3.81 (m, 4H), 2.18 (quint, J=7.6 Hz, 2H), 1.71-0.80 (m, 13H). MS: (M+H)⁺ 294.

### (l) (S)-Cyclopropyl-carbamic acid 1-(cyanomethyl-carbamoyl)-2-cyclohexyl-ethyl ester

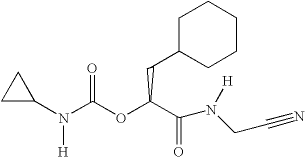

By proceeding in a manner similar to Example 3(a) above but using (R)-N-cyanomethyl-3-cyclohexyl-2-hydroxy-propionamide and cyclopropylamine there was prepared (S)-cyclopropyl-carbamic acid 1-(cyanomethyl-carbamoyl)-2-cyclohexyl-ethyl ester. ¹H NMR: (DMSO) 8.64 (t, J=5.2 Hz, 1H), 7.44 (br, 1H), 4.83-4.78 (m, 1H), 4.11 (d, J=5.2 Hz, 2H), 2.50-2.40 (m, 1H), 1.72-0.78 (m, 13H), 0.58-0.30 (m, 4H). MS: (M+H)⁺ 294.

### (m) (S)-Piperidine-1-carboxylic acid 1-(cyanomethyl-carbamoyl)-2-cyclohexyl-ethyl ester

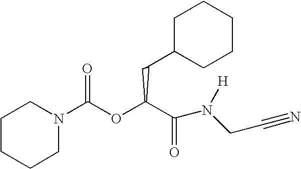

By proceeding in a manner similar to Example 3(a) above but using (R)-N-cyanomethyl-3-cyclohexyl-2-hydroxy-propionamide and piperidine there was prepared (S)-piperidine-1-carboxylic acid 1-(cyanomethyl-carbamoyl)-2-cyclohexyl-ethyl ester. ¹H NMR: (DMSO) 8.63 (t, J=5.2 Hz, 1H), 4.86-4.81 (m, 1H), 4.11 (d, J=5.6 Hz, 2H), 3.48-3.20 (m, 4H), 1.70-0.82 (m, 19H). MS: (M+H)⁺ 322.

### (n) (S)-(2-Methoxy-ethyl)-carbamic acid 1-(cyanomethyl-carbamoyl)-2-cyclohexyl-ethyl ester

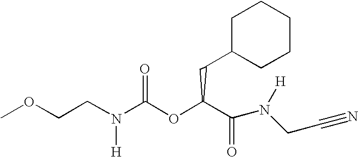

By proceeding in a manner similar to Example 3(a) above but using (R)-N-cyanomethyl-3-cyclohexyl-2-hydroxy-propionamide and 2-methoxyethylamine there was prepared (S)-(2-methoxy-ethyl)-carbamic acid 1-(cyanomethyl-carbamoyl)-2-cyclohexyl-ethyl ester. ¹H NMR: (DMSO) 8.66 (t, J=5.6 Hz, 1H), 7.27 (t, J=5.6 Hz, 1H), 4.85-4.80 (m, 1H), 4.12(d, J=5.6 Hz, 2H), 3.40-3.03 (m, 4H), 3.32 (s, 3H), 1.74-0.80 (m, 13H). MS: (M+H)⁺ 312.

### (o) (R)-3-Hydroxy-pyrrolidine-1-carboxylic acid (S)-1-(cyanomethyl-carbamoyl)-2-cyclohexyl-ethyl ester

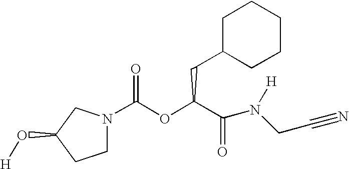

By proceeding in a manner similar to Example 3(a) above but using (R)-N-cyanomethyl-3-cyclohexyl-2-hydroxy-propionamide and (R)-3-hydroxy-pyrrolidine there was prepared (R)-3-hydroxy-pyrrolidine-1-carboxylic acid (S)-1-(cyanomethyl-carbamoyl)-2-cyclohexyl-ethyl ester. ¹H NMR: (DMSO) 8.64-8.56 (m, 1H), 4.98-4.80 (m, 2H), 4.29-4.20 (m, 1H), 4.11 (d, J=5.2 Hz, 2H), 3.57-3.12 (m, 4H), 1.91-0.82 (m, 15H). MS: (M+H)⁺ 324.

### (p) (S)-3-Hydroxy-pyrrolidine-1-carboxylic acid (S)-1-(cyanomethyl-carbamoyl)-2-cyclohexyl-ethyl ester

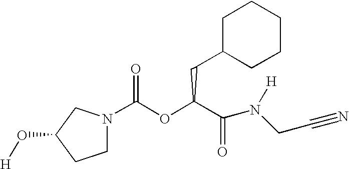

By proceeding in a manner similar to Example 3(a) above but using (R)-N-cyanomethyl-3-cyclohexyl-2-hydroxy-propionamide and (S)-3-hydroxy-pyrrolidine there was prepared (S)-3-hydroxy-pyrrolidine-1-carboxylic acid (S)-1-(cyanomethyl-carbamoyl)-2-cyclohexyl-ethyl ester. ¹H NMR: (DMSO) 8.63-8.55 (m, 1H), 4.98-4.90 (m, 1H), 4.85-4.80 (m, 1H), 4.28-4.19 (m, 1H), 4.13-4.09 (m, 2H), 3.54-3.09 (m, 4H), 1.93-0.81 (m, 15H). MS: (M+H)⁺ 324.

### (q) (S)-Morpholine-4-carboxylic acid 1-(cyanomethyl-carbamoyl)-3-cyclohexyl-propyl ester

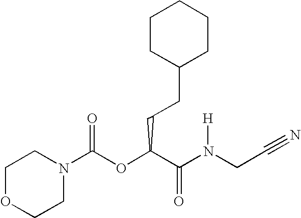

By proceeding in a manner similar to Example 3(a) above but using (R)-N-cyanomethyl-3-cyclohexyl-2-hydroxy-propionamide and morpholine there was prepared (S)-morpholine-4-carboxylic acid 1-(cyanomethyl-carbamoyl)-3-cyclohexyl-propyl ester. ¹H NMR: (DMSO) 8.61 (t, J=5.6 Hz, 1H), 4.79 (t, J=5.6 Hz, 1H), 4.13 (d, J=5.2 Hz, 2H), 3.59-3.26 (m, 8H), 1.73-1.55 (m, 7H), 1.23-1.06 (m, 6H), 0.88-0.76 (m, 2H). MS: (M+H)⁺ 338.

### EXAMPLE 4

### (a) Morpholine-4-carboxylic acid (R)-1-[(S)-1-(1-benzooxazol-2-yl-methanoyl)-propylcarbamoyl]-2-phenylmethanesulfonyl-ethyl ester, (Compound 11)

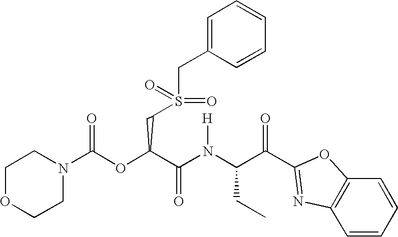

Step 1. (R)-2-Hydroxy-3-phenylmethanesulfonyl-propionic acid {2 g, 8.19 mmol, Reference Example 1(b)} was dissolved in CH₂Cl₂ (20 mL). 4-Methylmorpholine (3.8 mL) and then chloromethyl methyl ether (1.52 mL, 20 mmol) were added. After stirring at ambient temperature for 30 minutes, the reaction was quenched with water (50 mL) and extracted with ethyl acetate. The combined organic layers were washed with saturated aqueous NaHCO₃ solution and brine. The product was dried with MgSO₄, evaporated under vacuum and crystallized from ethyl acetate/hexane to yield 2-hydroxy-3-phenylmethanesulfonyl-propionic acid methoxymethyl ester (1.2 g; 4.16 mmol).

Step 2. Phosgene solution (2.07 mL, 1.93M in toluene) was added to CH₂Cl₂ (10 mL) and cooled to 0° C. under nitrogen. Quinoline (0.95 mL) was added followed by 2-hydroxy-3-phenylmethanesulfonyl-propionic acid methoxymethyl ester (250 mg, 0.87 mmol). The mixture was stirred at ambient temperature for 3 hours. Morpholine (0.35 mL, 4 mmol) was added and stirring was continued for 3 hours. The mixture was diluted with ethyl acetate (200 mL), washed sequentially with 1N HCl, brine, saturated aqueous NaHCO₃ solution and brine. The crude product was dried with MgSO₄, evaporated under vacuum and dissolved in 1,4-dioxane (20 mL). 1N HCl (10 mL) was added and the mixture was stirred at ambient temperature for 3 hours. Dioxane was evaporated under vacuum and the product was extracted with ethyl acetate. The combined ethyl acetate layers were washed with saturated aqueous NaHCO₃ solution (3×20 mL). The NaHCO₃ solution was acidified with 6N HCl and extracted with ethyl acetate. The combined ethyl acetate layers were washed with brine, dried with MgSO₄ and evaporated under vacuum to give (R)-morpholine-4-carboxylic acid 1-carboxy-2-phenylmethanesulfonyl-ethyl ester.

Step 3. (R)-Morpholine-4-carboxylic acid 1-carboxy-2-phenylmethanesulfonyl-ethyl ester was combined with EDC (250 mg, 1.3 mmol), HOBt (250 mg, 1.6 mmol), and (2S)-2-amino-1-benzooxazol-2-yl-butan-1-ol {250 mg, 1.2 mmol, Reference Example 17(a)}. Dichloromethane (4 mL) was added and then 4-methylmorpholine (0.5 mL). The mixture was stirred at ambient temperature for 2 hours. After dilution with ethyl acetate (150 mL), the solution was washed with 1N aqueous HCl, water, saturated aqueous NaHCO₃ solution and brine, dried with MgSO₄ and evaporated under vacuum. The crude product was dissolved in dry dichloromethane (10 mL) and Dess Martin Periodinane (500 mg, 1.2 mmol) was added. After stirring at ambient temperature for 1 hour, the mixture was diluted with ethyl acetate (150 mL) and treated with 0.26M Na₂S₂O₃ solution in saturated aqueous NaHCO₃. The organic phase was washed with saturated aqueous NaHCO₃ and brine, dried with MgSO4 and evaporated. The product was crystallized from ethyl acetate/hexane to yield morpholine-4-carboxylic acid (R)-1-[(S)-1-(1-benzooxazol-2-yl-methanoyl)-propylcarbamoyl]-2-phenylmethanesulfonyl-ethyl ester (190 mg; 0.35 mmol); ¹H NMR: (DMSO) 8.95 (d, J=6.6 Hz, 1H), 8.01 (d, J=7.9 Hz, 1H), 7.90 (d, J=7.9 Hz, 1H), 7.65 (t, J=7.5 Hz, 1H), 7.55 (t, J=7.9 Hz, 1H), 7.40-7.34 (m, 5H), 5.44-5.35 (m 1H), 5.26-5.16 (m, 1H), 4.60 (d, J=13.6 Hz, 1H), 4.47 (d, J=13.6 Hz, 1H), 3.71-3.28 (m, 10H), 2.10-1.94 (m, 1H), 1.81-1.65 (m, 1H), 0.98 (t, J=7.2 Hz, 3H); MS: (M⁺+1) 544.

### (b) Morpholine-4-carboxylic acid (R)-1-[(S)-1-(1-benzooxazol-2-yl-methanoyl)-propylcarbamoyl]-2-[2-(1,1-difluoro-methoxy)-phenylmethanesulfonyl]-ethyl ester, (Compound 14)

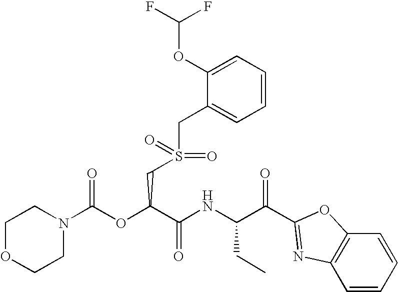

By proceeding in a manner similar to Example 4(a) above but using (R)-3-[2-(1,1-difluoro-methoxy)-phenylmethanesulfonyl]-2-hydroxy-propionic acid {Reference Example 1(a)} in step 1 there was prepared morpholine-4-carboxylic acid (R)-1-[(S)-1-(1-benzooxazol-2-yl-methanoyl)-propylcarbamoyl]-2-[2-(1,1-difluoro-methoxy)-phenylmethanesulfonyl-ethyl ester ¹H NMR: (DMSO) 8.95 (d, J=6.4 Hz, 1H), 8.01 (d, J=7.9 Hz, 1H), 7.90 (d, J=8.4 Hz, 1H), 7.65 (t, J=7.4 Hz, 1H), 7.54 (t, J=7.5 Hz, 1H), 7.52-7.43 (m, 2H), 7.31-7.21 (m, 2H), 7.11 (t, J_{H,F}=73 Hz, 1H), 5.43-5.37 (m 1H), 5.27-5.17 (m, 1H), 4.63 (d, J=13.5 Hz, 1H), 4.54 (d, J=13.5 Hz, 1H), 3.88-3.28 (m, 10H), 2.10-1.94 (m, 1H), 1.81-1.65 (m, 1H), 0.98 (t, J=7.6 Hz, 3H); MS: (M⁺+1) 610.

### (c) morpholine-4-carboxylic acid (R)-1-[(S)-1-(1-benzothiazol-2-yl-methanoyl)-propylcarbamoyl]-2-[2-(1,1-difluoro-methoxy)-phenylmethanesulfonyl]-ethyl ester, (Compound 15).

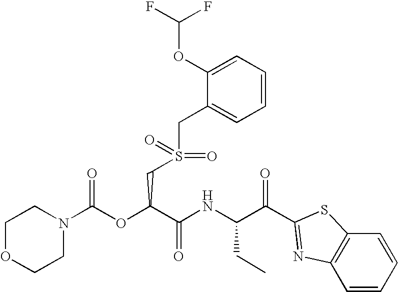

By proceeding in a manner similar to Example 4(a) above but using (R)-3-[2-(1,1-difluoro-methoxy)-phenylmethanesulfonyl]-2-hydroxy-propionic acid {Reference Example 1(a)} in step 1 and (2S)-2-amino-1-benzothiazol-2-yl-butan-1-ol {Reference Example 17(b)} in step 3 there was prepared morpholine-4-carboxylic acid (R)-1-[(S)-1-(1-benzothiazol-2-yl-methanoyl)-propylcarbamoyl]-2-[2-(1,1-difluoro-methoxy)-phenylmethanesulfonyl]-ethyl ester. ¹H NMR: (DMSO) 8.93 (d, J=6.4 Hz, 1H), 8.30-8.24 (m, 2H), 7.72-7.62 (m, 2H), 7.51-7.44 (m, 2H), 7.32-7.22 (m, 2H), 7.12 (t, J_{H,F}=73 Hz, 1H), 5.49-5.35 (m 2H), 4.64(d, J=13.5 Hz, 1H), 4.55 (d, J=13.5 Hz, 1H), 3.91-3.28 (m, 10H), 2.08-1.94 (m, 1H), 1.84-1.68 (m, 1H), 0.99 (t, J=7.6 Hz, 3H). MS: (M⁺+1) 626.

### (d) Pyrrolidine-1-carboxylic acid (R)-1-[(S)-1-(1-benzooxazol-2-yl-methanoyl)-propylcarbamoyl]-2-phenylmethanesulfonyl-ethyl ester (Compound 19).

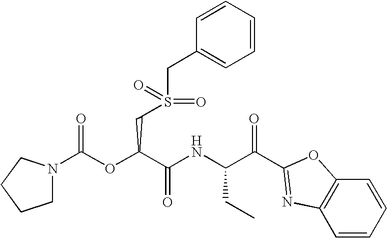

By proceeding in a manner similar to Example 4(a) above but using pyrrolidine in step 2 there was prepared pyrrolidine-1-carboxylic acid (R)-1-[(S)-1-(1-benzooxazol-2-yl-methanoyl)-propylcarbamoyl]-2-phenylmethanesulfonyl-ethyl ester. ¹H NMR: (DMSO) 8.90 (d, J=6.4 Hz, 1H), 7.99 (d, J=7.9 Hz, 1H), 7.89 (d, J=8.4 Hz, 1H), 7.65 (t, J=7.4 Hz, 1H), 7.54 (t, J=7.5 Hz, 1H), 7.40-7.33 (m, 5H), 5.41-5.33 (m 1H), 5.26-5.15 (m, 1H), 4.59 (d, J=13.5 Hz, 1H), 4.47 (d, J=13.5 Hz, 1H), 3.66-3.17 (m, 6H), 2.10-1.64 (m, 6H), 0.97 (t, J=7.2 Hz, 3H); MS: (M³⁰ 1) 528.

### (e) Dimethyl-carbamic acid (R)-1-[(S)-1-(1-benzooxazol-2-yl-methanoyl)-propylcarbamoyl]-2-phenylmethanesulfonyl-ethyl ester, (Compound 20).

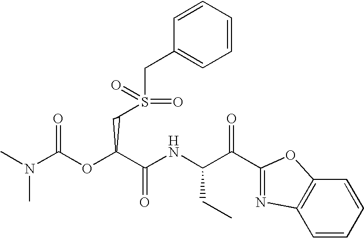

By proceeding in a manner similar to Example 4(a) above but using dimethylamine in step 2 there was prepared dimethyl-carbamic acid (R)-1-[(S)-1-(1-benzooxazol-2-yl-methanoyl)-propylcarbamoyl]-2-phenylmethanesulfonyl-ethyl ester. ¹H NMR: (DMSO) 8.91 (d, J=6.4 Hz, 1H), 7.99 (d, J=7.9 Hz, 1H), 7.90 (d, J=8.4 Hz, 1H), 7.65 (t, J=7.4 Hz, 1H), 7.54 (t, J=7.5 Hz, 1H), 7.40-7.33 (m, 5H), 5.39-5.33 (m 1H), 5.26-5.15 (m, 1H), 4.59 (d, J=13.5 Hz, 1H), 4.47 (d, J=13.5 Hz, 1H), 3.63 (dd, J=14.8 Hz, J=10.6 Hz, 1H), 3.42 (dd, J=14.8 Hz, J=2.5 Hz, 1H), 2.89 (s, 3H), 2.79 (s, 3H), 2.10-1.94 (m, 1H), 1.81-1.64 (m, 1H), 0.97 (t, J=7.2 Hz, 3H); MS: (M⁺+1) 502.

### (f) Morpholine-4-carboxylic acid (R)-1-[(S)-1-(1-benzylcarbamoyl-methanoyl)-propylcarbamoyl]-2-phenylmethanesulfonyl-ethyl ester, (Compound 25).

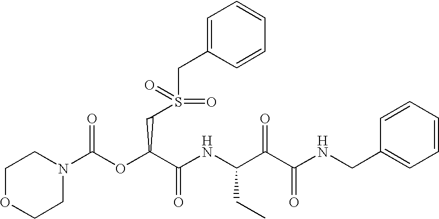

By proceeding in a manner similar to Example 4(a) above but using (R)-3-amino-2-hydroxy-pentanoic acid benzylamide TFA salt (Reference Example 19) in step 3 there was prepared morpholine-4-carboxylic acid (R)-1-[(S)-1-(1-benzylcarbamoyl-methanoyl)-propylcarbamoyl]-2-phenylmethanesulfonyl-ethyl ester. ¹H NMR: (DMSO) 9.27 (t, J=5.5 Hz, 1H), 8.67 (d, J=8.1 Hz, 1H), 7.40-7.20 (m, 10H), 5.42-5.34 (m 1H), 4.96-4.85 (m, 1H), 4.64-4.24 (m, 4H), 3.66-3.28 (m, 10H), 1.90-1.72 (m, 1H), 1.63-1.46 (m, 1H), 0.89 (t, J=7.2 Hz, 3H); MS: (M⁺+1) 560.

### (g) Morpholine-4-carboxylic acid (S)-1-[(S)-1-(oxazolo[4,5-b]pyridine-2-carbonyl)-propylcarbamoyl]-2-phenylmethanesulfonyl-ethyl ester

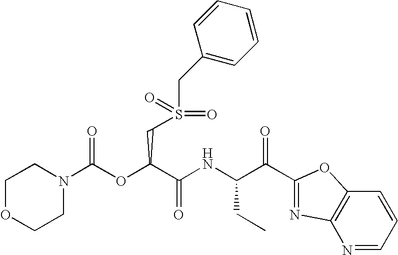

By proceeding in a manner similar to Example 4(a) above but using (S)-2-amino-1-oxazolo[4,5-b]pyridin-2-yl-butan-1-ol TFA salt (Reference Example 20) there was prepared morpholine-4-carboxylic acid (S)-1-[(S)-1-(oxazolo[4,5-b]pyridine-2-carbonyl)-propylcarbamoyl]-2-phenylmethanesulfonyl-ethyl ester. ¹H NMR: (DMSO) 9.00 (d, J=6.4 Hz, 1H), 8.73 (m, 1H), 8.39 (d, J=8.4 Hz, 1H), 7.69-7.64 (m, 1H), 7.45-7.30 (m, 5H), 5.37 (d, J=10.4 Hz, 1H), 5.19-5.13 (m, 1H), 4.57 (d, J=13.6 Hz, 1H),4.46 (d, J=13.6 Hz, 1H), 3.67-3.23 (m, 10H), 2.10-1.98 (m, 1H), 1.80-1.69 (m, 1H), 0.99 (t, J=7.0 Hz, 3H). MS: (M+H)⁺ 545.

### (h) Morpholine-4-carboxylic acid (S)-1-[(S)-1-(5-ethyl-[1,3,4]oxadiazole-2-carbonyl)-propylcarbamoyl]-2-phenylmethanesulfonyl-ethyl ester

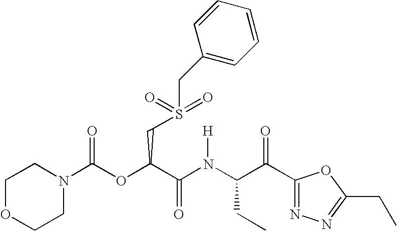

By proceeding in a manner similar to Example 4(a) above but using 2-amino-1-(5-ethyl-[1,3,4]oxadiazol-2-yl-butan-1-ol {Reference Example 11(m)}there was prepared morpholine-4-carboxylic acid (S)-1-[(S)-1-(5-ethyl-[1,3,4]oxadiazole-2-carbonyl)-propylcarbamoyl]-2-phenylmethanesulfonyl-ethyl ester. ¹H NMR: (DMSO) 8.95 (d, J=6.0 Hz, 1H), 7.41-7.33 (m, 5H), 5.35 (d, J=10.0 Hz, 1H), 4.97-4.91 (m, 1H), 4.63-4.45 (m, 2H), 3.64-3.23 (m, 10H), 2.96 q, J=7.2 Hz, 2H), 1.99-1.89 (m, 1H), 1.75-1.64 (m, 1H), 1.30 (t, J=7.6 Hz, 3H), 0.94 (t, J=7.2 Hz, 3H). MS: (M+H)⁺ 523.

### EXAMPLE 5

### (S)-2-{(R)-3-[2-(1,1-Difluoro-methoxy)-phenylmethanesulfonyl]-2-hydroxy-propanoylamino}-N-methoxy-N-methyl-butyramide, (Compound 32)

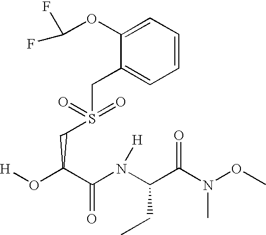

To a solution of (R)-3-[2-(1,1-difluoro-methoxy)-phenylmethanesulfonyl]-2-hydroxy-propionic acid {1.24 g, 4 mmol, Reference Example 1(a)} in CH₂Cl₂ (20 ml) was added HOBt (0.74 g, 4.8 mmol), EDC (1.15 g, 6 mmol), (R)-2-amino-N-methoxy-N-methyl-butyramide TFA salt (1.04 g, 4 mmol), prepared as in reference 2, and NMM (1.6 g, 16 mmol). After stirring for 14 hours at room temperature, the reaction mixture was diluted with 150 ml of ethyl acetate. The mixture was washed with saturated NaHCO₃ and brine before drying with anhydrous MgSO₄. This crude product was then filtered, concentrated and purified by flash column chromatography using silica gel with hexane/acetate as eluent to yield (S)-2-{(R)-3-[2-(1,1-difluoro-methoxy)-phenylmethanesulfonyl]-2-hydroxy-propanoylamino}-N-methoxy-N-methyl-butyramide (1.45 g), 1HNMR (CD3Cl): 7.6-7.5(d, J=7.67 Hz, 1H), 7.5-7.35(m, 2H), 7.31-7.15(m, 2H), 6.63(t, J=73.4 Hz, 1H), 5.0-4.85(br., 1H), 4.7-4.6(m, 1H), 4.55-4.48(m, 2H), 4.45-4.35(m, 1H), 3.80(s, 3H), 3.6-3.8(m, 1H), 3.35-3.2(m, 1H), 1.78(s, 3H), 2.0-1.5(m, 2H), 0.93(t, J=6.9 Hz, 3H); MS: 437.4.4(M−1), 439.4(M+1).

### EXAMPLE 6

### (R)-3-[2-(1,1-Difluoro-methoxy)-phenylmethanesulfonyl]-N-((S)-1-formyl-propyl)-2-hydroxy-propionamide. (Compound 23)

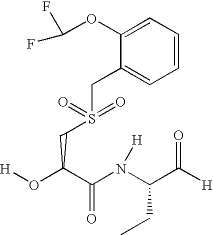

To a solution of (S)-2-{(R)-3-[2-(1,1-difluoro-methoxy)-phenylmethanesulfonyl]-2-hydroxy-propanoylamino}-N-methoxy-N-methyl-butyramide (1.3 g, 3 mmol, Example 5) in ethyl ether (50 mL) at 0° C. under N₂, was added 1N LAH solution of ethyl ether (3 ml). After stirring for 3 hours at 0° C., 1 ml of ethyl acetate and saturated NH4Cl solution was added. The crude product was then extracted with ether, washed with brine, dried with MgSO₄, filtered and concentrated. The residue was purified by flash column chromatography using silica gel with hexane/acetate as eluent to yield (R)-3-[2-(1,1-difluoro-methoxy)-phenylmethanesulfonyl]-N-((S)-1-formyl-propyl)-2-hydroxy-propionamide (0.66 g); ¹HNMR (DMSO): 9.43(s, 1H), 8.42(d, J=7.45 Hz, 1H), 7.6-7.0(m, 4H), 7.12(t, J=73.93 Hz, 1H), 6.52(d, J=6.45 Hz, 1H), 5.2-5.17(m, 1H), 4.65-4.53(m, 2H), 4.12-4.0(m, 1H), 3.63-3.55(m, 2H), 1.7-1.4(m, 2H), 0.89(t, J=6.8 Hz, 3H); MS: 378.2(M−1), 380.4(M+1).

### EXAMPLE 7

### (R)-N-[(S)-1-(1-benzooxazol-2-yl-methanoyl)-propyl]-2-hydroxy-3-phenyl-methanesulfonyl-propionamide, (Compound 5)

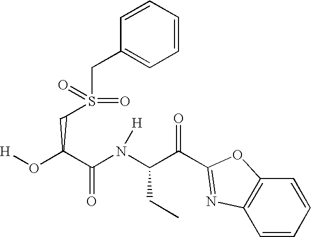

Step 1. To a solution of (R)-3-Phenylmethanesulfonyl-2-triisopropylsilanyloxy-propionic acid {556 mg, 1 mmol, Reference Example 3} in CH₂Cl₂ (10 mL) at room temperature was added HOBt (183 mg, 1.2 mmol), EDC (288 mg, 15 mmol), (S)-2-Amino-1-benzooxazol-2-yl-butanol (206 mg, 1 mml) and NMM (202 mg, 2 mmol). The mixture was then stirred overnight at room temperature before being diluted with ethyl acetate (100 mL), washed with saturated NaHCO₃, brine, dried with anhydrous MgSO₄, filtered and concentrated. The crude product was then purified by flash column chromatography using silica gel with hexane/acetate as eluent (to yield 180 mgs of product). This compound was dissolved in CH₂Cl₂, Dess-Martin Periodinane (196 mg, 0.46 mmol) was added at room temperature and the mixture was stirred for 2 hours. Saturated Na₂S₂O₃-NaHCO₃ solution (5 mL) was added and stirred for a further 30 minute before extraction with ethyl acetate and washing sequentially with saturated NaHCO₃ solution and brine. The crude product was then dried with anhydrous MgSO₄, filtered, concentrated and purified by flash column chromatography using silica gel with hexane/acetate as eluent to yield (R)-N-[(S)-1-(1-benzooxazol-2-yl-methanoyl)-propyl]-3-phenylmethanesulfonyl-2-triisopropylsilanyloxy-propionamide.

Step 2. (R)-N-[(S)-1-(1-Benzooxazol-2-yl-methanoyl)-propyl]-3-phenylmethanesulfonyl-2-triisopropylsilanyloxy-propionamide (120 mg, 0.2 mmol), in CH₃CN (10 mL), 48% HF/water solution (1 mL) were mixed and stirred at room temperature for 16 hours. Saturated NaHCO₃ solution was added carefully to adjust the pH to between 8 and 9. The product was extracted with ethyl acetate (100 mL), washed with brine and dried with magnesium sulfate. The solvent was removed and the product crystallized from acetate and hexane to yield (R)-N-[(S)-1-(1-benzooxazol-2-yl-methanoyl)-propyl}-2-hydroxy-3-phenyl-methanesulfonyl-propionamide as a white solid (85% yield); ¹H NMR: (DMSO) 8.29 (d, J=7.9 Hz, 1H), 7.74 (d, J=7.9 Hz, 1H), 7.59 (t, J=8.1 Hz, 1H), 7.46-7.35 (m, 7H), 6.52 (d, J=6.6 Hz, 1H), 5.08-4.99 (m, 1H), 4.58-4.47 (m, 3H), 3.35-3.28 (m, 2H), 2.05-1.90 (m, 1H), 1.81-1.65 (m, 1H), 0.91 (t, J=7.2 Hz, 3H); MS: (M⁺+1) 431.

### EXAMPLE 8

### (a) (S)-3-{3-[2-(1,1-Difluoro-methoxy)-phenylmethanesulfonyl]-propanoylamino}-2-oxo-pentanoic acid benzylamide, (Compound 27)

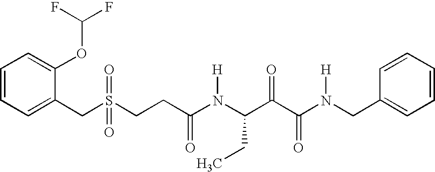

Step 1. A mixture of (R)-3-amino-2-hydroxy-pentanoic acid benzylamide TFA salt (70 mg, 0.22 mmol), 3-[2-(1,1-difluoro-methoxy)-phenylmethanesulfonyl]-propionic acid (64 mg, 0.22 mmol, Reference Example 19) HOBT (33 mg,0.22 mmol), EDC (63 mg, 0.325 mmol), 1 mL dichloromethane and N-methyl morpholine (48 μL, 0.434 mmol). The mixture was allowed to stir 16 hours. The product was extracted into 60 mL ethyl acetate and washed with two 10 mL portions of 1N HCl, 10 mL water, and two 10 mL portions of saturated NaHCO₃, dried over MgSO₄ and concentrated to give 105 mg of crude (R)-3-{3-[2-(1,1-difluoro-methoxy)-phenylmethanesulfonyl]-propanoylamino}-2-hydroxy-pentanoic acid benzylamide (0.21 mmol, 100% yield).

Step 2. To a 1 mL dichloromethane solution of 105 mg of (R)-3-{3-[2-(1,1-difluoro-methoxy)-phenylmethanesulfonyl]-propanoylamino}-2-hydroxy-pentanoic acid benzylamide (0.21 mmol) was added Dess Martin periodinane (179 mg, 0.42 mmol). The mixture was allowed to stir for 16 hours, then 10 mL of 0.26M Na₂S₂O₃ in saturated NaHCO₃ was added and the mixture was extracted with two 30 mL portions of ethyl acetate and washed with three 15 mL portions of saturated NaHCO₃. The organic layer was dried over MgSO₄ and concentrated. The product was purified by silica gel chromatography using 3:1 hexane:ethyl acetate eluent and crystallized from diethyl ether and hexane to give (S)-3-{3-[2-(1,1-difluoro-methoxy)-phenylmethanesulfonyl]-propanoylamino}-2-oxo-pentanoic acid benzylamide (28 mg, 0.054 mmol, 26% yield); ¹H NMR: (CDCl₃) 7.0-7.47 (m, 9H), 6.49 (m, 1H), 6.24 (m, 1H), 5.22 (m, 1H), 4.40 (m, 2H), 4.30 (m, 3H), 3.23 (m, 2H), 2.70 (m, 2H), 2.01 (m, 1H), 1.68 (m, 1H), 0.85 (m, 3H); MS: (M⁺+1) 499.4, 496.53.

The following compounds were prepared by the method of Example 8:

N-[(S)-1-(1-Benzooxazol-2-yl-methanoyl)-propyl}-3-[2-(1,1-difluoro-methoxy)-phenylmethanesulfonyl]-propionamide (Compound 26); ¹H NMR: (CDCl₃) 7.85 (d, J=7.6 Hz, 1H), 7.7-7.0 (m, 7H), 6.51 (m, 2H), 5.60 (m, 1H), 4.34 (m, 3H), 3.29 (m, 2H), 2.80 (m, 2H), 2.13 (m, 1H), 1.87 (m, 1H), 0.96 (m, 3H); MS: (M⁺+1) 481, 480.48;

N-[(S)-1-(1-Benzooxazol-2-yl-methanoyl)-3-phenyl-propyl]-3-p-tolylmethanesulfonyl-propionamide (Compound 30); ¹H NMR: (CDCl₃) 7.9 (m, 1H), 7.62 (m, 1H), 7.56 (td, J=6.9, 1.2 Hz, 1H), 7.47 (td, J=7.1, 1.2 Hz, 1H), 7.3-7.1 (m, 9H), 6.47 (d, J=7.7 Hz, 1H), 5.71 (m, 1H), 4.22 (s, 2H), 3.20 (m, 2H), 2.71 (m, 4H), 2.47 (m, 1H), 2.33 (s, 3H), 2.21 (m, 1H); MS: (M⁺+1) 505.2, 504.60.

3-(2-Difluoromethoxy-phenylmethanesulfonyl)-N-(1-ethyl-2,3-dioxo-3-pyrrolidin-1-yl-propyl)-propionamide;

3-(2-Difluoromethoxy-phenylmethanesulfonyl)-N-(1-ethyl-3-morpholin-4-yl-2,3-dioxo-propyl)-propionamide;

3-(2-Difluoromethoxy-phenylmethanesulfonyl)-N-(1-ethyl-2,3-dioxo-3-piperazin-1-yl-propyl)-propionamide;

3-(2-Difluoromethoxy-phenylmethanesulfonyl)-N-[3-(1,1-dioxo-116-thiomorpholin-4-yl)-1-ethyl-2,3-dioxo-propyl]-propionamide;

3-(2-Difluoromethoxy-phenylmethanesulfonyl)-N-[1-ethyl-3-(4-methyl-sulfonyl-piperazin-1-yl)-2,3-dioxo-propyl}-propionamide;

3-[3-(2-Difluoromethoxy-phenylmethanesulfonyl)-propionylamino]-2-oxo-pentanoic acid dimethylamide;

3-[3-(2-Difluoromethoxy-phenylmethanesulfonyl)-propionylamino]-2-oxo-pentanoic acid cyclopentyl-ethyl-amide;

3-[3-(2-Difluoromethoxy-phenylmethanesulfonyl)-propionylamino]-2-oxo-pentanoic acid phenylamide:

3-[3-(2-Difluoromethoxy-phenylmethanesulfonyl)-propionylamino]-2-oxo-pentanoic acid pyridin-3-ylamide;

3-[3-(2-Difluoromethoxy-phenylmethanesulfonyl)-propionylamino]-2-oxo-pentanoic acid (tetrahydro-pyran-4-yl)-amide;

3-[3-(2-Difluoromethoxy-phenylmethanesulfonyl)-propionylamino]-2-oxo-pentanoic acid (1-benzoyl-piperidin-4-yl)-amide; and

3-[3-(2-Difluoromethoxy-phenylmethanesulfonyl)-propionylamino]-2-oxo-pentanoic acid (2-morpholin-4-yl-ethyl)-amide.

### EXAMPLE 9

### (R)-N-[(S)-1-(1-Benzooxazol-2-yl-methanoyl)-propyl}-2-(2-nitro-phenylamino)-3-phenylmethanesulfonyl-propionamide, (Compound 28)

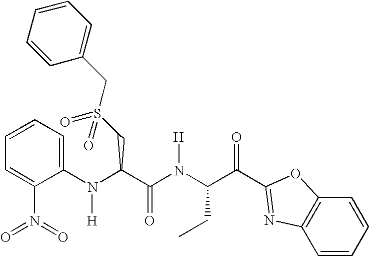

Step 1. 3-Benzylsulfanyl-2-(2-nitro-phenylamino)-propionic acid (350 mg, 1.05 mmol, Reference Example 5) was dissolved in 20 mL methanol, treated with a 20 mL aqueous solution of Oxone® (970 mg, 0.12 mmol), and stirred for 72 hours. Water (300 mL) was added and the precipitate was filtered and dried to give 2-(2-nitro-phenylamino)-3-phenylmethanesulfonyl-propionic acid (215 mg, 0.59 mmol, 56% yield)

Step 2. A mixture of 2-(2-nitro-phenylamino)-3-phenylmethanesulfonyl-propionic acid (215 mg, 0.59 mmol), HOBT (136 mg, 0.148 mmol), EDC (408 mg, 2.13 mmol), (S)-2-amino-1-benzooxazol-2-yl-butan-1-ol (122 mg, 0.59 mmol, {Reference Example 17(a)}, 2.4 mL dichloromethane and N-methyl morpholine (97 □L, 0.89 mmol) was allowed to stir 16 hours. The product was extracted into 20 mL ethyl acetate and washed with three 5 mL portions of 1N HCl, and one 30 mL portion of saturated NaHCO₃, dried over MgSO₄ and concentrated to give (R)-N-[(S)-1-(1-benzooxazol-2-yl-1-hydroxy-methyl)-propyl]-2-(2-nitro-phenylamino)-3-phenylmethane-sulfonyl-propionamide (223 mg, 0.40 mmol, 45% yield).

Step 3. (R)-N-[(S)-1-(1-Benzooxazol-2-yl-1-hydroxy-methyl)-propyl]-2-(2-nitro-phenylamino)-3-phenylmethane-sulfonyl-propionamide (223 mg, 0.4 mmol) was dissolved in 1.6 mL dichloromethane and treated with Dess Martin periodinane (342 mg, 0.80 mmol). The mixture was allowed to stir for 16 hours, then 20 mL of 0.26M Na₂S₂O₃ in saturated NaHCO₃ was added and the mixture was extracted with two 30 mL portions of ethyl acetate and washed with three 5 mL portions of saturated NaHCO₃. The organic layer was dried over MgSO₄ and concentrated. The crude product was dissolved in a minimum amount of hot ethyl acetate and crystallized by addition of dry diethyl ether. This crystallization was repeated to give clean (R)-N-[(S)-1-(1-Benzooxazol-2-yl-methanoyl)-propyl}-2-(2-nitro-phenylamino)-3-phenylmethanesulfonyl-propionamide (97 mg, 0.176 mmol, 44% yield); ¹H NMR: (DMSO) 8.67 (m, 1H), 8.12 (m, 1H), 7.81 (m, 1H), 7.65-7.35 (m, 10H), 6.78 (m, 2H), 5.51 (m, 1H), 4.68 (m, 1H), 4.37 (s, 2H), 3.62 (m, 1H), 3.38 (m, 1H), 2.15 (m, 1H), 1.91 (m, 1H), 0.98 (m, 3H); MS: (M⁺+1) 551.0, 550.58.

The following compound was prepared by the method of Example 9:

N-[1-(Benzooxazole-2-carbonyl)-propyl}-3-phenylmethanesulfonyl-2-(pyrimidin-2-ylamino)-propionamide.

### EXAMPLE 10

### (R)-N-[(S)-1-(1-Benzooxazol-2-yl-methanoyl)-butyl]-2-(5-nitro-thiazol-2-ylamino)-3-phenylmethanesulfonyl-propionamide, (Compound 29)

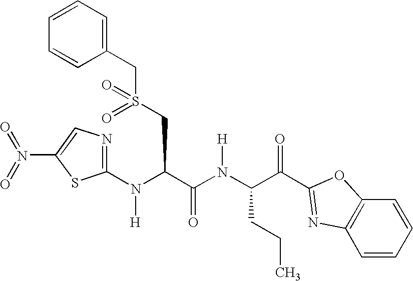

Step 1. A mixture of (R)-3-benzylsulfanyl-2-(5-nitro-thiazol-2-ylamino)-propionic acid (42 mgmg, 0.123 mmol, Reference Example 6) HOBT (28 mg, 0.148 mmol), EDC (29 mg, 0.148 mmol), (S)-2-amino-1-benzooxazol-2-yl-pentan-1-ol {27 mg, 0.123 mmol, Reference Example 17(c)}, 1 mL dichloromethane and N-methyl morpholine (14 □L, 0.123 mmol) was allowed to stir for 16 hours. The product was extracted into 60 mL ethyl acetate and washed with one 30 mL portion of 1N HCl, and one 30 mL portion of saturated NaHCO₃, dried over MgSO₄ and concentrated to give (R)-N-[(S)-1-(1-benzooxazol-2-yl-1-hydroxy-methyl)-butyl]-3-benzylsulfanyl-2-(5-nitro-thiazol-2-ylamino)-propionamide (41.8 mg, 0.077 mmol, 63% yield).

Step 2. (R)-N-[(S)-1-(1-Benzooxazol-2-yl-1-hydroxy-methyl)-butyl]-3-benzylsulfanyl-2-(5-nitro-thiazol-2-ylamino)-propionamide (41.8 mg, 0.077 mmol) was dissolved in 0.5 mL methanol, treated with a 0.5 mL aqueous solution of Oxone® (43 mg, 0.069 mmol), and stirred for 1 hour. Methanol was removed under reduced pressure and 2 mL water was added. The mixture was extracted with two 10 mL portions of ethyl acetate, dried over MgSO₄, and concentrated. It was then dissolved in 0.5 mL dichloromethane and treated with Dess Martin periodinane (65 mg, 0.154 mmol). The mixture was allowed to stir for 16 hours, then 5 mL of 0.26M Na₂S₂O₃ in saturated NaHCO₃ was added and the mixture was extracted with two 10 mL portions of ethyl acetate and washed with three 5 mL portions of saturated NaHCO₃. The organic layer was dried over MgSO₄ and concentrated. The product was purified by triturating with diethyl ether to give (R)-N-[(S)-1-(1-benzooxazol-2-yl-methanoyl)-butyl]-2-(5-nitro-thiazol-2-ylamino)-3-phenylmethanesulfonyl-propionamide (28 mg, 054 mmol, 26% yield); ¹H NMR: (CDCl₃) 7.96 (s, 1H), 7.87 (m, 1H), 7.7-7.3 (m, 9H), 5.57 (m, 1H), 5.06 (m, 1H), 4.47 (m, 2H), 3.75 (m, 1H), 3.48 (m, 1H), 2.09 (m, 1H), 1.85 (m, 1H), 1.43 (m, 1H), 1.24 (m, 1H), 0.94 (m, 3H); MS: (M⁺+1) 572.2, 571.63.

### EXAMPLE 11

### (a) (2S) (4,4-Difluoro-2-hydroxy-5-phenyl-pentanoic acid (1(S)-cyano-3-phenyl-propyl)-amide, (Compound 33)

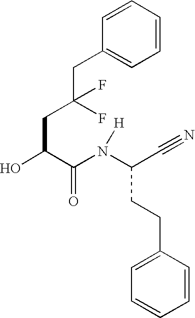

To a mixture of amino-acetonitrile hydrochloride (0.37 mmol, 72.6 mg), (2S)-4,4-difluoro-2-hydroxy-5-phenyl-pentanoic acid (1.0 eq., 0.37 mmol, 85.0 mg, Reference Example 7) and N,N-diispropylethylamine (2.2 eq., 0.81 mmol, 105.2 mg) in dry dichloromethane (4 mL) under nitrogen was added PyBOP® (1.1 eq., 0.41 mmol, 212 mg). The mixture was stirred at room temperature for 15.5 hours and then concentrated in vacuum. The residue was diluted with ethyl acetate (30 ml) and the mixture was washed with water (30 mL), then with sodium bicarbonate (30 mL) and then with water (30 mL). The organic layer was dried over MgSO4 and then concentrated in vacuum. The residue was purified over 10 g silica gel, eluting with a mixture of ethyl acetate and heptane (1:2, v/v) to afford (2S) (4,4-difluoro-2-hydroxy-5-phenyl-pentanoic acid (1(S)-cyano-3-phenyl-propyl)-amide as a light tan solid (67.4 mg, 48.9%). ¹H NMR (CDCl₃) 7.3 (m, 10H), 7.1 (d, J=7 Hz, 1H), 4.8 (q, J=7.4 Hz, 1H), 4.53 (bd, J=9.5 Hz, 1H), 3.2 (dt, J=16.2, 4.2 Hz, 2H), 2.96 (s, 1H), 2.85 (m, 2H), 2.5 (m, 1H), 2.3-0.9 (m, 3H). LC/MS 89% parent (M+1).

### (b) N-(1(S)-cyano-3-phenyl-propyl)-2-(S)-(2-morpholin-4-yl-2-oxo-ethoxy)-4-phenyl-butyramide (Compound 34)

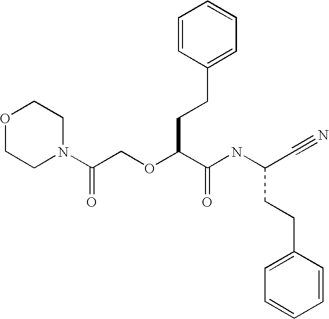

By proceeding in a manner similar to Example 11(a) above but using (S)-2-amino-4-phenyl-butyronitrile hydrochloride and 2-(S)-(2-morpholin-4-yl-2-oxo-ethoxy)-4-phenyl-butyric acid [Reference Example 8] there was prepared N-(1(S)-cyano-3-phenyl-propyl)-2-(S)-(2-morpholin-4-yl-2-oxo-ethoxy)-4-phenyl-butyramide as an oil. ¹H NMR (CDCl₃) 9.4 (d, J=8.2 Hz, 1H), 7.3 (m, 10H), 4.75 (q, J=7.5 Hz, 1H), 4.63 (d, J=15.1 Hz, 1H), 3.95 (d, J=15.3 Hz, 1H), 3.87 (dd, J=8.2, 3.9 Hz, 1H), 3.7 (m, 6H), 3.32 (m, 2H), 2.85 (m, 4H), 2.1 (m, 3H), 2.05 (m, 1H). LC/MS 100% (M+1) 450.

### (c) N-(1-(S)-cyano-3-phenyl-propyl)-2-(S)-fluoro-4-phenyl-butyramide, (Compound 35)

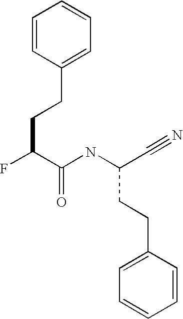

By proceeding in a manner similar to Example 11(a) above but using (S)-2-amino-4-phenyl-butyronitrile hydrochloride and (2S)-2-fluoro-4-phenyl-butyric acid (Reference Example 9) there was prepared N-(1-(S)-cyano-3-phenyl-propyl)-2-(S)-fluoro-4-phenyl-butyramide as a light tan solid. ¹H NMR (CDCl₃) 7.3 (m, 10H), 6.6 (bs, 1H), 4.95 (ddd, J=49.2, 8.2, 3.5 Hz, 1H), 4.8 (m, 1H), 3.8 (m, 4H), 2.3 (m, 1H), 2.2 (m, 3H). MS (CI, M+1) 325.

### (d) N-(1-(S)-cyano-3-phenyl-propyl)-2,2-difluoro-4-phenyl-butyramide, (Compound 36)

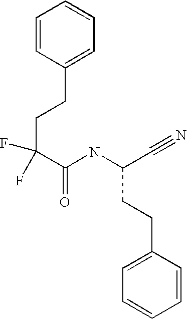

By proceeding in a manner similar to Example 11(a) above but using (S)-2-amino-4-phenyl-butyronitrile hydrochloride and 2,2-difluoro-4-phenyl-butyric acid there was prepared N-1-(S)-cyano-3-phenyl-propyl)-2,2-difluoro-4-phenyl-butyramide as a white solid. ¹H NMR (CDCl₃) 7.3 (m, 10H), 6.6 (d, J=8.1 Hz, 1H), 4.83 (q, J=7.4 Hz, 1H), 2.88 (dt, J=7.5, 2.5 Hz, 2H), 2.79 (t, J=8 Hz, 2H), 2.4 (m, 2H), 2.2 (q, J=7.5 Hz, 2H). LC/MS 50% (M=1) 343.

### (e) N-(1-(S)-cyano-3-phenyl-propyl)-2-(S)-hydroxy-4-phenyl-butyramide, (Compound 37)

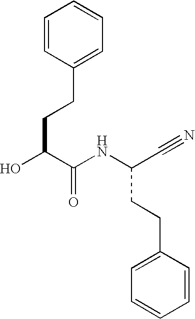

By proceeding in a manner similar to Example 11(a) above but using (S)-2-amino-4-phenyl-butyronitrile hydrochloride and (2S)-2-hydroxy-4-phenyl-butyric acid there was prepared N-(1-(S)-cyano-3-phenyl-propyl)-2-(S)-hydroxy-4-phenyl-butyramide as a white solid. ¹H NMR (CDCl₃) 7.3 (m, 10H), 6.9 (d, J=8.4 Hz, 1H), 4.86 (q, J=7.4 Hz, 1H), 4.2 (m, 1H), 2.84 (t, J=7.1 Hz, 2H), 2.77 (t, J=7.8 Hz, 2H), 2.5 (d, J=4.7 Hz, H), 2.2 (m, 3H), 1.95 (m, 1H). LC/MS 49% (M+1) 323.

### (f) N-(1-(S)-cyano-3-phenyl-propyl)-2-(R)-hydroxy-4-phenyl-butyramide, (Compound 38)

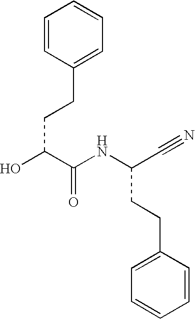

By proceeding in a manner similar to Example 11(a) above but using (S)-2-amino-4-phenyl-butyronitrile hydrochloride and (2R)-2-hydroxy-4-phenyl-butyric acid there was prepared N-(1-(S)-cyano-3-phenyl-propyl)-2-(R)-hydroxy-4-phenyl-butyramide as a white solid. ¹H NMR (CDCl₃) 7.4-7.1 (m, 10H), 6.9 (d, J=8.7 Hz, 1H), 4.87 (q, J=7.3 Hz, 1H), 4.1 (m, 1H), 2.85 (t, J=7.5 Hz, 2H), 2.77 (t, J=8.4 Hz, 2H), 2.3 (d, J=5.1 Hz, 1H), 2.2 (m, 3H), 2.0 (m, 1H). LC/MS 94% (M+1) 323.

### (g) N-(1-(S)-cyano-3-phenyl-propyl)-2-(R)-methoxy-4-phenyl-butyramide, (Compound 39)

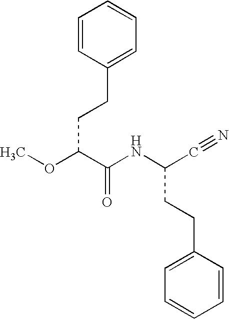

By proceeding in a manner similar to Example 11(a) above but using (S)-2-amino-4-phenyl-butyronitrile hydrochloride (0.407 mmol, 80 mg) and 2(R)-methoxy-4-phenyl-butyric acid (Reference Example 10) there was prepared N-(1-S)-cyano-3-phenyl-propyl)-2-(R)-methoxy-4-phenyl-butyramide as a white solid (91.8 mg, 67%). ¹H NMR (CDCl₃) 7.2 (m, 10H), 6.8 (d, J=8.5 Hz, 1H), 4.86 (q, J=7.5 Hz, 1H), 3.67 (dd, J=6.5,4.5 Hz, 1H), 3.35 (s, 3H), 2.85 (m, 2H), 2.68 (t, J=8.0 Hz, 2H), 2.2-2.0 (m, 4H). LC/MS 84% (M⁺1) 337.

### (h) 2,2-Difluoro-5-phenyl-pentanoic acid (1-cyano-cyclopropyl)-amide, (Compound 40)

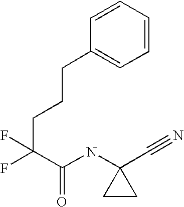

By proceeding in a manner similar to Example 11(a) above but using 2,2-difluoro-5-phenyl-pentanoic acid and 1-amino-cyclopropanecarbonitrile hydrochloride there was prepared 2,2-difluoro-5-phenyl-pentanoic acid (1-cyano-cyclopropyl)-amide. ¹H NMR (CDCl₃) δ 1.32 (m, 2H), 1.64 (m, 2H), 1.82 (m, 2H), 2.12 (m, 2H), 2.8-2.56 (m, 2H), 6.82 (m, 1H), 7.36-7.15 (m, 5H). MS (ES-) 277.

### (i) N-(1-(S)-Cyano-3-phenyl-propyl)-4-phenyl-butyramide, (Compound 41)

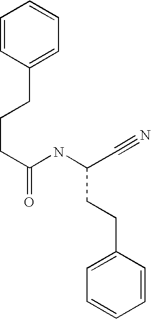

By proceeding in a manner similar to Example 11(a) above but using (S)-2-amino-4-phenyl-butyronitrile hydrochloride and 4-phenylbutyric acid there was prepared N-(1-(S)-cyano-3-phenyl-propyl)-4-phenyl-butyramide as a colorless oil. ¹H NMR (CDCl₃): δ 7.3 (m, 10 H), 6.0 (d, J=8.3 Hz, 1H), 4.9 (q, J=7.4 Hz, 1H), 2.8 (m, 2H), 2.65 (t, J=7.4 Hz, 2H), 2.15 (m, 4H), 1.95 (m, 2H). LC/MS 100% (M+1) 307.

### EXAMPLE 12

### 2,2-difluoro-5-phenyl-pentanoic acid ((S)-1-cyano-3-phenyl-propyl)-amide, (Compound 42)

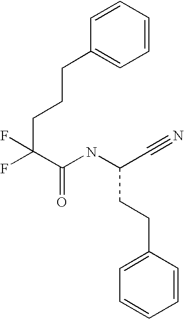

A mixture of 2,2-difluoro-5-phenyl-pentanoic acid (109 mg, 0.509 mmol), (S)-2-amino-4-phenyl-butyronitrile hydrochloride (103 mg, 0.523 mmol) and HATU (206 mg, 0.542 mmol) in DMF (4 mL) and stirred at room temperature for 5 hours then evaporated under reduced pressure. The residue was taken in ethyl acetate washed with 1N HCl, sodium bicarbonate and then water. Organic extract was dried over Na₂SO₄ and then evaporated under vacuum to give orange oil. The residue was subjected to mplc, eluting with a mixture of ethyl acetate and heptane (1:9, v/v) to give 2,2-difluoro-5-phenyl-pentanoic acid ((S)-1-cyano-3-phenyl-propyl)-amide as colorless oil (82 mg). ¹H NMR (CDCl₃) 7.3-7.1 (m, 10H), 6.9 (bs, 1H), 4.80 (q, J=7.5 Hz, 1H), 2.80 (dt, J=7.3, 2.7 Hz, 2H), 2.65 (t, J=7.5 Hz, 2H), 2.2-2.0 (m, 4H), 1.8 (m, 2H). MS 357 (MH⁺), 379 (M+Na).

### EXAMPLE 13

### (a) N-(4-Cyano-1-ethyl-piperidin-4-yl)-3-cyclohexyl-propionamide

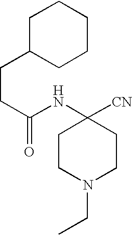

Step 1. To a stirred solution of 1-ethyl-4-piperidone(25 g, 0.197 mol) in 300 ml of diethyl ether, and NH₄Cl(22.3 g, 0.41 mol), was added NaCN(14.5 g, 0.295 mol, in 70 ml water) drop-wise at room temperature. After stirring for 24 h the diethyl ether was separated and the water phase was extracted with n-BuOH, then washed with brine and dried. After removal of most of the n-BuOH under reduced pressure, the residue was diluted with 50 ml of diethyl ether and then acidified with 2N HCl in diethyl ether solution at 0° C. The solid was dried under vacuum to yield 45 g of 4-amino-1-ethyl-piperidine-4-carbonitrile HCl salt. Step 2. To a stirred mixture of 3-cyclohexyl-propionic acid (156 mg, 1 mmol), 4-amino-1-ethyl-piperidine-4-carbonitrile HCl salt (227, 1 mmol, prepared as in step 1 above), and HATU (570 mg, 1.5 mmol) in MeCl₂ (5 ml), was added N,N-diisopropylethylamine (516 mg, 4 mmol) at room temperature. After stirring for 14 hours, the reaction mixture was extracted with ethyl acetate. The organic layer was washed with saturated NaHCO₃, brine, dried with MgSO₄ and concentrated to yield N-(4-Cyano-1-ethyl-piperidin-4-yl)-3-cyclohexyl-propionamide (170 mg). LC-MS: elution time=2.25 min. 290.2(M−1), 292.2(M+1). (MS: API 150EX. LC: HP Agilent 1100 Series. Column: Phenomenex, 5 u ODS3 100A 100×3 mm.; Flow Rate: 2 ml/min. Two solvent gradient: Solvent A, 99% water, 1% acetonitrile, 0.1% AcOH. Solvent B, 99% acetonitrile, 1% water, 0.1% AcOH. Gradient from 100% A, 0% B to 0% A, 100% B from t=0 to t=6 min. Then gradient back to 100% A, 0% B from t=7 to t=15 min.).

### (b) N-(4-Cyano-1-ethyl-piperidin-4-yl)-3-(2-difluoromethoxy-phenylmethanesulfonyl)-propionamide

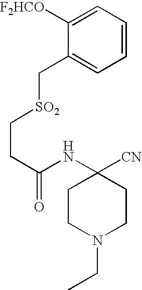

By proceeding in a similar manner to Example 13(a) but using 3-(2-difluoromethoxy-phenylmethanesulfonyl)-propionic acid (294 mg, 1 mmol) and 4-amino-1-ethyl-piperidine-4-carbonitrile HCl salt(227, 1 mmol) there was N-(4-cyano-1-ethyl-piperidin-4-yl)-3-(2-difluoromethoxy-phenylmethanesulfonyl)-propionamide 260 mg). LC-MS: R_{T}=1.96 min., 428.2(M−1), 430.3(M+1). MS: API 150EX. (LC: Agilent 1100Series, Column: Phenomenex, 5 u ODS3 100A 100×3 mm. Flow Rate: 2 ml/min. Two solvent gradient: Solvent A, 99% water, 1% acetonitrile, 0.1% AcOH. Solvent B, 99% actonitrile, 1% water, 0.1% AcOH. Gradient from 100% A, 0% B to 0% A, 100% B from t=0 to t=6 min. Then gradient back to 100% A, 0% B from t=7 to t=15 min.).

### EXAMPLE 14

### (S)-tert-Butyl-carbamic acid 1-(cyanomethyl-carbamoyl)-2-cyclohexyl-ethyl ester

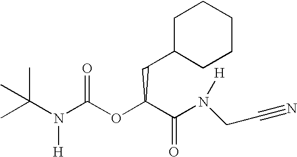

(S)-N-Cyanomethyl-3-cyclohexyl-2-hydroxy-propionamide (53 mg, 0.252 mmol) was dissolved in dichloromethane (1 mL). Triethylamine (0.1 mL) was added and then tert.-butyl isocyanate (0.034 mL, 0.3 mmol). The mixture was stirred at room temperature overnight. After dilution with ethyl acetate (100 mL), the solution was washed with 1N aqueous. HCl, brine, sat. aqueous NaHCO₃, and brine, dried with MgSO₄ and evaporated under vacuum. Flash chromatography on silica gel (hexane/ethyl acetate 1:1) gave (S)-tert-Butyl-carbamic acid 1-(cyanomethyl-carbamoyl)-2-cyclohexyl-ethyl ester (63 mg, 0.204 mmol) as a white solid.

### EXAMPLE 15

### (a) (R)-Carbamic acid 1-(cyanomethyl-carbamoyl)-2-(2-difluoromethoxy-phenylmethanesulfonyl)-ethyl ester

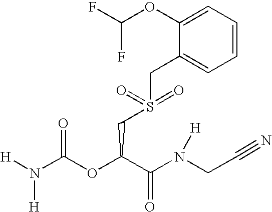

(R)-N-Cyanomethyl-3-(2-(1,1-difluoromethoxy)-phenylmethanesulfonyl)-2-hydroxy-propionamide {100 mg, 0.287 mmol, Example 1(a)} was dissolved in dichloromethane (2 mL) and THF (1 mL). Trichloroacetyl isocyanate (0.051 mL, 0.43 mmol) was added and the mixture was stirred for 1 h. The solvents were removed under vacuum and the residue was dissolved in 1,4-dioxane (10 mL). 1N aqueous. HCl (5 mL) was added and the mixture was heated at 70° C. for 4 h. After cooling to room temperature, the mixture was extracted with ethyl acetate. The combined organic layers were washed with brine, dried with MgSO₄ and evaporated under vacuum. Flash chromatography on silica gel (hexane/ethyl acetate 1:3) gave (R)-carbamic acid 1-(cyanomethyl-carbamoyl)-2-(2-difluoromethoxy-phenylmethanesulfonyl)-ethyl ester (35 mg, 0.089 mmol) as a white solid. ¹H NMR: (DMSO) 8.90 (t, J=4.8 Hz, 1H), 7.48-7.43 (m, 2H), 7.30-7.21 (m, 2H), 7.11 (t, J_{H,F}=73.6 Hz, 1H), 6.98-6.76 (br, 2H), 5.28-5.23 (m, 1H), 4.60 (s, 2H), 4.15 (d, J=4.8 Hz, 2H), 3.70 (dd, J=10.0 Hz, J=14.8 Hz, 1H), 3.54 (d, J=14.4 Hz, 1H). MS: (M+H)⁺ 392.

### (b) (S)-Carbamic acid 1-(cyanomethyl-carbamoyl)-2-cyclohexyl-ethyl ester

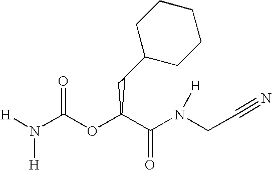

By proceeding in a manner similar to Example 8(a) above but using (R)-N-cyanomethyl-3-cyclohexyl-2-hydroxy-propionamide there was prepared (S)-Carbamic acid 1-(cyanomethyl-carbamoyl)-2-cyclohexyl-ethyl ester. ¹H NMR: (DMSO) 8.63 (t, J=5.6 Hz, 1H), 6.63 (br, 2H), 4.81-4.77 (m, 1H), 4.11 (d, J=5.2 Hz, 2H), 1.74-0.81 (m, 13H). MS: (M+H)⁺ 254.

### EXAMPLE 16

### (a) (R)-Morpholine-4-carboxylic acid 1-(1-cyano-cyclotpropylcarbamoyl)-2-phenylmethanesulfonyl-ethyl ester

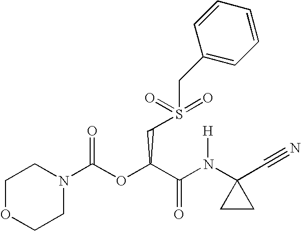

DMF was added to a mixture of (R)-morpholine-4-carboxylic acid 1-carboxy-2-phenylmethanesulfonyl-ethyl ester {from step 2 in Example 4(a)} (60 mg, 0.168 mmol), HATU (200 mg, 0.52 mmol), and 1-amino-cyclopropanecarbonitrile hydrochloride (100 mg, 0.84 mmol). 4-Methylmorpholine (0.5 mL) was added and the mixture was stirred overnight. The mixture was diluted with ethyl acetate (100 mL), washed with 1N aqueous. HCl, brine, sat. aqueous. NaHCO₃, brine, dried with MgSO₄ and evaporated under vacuum. Flash chromatography on silica gel (hexane/ethyl acetate 1:2) gave (R)-morpholine-4-carboxylic acid 1-(1-cyano-cyclopropylcarbamoyl)-2-phenylmethanesulfonyl-ethyl ester (7 mg, 0.017 mmol). ¹HNMR: (DMSO) 9.16 (s, 1H), 7.40-7.32 (m, 5H), 5.24-5.19 (m, 1H), 4.55 (d, J=13.2 Hz, 1H), 4.48 (d, J=13.2 Hz, 1H), 3.63-3.25 (m, 10H), 1.51-1.39 (m, 2H), 1.20-1.07 (m, 2H). MS: (M+H)⁺422.

### (b) (R)-Morpholine-4-carboxylic acid 1-(4-cyano-tetrahydro-pyran-4-ylcarbamoyl)-2-phenylmethanesulfonyl-ethyl ester

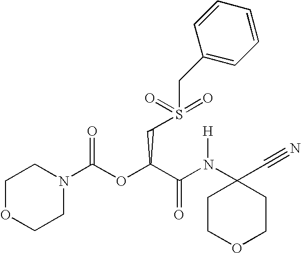

By proceeding in a manner similar to Example 16(a) above but using 4-amino-tetrahydropyran-4-carbonitrile hydrochloride {prepared according to Example 13(a) step1 but using tetrahydropyran-4-one} there was prepared (R)-morpholine-4-carboxylic acid 1-(4-cyano-tetrahydro-pyran-4-ylcarbamoyl)-2-phenylmethanesulfonyl-ethyl ester. LC-MS: elution time=3.20 min. 464.4(M−1), 466.2(M+1). (MS: API 150EX. LC: HP Agilent 1100 Series. Column: Phenomenex, 5 u ODS3 100 A 100×3 mm.; Flow Rate: 2 ml/min. Two solvent gradient: Solvent A, 99% water, 1% acetonitrile, 0.1% AcOH. Solvent B, 99% actonitrile, 1% water, 0.1% AcOH. Gradient from 100% A, 0% B to 0% A, 100% B from t=0 to t=6 min. Then gradient back to 100% A, 0% B from t=7 to t=15 min.)

### EXAMPLE 17

### 3-Cyclohexyl-2-hydroxy-N-[1-(oxazolo[4,5-b]pyridine-2-carbonyl)-propyl]-propionamide

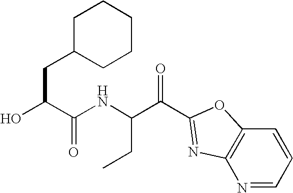

Step 1. To a stirred solution of [1-(hydroxy-oxazolo[4,5-b]pyridin-2-yl-methyl)-propyl]-carbamic acid tert-butyl ester (3.11 g, 10 mmol, prepared as described in Reference Example 20 step2.) in dioxane (4 ml) was added HCl (4N solution in 5 ml of dioxane) at room temperature. After 2 hours, ethyl ether(50 ml) was added and the reaction mixture was filtered. The resultant solid was washed with an additional 20 ml of ethyl ether and dried under vacuum to yield 3 g of 2-amino-1-oxazolo[4,5-b]pyridin-2-yl-butan-1-ol HCl salt.

Step 2. To a stirred mixture of 3-cyclohexyl-2-hydroxy-propionic acid (155 mg, 0.9 mmol), 2-amino-1-oxazolo[4,5-b]pyridin-2-yl-butan-1-ol HCl salt, and HOBt (168 mg, 1.1 mmol) in MeCN (5 ml), was added EDC (270 mg, 1.4 mmol) and N-methylmorpholine (0.45 ml) at 23° C. After stirring for 14 hours, the reaction mixture was extracted with ethyl acetate. The organic layer was washed with saturated NaHCO₃, brine, dried with MgSO₄ and concentrated to yield 293 mg of 3-cyclohexyl-2-hydroxy-N-[1-(hydroxy-oxazolo[4,5-b]pyridin-2-yl-methyl)-propyl}-propionamide which was used in step 3 following without further purification. MS: 360.3(M−1), 362.3(M+1), 384.2(M+Na).

Step 3. To a stirred solution of 3-cyclohexyl-2-hydroxy-N-[1-(hydroxy-oxazolo[4,5-b]pyridin-2-yl-methyl)-propyl]-propionamide (300 mg, 0.83 mmol) in MeCl₂(20 ml), was added MnO₂(1.44 g, 16.6 mmol) at room temperature. After stirring for 30 min. the mixture was filtered to remove MnO₂, and washed with 20 ml of MeCl₂. The solvent was removed under vacuum and the residue was purified by silica gel column chromatography to yield 3-cyclohexyl-2-hydroxy-N-[1-(oxazolo[4,5-b]pyridine-2-carbonyl)-propyl]-propionamide (40 mg). H¹NMR (DMSO-□): 8.71(1H, dd, NH, diastereomer), 8.38(1H, dd,), 8.28(1H, m), 7.7-7.6(1H, m), 5.5-5.4(1H, m), 5.2-5.1(1H, m), 3.95-3.991H, br., OH), 2.1-1.95(1H, m), 1.85-1.75(1, m), 1.7-0.8(16H, m).

MS: 358.1 (M−1), 360.1 (M+1), 382(M+Na).

### EXAMPLE 18

### (a) (R)-N-[1-(Benzothiazole-2-carbonyl)-butyl]-2-isopropylamino-3-phenylmethanesulfonyl-propionamide

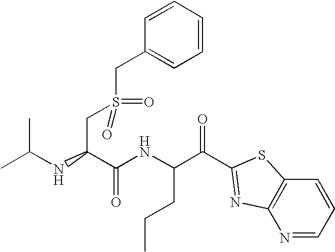

A solution of (R)-N-[1-(benzothiazol-2-yl-hydroxy-methyl)-butyl]-2-isopropylamino-3-phenylmethanesulfonyl-propionamide {30 mg, 0.06 mmol, Example 30(a)} in dichloromethane (10 mL) was treated with Dess-Martin-periodinane (51 mg, 0.12 mmol). This mixture was stirred at room temperature for 45 minutes then treated with resin-bound thiosulfate (400 mg, 0.6 mmol) and stirring was continued for a further 24 hours then the mixture was treated with AP-Trisamine (270 mg, 0.6 mmol). After stirring for a further 24 hours the reaction mixture was filtered and the filtrate was evaporated to give (R)-N-[1-(benzothiazole-2-carbonyl)-butyl]-2-isopropylamino-3-phenylmethanesulfonyl-propionamide (23 mg, 75%) as mixture of diastereomers. ¹H NMR (CDCl₃, 300 MHz): 8.29-8.27 (m, 1H), 8.23-8.19 (m, 1H), 8.01-7.98 (m, 1H), 7.63-7.36 (m, 7H), 5.80-5.74 (m, 1H), 4.36-4.31 (m, 2H),[3.79 (dd, J=9.5 Hz, 3 Hz), 3.73 (dd, J=9 Hz, 2.5 Hz) 1H], 3.41-3.34 (m, 1H), 3.20-3.01 (m, 1H), 2.89-2.85 (m, 1H), 2.14-2.06 (m, 1H), 1.88-1.78 (m, 1H), 1.52-1.25 (m, 3H), 1.12-1.06 (m, 6H), [0.96 (t, 7.5 Hz) 0.95 (t, J=7.5 Hz) 1H]. LC/MS m/z=502 (M+H).

### (b) (R)-N-[1-(Benzothiazole-2-carbonyl)-butyl]-3-phenylmethanesulfonyl-2-(tetrahydro-pyran-4-ylamino)-propionamide

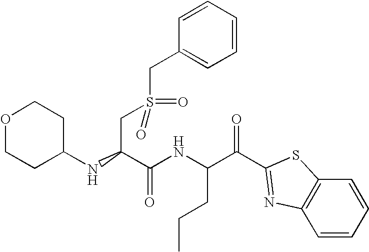

By proceeding in a similar manner to Example 18(a) but using (R)-N-[1-(benzothiazol-2-yl-hydroxy-methyl)-butyl]-3-phenylmethanesulfonyl-2-(tetrahydro-pyran-4-ylamino)-propionamide {0.11 mmol, Example 29(b)} and subjecting the crude product to HPLC there was prepared (R)-N-[1-(benzothiazole-2-carbonyl)-butyl]-3-phenylmethanesulfonyl-2-(tetrahydro-pyran-4-ylamino)-propionamide (10 mg, 16%). LC/MS retention time 2.92 min (TIC), m/z=544 (M+H) (determined with method A).

### (c) (R)-N-1-(Benzothiazole-2-carbonyl)-butyl]-2-dibenzylamino-3-phenylmethanesulfonyl-propionamide

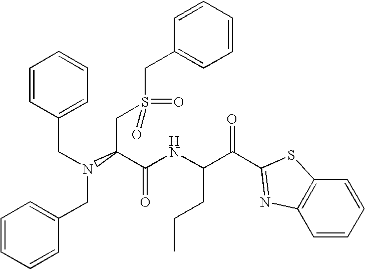

By proceeding in a similar manner to Example 18(a) but using (R)-N-[1-(benzothiazol-2-yl-hydroxy-methyl)-butyl]-2-dibenzylamino-3-phenylmethanesulfonyl-propionamide {0.11 mmol, Example 29(a)} and subjecting the crude product to HPLC there was prepared (R)-N-[1-(benzothiazole-2-carbonyl)-butyl]-2-dibenzylamino-3-phenylmethanesulfonyl-propionamide (4 mg) as mixture of diastereomers. ¹H NMR (CDCl₃, 300 MHz): 8.33-7.89 (m, 3H), 7.61-7.55 (m, 2H), 7.47-7.29 (m, 15H), 5.75 (m, 1H), [4.54 (d, J=14 Hz), 4.51 (d, J=13.5 Hz), 1H], [4.27 (d, J=14 Hz), 4.25 (d, J=13.5 Hz), 1H], 4.11-3.95 (m, 2H), [3.78 (d, J=13 Hz), 3.76 (d, J=13 Hz), 2H], [3.51 (d, J=13 Hz), 3.50 (d, J=13 Hz), 2H], 3.19-3.13 (m, 1H), 2.10-1.77 (m, 2H), 1.51-1.37 (m, 2H), 0.91-084 (m, 3H). LC/MS m/z=640 (M+H).

### (d) (R)-N-[1-(Benzothiazole-2-carbonyl)-butyl]-2-dimethylamino-3-phenylmethanesulfonyl-propionamide

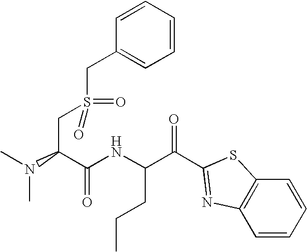

By proceeding in a similar manner to Example 18(a) but using (R)-N-[1-(benzothiazol-2-yl-hydroxy-methyl)-butyl]-2-dimethylamino-3-phenylmethanesulfonyl-propionamide {30 mg, 0.06 mmol, Example 30(b)}, and subjecting the crude product to HPLC there was prepared (R)-N-[1-(benzothiazole-2-carbonyl)-butyl]-2-dimethylamino-3-phenylmethanesulfonyl-propionamide (11 mg, 38%).

LC/MS retention time 2.98 min (TIC), m/z488 (M+H) (determined with method A).

### EXAMPLE 19

### (a) (R)-N-[(S)-1-(Benzoxazole-2-carbonyl)-butyl]-3-phenylmethanesulfonyl-2-(tetrahydro-pyran-4-ylamino)-propionamide

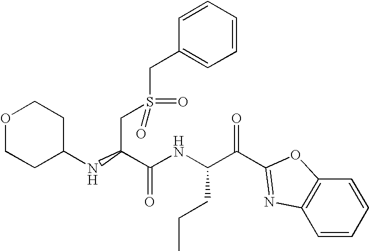

A solution of (R)-N-[(S)-1-(benzoxazol-2-yl-hydroxy-methyl)-butyl]-3-phenylmethanesulfonyl-2-(tetrahydro-pyran-4-ylamino)-propionamide {0.22 mmol, Example 31(a)} in dichloromethane (10 mL) was treated with Dess-Martin-periodinane (187 mg, 0.44 mmol). This mixture was agitated at room temperature overnight then treated with resin-bound thiosulfate (1.47 g, 2.2 mmol) and stirring was continued for a further 24 hours then the mixture was treated with Silicycle Triamine (611 mg, 2.2 mmol). After agitating for a further 24 hours the reaction mixture was filtered. The filtrate was evaporated and the residue was subjected to HPLC to give (R)-N-[(S)-1-(benzoxazole-2-carbonyl)-butyl]-3-phenylmethanesulfonyl-2-(tetrahydro-pyran-4-ylamino)-propionamide (9 mg, 8%). LC/MS retention time 3.0 min (TIC), m/z=528 (M+H) (determined with method B).

### (b) (R)-N-[(S)-1-(Benzoxazole-2-carbonyl)-butyl]-2-(1-methyl-piperidin-4-ylamino)-3-phenylmethanesulfonyl-propionamide

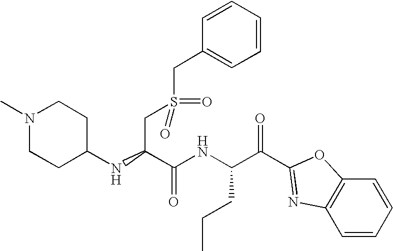

By proceeding in a similar manner to Example 19(a) but using (R)-N-[(S)-1-(benzoxazol-2-yl-hydroxy-methyl)-butyl]-2-(1-methyl-piperidin-4-ylamino)-3-phenylmethanesulfonyl-propionamide {0.22 mmol, Example 31(b)} there was prepared (R)-N-[(S)-1-(benzoxazole-2-carbonyl)-butyl]-2-(1-methyl-piperidin-4-ylamino)-3-phenylmethanesulfonyl-propionamide (7 mg, 6%).

LC/MS retention time 2.7 min (TIC), m/z=541 (M+H) (determined with method A).

### (c) (R)-N-[(S)-1-(Benzoxazole-2-carbonyl)-butyl]-2-(bis-thiophen-2-ylmethyl-amino)-3-phenylmethanesulfonyl-propionamide

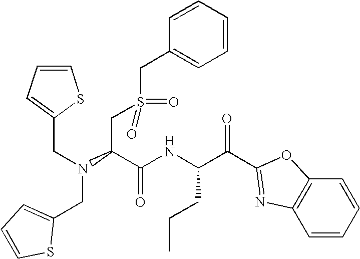

By proceeding in a similar manner to Example 19(a) but using (R)-N-[(S)-1-(benzoxazol-2-yl-hydroxy-methyl)-butyl]-2-(bis-thiophen-2-ylmethyl-amino)-3-phenylmethanesulfonyl-propionamide {0.22 mmol, Example 31(c)} there was prepared (R)-N-[(S)-1-(benzoxazole-2-carbonyl)-butyl]-2-(bis-thiophen-2-ylmethyl-amino)-3-phenylmethanesulfonyl-propionamide (5.3 mg, 4%)

LC/MS retention time 3.7 min (TIC), m/z=636 (M+H) (determined with method A).

### (d) (R)-N-[(S)-1-(Benzoxazole-2-carbonyl)-butyl]-2-dibenzylamino-3-phenylmethanesulfonyl-propionamide

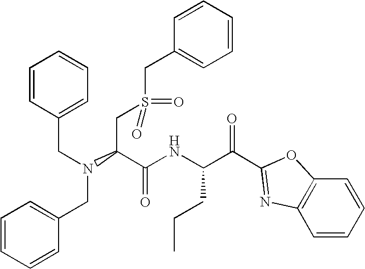

By proceeding in a similar manner to Example 19(a) but using (R)-N-[(S)-1-(benzoxazol-2-yl-hydroxy-methyl)-butyl]-2-dibenzylamino-3-phenylmethanesulfonyl-propionamide {0.22 mmol, Example 31(d)} there was prepared (R)-N-[(S)-1-(benzoxazole-2-carbonyl)-butyl]-2-dibenzylamino-3-phenylmethanesulfonyl-propionamide (3.8 mg, 3%). LC/MS retention time 4.14 min (TIC), m/z=624 (M+H) (determined with method B).

### (e) (S)-N-[(S)-1-(Benzoxazole-2-carbonyl)-butyl]-2-(tetrahydro-pyran-4-ylamino)-3-thiophen-2-yl-propionamide

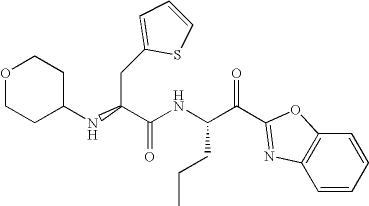

By proceeding in a similar manner to Example 19(a) but using (S)-N-[(S)-1-(benzoxazol-2-yl-hydroxy-methyl)-butyl]-2-(tetrahydro-pyran-4-ylamino)-3-thiophen-2-yl-propionamide {0.22 mmol, Example 31(e)} there was prepared (S)-N-[(S)-1-(benzoxazole-2-carbonyl)-butyl]-2-(tetrahydro-pyran-4-ylamino)-3-thiophen-2-yl-propionamide (6.5 mg, 6%). LC/MS retention time 2.92 min (TIC), m/z=456 (M+H) (determined with method B).

### (f) (S)-N-[(S)-1-(Benzoxazole-2-carbonyl)-butyl]-2-isopropylamino-3-thiophen-2-yl-propionamide

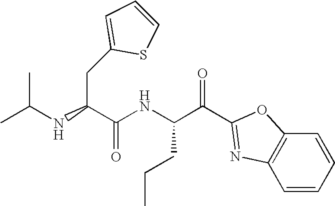

By proceeding in a similar manner to Example 19(a) but using (S)-N-[(S)-1-(Benzoxazol-2-yl-hydroxy-methyl)-butyl]-2-isopropylamino-3-thiophen-2-yl-propionamide {0.22 mmol, Example 31(f)), there was prepared (S)-N-[(S)-1-(benzoxazole-2-carbonyl)-butyl]-2-isopropylamino-3-thiophen-2-yl-propionamide (10.6 mg, 12%). LC/MS retention time 2.99 min (TIC), m/z=414 (M+H) (determined with method B).

### EXAMPLE 20

### (a) (R)-N-[1-(Benzothiazole-2-carbonyl)-butyl]-3-phenylmethanesulfonyl-2-(tetrahydro-pyran-4-ylamino)-propionamide

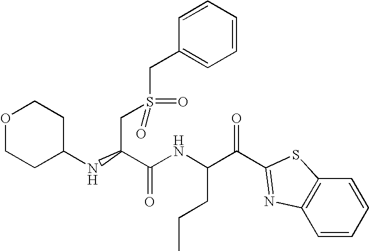

A solution of (R)-N-[1-(benzothiazol-2-yl-hydroxy-methyl)-butyl]-3-phenylmethanesulfonyl-2-(tetrahydro-pyran-4-ylamino)-propionamide {0.22 mmol, Example 32(a)} in dichloromethane (10 mL) was treated with Dess-Martin-periodinane (187 mg (0.44 mmol). After stirring at room temperature for 30 minutes the reaction mixture was treated with saturated sodium thiosulfate solution (50 ml) and saturated sodium bicarbonate solution (50 ml). The phases were separated and the aqueous phase extracted with dichloromethane. The combined organic phases were washed with brine, then dried over magnesium sulfate and then evaporated. The residue was subjected to flash chromatography using a silica gel cartridge to give (R)-N-[1-(benzothiazole-2-carbonyl)-butyl]3-phenylmethanesulfonyl-2-(tetrahydro-pyran-4-ylamino)-propionamide (46 mg, 38%) as mixture of diastereoisomers. The two diastereomers were separated by silica gel column chromatography eluting with 1:1 v/v heptane-ethyl acetate mixture.

Diastereoisomer A:

¹H NMR (CDCl₃, 300 MHz): 8.23-8.20 (m, 2H), 8.00 (dd, J=7 Hz, 2 Hz, 1H), 7.63-7.53 (m, 2H), 7.48-7.40 (m, 5H), 5.80 (m, 1H), 4.38 (d, J=14 Hz, 1H), 4.32 (d, J=14 Hz, 1H), 3.97-3.90 (m, 2H), 3.80 (dd, J=9.5 Hz, 3 Hz, 1H), 3.43-3.30 (m, 3H), 3.13 (dd, J=14.5 Hz, 9.5 Hz, 1H), 2.70 (m, 1H), 2.27 (m, 1H), 2.09 (m, 1H), 1.91-1.76 (m, 3H), 1.52-1.37 (m, 4H), 0.95 (t, J=7.5 Hz, 3H). LC/MS m/z=544 (M+H)

Diastereoisomer B:

¹H NMR (CDCl₃, 300 MHz): 8.22-8.19 (m, 2H), 8.01-7.98 (m, 1H), 7.63-7.53 (m, 2H), 7.44-7.37 (m, 5H), 5.74 (m, 1H), 4.35-4.31 (m, 2H), 3.99-3.94 (m, 2H), 3.86 (dd J=9.5 Hz, 3 Hz, 1H), 3.49-3.33 (m, 3H), 3.08 (dd, J=14.5 Hz, 9.5 Hz), 2.75-2.70 (m, 1H), 2.22 (m, 1H), 2.15-2.06 (m, 1H), 1.91-1.75 (m, 3H), 1.53-1.37 (m, 4H), 0.96 (t, J=7.5 Hz, 3H). LC/MS m/z=544 (M+H)

### (b) (R)-N-[(S)-1-(Benzoxazole-2-carbonyl)-butyl]-3-phenylmethanesulfonyl-2-(tetrahydro-pyran-4-ylamino)-propionamide

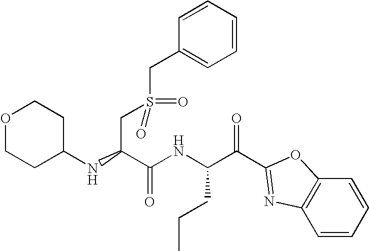

By proceeding in a similar manner to Example 20(a) but using (R)-N-[(S)-1-(benzoxazol-2-yl-hydroxy-methyl)-butyl]-3-phenylmethanesulfonyl-2-(tetrahydro-pyran-4-ylamino)-propionamide {0.22 mmol, Example 32(b)} there was prepared (R)-N-[(S)-1-(benzoxazole-2-carbonyl)-butyl]-3-phenylmethanesulfonyl-2-(tetrahydro-pyran-4-ylamino)-propionamide (48 mg, 41%). ¹H NMR (CDCl₃, 300 MHz): 8.22 (d, J=8.5 Hz, 1H), 7.92 (d, J=8 Hz, 1H), 7.68 (d, J=8.5 Hz, 1H), 7.60-7.40 (m, 7H), 5.68-5.61 (m, 1H), 4.37 (d, J=14 HZ, 1H), 4.31 (d, J=14 Hz, 1H), 3.97-3.91 (m, 2H), 3.80 (dd, J=9.5 Hz, 3 Hz, 1H), 3.43-3.32 (m, 3H), 3.12 (dd, J=14.5 Hz, 9.5 Hz, 1H), 2.73-2.66 (m, 1H), 2.26 (m, 1H), 2.13-2.05 (m, 1H), 1.89-1.77 (m, 3H), 1.52-1.39 (m, 4H), 0.97 (t, J=7.5 Hz, 3H). LC/MS m/z-528 (M+H).

### EXAMPLE 21

### (a) (R)-N-[(S)-1-(Benzoxazole-2-carbonyl)-butyl]-2-isopropylamino-3-phenylmethanesulfonyl-propionamide

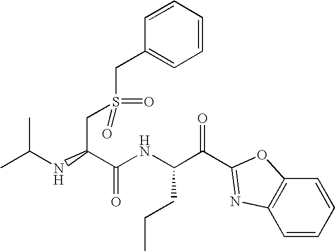

A solution of (R)-N-[(S)-1-(benzoxazol-2-yl-hydroxy-methyl)-butyl]-2-isopropylamino-3-phenylmethanesulfonyl-propionamide {30 mg, 0.063 mmol, Example 31(g)} in dichloromethane (10 mL) was treated with Dess-Martin-periodinane (53 mg, 0.126 mmol) and this mixture was stirred at room temperature for 1 hour then subjected to The Mettler-Toledo Allex™ liquid handler work-up as described below:

Dichloromethane (15 ml) was added to the reaction mixture, followed by a 1:1 mixture (8 ml) of saturated sodium thiosulfate solution and saturated sodium bicarbonate solution. The phases were separated and the organic phase washed with another 5 ml of the thiosulfate/bicarbonate solution. The organic phase was then washed with brine and then dried over magnesium sulfate. The crude product was subjected to flash chromatography using a silica gel cartridge to give (R)-N-[(S)-1-(benzoxazole-2-carbonyl)-butyl]-2-isopropylamino-3-phenylmethanesulfonyl propionamide (6.2 mg, 20%). LC/MS retention time 2.7 min (TIC), m/z=486 (M+H) (determined with method C).

### (b) (R)-N-[(S)-1-(Benzoxazole-2-carbonyl)-butyl]2-[(2-methoxy-ethyl)-(tetrahydro-pyran-4-yl)-amino]-3-phenylmethanesulfonyl-propionamide

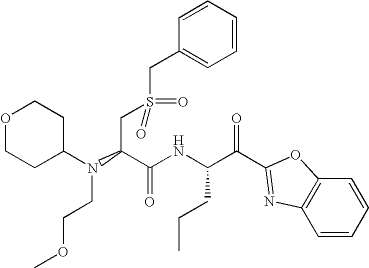

By proceeding in a similar manner to Example 21(a) but using (R)-N-[(S)-1-(Benzoxazol-2-yl-hydroxy-methyl)-butyl]-2-[(2-methoxy-ethyl)-(tetrahydro-pyran-4-yl)-amino]-3-phenylmethanesulfonyl-propionamide {80 mg, 0.136 mmol, Example 32(d)} there was prepared (R)-N-[(S)-1-(Benzoxazole-2-carbonyl)-butyl]-2-[(2-methoxy-ethyl)-(tetrahydro-pyran-4-yl)-amino]-3-phenylmethanesulfonyl-propionamide (7 mg, 9%). LC/MS retention time 3.5 min (TIC), m/z=586 (M+H) (determined with method C).

### (c) (R)-N-[(S)-1-(Benzoxazole-2-carbonyl)-butyl]-2-cyclohexylamino-3-phenylmethanesulfonyl-propionamide

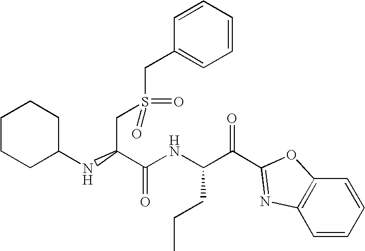

By proceeding in a similar manner to Example 21(a) but using (R)-N-[(S)-1-(Benzoxazol-2-yl-hydroxy-methyl)-butyl]-2-cyclohexylamino-3-phenylmethanesulfonyl-propionamide {48 mg, 0.091 mmol, Example 32(e)} there was prepared (R)-N-[(S)-1-(benzoxazole-2-carbonyl)-butyl]-2-cyclohexylamino-3-phenylmethanesulfonyl-propionamide (7.9 mg, 16%). LC/MS retention time 2.99-3.02 min (TIC), m/z=526 (M+H) (determined with method C).

### (d) (R)-N-[(S)-1-(Benzoxazole-2-carbonyl)-butyl]-2-dimethylamino-3-phenylmethanesulfonyl-propionamide

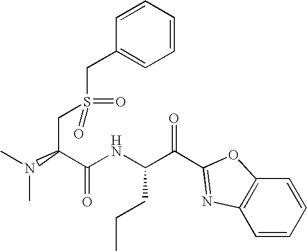

By proceeding in a similar manner to Example 21(a) but using (R)-N-[(S)-1-(Benzoxazol-2-yl-hydroxy-methyl)-butyl]-2-dimethylamino-3-phenylmethanesulfonyl-propionamide {10 mg, 0.021 mmol, Example 32(f)} there was prepared (R)-N-[(S)-1-(Benzoxazole-2-carbonyl)-butyl]-2-dimethylamino-3-phenylmethanesulfonyl-propionamide (2.5 mg, 24%). LC/MS retention time 2.82 min (TIC), m/z472 (M+H) (determined with method C).

### EXAMPLE 22

### (1S)-N-[1-(Benzooxazole-2-carbonyl)-butyl]-2-(S)-fluoro-4-phenyl-butyramide

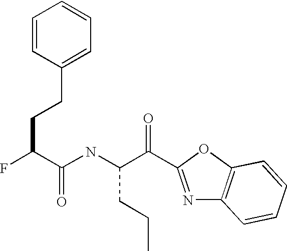

Step 1. To a mixture of (S)-2-amino-1-benzoxazol-2-yl-pentan-1-ol {0.549 mmol, 121 mg, Reference Example 17(c)}, (S)-2-fluoro-4-phenyl-butyric acid (1.0 eq., 0.549 mmol, 100 mg, Reference Example 9) and N,N-diispropylethylamine (1.1 eq., 0.604 mmol, 78 mg) in dry dichloromethane (5 mL) under nitrogen was added PyBOP® (1.1 eq., 0.603 mmol, 285 mg). The mixture was stirred at room temperature for 23.5 hr, then concentrated in vacuum. The residue was diluted with ethyl acetate (20 mL) and washed with sodium bicarbonate (30 mL) then water (30 mL). The organic layer was dried (MgSO₄) and concentrated in vacuum. The residue was purified by silica gel column chromatography, eluting with ethyl acetate and heptane (1:2) to afford (S)-N-[(S)-1-(Benzoxazol-2-yl-hydroxy-methyl)-butyl]-2-fluoro-4-phenyl-butyramide as mixture of diastereoisomers (167.8 mg, 79.5%).

Step 2. To a solution of (S)-N-[(S)-1-(Benzoxazol-2-yl-hydroxy-methyl)-butyl]-2-fluoro-4-phenyl-butyramide in dry dichloromethane (5 mL) under nitrogen was added a 15% (wt in dichloromethane, 2.0 eq, 0.863 mmol, 2.44 g) of 1,1,1-triacetoxy-1,1-dihydro-1,2-benziodoxol-3(1H)-one (Dess-Martin periodinane). The mixture was stirred at room temperature for 2 hr, then quenched by adding a solution of Na₂S₂O₃ (4.0 eq., 1.73 mmol, 273 mg) in saturated Sodium bicarbonate solution (30 ml). The organic layer was dried (MgSO4) and concentrated in vacuum. The residue was purified over 10 g silica gel, eluting with ethyl acetate and heptane (1:3) to afford (1S)-N-[1-(Benzooxazole-2-carbonyl)-butyl]-2-(S)-fluoro-4-phenyl-butyramide as a light yellow solid (156 mg, 94%). ¹H NMR (CDCl₃) 7.95 (d, J=7.9 Hz, 1H), 7.7 (d, J=8.2 Hz, 1H), 7.6 (t, J=7.3 Hz, 1H), 7.51 (t, J=7.4 Hz, 1H), 7.2 (m, 6H), 5.8 (m, 1H), 4.95 (ddd, J=49.4, 8, 3.5 Hz, 1H), 2.8 (m, 2H), 2.4 (m, 1H), 2.2 (m, 2H), 1.85 (m, 1H), 1.5 (m, 2H), 1.0 (t, J=7.3 Hz, 3H). LC/MS 86% (M+1) 383.

### EXAMPLE 23

### 2,2-Difluoro-5-phenyl-pentanoic acid [(S)-1-(benzoxazole-2-carbonyl)-butyl]-amide

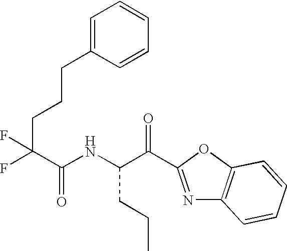

Step 1. A solution 2,2-Difluoro-5-phenyl-pentanoic acid (182 mg, 0.85 mmol) in DMF (10 mL) was treated with (S)-2-amino-1-benzoxazol-2-yl-pentan-1-ol (187 mg, 0.85 mmol), HATU (323 mg, 0.85 mmol) and N,N-Diisopropylethylamine (0.162 mL) and stirred at room temperature for 5½ hrs. DMF evaporate off, crude taken up in ethyl acetate and washed with 1N HCl, saturated NaHCO₃ and brine. Dried over Na₂SO₄ and evaporated under reduced pressure to give an oil. Purification by column chromatography eluting with 1:1 mixture of ethyl acetate and heptane gave 2,2-Difluoro-5-phenyl-pentanoic acid [(S)-1-(benzoxazol-2-yl-hydroxy-methyl)-butyl]-amide as orange oil (216 mg).

MS 417 (MH⁺).

Step 2. A solution of 2,2-Difluoro-5-phenyl-pentanoic acid [(S)-1-(benzoxazol-2-yl-hydroxy-methyl)-butyl]-amide (216 mg, 0.52 mmol) in dichloromethane (10 mL) was treated with 1,1,1-triacetoxy-1,1-dihydro-1,2-benziodoxol-3(1 H)-one (Dess-Martin periodinane) (220 mg, 0.52 mmol) for 1 hr at room temperature. The reaction mixture was washed with 0.5 M Na₂S₂O₃, saturated NaHCO₃, and water and dried over Na₂SO₄. Solvent evaporated under reduced pressure and crude purified by flash chromatography eluting with mixture of ethyl acetate and heptane to give 2,2-Difluoro-5-phenol-pentanoic acid [(S)-1-(benzoxazole-2-carbonyl)-butyl]-amide as off white solid (90 mg).

¹H NMR (CDCl₃) 7.93 (d, J=8 Hz, 1H), 7.68 (d, J=8 Hz, 1H), 7.59 (t, J=8 Hz, 1H), 7.49 (t, J=8 Hz, 1H), 7.3-7.11 (m, 5H), 5.72 (m, 1H), 2.67 (t, J=7.5 Hz, 2H), 2.22-2.07 (m, 3H), 1.92-1.77 (m, 3H), 1.55-1.26 (m, 2H), 0.96 (t, J=7.4 Hz, 3H). LC/MS 415(M+1).

### EXAMPLE 24

### (a) Morpholine-4-carboxylic acid (S)-1-[(S)-1-(benzooxazole-2-carbonyl)-propylcarbamoyl]-2-cyclohexyl-ethyl ester

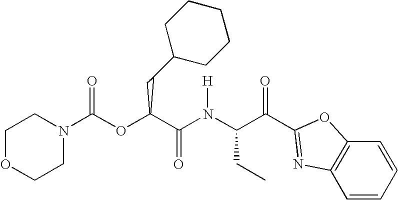

Step 1. (S)-3-Cyclohexyl-2-hydroxy-propionic acid (3 g, 17.4 mmol) was dissolved in methanol (30 mL). Trimethylorthoformate (5 mL) and p-toluenesulfonic acid monohydrate (100 mg) was added. The mixture was stirred at ambient temperature overnight. Water (50 mL) was added and stirring was continued for 2 h. Methanol was removed under vacuum and the aqueous residue was extracted with ethyl acetate (3×50 mL). The combined organic layers were washed with sat. aqueous NaHCO₃ and brine, dried with MgSO₄ and evaporated. (S)-3-Cyclohexyl-2-hydroxy-propionic acid methyl ester was obtained as a colorless liquid (3.1 g; 16.7 mmol).

Step 2. (S)-3-Cyclohexyl-2-hydroxy-propionic acid methyl ester (1 g, 5.37 mmol) was dissolved in dichloromethane (20 mL). Pyridine (0.57 mL, 7 mmol) was added and the solution was cooled to 0° C. under nitrogen. Trichloromethylchloroformate (0.66 mL, 5.5 mmol) was added and the mixture was stirred for 30 min at room temperature. Morpholine (0.5 mL) was added and stirring was continued for 2 h. After dilution with ethyl acetate (200 mL), the solution was washed with 1N aqueous. HCl and brine, dried with MgSO₄ and evaporated under vacuum. The residue was dissolved in methanol (50 mL) and 1N aqueous. NaOH solution (20 mL) was added. The mixture was stirred at room temperature for 4 h. Methanol was removed under vacuum and the aqueous residue was washed with diethylether. The aqueous layer was acidified with 1N aqueous HCl and extracted with ethyl acetate (3×100 mL). The combined organic layers were washed with brine, dried with MgSO₄ and evaporated under vacuum. The crude (S)-morpholine-4-carboxylic acid 1-carboxy-2-cyclohexyl-ethyl ester was used without further purification.

Step 3. By proceeding in a similar manner to that described in step3 Example 4(a) but using (S)-morpholine-4-carboxylic acid 1-carboxy-2-cyclohexyl-ethyl ester there was prepared morpholine-4-carboxylic acid (S)-1-[(S)-1-(benzooxazole-2-carbonyl)-propylcarbamoyl]-2-cyclohexyl-ethyl ester.

¹H NMR: (DMSO) 8.61 (d, J=6.4 Hz, 1H), 7.97 (d, J=8.0 Hz, 1H), 7.87 (d, J=8.0 Hz, 1H), 7.61 (t, J=8.0 Hz, 1H), 7.52 (t, J=8.0 Hz, 1H), 5.15-5.09 (m, 1H), 4.91-4.86 (m, 1H) 3.56-3.20 (m, 8H), 2.05-1.93 (m, 1H), 1.79-0.78 (m, 14H), 0.96 (t, J=7.2 Hz, 3H). MS: (M+H)⁺ 4.72.

By proceeding in a similar manner to Example 24(a) there was prepared:

### (b) Morpholine-4-carboxylic acid (S)-2-cyclohexyl-1-[(S)-1-(oxazolo[4,5-b]pyridine-2-carbonyl)-propylcarbamoyl]-ethyl ester

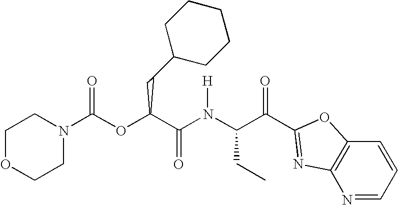

¹H NMR: (DMSO) 8.73-8.69 (m, 2H), 8.38 (d, J=8.0 Hz, 1H), 7.67-7.62 (m, 1H), 5.08-5.02 (m, 1H), 4.88-4.83 (m, 1H), 3.57-3.20 (m, 8H), 2.07-1.95 (m, 1H), 1.79-0.75 (m, 14H), 0.97 (t, J=7.2 Hz, 3H). MS: (M+H)⁺ 473;

### (c) Morpholine-4-carboxylic acid (S)-2-cyclohexyl-1-[(S)-1-(5-ethyl-[1,3,4]oxadiazole-2-carbonyl)-propylcarbamoyl]-ethyl ester

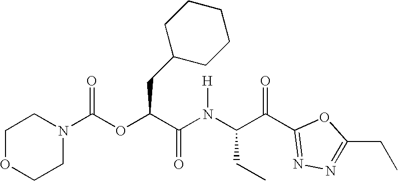

¹H NMR: (DMSO) 8.62 (d, J=4.8 Hz, 1H), 4.94-4.84 (m, 2H), 3.57-3.20 (m, 8H), 2.95 (q, J=7.2 Hz, 2H), 1.98-1.87 (m, 1H), 1.74-0.82 (m, 14H), 1.29 (t, J=7.2 Hz, 3H), 0.93 (t, J=7.2 Hz, 3H). MS: (M+H)⁺ 451;

### (d) Morpholine-4-carboxylic acid (S)-2-cyclohexyl-1-[(S)-1-(5-phenyl-[1,3,4oxadiazole-2-carbonyl)-propylcarbamoyl]-ethyl ester

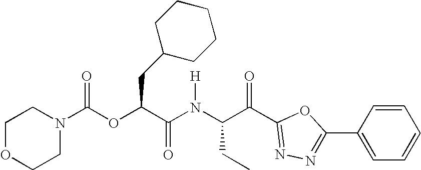

¹H NMR: (DMSO) 8.69 (d, J=6.0 Hz, 1H), 8.07 (d, J=8 Hz, 2H), 7.70-7.59 (m, 3H), 4.99-4.92 (m, 1H), 4.88-4.83 (m, 1H), 3.57-3.20 (m, 8H), 2.03-1.92 (m, 1H), 1.77-0.77 (m, 14H), 0.96 (t, J=7.2 Hz, 3H). MS: (M+H)⁺ 499;

### (e) Morpholine-4-carboxylic acid (S)-1-[(S)-1-(benzooxazole-2-carbonyl)-propylcarbamoyl]-3-cyclohexyl-propyl ester

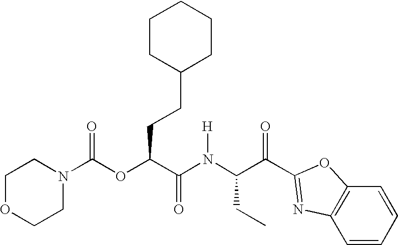

¹H NMR: (DMSO) 8.60 (d, J=6.8 Hz, 1H), 7.97 (d, J=8.0 Hz, 1H), 7.87 (d, J=8.0 Hz, 1H), 7.61 (t, J=8.0 Hz, 1H), 7.52 (t, J=8.0 Hz, 1H), 5.13-5.06 (m, 1H), 4.81-4.76 (m, 1H), 3.56-3.21 (m, 8H), 2.05-1.93 (m, 1H), 1.79-1.46 (m, 8H), 1.19-0.90 (m, 6H), 0.96 (t, J=7.2 Hz, 3H), 0.77-0.62 (m, 2H). MS: (M+H)⁺ 486;

### EXAMPLE 25

### 4-[4,4-Dimethyl-2-(morpholine-4-carbonyloxy)-pentanoylamino]-3-oxo-azepane-1-carboxylic acid benzyl ester

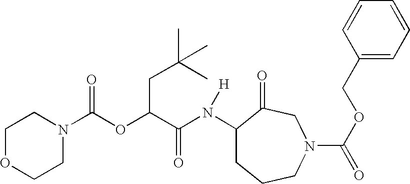

Sodium hydride (60% in mineral oil, 10 g, 250 mmol) was suspended in dry DMF. Allyl-carbamic acid benzyl ester (19.1 g, 100 mmol) was added dropwise at ambient temperature. After stirring for 5 min, 5-bromo-1-pentene (25 g, 168 mmol) was added dropwise. Stirring was continued at 50° C. for 1 h. The reaction was quenched with water and then partitioned between diethylether and water. The ether layer was washed with water and brine, dried with MgSO₄ and evaporated under vacuum. Flash chromatography (ethyl acetate/hexane 1:9) gave 15.5 g allyl-pent-4-enyl-carbamic acid benzyl ester.

Allyl-pent-4-enyl-carbamic acid benzyl ester (15.5 g, 59.8 mmol) was dissolved in dichloromethane and bis(tricyclohexylphosphine)benzylidene ruthenium(IV) dichloride (1 g) was added. The mixture was refluxed under a nitrogen atmosphere until TLC analysis showed complete reaction. The solvent was evaporated under vacuum and the residue was purified by flash chromatography (ethyl acetate/hexane 1:9). Yield: 7.8 g 2,3,4,7-Tetrahydro-azepine-1-carboxylic acid benzyl ester.

To a solution of 2,3,4,7-tetrahydro-azepine-1-carboxylic acid benzyl ester (4.5 g, 19.45 mmol) in dichloromethane (50 mL) was added m-chloroperbenzoic acid (60 mmol). The mixture was stirred at ambient temperature for 16 h. Sat aqueous K₂C0₃ solution was added and the mixture was extracted with dichloromethane. The combined organic layers were washed with sat. aqueous NaHCO₃ and brine, dried with MgSO₄ and evaporated under vacuum. The crude epoxide was dissolved in a 8:1 methanol/water mixture (100 mL). Ammonium chloride (3.2 g, 60 mmol) and sodium azide (3.9 g, 60 mmol) was added and the mixture was heated at 60° C. for 48 h. Most of the solvent was removed under vacuum. The residue was extracted with ethyl acetate. The combined organic layers were washed with sat. aqueous NaHCO₃ (200 mL) and brine (200 mL), dried with MgSO₄ and evaporated under vacuum. Flash chromatography of the residue (hexane/ethyl acetate 3:1) gave 3.3 g of 4-azido-3-hydroxy-azepane-1-carboxylic acid benzyl ester.

To a solution of 4-azido-3-hydroxy-azepane-1-carboxylic acid benzyl ester (3.3 g, 11.37 mmol) in methanol (50 mL) was added triethylamine (5 mL) and 1,3-propanedithiol (3.42 mL, 35 mmol). The mixture was stirred at ambient temperature until TLC analysis showed complete consumption of the starting material. A white precipitate was removed by filtration and the filtrate was evaporated to dryness. The residue was triturated with a 1:1 hexane/diethylether mixture to remove excess dithiol and dried under vacuum. Crude 4-amino-3-hydroxy-azepane-1-carboxylic acid benzyl ester (150 mg, 0.57 mmol), morpholine-4-carboxylic acid 1-carboxy-3,3-dimethyl-butyl ester (120 mg, 0.46 mmol), EDC (400 mg, 2.1 mmol), and HOBt (400 mg, 2.5 mmol) were combined. Dichloromethane (5 mL) was added and then 4-methylmorpholine (0.5 mL). The mixture was stirred at ambient temperature for 2 h. After dilution with ethyl acetate (100 mL) the solution was washed with 1N HCl, sat. aqueous NaHCO₃ and brine, dried with MgSO₄ and evaporated under vacuum. The residue was dissolved in DMSO (5 mL). Triethylamine (0.3 mL) and then SO₃ pyridine complex (150 mg) were added and the mixture was stirred at ambient temperature for 2 h. After dilution with ethyl acetate (100 mL), the solution was washed with water (50 mL) and brine, dried with MgSO₄ and evaporated under vacuum. The residue was purified by flash chromatography on silica gel and gave 4-[4,4-Dimethyl-2-(morpholine-4-carbonyloxy)-pentanoylamino]-3-oxo-azepane-1-carboxylic acid benzyl ester (95 mg, 0.189 mmol) as a white solid.

2:1 mixture of diastereomers, ¹H NMR: (DMSO) 8.14-8.08 (m, 1H), 7.40-7.25 (m, 5H), 5.18-4.89 (m, 3H), 4.51-4.33 (m, 2H), 4.01-3.76 (m, 2H), 3.60-3.25 (m, 8H), 2.95-2.79 (m, 1H), 1.84-1.54 (m, 6H), 0.92/0.91 (s, 9H). MS: (M+H)⁺ 504. LC/MS m/z474(M+H)

### EXAMPLE 26

### (a) (R)-N-[(S)-1-(Benzoxazole-2-carbonyl)-butyl]-3-cyclopropylmethanesulfonyl-2-(tetrahydro-pyran-4-ylamino)-propionamide

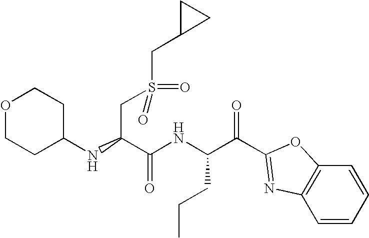

Step 1. (R)-2-Amino-N-[(S)-1-(benzoxazol-2-yl-hydroxy-methyl)-butyl]-3-cyclopropylmethanesulfonyl-propionamide {90 mg, 0.22 mmol, Reference Example 11(f)} was dissolved in 5% acetic acid in acetonitrile (10 ml). Tetrahydro-4H-pyran-4-one (110 mg, 1.1 mmol) was added, followed by (polystyrylmethyl)trimethylammonium cyanoborohydride (107 mg, 1.1 mmol). The resulting reaction mixture was stirred for four hours and then filtered under suction. The solvents were evaporated under high vacuum. The residue was dissolved in 5 ml dichloromethane, Silicycle Triamine (940 mg, 2.2 mmol) was added and the reaction mixture stirred for four hours. It was filtered under suction and the filtrate concentrated under reduced pressure to give (R)-N-[(S)-1-(Benzoxazol-2-yl-hydroxy-methyl)-butyl]-3-cyclopropylmethanesulfonyl-2-(tetrahydro-pyran-4-ylamino)-propionamide (89 mg, 0.18 mmol, 82%).

Step 2. (R)-N-[(S)-1-(Benzoxazol-2-yl-hydroxy-methyl)-butyl]-3-cyclopropylmethanesulfonyl-2-(tetrahydro-pyran-4-ylamino)-propionamide (89 mg, 0.18 mmol) was dissolved in 10 ml dichloromethane. The Dess-Martin-periodinane (153 mg, 0.36 mmol) was added and the resulting reaction mixture stirred for two hours. The reaction mixture was poured into a 1/1-mixture of saturated sodium bicarbonate solution and saturated sodium thiosulfate solution. The aqueous phase was extracted with dichloromethane. The combined organic phases were washed with saturated sodium bicarbonate solution and brine. The organic phase was dried with magnesium sulfate and the dichloromethane evaporated under reduced pressure. The crude product was purified via flash chromatography (heptane/ethyl acetate 1/1 to elute) to give (R)-N-[(S)-1-(benzoxazole-2-carbonyl)-butyl]-3-cyclopropylmethanesulfonyl-2-(tetrahydro-pyran-4-ylamino)-propionamide (24 mg, 0.049 mmol, 27%). ¹H NMR (CDCl₃, 300 MHz): 8.29 (d, J=8.5 Hz, 1H), 7.93 (d, J=8 Hz, 1H), 7.68 (d, J=8 Hz, 1H), 7.59-7.46 (m, 2H), 5.67 (m, 1H), 3.99-3.93 (m, 2H), 3.84 (dd, J=9.5 Hz, 2.5 Hz, 1H), 3.56 (dd, J=14.5 Hz, 2.5 Hz, 1H), 3.42-3.33 (m, 2H), 3.24 (dd, J=14.5 Hz, 9.5 Hz, 1H), 3.02-2.99 (m, 2H), 2.78-2.71 (m, 1H), 2.13-2.07 (m, 1H), 1.95-1.78 (m, 3H), 1.55-1.41 (m, 5H), 1.23-1.16 (m, 1H), 1.00 (t, J=7.5 Hz, 3H), 0.81-0.74 (m, 2H), 0.48-0.43 (m, 2H). LC/MS m/z=492 (M+H)

### (b) (R)-N-[1-(benzoxazole-2-carbonyl)-butyl]-2-cyclohexylamino-3-cyclopropylmethanesulfonyl-propionamide

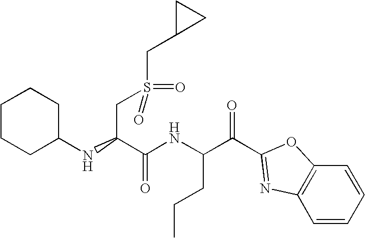

By proceeding in a similar manner to Example 26(a) but using cyclohexanone there was prepared (R)-N-[1-(benzoxazole-2-carbonyl)-butyl]2-cyclohexylamino-3-cyclopropylmethanesulfonyl-propionamide (predominantly as one diastereomer). ¹H NMR (CDCl₃, 300 MHz): 8.37 (d, J=8.5 Hz, 1H), 7.92 (d, J=8 Hz, 1H), 7.67 (d, J=8 Hz, 1H), 7.59-7.36 (m, 2H), 5.65 (m, 1H), 3.79 (dd, J=9.5 Hz, 2.5 Hz, 1H), 3.54 (dd, J=14.25 Hz, 2.5 Hz, 1H), 3.24 (dd, J=14.25 Hz, 9.5 Hz, 1H), 3.02-2.95 (m, 2H), 2.49 (m, 1H), 2.12-2.07 (m, 1H), 1.96-1.17 (m, 15H), 0.98 (t, J=7 Hz, 3H), 0.80-0.72 (m, 2H), 0.48-0.43 (m, 2H). LC/MS m/z=490 (M+H)

### (c) (R)-N-[1-(Benzoxazole-2-carbonyl)-butyl]-2-cycloheptylamino-3-cyclopropylmethanesulfonyl-propionamide

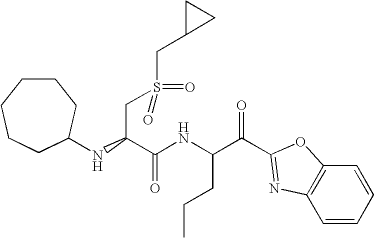

By proceeding in a similar manner to Example 26(a) but using cycloheptanone there was prepared (R)-N-[1-(benzoxazole-2-carbonyl)-butyl]-2-cycloheptylamino-3-cyclopropylmethanesulfonyl-propionamide ¹H NMR (CDCl₃, 300 MHz): [8.36 (d, J=8.5 Hz), 8.28 (d, J=8.5 Hz), 1H], [8.05 (dd, J=8 Hz, 1 Hz), 7.97 (dd, J=8.5 Hz, 1.5 Hz), 1H], [7.92 (d, J=8.5 Hz), 7.67 (d, J=8 Hz), 1H], 7.59-7.48 (m, 1H), [7.44 (ddd, J=8 Hz, 7.5 Hz, 1 Hz), 7.19 (ddd, J=8 Hz, 7.5 Hz, 1 Hz), 1H], [5.65 (m), 5.62 (m), 1H], [3.82 (dd, J=10 Hz, 3 Hz), 3.75 (dd, J=9 Hz, 3 Hz), 1H], [3.55 (dd, J=14.5 Hz, 3 Hz), 3.49 (dd, J=14.5 Hz, 3 Hz), 1H], 3.27 (dd, J=14.5 Hz, 9 Hz, 1H), 3.03-2.96 (m, 2H), 2.72 (m, 1H), 2.14-2.05 (m, 1H), 1.91-1.39 (m, 16H), 1.23-1.17 (m, 1H), [0.99 (t, J=7.25 Hz), 0.98 (t, J=7.25 Hz), 1H], 0.79-0.7 (m, 2H), 0.48-0.44 (m, 2H). LC/MS m/z=504 (M+H).

### (d) (R)-3-Phenylmethanesulfonyl-N-[(S)-3-phenyl-1-(thiazole-2-carbonyl)-propyl]-2-(tetrahydro-pyran-4-ylamino)-propionamide

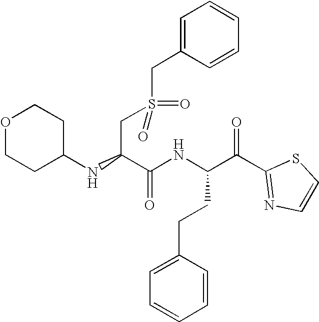

By proceeding in a similar manner to Example 26(a) but using (R)-2-Amino-N-[(S)-1-(hydroxy-thiazol-2-yl-methyl)-3-phenyl-propyl]-3-phenylmethanesulfonyl-propionamide {Reference Example 11(k)} there was prepared (R)-3-phenylmethanesulfonyl-N-[(S)-3-phenyl-1-(thiazole-2-carbonyl)-propyl]-2-(tetrahydro-pyran-4-ylamino)-propionamide. ¹H NMR (CDCl₃, 300 MHz): 8.27 (d, J=9 Hz, 1H), 8.06 (d, J=3 Hz, 11 H), 7.73 (d, J=3 Hz, 1H), 7.47-7.39 (m, 5H), 7.25-7.11 (m, 5H), 5.72 (m, 1H), 4.36 (d, J=14 Hz, 1H), 4.31 (d, J=14 Hz, 1H), 3.97-3.90 (m, 2H), 3.76 (dd, J=9.5 Hz, 3 Hz, 1H), 3.40-3.31 (m, 3H), 3.01 (dd, J=14.5 Hz, 9.5 Hz, 1H), 2.76-2.62 (m, 3H), 2.51-2.40 (m, 1H), 2.22-2.09 (m, 1H), 1.87-1.75 (m, 2H), 1.53-1.38 (m, 3H) LC/MS m/z=556 (M+H);

### (e) (R)-N-[(S)-1-(Benzoxazole-2-carbonyl)-3-phenyl-propyl}-3-cyclolpropylmethanesulfonyl-2-(tetrahydro-pyran-4-ylamino)-propionamide

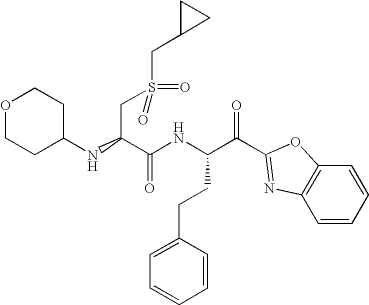

By proceeding in a similar manner to Example 26(a) but using (R)-2-amino-N-[(S)-1-(hydroxy-thiazol-2-yl-methyl)-3-phenyl-propyl]-3-phenylmethanesulfonyl-propionamide {Reference Example 11(j)} there was prepared (R)-N-[(S)-1-(Benzoxazole-2-carbonyl)-3-phenyl-propyl}-3-cyclopropylmethanesulfonyl-2-(tetrahydro-pyran-4-ylamino)-propionamide. ¹H NMR (CDCl₃, 300 MHz): 8.36 (d, J=8.5 Hz, 1H), 7.92 (d, J=8 Hz, 1H), 7.67 (d, J=8 Hz, 1H), 7.60-7.46 (m, 2H), 7.25-7.16 (m, 5H), 5.72 (m, 1H), 3.99-3.93 (m, 2H), 3.81 (dd, J=9.5 Hz, 3 Hz, 1H), 3.52 (dd, J=14 Hz, 3 Hz, 1H), 3.41-3.33 (m, 2H), 3.15 (dd, J=14 Hz, 9.5 Hz, 1H), 3.01-2.70 (m, 2H), 2.81-2.70 (m, 3H), 2.53 (m, 1H), 2.27-2.23 (m, 1H), 1.94-1.44 (m, 5H), 1.22-1.17 (m, 1H), 0.80-0.74 (m, 2H), 0.47-0.42 (m, 2H). LC/MS m/z=554 (M+H);

### (f) (R)-3-Cyclopropylmethanesulfonyl-N-[1-(5-ethyl-1,2,4-oxadiazole-3-carbonyl)-propyl}-2-(tetrahydro-pyran-4-ylamino)-propionamide

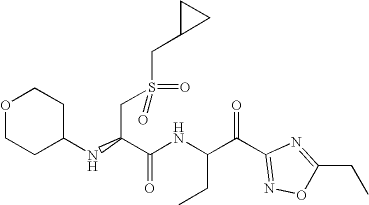

By proceeding in a similar manner to Example 26(a) but using (R)-2-Amino-3-cyclopropylmethanesulfonyl-N-{(S)-1-[(5-ethyl-1,2,4-oxadiazol-3-yl)-hydroxy-methyl]-propyl}-propionamide {Reference Example 11(h)} there was prepared (R)-3-cyclopropylmethanesulfonyl-N-[1-(5-ethyl-1,2,4-oxadiazole-3-carbonyl)-propyl]-2-(tetrahydro-pyran-4-ylamino)-propionamide. ¹H NMR (CDCl₃, 300 MHz): [8.28 (d, J=8.5 Hz), 8.15 (d, J=8 Hz), 1H], [5.40 (m), 5.33 (m), 1H], 3.99-3.95 (m, 2H), [3.90 (dd, J=10 Hz, 3 Hz), 3.84 (dd, J=9.5 Hz, 3 Hz), 1H], [3.55 (dd, J=14 Hz, 3 Hz), 3.47 (dd, J=14 hz, 11 Hz), 1H], 3.45-3.33 (m, 2H), 3.23 (dd, 14 Hz, 10 Hz, 1H), 3.07-2.94 (m, 4H), 2.82-2.71 (m, 1H), 2.19-2.08 (m, 1H), 1.95-1.77 (m, 5H), 1.58-1.43 (m, 1H), 1.45 (t, J=7.5 Hz, 3H), 1.23-1.14 (m, 1H), [1.00 (t, J=7.5 Hz), 0.97 (t, J=7.5 Hz), 3H], 0.81-0.73 (m, 2H), 0.48-0.41 (m, 2H). LC/MS m/z=457 (M+H);

### (g) (R)-3-Phenylmethanesulfonyl-N-[1-(3-phenyl-1,2,4-oxadiazole-5-carbonyl)-propyl]-2-(tetrahydro-pyran-4-ylamino)-propionamide

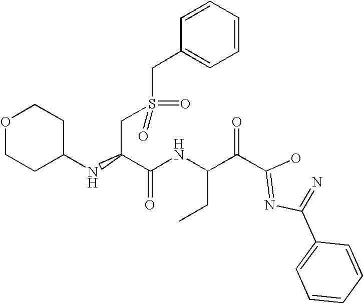

By proceeding in a similar manner to Example 26(a) but using (R)-2-Amino-N-{1-[hydroxy-(3-phenyl-1,2,4-oxadiazol-5-yl)-methyl]-propyl}-3-phenylmethanesulfonyl-propionamide {Reference Example 11(g)} there was prepared (R)-3-phenylmethanesulfonyl-N-[1-(3-phenyl-1,2,4-oxadiazole-5-carbonyl)-propyl}-2-(tetrahydro-pyran-4-ylamino)-propionamide. ¹H NMR (CDCl₃, 300 MHz): [8.15 (d, J=8 Hz), 8.14 (d, J=8 Hz), 1H], 7.61-7.39 (m, 10 H), [5.46 (m), 5.40 (m), 1H], 4.34-4.28 (m, 2H), 4.09-3.93 (m, 2H), [3.87 (dd, J=9.5 Hz, 3 Hz), 3.81 (dd, J=9.5 Hz, 3 Hz), 1H], 3.41-3.32 (m, 3H), [3.16 (dd, J=13.5 Hz, 10 Hz), 3.11 (dd, J=14 Hz, 9.5 Hz), 1H], 2.75-2.68 (m, 1H), 2.23-2.13 (m, 1H), 1.96-1.43 (m, 6H), 1.06-0.99 (m, 3H), LC/MS m/z=541 (M+H).

### (h) (R)-N-[1-(3-Cyclopropyl-1,2,4-oxadiazole-5-carbonyl)-propyl}-3-phenylmethanesulfonyl-2-(tetrahydro-pyran-4-ylamino)-propionamide

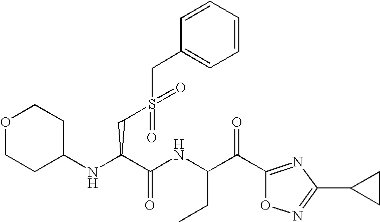

By proceeding in a similar manner to Example 26(a) but using (R)-2-Amino-3-phenylmethanesulfonyl-N-{(S)-1-[(3-cyclopropyl-1,2,4-oxadiazol-5-yl)-hydroxy-methyl]-propyl}-propionamide {Reference Example 11(l)} there was prepared (R)-N-[1-(3-cyclopropyl-1,2,4-oxadiazole-5-carbonyl)-propyl]-3-phenylmethanesulfonyl-2-(tetrahydro-pyran-4-ylamino)-propionamide. ¹H NMR (CDCl₃, 300 MHz): [8.19 (d, J=8.5 Hz), 8.11 (d, J=7.5 Hz), 1H], 7.46-7.40 (m, 5H), [5.33 (m), 5.27 (m), 1H], 4.55-4.35 (m, 2H), 3.99-3.95 (m, 2H), [3.88 (dd, J=10 Hz, 3 Hz), 3.83 (dd, J=9.5 Hz, 3 Hz), 1H], 3.44-3.34 (m, 3H), 3.18-3.07 (m, 1H), 2.78-2.67 (m, 1H), 2.24-2.17 (m, 1H), 2.15-2.08 (m, 1H), 1.89-1.72 (m, 3H), 1.55-1.43 (m, 2H), 1.20-1.11 (m, 4H), [0.98 (t, J=7.5 Hz), 0.97 (t, J=7.5 Hz), 3H]. LC/MS m/z=505 (M+H).

### EXAMPLE 27

### (a) {(R)-1-[1-(Benzothiazol-2-yl-hydroxy-methyl)-butylcarbamoyl]-2-phenylmethanesulfonyl-ethyl]-carbamic acid tert-butyl ester

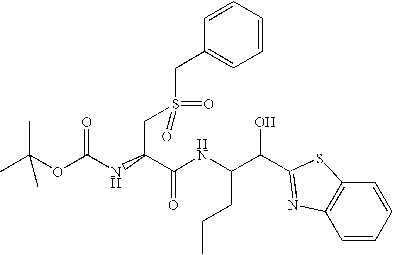

N-cyclohexylcarbodiimide, N′-methyl polystyrene (1.74 g, 3.4 mmol) suspended in a mixture of dichloromethane (10 ml) and dimethylformamide (2 mL) was treated with hydroxybenzotriazole (391 mg, 2.89 mmol) and L-N-boc-benzylsulfonylalanine (876 mg, 2.55 mmol). This mixture was stirred at room temperature for 30 minutes, then treated with 2-amino-1-benzothiazol-2-yl-pentan-1-ol {400 mg, 1.7 mmol, Reference Example 17(d)}) and after stirring for a further 2 hours the mixture was then treated with Silicycle-Triamine (2.36 g, 8.5 mmol). The reaction mixture was stirred for 2 hours and then filtered. The filtrate was evaporated to give the title compound (888 mg, 93%). LC/MS m/z=562.

### (b) {(R)-1-[(S)-1-(Benzoxazol-2-yl-hydroxy-methyl)-butylcarbamoyl]-2-phenylmethanesulfonyl-ethyl}-carbamic acid tert-butyl ester

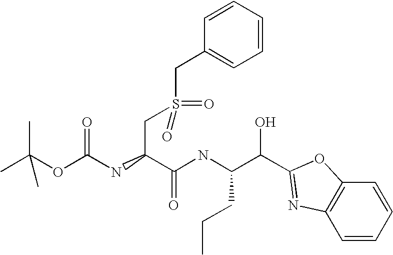

By proceeding in a manner similar to Example 27(a) above but using L-N-boc-benylsulfonylalanine (876 mg, 2.55 mmol) and (2S)-2-amino-1-benzooxazol-2-yl-pentan-1-ol {374 mg, 1.7 mmol, Reference Example 17(c)} there was prepared {(R)-1-[(S)-1-(benzoxazol-2-yl-hydroxy-methyl)-butylcarbamoyl]-2-phenylmethanesulfonyl-ethyl}-carbamic acid tert-butyl ester (908 mg, 98%).

### (c) {(S)-1-[(S)-1-(Benzoxazol-2-yl-hydroxy-methyl)-butylcarbamoyl]-2-thiophen-2-yl-ethyl}-carbamic acid tert-butyl ester

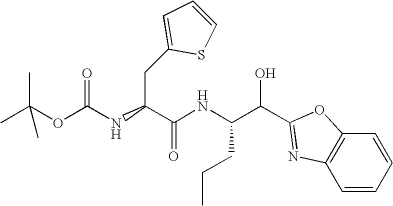

By proceeding in a manner similar to Example 27(a) above but using Resin-bound diimide (1.76 g, 3.4 mmol) suspended in dichloromethane (10 mL), hydroxybenzotriazole (391 mg, 2.89 mmol), (2S)-2-tert-butoxycarbonylamino-3-thiophen-2-yl-propionic acid (692 mg, 2.55 mmol), (2S)-2-amino-1-benzooxazol-2-yl-pentan-1-ol {374 mg, 1.7 mmol, Reference Example 17(c)} and Silicycle-Triamine (2.36 g, 8.5 mmol) there was prepared {(S)-1-[(S)-1-(Benzoxazol-2-yl-hydroxy-methyl)-butylcarbamoyl]-2-thiophen-2-yl-ethyl}-carbamic acid tert-butyl ester (790 mg (1.67 mmol, 98%). LC/MS:m/z=562 (M+H).

### (d) {(R)-1-[1-(Benzothiazol-2-yl-hydroxy-methyl)-butylcarbamoyl]-2-phenylmethanesulfonyl-ethyl}-carbamic acid tert-butyl ester

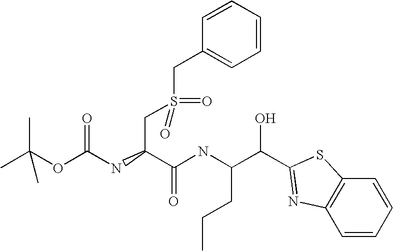

By proceeding in a manner similar to Example 27(a) above but using Resin-bound diimide (741 mg, 1.26 mmol), hydroxybenzotriazole (144 mg, 1.07 mmol), L-N-boc-benzylsulfonylalanine (326 mg, 0.95 mmol), 2-amino-1-benzothiazol-2-yl-pentan-1-ol {150 mg, 0.63 mmol, Reference Example 17(d)} and Silicycle-Triamine (2.36 g, 8.5 mmol) there was prepared {(R)-1-[1-(benzothiazol-2-yl-hydroxy-methyl)-butylcarbamoyl]-2-phenylmethanesulfonyl-ethyl}-carbamic acid tert-butl ester, LC/MS m/z=562 (M+H), which was used without further purification

### (e) {(R)-1-[(S)-1-(Benzoxazol-2-yl-hydroxy-methyl)-butylcarbamoyl]-2-phenylmethanesulfonyl-ethyl}-carbamic acid tert-butyl ester

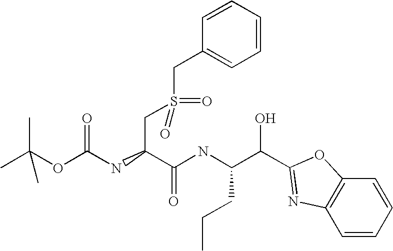

By proceeding in a manner similar to Example 27(a) above but using Resin-bound diimide (1.76 g, 3.4 mmol), hydroxybenzotriazole (391 mg, 2.89 mmol), L-N-boc-benzylsulfonylalanine (876 mg, 2.55 mmol), (2S)-2-amino-1-benzooxazol-2-yl-pentan-1-ol {374 mg, 1.7 mmol, Reference Example 17(c)} and Silicycle-Triamine (2.36 g, 8.5 mmol) there was prepared {(R)-1-[(S)-1-(benzoxazol-2-yl-hydroxy-methyl)-butylcarbamoyl]-2-phenylmethanesulfonyl-ethyl}-carbamic acid tert-butyl ester, LC/MS m/z=546 (M+H), 490 (M=H-butene), which was used directly in the next reaction.

### {(R)-1-[(S)-1-(Benzoxazol-2-yl-hydroxy-methyl)-butylcarbamoyl]-2-cyclopropylmethanesulfonyl-ethyl}-carbamic acid tert-butyl ester

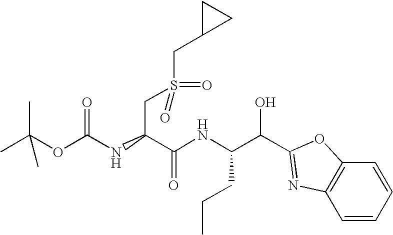

By proceeding in a manner similar to Example 27(a) above but using a suspension of Resin-bound diimide (1.07 g, 1.82 mmol) in dichloromethane (20 ml), hydroxybenzotriazole (209 mg, 1.55 mmol) and (R)-2-tert-butoxycarbonylamino-3-cyclopropylmethanesulfonyl-propionic acid (420 mg, 1.365 mmol, Reference Example 22), (S)-2-amino-1-benzoxazol-2-yl-pentan-1-ol {200 mg 0.91 mmol, Reference Example 17(c)} and Silicycle-Triamine (2.8 g, 9.1 mmol) there was prepared {(R)-1-[(S)-1-(benzoxazol-2-yl-hydroxy-methyl)-butylcarbamoyl]-2-cyclopropylmethanesulfonyl-ethyl}-carbamic acid tert-butyl ester (450 mg, 97%). LC/MS m/z=532(M+Na), 510 (M+H), 454 (M+H-isobutene).

### (g) (R)-1-{1-[Hydroxy-(3-phenyl-1,2,4-oxadiazol-5-yl)-methyl]-propylcarbamoyl}-2-phenylmethanesulfonyl-ethyl)-carbamic acid tert-butyl ester

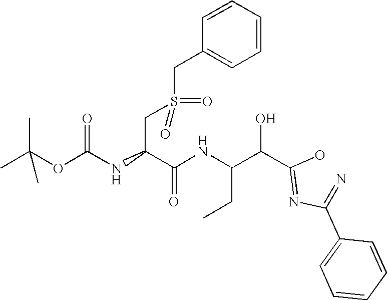

By proceeding in a manner similar to Example 27(f) above but using L-N-boc-benzylsulfonylalanine and (R)-2-tert-butoxycarbonylamino-3-phenylmethanesulfonyl-propionic acid and (S)-2-amino-1-(3-phenyl-[1,2,4]oxadiazol-5-yl)-butan-1-ol (Reference Example 21) there was prepared (R)-1-{1-[hydroxy-(3-phenyl-1,2,4-oxadiazol-5-yl)-methyl]-propylcarbamoyl}-2-phenylmethanesulfonyl-ethyl)-carbamic acid tert-butyl ester. LC/MS m/z=545(M+Na), 467 (M+H-isobutene), 423 (M+H-Boc).

### (i) ((R)-2-Cyclopropylmethanesulfonyl-1-{(S)-1-[(5-ethyl-1,2,4-oxadiazol-3-yl)-hydroxy-methyl]-propylcarbamoyl}-ethyl)-carbamic acid tert-butyl ester

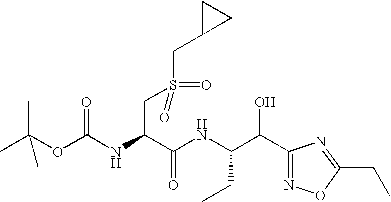

By proceeding in a manner similar to Example 27(f) above but using 2-amino-1-(5-ethyl-[1,2,4]-oxadiazol-3-yl-butan-1-ol (Reference Example 23) there was prepared ((R)-2-cyclopropylmethanesulfonyl-1-{(S)-1-[(5-ethyl-1,2,4-oxadiazol-3-yl)-hydroxy-methyl]-propylcarbamoyl}-ethyl)-carbamic acid tert-butyl ester. LC/MS m/z=497(M+Na), 419 (M+H-isobutene), 375 (M+H-Boc).

### (j) {(R)-1-[1-(Benzoxazol-2-yl-hydroxy-methyl)-butylcarbamoyl]-2-phenylmethanesulfonyl-ethyl}-carbamic acid tert-butyl ester

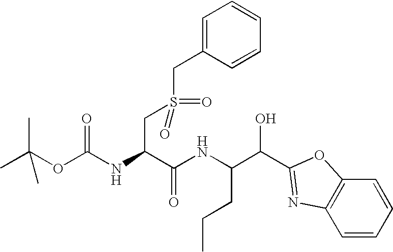

By proceeding in a manner similar to Example 27(f) above but using L-N-boc-benzylsulfonylalanine and (S)-2-amino-1-benzoxazol-2-yl-pentan-1-ol {Reference Example 17(c)} there was prepared {(R)-1-[1-(benzoxazol-2-yl-hydroxy-methyl)-butylcarbamoyl]-2-phenylmethanesulfonyl-ethyl}-carbamic acid tert-butyl ester. LC/MS m/z=546(M+H), 490 (M+H-isobutene).

### (k) {(R)-1-[(S)-1-(Benzoxazol-2-yl-hydroxy-methyl)-3-phenyl-propylcarbamoyl]-2-cyclopropylmethanesulfonyl-ethyl}-carbamic acid tert-butyl ester

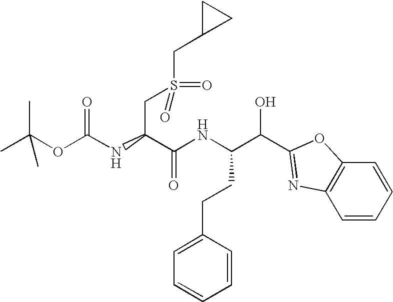

By proceeding in a manner similar to Example 27(f) above but using (2S)-2-amino-4-phenyl-1-benzoxazol-2-yl-butan-1-ol there was prepared {(R)-1-[(S)-1-(benzoxazol-2-yl-hydroxy-methyl)-3-phenyl-propylcarbamoyl]-2-cyclopropylmethanesulfonyl-ethyl}-carbamic acid tert-butyl ester. LC/MS m/z=572(M+H), 516 (M+H-isobutene).

### (l) {(R)-1-[(S)-1-(Hydroxy-thiazol-2-yl-methyl)-3-phenyl-propylcarbamoyl]-2-phenylmethanesulfonyl-ethyl}-carbamic acid tert-butyl ester

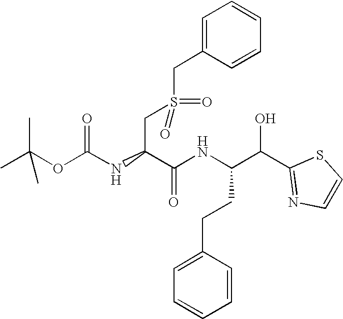

By proceeding in a manner similar to Example 27(f) above but using L-N-boc-benzylsulfonylalanine and (2S)-2-amino-4-phenyl-1-thiazol-2-yl-butan-1-ol (Reference Example 13) there was prepared {(R)-1-[(S)-1-(hydroxy-thiazol-2-yl-methyl)-3-phenyl-propylcarbamoyl]-2-phenylmethanesulfonyl-ethyl}-carbamic acid tert-butyl ester. LC/MS m/z=574(M+H).

### (m) {(R)-1-[(S)-1-(Benzoxazol-2-yl-hydroxy-methyl)-butylcarbamoyl]-2-cyclopropylmethanesulfonyl-ethyl}-carbamic acid tert-butyl ester

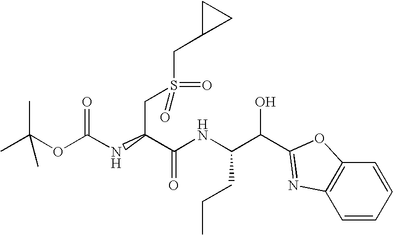

By proceeding in a manner similar to Example 27(f) above but using N-Cyclohexylcarbodiimide, N′-methyl polystyrene (1.07 g, 1.82 mmol) suspended in dichloromethane (20 mL), hydroxybenzotriazole (209 mg, 1.55 mmol), (R)-2-tert-butoxycarbonylamino-3-cyclopropylmethanesulfonyl-propionic acid (420 mg, 1.365 mmol, Reference Example 22), (S)-2-amino-1-benzoxazol-2-yl-pentan-1-ol {200 mg 0.91 mmol, Reference Example 17(c)} and Silicycle-Triamine (2.8 g, 9.1 mmol) there was prepared {(R)-1-[(S)-1-(benzoxazol-2-yl-hydroxy-methyl)-butylcarbamoyl]-2-cyclopropylmethanesulfonyl-ethyl}-carbamic acid tert-butyl ester (450 mg, 0.88 mmol, 97%). LC/MS m/z=532(M+Na), 510 (M+H), 454 (M+H-isobutene).

### (n) (R)-1-{1-[Hydroxy-(3-phenyl-1,2,4-oxadiazol-5-yl)-methyl]-propylcarbamoyl}-2-phenylmethanesulfonyl-ethyl)-carbamic acid tert-butyl ester

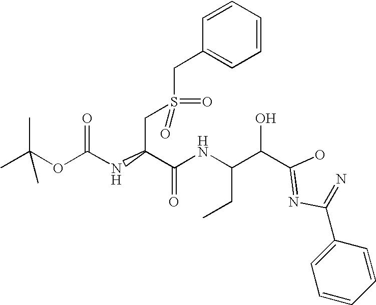

By proceeding in a manner similar to Example 27(m) above but using L-N-boc-benzylsulfonylalanine and (S)-2-amino-1-(3-phenyl-[1,2,4]oxadiazol-5-yl)-butan-1-ol (Reference Example 21) there was prepared (R)-1-{1-[hydroxy-(3-phenyl-1,2,4-oxadiazol-5-yl)-methyl]-propylcarbamoyl}-2-phenylmethanesulfonyl-ethyl)-carbamic acid tert-butyl ester. LC/MS m/z=545(M+Na), 467 (M+H-isobutene), 423 (M+H-Boc).

### (o) ((R)-2-Cyclopropylmethanesulfonyl-1-{(S)-1-[(5-ethyl-1,2,4-oxadiazol-3-yl)-hydroxy-methyl]-propylcarbamoyl}-ethyl)-carbamic acid tert-butyl ester

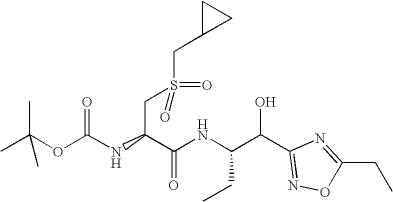

By proceeding in a manner similar to Example 27(m) above but using (S)-2-amino-1-(5-ethyl-[1,2,4]oxadiazol-3-yl)-butan-1-ol there was prepared ((R)-2-Cyclopropylmethanesulfonyl-1-{(S)-1-[(5-ethyl-1,2,4-oxadiazol-3-yl)-hydroxy-methyl]-propylcarbamoyl}-ethyl)-carbamic acid tert-butyl ester. LC/MS m/z=497(M+Na), 419 (M+H-isobutene), 375 (M+H-Boc)

### (p) {(R)-1-[1-(Benzoxazol-2-yl-hydroxy-methyl)-butylcarbamoyl]-2-phenylmethanesulfonyl-ethyl]-carbamic acid tert-butyl ester

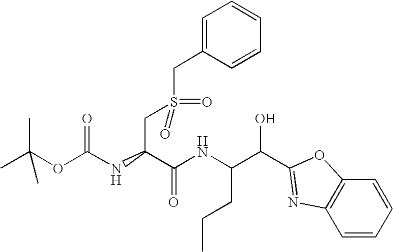

By proceeding in a manner similar to Example 27(m) above but using L-N-boc-benzylsulfonylalanine and (S)-2-amino-1-benzoxazol-2-yl-pentan-1-ol {200 mg 0.91 mmol, Reference Example 17(c)} there was prepared {(R)-1-[1-(Benzoxazol-2-yl-hydroxy-methyl)-butylcarbamoyl]-2-phenylmethanesulfonyl-ethyl}-carbamic acid tert-butyl ester. LC/MS m/z=546(M+H), 490 (M+H-isobutene)

### (q) {(R)-1-[(S)-1-(Benzoxazol-2-yl-hydroxy-methyl)-3-phenyl-propylcarbamoyl]-2-cyclopropylmethanesulfonyl-ethyl}-carbamic acid tert-butyl ester

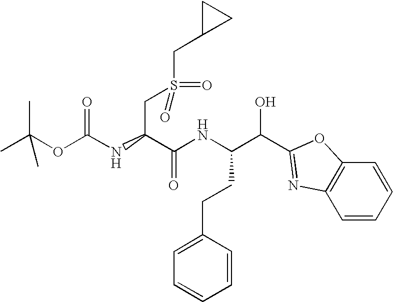

By proceeding in a manner similar to Example 27(m) above but using (2S)-2-amino-4-phenyl-1-benzoxazol-2-yl-butan-1-ol there was prepared {(R)-1-[(S)-1-(Benzoxazol-2-yl-hydroxy-methyl)-3-phenyl-propylcarbamoyl]-2-cyclopropylmethanesulfonyl-ethyl}-carbamic acid tert-butyl ester. LC/MS m/z=572(M+H), 516 (M+H-isobutene).

### (r) {(R)-1-[(S)-1-(Hydroxy-thiazol-2-yl-methyl)-3-phenyl-propylcarbamoyl]-2-phenylmethanesulfonyl-ethyl}-carbamic acid tert-butl ester

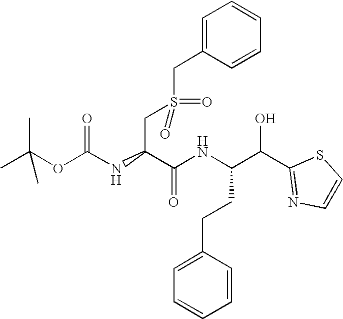

By proceeding in a manner similar to Example 27(m) above but using L-N-boc-benzylsulfonylalanine and (2S)-2-amino-4-phenyl-1-thiazol-2-yl-butan-1-ol (Reference Example 13) there was prepared {(R)-1-[(S)-1-(Hydroxy-thiazol-2-yl-methyl)-3-phenyl-propylcarbamoyl]-2-phenylmethanesulfonyl-ethyl}-carbamic acid tert-butyl ester. LC/MS m/z=5 74(M+H)

### (s) ((R)-2-phenylmethanesulfonyl-1-{(S)-1-[(3-cyclopropyl-1,2,4-oxadiazol-5-yl)-hydroxy-methyl]-propylcarbamoyl}-ethyl)-carbamic acid tert-butyl ester

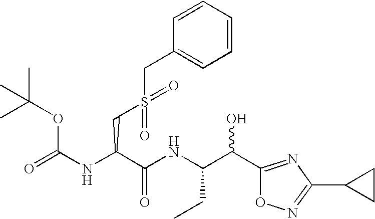

By proceeding in a manner similar to Example 27(m) above but using L-N-boc-benzylsulfonylalanine and (S)-2-amino-1-(3-cyclopropyl-1,2,4-oxadiazol-5-yl)-butan-1-ol (Reference Example 14) there was prepared ((R)-2-phenylmethanesulfonyl-1-{(S)-1-[(3-cyclopropyl-1,2,4-oxadiazol-5-yl)-hydroxy-methyl]-propylcarbamoyl}-ethyl)-carbamic acid tert-butyl ester.

### EXAMPLE 28

### (R)—N-[1-(Benzoxazole-2-carbonyl)-butyl]-2-[cyclopropylmethyl-(tetrahydro-pyran-4-ylmethyl)-amino]-3-phenylmethanesulfonyl-propionamide

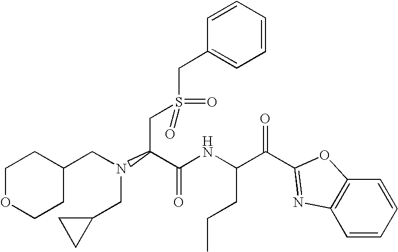

Step 1. (R)-2-Amino-N-[1-(benzoxazol-2-yl-hydroxy-methyl)-butyl]-3-phenylmethanesulfonyl-propionamide {200 mg, 0.448 mmol, Reference Example 11(i)} was dissolved in 5% acetic acid in acetonitrile (10 ml). Tetrahydro-pyran-4-carbaldehyde (51 mg, 0.448 mmol) was added and the reaction mixture stirred for 16 hours. (Polystyrylmethyl)trimethylammonium cyanoborohydride (218 mg, 0.896 mmol) was added and the reaction mixture stirred for 3 hours. Cyclopropanecarbaldehyde (157 mg, 2.24 mmol) was added and stirring continued for 3 hours. The mixture was filtered under suction and the filtrate concentrated under high vacuum.

Step 2. The residue was dissolved in 10 ml dichloromethane. The Dess-Martin-periodinane (380 mg, 0.896 mmol) was added and the resulting reaction mixture stirred for two hours. The reaction mixture was poured into a 1/1-mixture of saturated sodium bicarbonate solution and saturated sodium thiosulfate solution. The aqueous phase was extracted with dichloromethane.

The combined organic phases were washed with saturated sodium bicarbonate solution and brine. The organic phase was dried with magnesium sulfate and the dichloromethane evaporated under reduced pressure. The crude product was purified via flash chromatography (heptane/ethyl acetate 2/1 followed by heptane/ethyl acetate 1/1 to elute) to give R)—N-[1-(benzoxazole-2-carbonyl)-butyl]-2-[cyclopropylmethyl-(tetrahydro-pyran-4-ylmethyl)-amino]-3-phenylmethanesulfonyl-propionamide as mixture of diastereomers. (83 mg, 0.139 mmol, 31%). LC/MS m/z=596 (M+H) retention time 3.84 (method C).

### EXAMPLE 29

### (a) (R)—N-[1-(benzothiazol-2-yl-hydroxy-methyl)-butyl]-2-dibenzylamino-3-phenylmethanesulfonyl-propionamide

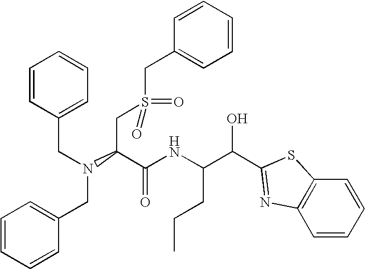

(R)-2-Amino-N-[1-(benzothiazol-2-yl-hydroxy-methyl)-butyl]-3-phenylmethanesulfonyl-propionamide {50 mg, 0.1 mmol, Reference Example 11(a)} was dissolved in a mixture of acetonitrile (5 ml) and acetic acid (1 ml). Benzaldehyde (56 μl, 0.55 mmol, 5 equivalents) and resin bound cyanoborohydride (54 mg, 0.22 mmol, 2 equivalents) were added. The reaction mixture was stirred overnight, filtered under suction and the filtrate evaporated to give the (R)—N-[1-(benzothiazol-2-yl-hydroxy-methyl)-butyl]-2-dibenzylamino-3-phenylmethanesulfonyl-propionamide which was used without further purification in the preparation of Example 18(c).

### (b) (R)—N-[1-(Benzothiazol-2-yl-hydroxy-methyl)-butyl]-3-phenylmethanesulfonyl-2-(tetrahydro-pyran-4-ylamino)-propionamide

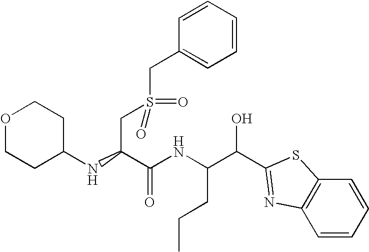

By proceeding in a manner similar to Example 29(a) above but using tetrahydro-4H-pyran-4-one (51 μl, 0.55 mmol, 5 equivalents) there was prepared (R)—N-[1-(benzothiazol-2-yl-hydroxy-methyl)-butyl]-3-phenylmethanesulfonyl-2-(tetrahydro-pyran-4-ylamino)-propionamide. LC/MS m/z=546 (M+H)

### EXAMPLE 30

### (a) (R)—N-[1-(Benzothiazol-2-yl-hydroxy-methyl)-butyl]-2-isopropylamino-3-phenylmethanesulfonyl-propionamide

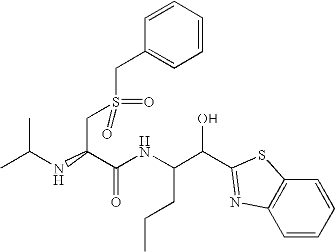

(R)-2-Amino-N-[1-(benzothiazol-2-yl-hydroxy-methyl)-butyl]-3-phenylmethanesulfonyl-propionamide {50 mg, 0.11 mmol, Reference Example 11(a)} was dissolved in a mixture of acetonitrile (5 ml) and acetic acid (1 ml). Acetone (500 μl) and resin bound cyanoborohydride (54 mg, 0.22 mmol, 2 equivalents) were added. The reaction mixture was stirred overnight, filtered under suction and concentrated under vacuum. The residue was dissolved in dichloromethane and AP Trisamine (Argonaut Technology) (550 mg, 1.2 mmol) was added. The mixture was stirred for two hours, filtered under suction and the filtrate concentrated under vacuum to give (R)—N-[1-(benzothiazol-2-yl-hydroxy-methyl)-butyl]-2-isopropylamino-3-phenylmethanesulfonyl-propionamide (30 mg, 0.06 mmol, 54%). LC/MS m/z=504 (M+H).

### (b) (R)—N-[1-(Benzothiazol-2-yl-hydroxy-methyl)-butyl]-2-dimethylamino-3-phenylmethanesulfonyl-propionamide

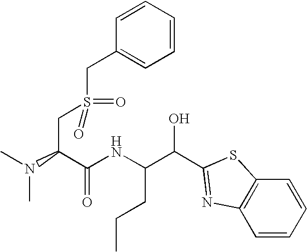

By proceeding in a manner similar to Example 30(a) above but using formaldehyde solution (75 μl, 1 mmol, 37 w-% aqueous solution) there was prepared (R)—N-[1-(benzothiazol-2-yl-hydroxy-methyl)-butyl]-2-dimethylamino-3-phenylmethanesulfonyl-propionamide (30 mg, 54%). LC/MS m/z=490 (M+H).

### EXAMPLE 31

### (a) (R)—N—[(S)-1-(Benzoxazol-2-yl-hydroxy-methyl)-butyl]-3-phenylmethanesulfonyl-2-(tetrahydro-pyran-4-ylamino)-propionamide

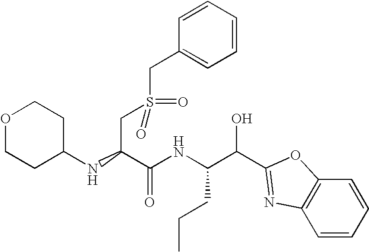

A solution of (R)-2-amino-N—[(S)-1-(benzoxazol-2-yl-hydroxy-methyl)-butyl]-3-phenylmethanesulfonyl-propionamide {100 mg, 0.22 mmol, Reference Example 11(c)} in a mixture of acetonitrile (5 mL) and acetic acid (1 mL) was treated with tetrahydro-4H-pyran-4-one (101 μl, 1.1 mmol). After agitating at room temperature for 3 hours the mixture was then treated with resin-bound cyanoborohydride (108 mg, 0.44 mmol) and agitation was continued overnight. The reaction mixture was filtered and the filtrate was evaporated. The residue was dissolved in dichloromethane (10 mL) and the solution was treated with Silicycle Triamine (611 mg, 2.2 mmol), then agitated for 2 hours and then filtered. The solution of (R)—N—[(S)-1-(benzoxazol-2-yl-hydroxy-methyl)-butyl]-3-phenylmethanesulfonyl-2-(tetrahydro-pyran-4-ylamino)-propionamide was used directly in the preparation of Example 20(b).

### (b) (R)—N-[(S)-1-(Benzoxazol-2-yl-hydroxy-methyl)-butyl]-2-(1-methyl-piperidin-4-ylamino)-3-phenylmethanesulfonyl-propionamide

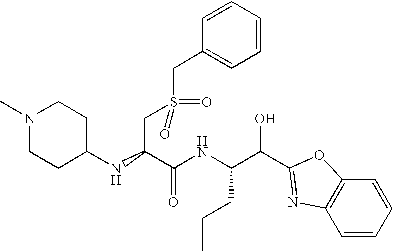

By proceeding in a manner similar to Example 31(a) above but using 1-methyl-4-piperidone (136 μl, 1.1 mmol) there was prepared (R)—N—[(S)-1-(Benzoxazol-2-yl-hydroxy-methyl)-butyl]-2-(1-methyl-piperidin-4-ylamino)-3-phenylmethanesulfonyl-propionamide was used directly in the preparation of Example 19(b).

### (c) (R)—N—[(S)-1-(Benzoxazol-2-yl-hydroxy-methyl)-butyl]-2-(bis-thiophen-2-ylmethyl-amino)-3-phenylmethanesulfonyl-propionamide

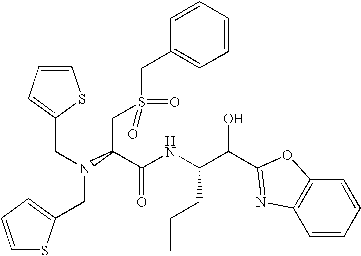

By proceeding in a manner similar to Example 31(a) above but using 2-thiophenecarboxaldehyde (20 μl, 0.22 mmol) there was prepared (R)—N—[(S)-1-(benzoxazol-2-yl-hydroxy-methyl)-bulyl]-2-(bis-thiophen-2-ylmethyl-amino)-3-phenylmethanesulfonyl-propionamide was used directly in the preparation of Example 19(c).

### (d) (R)—N—[(S)-1-(Benzoxazol-2-yl-hydroxy-methyl)-butyl]-2-dibenzylamino-3-phenylmethanesulfonyl-propionamide

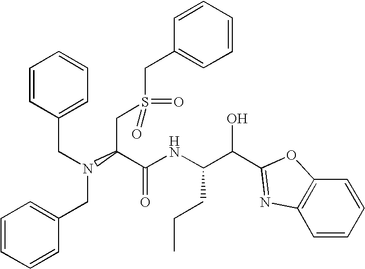

By proceeding in a manner similar to Example 31(a) above but using benzaldehyde (22 μl, 0.22 mmol) there was prepared (R)—N—[(S)-1-(benzoxazol-2-yl-hydroxy-methyl)-butl]-2-dibenzylamino-3-phenylmethanesulfonyl-propionamide which was used directly in the preparation of Example 19(d).

### (e) (S)—N—[(S)-1-(Benzoxazol-2-yl-hydroxy-methyl)-butyl]-2-(tetrahydro-pyran-4-ylamino)-3-thiolphen-2-yl-propionamide

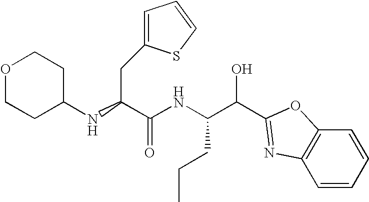

By proceeding in a manner similar to Example 317(a) above but using (S)-2-amino-N—[(S)-1-(benzoxazol-2-yl-hydroxy-methyl)-butyl]-3-thiophen-2-yl-propionamide {82 mg, 0.22 mmol, Reference Example 11(b)} and tetrahydro-4H-pyran-4-one (101 μI, 1.1 mmol) there was prepared (S)—N—[(S)-1-(Benzoxazol-2-yl-hydroxy-methyl)-butyl]-2-(tetrahydro-pyran-4-ylamino)-3-thiophen-2-yl-propionamide which was used directly in the preparation of Example 19(e).

### (f) (S)—N—[(S)-1-(Benzoxazol-2-yl-hydroxy-methyl)-butyl]-2-isopropylamino-3-thiophen-2-yl-propionamide

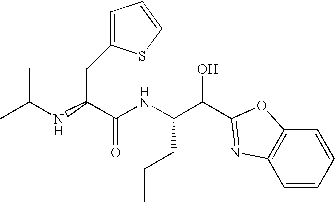

By proceeding in a manner similar to Example 31(a) above but using (S)-2-amino-N—[(S)-1-(benzoxazol-2-yl-hydroxy-methyl)-butyl]-3-thiophen-2-yl-propionamide {82 mg, 0.22 mmol, Reference Example 11(b)} and acetone (100 μl) there was prepared (S)—N—[(S)-1-(benzoxazol-2-yl-hydroxy-methyl)-butyl]-2-isopropylamino-3-thiophen-2-yl-propionamide which was used directly in the preparation of Example 19(f).

### (g) (R)—N—[(S)-1-(Benzoxazol-2-yl-hydroxy-methyl)-butyl]-2-isopropylamino-3-phenylmethanesulfonyl-propionamide

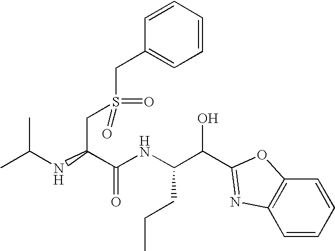

By proceeding in a manner similar to Example 31(a) above but using acetone (500 μl) there was prepared (R)—N—[(S)-1-(benzoxazol-2-yl-hydroxy-methyl)-butyl]-2-isopropylamino-3-phenylmethanesulfonyl-propionamide (30.5 mg, 29%). LC/MS m/z=488 (M+H).

### EXAMPLE 32

### (a) (R)—N-[1-(Benzothiazol-2-yl-hydroxy-methyl)-butyl]-3-phenylmethanesulfonyl-2-(tetrahydro-pyran-4-ylamino)-propionamide

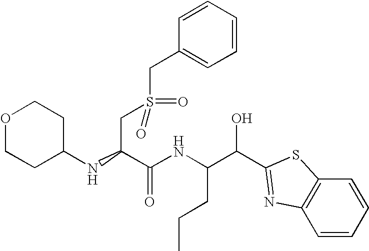

A solution of (R)-2-amino-N-[1-(benzothiazol-2-yl-hydroxy-methyl)-butyl]-3-phenylmethanesulfonyl-propionamide {100 mg, 0.22 mmol, Reference Example 11(a)} in a mixture of acetonitrile and acetic acid (10 mL, 95:5, v/v) was treated with tetrahydro-4H-pyran-4-one (101 μl, 1.1 mmol) and resin-bound cyanoborohydride (108 mg, 0.44 mmol). This mixture was stirred at room temperature overnight then evaporated. The residue was dissolved in dichloromethane and the solution was treated with Silicycle Triamine (611 mg, 2.2 mmol) then stirred at room temperature for 2 hours then filtered. The filtrate was evaporated to give (R)—N-[1-(benzothiazol-2-yl-hydroxy-methyl)-butyl]-3-phenylmethanesulfonyl-2-(tetrahydro-pyran-4-ylamino)-propionamide, LC/MS m/z=546 (M+H), which was used directly in the preparation of Example 18(b).

### (b) (R)—N—[(S)-1-(Benzoxazol-2-yl-hydroxy-methyl)-butyl]-3-phenylmethanesulfonyl-2-(tetrahydro-pyran-4-ylamino)-propionamide

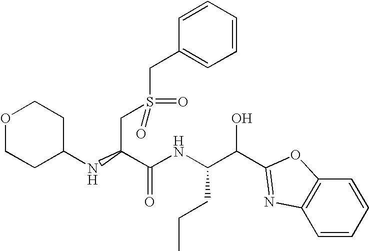

By proceeding in a manner similar to Example 32(a) above but using (R)-2-amino-N—[(S)-1-(benzoxazol-2-yl-hydroxy-methyl)-butyl]-3-phenylmethanesulfonyl-propionamide {98 mg, 0.22 mmol, Reference Example 11(c)} there was prepared (R)—N—[(S)-1-(benzoxazol-2-yl-hydroxy-methyl)-butyl]-3-phenylmethanesulfonyl-2-(tetrahydro-pyran-4-ylamino)-propionamide, LC/MS m/z=530 (M+H), which was used directly in the preparation of Example 19(a).

### (c) (R)—N—[(S)-1-(Benzoxazol-2-yl-hydroxy-methyl)-butyl]-3-phenylmethanesulfonyl-2-(tetrahydro-pyran-4-ylamino)-propionamide

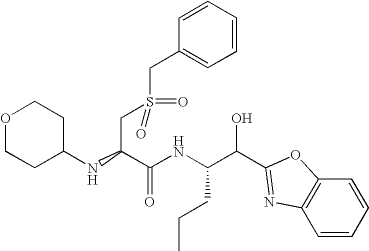

By proceeding in a manner similar to Example 32(a) above but using (R)-2-amino-N—[(S)-1-(benzoxazol-2-yl-hydroxy-methyl)-butyl]-3-phenylmethanesulfonyl-propionamide {Reference Example 11(c)} there was prepared (R)—N—[(S)-1-(benzoxazol-2-yl-hydroxy-methyl)-butyl]-3-phenylmethanesulfonyl-2-(tetrahydro-pyran-4-ylamino)-propionamide (106 mg, 91%). LC/MS m/z=530 (M+H).

### (d) (R)—N—[(S)-1-(Benzoxazol-2-yl-hydroxy-methyl)-butyl]-2-[(2-methoxy-ethyl)-(tetrahydro-pyran-4-yl)-amino]-3-phenylmethanesulfonyl-propionamide

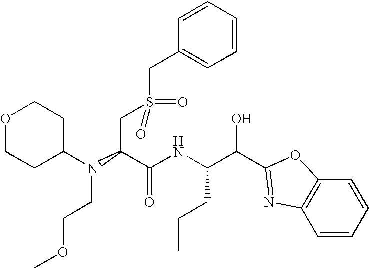

By proceeding in a manner similar to Example 32(a) above but using (R)—N—[(S)-1-(benzoxazol-2-yl-hydroxy-methyl)-butyl]-3-phenylmethanesulfonyl-2-(tetrahydro-pyran-4-ylamino)-propionamide {53 mg, 0.1 mmol, Reference Example32(c)} and 2-methoxyethanal (53 mg, 0.55 mmol) there was prepared (R)—N—[(S)-1-(benzoxazol-2-yl-hydroxy-methyl)-butyl]-2-[(2-methoxy-ethyl)-(tetrahydro-pyran-4-yl)-amino]-3-phenylmethanesulfonyl-propionamide (56 mg, 95%).

LC/MS m/z=588 (M+H)

### (e) (R)—N—[(S)-1-(Benzoxazol-2-yl-hydroxy-methyl)-butyl]-2-cyclohexylamino-3-phenylmethanesulfonyl-propionamide

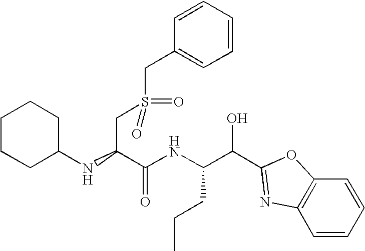

By proceeding in a manner similar to Example 32(a) above but using (R)-2-amino-N—[(S)-1-(benzoxazol-2-yl-hydroxy-methyl)-butyl]-3-phenylmethanesulfonyl-propionamide {49 mg, 0.11 mmol, Reference 11(c)} and cyclohexanone (52 μl, 0.5 mmol) there was prepared (R)—N—[(S)-1-(Benzoxazol-2-yl-hydroxy-methyl)-butyl]-2-cyclohexylamino-3-phenylmethanesulfonyl-propionamide (48 mg, 83%).

### (f) (R)—N—[(S)-1-(Benzoxazol-2-yl-hydroxy-methyl)-butyl]-2-dimethylamino-3-phenylmethanesulfonyl-propionamide

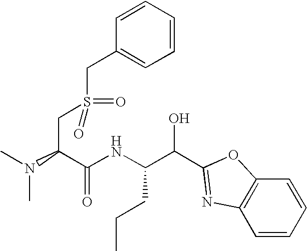

By proceeding in a manner similar to Example 32(a) above but using (R)-2-amino-N—[(S)-1-(benzoxazol-2-yl-hydroxy-methyl)-butyl]-3-phenylmethanesulfonyl-propionamide {49 mg, 0.11 mmol, Reference Example 11(c)} and formaldehyde (75 μl, 1 mmol, 37 w-% in water), there was prepared (R)—N—[(S)-1-(benzoxazol-2-yl-hydroxy-methyl)-butyl]-2-dimethylamino-3-phenylmethanesulfonyl-propionamide (10 mg, 19%). LC/MS m/z=474 (M+H).

### EXAMPLE 33

The following compounds of Formula 1 are provided by methods described in the application:

### (a) N-Cyanomethyl-3-cyclohexyl-propionamide

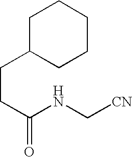

¹H NMR: (CDCl₃) 6.22 (br s, 1H), 4.20 (s, 2H), 2.23 (m, 2H), 1.65 (m, 5H), 1.50 (m, 2H), 1.10-1.30 (m, 4H), 0.90 (m, 2H); LC-MS: t=3.67 min., 193.0(M−1), 195.1(M+1). MS: API 150EX. (LC: Agilent 1100Series, Column: Phenomenex, 5 u ODS3 100A 100×3 mm. Flow Rate: 2 ml/min. Two solvent gradient: Solvent A, 99% water, 1% acetonitrile, 0.1% AcOH. Solvent B, 99% actonitrile, 1% water, 0.1% AcOH. Gradient from 100% A, 0% B to 0% A, 100% B from t=0 to t=6 min. Then gradient back to 100% A, 0% B from t=7 to t=15 min.);

### (b) N-Cyanomethyl-3-(2-difluoromethoxy-phenylmethanesulfonyl)-propionamide

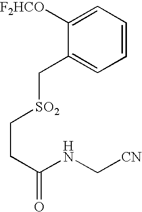

¹H NMR: (CDCl₃) 7.52 (d, 1H, J=8 Hz), 7.43 (t, 1H, J=8Hz), 7.29 (d, 1H, J=8 Hz), 7.20 (d, 1H, J=8 Hz), 6.40 (m, 1H), 4.41 (s,2H), 4.16 (d, 2H, J=6 Hz), 3.72 (s, 1H), 3.34 (t, 2H, J=8 Hz), 2.77 (t, 2H, J=8 Hz); LC-MS: t=3.02 min., 331.1(M−1), 333.1(M+1). MS: API 150EX. (LC: Agilent 1100Series, Column: Phenomenex, 5 u ODS3 100A 100×3 mm. Flow Rate: 2 ml/min. Two solvent gradient: Solvent A, 99% water, 1% acetonitrile, 0.1% AcOH. Solvent B, 99% actonitrile, 1% water, 0.1% AcOH. Gradient from 100% A, 0% B to 0% A, 100% B from t=0 to t=6 min. Then gradient back to 100% A, 0% B from t=7 to t=15 min.).

### (c) 3-(3-Cyclohexyl-propionylamino)-2-oxo-5-phenyl-pentanoic acid thiazol-2-ylamide

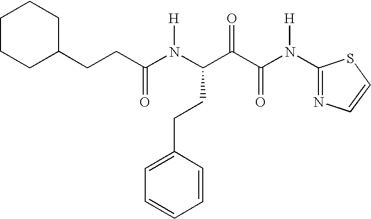

data for the compound as drawn and for it's enol and hydrate forms: LC-MS: t=4.74 min. 426.4(M−1), 428.2(M+1); 4.97 min, 426.2 (M−1), 428.2 (M+1); 5.57 min, 426.3(M−1), 427.9 (M+1). MS: API 150EX. (LC: Agilent 1100Series, Column: Phenomenex, 5 u ODS3 100A 100×3 mm. Flow Rate: 2 ml/min. Two solvent gradient: Solvent A, 99% water, 1% acetonitrile, 0.1% AcOH. Solvent B, 99% acetonitrile, 1% water, 0.1% AcOH. Gradient from 100% A, 0% B to 0% A, 100% B from t=0 to t=6 min. Then gradient back to 100% A, 0% B from t=7to t=15 min.)

### (d) 3-Cyclohexyl-N-(1-formyl-3-phenyl-propyl)-propionamide

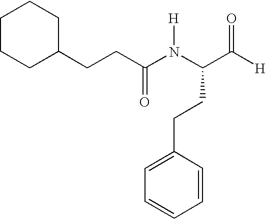

LC-MS: t=4.57 min., 300.4(M−1), 302.3(M+1). MS: API 150EX. (LC: Agilent 1100Series, Column: Phenomenex, 5 u ODS3 100A 100×3 mm. Flow Rate: 2 ml/min. Two solvent gradient: Solvent A, 99% water, 1% acetonitrile, 0.1% AcOH. Solvent B, 99% actonitrile, 1% water, 0.1% AcOH. Gradient from 100% A, 0% B to 0% A, 100% B from t=0 to t=6 min. Then gradient back to 100% A, 0% B from t=7 to t=15 min.)

### (f) 3-(2-Difluoromethoxy-phenylmethanesulfonyl)-N—[(S)-1-(5-ethyl-[1,3,4]oxadiazole-2-carbonyl)-propyl]-propionamide

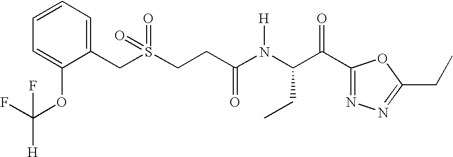

LC-MS: R_{T}=2.32 min., 460.3(M+1) 482.2(M+23) MS: API 150EX. (LC: Agilent 1100Series, Column: Phenomenex, 5 u ODS3 100A 100×3 mm. Flow Rate: 2 ml/min. Two solvent gradient: Solvent A, 99% water, 1% acetonitrile, 0.1% AcOH. Solvent B, 99% actonitrile, 1% water, 0.1% AcOH. Gradient from 100% A, 0% B to 0% A, 100% B from t=0 to t=2.5 min. Then gradient back to 100% A, 0% B from t=3.0 to t=3.5 min. Then gradient held at 100% A, 0% B from t=3.5 to 5 min.)

### (g) N—[(S)-1-(Benzooxazole-2-carbonyl)-propyl]-2-(2-cyano-phenylamino)-3-cyclohexyl-propionamide

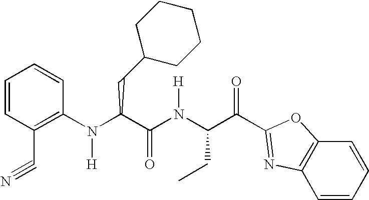

¹HNMR: (CDCl₃) 7.83 (d, 1H, J=8 Hz), 7.59 (d, 1H, J=8 Hz), 7.43-7.58 (m, 2H), 7.02-7.25(m, 4H), 6.59 (t, 1H, J=8 Hz), 6.49 (d,1H,J=8 Hz), 5.40-5.47 (m, 1H), 4.77 (m, 1H), 3.83-3.88 (m, 1H), 2.12-2.22 (m, 1H), 1.85-2.00 (m, 2H), 1.55-1.83 (m. 8H), 1.12-1.35 (m,4H), 0.95-1.10 (m, 3H); LC-MS: t=2.97 min., 457.5(M−1), 459.3(M+1), 481.4(M+23) MS: API 150EX. (LC: Agilent 1100Series, Column: Phenomenex, 5 u ODS3 100A 100×3 mm. Flow Rate: 2 ml/min. Two solvent gradient: Solvent A, 99% water, 1% acetonitrile, 0. 1% AcOH. Solvent B, 99% actonitrile, 1% water, 0.1% AcOH. Gradient from 100% A, 0% B to 0% A, 100% B from t=0 to t=2.5 min. Then gradient back to 100% A, 0% B from t=3.0 to t=3.5 min. Then gradient held at 100% A, 0% B from t=3.5 to 5 min.)

### (h) N-Cyanomethyl-3-cyclohexyl-2-(4-methoxy-phenoxy)-propionamide (Compound 1)

¹HNMR: (CDCl₃) 7.42-7.36 (m, 5H), 6.90 (t, 1H), 4.55 (d, 1H), 4.51 (d, 1H), 4.22 (dd, 1H), 4.16 (dd, 1H), 4.00 (t, 1H), 1.70-0.80 (m, 13H); MS: (M⁺+1) 301;

### (i) 2-Benzyloxy-N-cyanomethyl-3-cyclohexyl-propionamide (Compound 2)

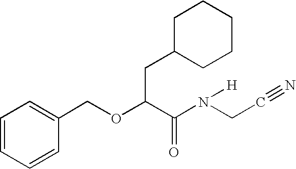

using 2(R)-benzyloxy-4-phenyl-butyric acid as starting material. ¹H NMR: (CDCl₃) δ 6.84-6.80 (m, 4H), 6.75 (t, 1H), 4.55 (dd, 1H), 4.24 (dd, 1H), 4.12 (dd, 1H), 3.78 (s, 3H), 1.80-0.85 (m, 13H); MS: (M−1) 315.

### (j) (R)—N—[(S)-1-(1-benzooxazol-2-yl-methanoyl)-butyl]-2-benzyloxy-3-phenylmethanesulfonyl-propionamide (Compound 3)

¹H NMR: (CDCl₃) 7.89 (d, 1H), 7.68 (d, 1H), 7.60-7.32 (m, 13H), 5.70 (m, 1H), 4.79 (d, 1H), 4.77 (d, 1H), 4.53 (dd, 1H), 4.33 (d, 1H), 4.30 (d, 1H), 3.38 (dd, 1H), 3.25 (dd, 1H), 2.15-2.05 (m, 1H), 1.84-75 (m, 1H), 1.45-1.30 (m, 2H), 0.93 (t, 3H); MS: (M⁺+1) 535, (M−1) 533;

### (k) (R)—N—[(S)-1-(1-benzooxazol-2-yl-methanoyl)-propyl]-2-methoxymethoxy-3-phenylmethanesulfonyl-propionamide (Compound 9)

¹H NMR (DMSO): 8.87(d, J=6.91 Hz, 1H), 7.99(d, J=7.91 Hz, 1H), 7.89(d, J=8.15 Hz, 1H), 7.64(t, J=8.1 Hz, 1H), 7.54(t, J=8.1 Hz, 1H), 7.4-7.3(m, 5H), 5.3-5.2(m, 1H), 4.7-4.65(m, 1H), 4.65-4.63(m, 2H), 4.55-4.50(m, 2H), 3.53-3.26(m, 2H), 3.34(s, 3H), 2.11-1.98(m, 1H), 1.81-1.69(m, 1H), 0.97(t, J=7.15 Hz, 3H); MS: 473(M−1), 497(M+23);

### (l) (S)—N—[(S)-1-(1-benzooxazol-2-yl-methanoyl)-butyl]-2-hydroxy-3-phenyl-propionamide (Compound 10)

### (m) (R)—N—[(S)-1-(1-benzooxazol-2-yl-methanoyl)-propyl]-3-phenylmethanesulfonyl-2-triisopropylsilanyloxy-propionamide (Compound 12)

¹H NMR (CD₃Cl): 7.93(d, J=8.15 Hz, 1H), 7.6(d, J=8.1 Hz, 1H), 7.6-7.4(m, 3H), 7.4-7.3(m, 5H), 5.85-5.73(m, 1H), 4.85-4.74(m, 1H), 4.5-4.3(m, 2H), 3.47-3.35(m, 2H), 2.35-2.15(m, 1H), 2.15-1.95(m, 1H), 1.3-0.8(m, 24H); MS: 609.4(M+23);

### (n) (R)—N—[(S)-1-(1-benzothiazol-2-yl-methanoyl)-propyl]-2-hydroxy-3-phenylmethanesulfonyl-propionamide (Compound 13)

¹H NMR (CD₃Cl): 8.21(d, J=8.67 Hz, 1H), 7.98(d, J=8.6 Hz, 1H), 7.7-7.55(m, 3H), 7.45-7.3(m, 5H), 5.8-5.7(m, 1H), 4.75-4.6(m, 1H), 4.4-4.3(m, 2H), 4.08(br, 1H), 3.62-3.5(m, 1H), 3.3-3.1(m, 1H), 2.3-2.15(m, 1H), 2.05-1.9(m, 1H), 0.997(t, J=7.4 Hz, 3H); MS: 469.2(M+23);

### (o) (R)-2-hydroxy-3-phenylmethanesulfonyl-N—[(S)-1-(1-pyridazin-3-yl-methanoyl)-butyl]-propionamide (Compound 16)

¹H NMR (CD₃Cl): 9.35(dd, J=4.93 Hz, J=1.72 Hz, 1H), 8.14(dd, J=1.72 Hz, J=8.39 Hz, 1H), 7.69(dd, J=4.93 Hz, J=8.39 Hz, 1H), 7.65(d, J=7.6 Hz, 1H), 7.5-7.36(m, 5H), 6.04-5.96(m, 1H), 4.75-4.63(m, 1H), 4.45-4.3(m, 3H), 3.53(dd, J=2.48 Hz, J=14.85 Hz, 1H), 3.22(dd, J=14.82 Hz, J=2.48 Hz, 1H), 2.2-2.07(m, 1H), 1.81-1.65(m, 1H), 1.6-1.2(m, 2H), 0.93(t, J=7.18 Hz, 3H); MS: 403.6(M−1), 428(M+23);

### (p) (S)-3-((R)-2-hydroxy-3-phenylmethanesulfonyl-propanoylamino)-2-oxo-pentanoic acid benzylamide (Compound 18)

¹H NMR (CD₃Cl): 7.45-7.25(m, 10H), 5.34-5.26(m, 1H), 4.7-4.6(m, 1H), 4.47(d, J=6.18 Hz, 2H), 4.4-4.3(m, 2H), 4.15-4.05(m, 1H), 3.55-3.45(m, 1H), 3.25-3.13(m, 1H), 22.2-2.0(m, 1H), 1.8-1.6(m, 1H), 1.61(s, 2H), 0.95(t, J=6.91 Hz, 3H); MS: 469.2(M+23);

### (q) (R)—N—[(S)-1-(1-benzooxazol-2-yl-methanoyl)-propyl]-3-[2-(1,1-difluoro-methoxy)-phenylmethanesulfonyl-2-hydroxy-propionamide (Compound 21)

¹H NMR (CD₃Cl): 7.91(d, J=7.91 Hz, 1H), 7.75(d, J=7.9 Hz, 1H), 7.7-7.2(m, 6H), 6.63(t, J=73.41 Hz, 1H), 5.7-5.58(m, 1H), 5.4-5.29(m, 1H), 4.7-4.6(m, 1H), 4.51(s, 2H), 4.19(br, 1H), 3.72-3.63(m, 1H), 3.35-3.2(m, 1H), 2.3-2.0(m, 1H), 2.0-1.7(m, 1H), 0.99(t, J=6.9 Hz, 3H); MS: 495.5(M−1), 497.2(M+1);

### (r) (R)—N—[(S)-1-(1-benzothiazol-2-yl-methanoyl)-propyl]-3-[2-(1,1-difluoro-methoxy)-phenylmethanesulfonyl]-2-hydroxy-propionamide (Compound 22)

¹H NMR (CD₃Cl): 8.21(d, J=8.15 Hz, 1H), 7.99(d, J=8.1 Hz, 1H), 7.73-7.2(m, 6H), 6.63(t, J=73.4 Hz, 1H), 5.85-5.75(m, 1H), 5.3(s, 1H), 4.78-4.7(m, 1H), 4.56-4.4(m, 2H), 4.19-4.09(m, 1H), 3.7-3.6(m, 1H), 3.35-3.2(m, 1H), 2.28(s, 2H), 1.27(t, J=6.9 Hz, 3H); MS; 511.4(M−1), 513.6(M+1); and

### (s) (2R,5S)-2-[2-(1,1-difluoro-methoxy)-phenylmethanesulfonylmethyl]-6-ethoxy-5-ethyl-morpholin-3-one (Compound 24).

### ENZYME ASSAY EXAMPLE

### Cathepsin S Assay

Solutions of test compounds in varying concentrations were prepared in 10 □L of dimethyl sulfoxide (DMSO) and then diluted into assay buffer (40 □L, comprising: MES, 50 mM (pH 6.5); EDTA, 2.5 mM; and NaCl, 100 mM). Human cathepsin S (0.158 pMoles in 25 □L of assay buffer) was added to the dilutions. The assay solutions were mixed for 5-10 seconds on a shaker plate, covered and incubated for 30 minutes at ambient temperature. Z-Val-Val-Arg-AMC (9 nMoles in 25 □L of assay buffer) was added to the assay solutions and hydrolysis was followed spectrophotometrically at (□ 460 nm) for 5 minutes. Apparent inhibition constants (Kᵢ) were calculated from the enzyme progress curves using standard mathematical models.

### ENZYME ASSAY EXAMPLE

### Cathepsin B Assay

Solutions of test compounds in varying concentrations were prepared in 10 □L of dimethyl sulfoxide (DMSO) and then diluted into assay buffer (40 □L, comprising: N,N-bis(2-hydroxyethyl)-2-aminoethanesulfonic acid (BES), 50 mM (pH 6); polyoxyethylenesorbitan monolaurate, 0.05%; and dithiothreitol (DTT), 2.5 mM). Human cathepsin B (0.025 pMoles in 25 □L of assay buffer) was added to the dilutions. The assay solutions were mixed for 5-10 seconds on a shaker plate, covered and incubated for 30 minutes at ambient temperature. Z-FR-AMC (20 nMoles in 25 □L of assay buffer) was added to the assay solutions and hydrolysis was followed spectrophotometrically at (□ 460 nm) for 5 minutes. Apparent inhibition constants (Kᵢ) were calculated from the enzyme progress curves using standard mathematical models.

### ENZYME ASSAY EXAMPLE

### Cathepsin K Assay

Solutions of test compounds in varying concentrations were prepared in 10 □L of dimethyl sulfoxide (DMSO) and then diluted into assay buffer (40 □L, comprising: MES, 50 mM (pH 5.5); EDTA, 2.5 mM; and DTT, 2.5 mM). Human cathepsin K (0.0906 pMoles in 25 □L of assay buffer) was added to the dilutions. The assay solutions were mixed for 5-10 seconds on a shaker plate, covered and incubated for 30 minutes at ambient temperature. Z-Phe-Arg-AMC (4 nMoles in 25 □L of assay buffer) was added to the assay solutions and hydrolysis was followed spectrophotometrically at (□ 460 nm) for 5 minutes. Apparent inhibition constants (Kᵢ) were calculated from the enzyme progress curves using standard mathematical models.

### ENZYME ASSAY EXAMPLE

### Cathepsin L Assay

Solutions of test compounds in varying concentrations were prepared in 10 □L of dimethyl sulfoxide (DMSO) and then diluted into assay buffer (40 □L, comprising: MES, 50 mM (pH 5.5); EDTA, 2.5 mM; and DTT, 2.5 mM). Human cathepsin L (0.05 pMoles in 25 □L of assay buffer) was added to the dilutions. The assay solutions were mixed for 5-10 seconds on a shaker plate, covered and incubated for 30 minutes at ambient temperature. Z-Phe-Arg-AMC (1 nMoles in 25 □L of assay buffer) was added to the assay solutions and hydrolysis was followed spectrophotometrically at (□ 460 nm) for 5 minutes. Apparent inhibition constants (Kᵢ) were calculated from the enzyme progress curves using standard mathematical models.

According to applicants' assays conducted as described above, the apparent inhibition constants (Kᵢ) for the following listed compounds of the invention, against Cathepsin S, were about or below 0.01 □M:

morpholine-4-carboxylic acid (R)-1-(cyanomethyl-carbamoyl)-2-[2-(1,1-difluoro-methoxy)-phenylmethanesulfonyl]-ethyl ester, (Compound 31), Example 3(a);

morpholine-4-carboxylic acid (R)-1-[(S)-1-(1-benzooxazol-2-yl-methanoyl)-propylcarbamoyl]-2-phenylmethanesulfonyl-ethyl ester, (Compound 11), Example 4(a);

morpholine-4-carboxylic acid (R)-1-[(S)-1-(1-benzooxazol-2-yl-methanoyl)-propylcarbamoyl]-2-[2-(1,1-difluoro-methoxy)-phenylmethanesulfonyl]-ethyl ester, (Compound 14), Example 4(b);

morpholine-4-carboxylic acid (R)-1-[(S)-1-(1-benzothiazol-2-yl-methanoyl)-propylcarbamoyl]-2-[2-(1,1-difluoro-methoxy)-phenylmethanesulfonyl]-ethyl ester, (Compound 15), Example 4(c);

pyrrolidine-1-carboxylic acid (R)-1-[(S)-1-(1-benzooxazol-2-yl-methanoyl)-propylcarbamoyl]-2-phenylmethanesulfonyl-ethyl ester, (Compound 19). Example 4(d);

dimethyl-carbamic acid (R)-1-[(S)-1-(1-benzooxazol-2-yl-methanoyl)-propylcarbamoyl]-2-phenylmethanesulfonyl-ethyl ester, (Compound 20), Example 4(e);

morpholine-4-carboxylic acid (R)-1-[(S)-1-(1-benzylcarbamoyl-methanoyl)-propylcarbamoyl]-2-phenylmethanesulfonyl-ethyl ester, (Compound 25). Example 4(f);

morpholine-4-carboxylic acid (S)-1-[(S)-1-(oxazolo[4,5-b]pyridine-2-carbonyl)-propylcarbamoyl]-2-phenylmethanesulfonyl-ethyl ester, Example 4(g);

morpholine-4-carboxylic acid (S)-1-[(S)-1-(5-ethyl-[1,3,4]oxadiazole-2-carbonyl)-propylcarbamoyl]-2-phenylmethanesulfonyl-ethyl ester, Example 4(h);

(R)-3-[2-(1,1-difluoro-methoxy)-phenylmethanesulfonyl]-N—((S)-1-formyl-propyl)-2-hydroxy-propionamide. (Compound 23), Example 6;

(R)—N—[(S)-1-(1-benzooxazol-2-yl-methanoyl)-propyl]-2-hydroxy-3-phenyl-methanesulfonyl-propionamide, (Compound 5), Example 7;

(S)-3-{3-[2-(1,1-difluoro-methoxy)-phenylmethanesulfonyl]-propanoylamino}-2-oxo-pentanoic acid benzylamide, (Compound 27), Example 8(a);

(R)—N—[(S)-1-(1-benzooxazol-2-yl-methanoyl)-propyl]-2-(2-nitro-phenylamino)-3-phenylmethanesulfonyl-propionamide, (Compound 28), Example 9;

(R)—N—[(S)-1-(1-benzooxazol-2-yl-methanoyl)-butyl]-2-(5-nitro-thiazol-2-ylamino)-3-phenylmethanesulfonyl-propionamide, (Compound 29), Example 10;

(R)—N—[(S)-1-(benzoxazole-2-carbonyl)-butyl]-3-phenylmethanesulfonyl-2-(tetrahydro-pyran-4-ylamino)-propionamide; Example 19(a);

(R)—N—[(S)-1-(benzoxazole-2-carbonyl)-butyl]-2-isopropylamino-3-phenylmethanesulfonyl propionamide, Example 21(a);

(R)—N—[(S)-1-(benzoxazole-2-carbonyl)-butyl]-2-[(2-methoxy-ethyl)-(tetrahydro-pyran-4-yl)-amino]-3-phenylmethanesulfonyl-propionamide, Example 21(b);

(R)—N—[(S)-1-(benzoxazole-2-carbonyl)-butyl]-2-cyclohexylamino-3-phenylmethanesulfonyl-propionamide, Example 21(c);

morpholine-4-carboxylic acid (S)-2-cyclohexyl-1-[(S)-1-(oxazolo[4,5-b]pyridine-2-carbonyl)-propylcarbamoyl]-ethyl ester, Example 24(b);

3-(2-difluoromethoxy-phenylmethanesulfonyl)-N—[(S)-1-(oxazolo[4,5-b]pyridine-2-carbonyl)-propyl]-propionamide, Example 33(e);

(S)-3-((R)-2-hydroxy-3-phenylmethanesulfonyl-propanoylamino)-2-oxo-pentanoic acid benzylamide (Compound 18), Example 33(p);

(R)—N—[(S)-1-(1-benzooxazol-2-yl-methanoyl)-propyl]-3-[2-(1,1-difluoro-methoxy)-phenylmethanesulfonyl]-2-hydroxy-propionamide (Compound 21), Example 33(q);

Moreover, the compounds of the present invention were observed to have varying degrees of selective inhibitory action on cathepsin S protease. For example, the above listed 22 compounds were found to inhibit cathepsin S protease activity at concentrations that are more than 75 fold less than those concentrations required to produce an equiactive inhibition on cathepsin K protease.

### EXAMPLE

### Representative Pharmaceutical Formulations Containing a Compound of Formula I

ORAL FORMULATION

| | | |
|---|---|---|
| Compound of Formula I | 10-100 | mg |
| Citric Acid Monohydrate | 105 | mg |
| Sodium Hydroxide | 18 | mg |
| Flavoring | | |
| Water | q.s. to 100 | mL |

INTRAVENOUS FORMULATION

| Compound of Formula I | 0.1-10 | mg |
|---|---|---|
| Dextrose Monohydrate | q.s. to make isotonic | |
| Citric Acid Monohydrate | 1.05 | mg |
| Sodium Hydroxide | 0.18 | mg |
| Water for Injection | q.s. to 1.0 | mL |

TABLET FORMULATION

| | |
|---|---|
| Compound of Formula I | 1% |
| Microcrystalline Cellulose | 73% |
| Stearic Acid | 25% |
| Colloidal Silica | 1%. |

We claim:

1. A compound of Formula I: 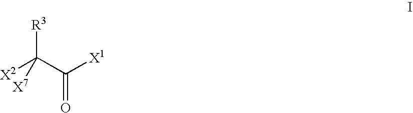
in which:
X¹ is —NHC(R¹)(R²)X³ or —NHX⁴;
X² is hydrogen, fluoro, —OH, —OR⁴, —NHR¹⁵ or —NR¹⁷R¹⁸ and X⁷ is hydrogen or X² and X⁷ both represent fluoro;
X³ is cyano, —C(R⁷)(R⁸)R¹⁶, —C(R⁶)(OR⁶)₂, —CH₂C(O)R¹⁶, —CH═CHS(O)₂R⁵, —C(O)CF₂C(O)NR⁵R⁵, —C(O)C(O)NR⁵R⁶, —C(O)C(O)OR⁵, —C(O)CH₂OR⁵, —C(O)CH₂N(R⁶)SO₂R⁵ or —C(O)C(O)R⁵; wherein R⁵ is hydrogen, (C₁₋₄)alkyl, (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl; R⁶ is hydrogen, hydroxy or (C₁₋₆)alkyl; or where X³ contains an —NR⁵R⁶ group, R⁵ and R⁶ together with the nitrogen atom to which they are both attached, form hetero(C₃₋₁₀)cycloalkyl, hetero(C₅₋₁₀)aryl or hetero(C₈₋₁₀)bicycloaryl; R⁷ is hydrogen or (C₁₋₄)alkyl and R⁸ is hydroxy or R⁷ and R⁸ together form oxo; R¹⁶ is hydrogen, —X⁴, —CF₃, —CF₂CF₂R⁹ or —N(R⁶)OR⁶; R⁹ is hydrogen, halo, (C₁₋₄)alkyl, (C₅₋₁₀)aryl(C₀₋₆) alkyl or (C₅₋₁₀)heteroaryl(C₀₋₆)alkyl, with the proviso that when X³ is cyano, then X² is hydrogen, fluoro, —OH, —OR⁴ or —NR¹⁷R¹⁸ and X⁷ is hydrogen or X² and X⁷ both represent fluoro;
X⁴ comprises a heteromonocyclic ring containing 4 to 7 ring member atoms or a fused heterobicyclic ring system containing 8 to 14 ring member atoms and any carbocyclic ketone, iminoketone or thioketone derivative thereof, with the proviso that when —X⁴ is other than a heteromonocyclic ring containing 5 ring member atoms, wherein no more than two of the ring member atoms comprising the ring are heteroatoms, then X² is fluoro, —OH, —OR⁴, —NHR¹⁵ or —NR¹⁷R¹⁸ and X⁷ is hydrogen or X² and X⁷ both represent fluoro;
wherein within R⁵, X³ or X⁴ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R^{12, —X}⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵ is a bond (C₁₋₆)alkylene;
R¹² at each occurrence independently is hydrogen, (C₁₋₆)alkyl or halo-substituted(C₁₋₆)alkyl;
R¹³ is (C₁₋₆)alkyl or halo-substituted(C₁₋₆)alkyl; and R¹⁴ is (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl;
R¹ is hydrogen or (C₁₋₆)alkyl and R² is selected from a group consisting of hydrogen, cyano, —X⁵NR¹²R¹², —X⁵NR¹²C(O)R¹², —X⁵NR¹²C(O)OR¹², —R¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵S(O)₂R¹³,—R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵, R¹², R¹³ and R¹⁴ are as defined above; or R¹ and R² taken together with the carbon atom to which both R¹ and R² are attached form (C₃₋₈)cycloalkylene or (C₃₋₈)heterocycloalkylene; wherein within said R² any heteroaryl, aryl, cycloalkyl, heterocycloalkyl, cycloalkylene or heterocycloalkylene is unsubstituted or substituted with 1 to 3 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵S(O)₂R¹³ and —X⁵C(O)R¹³, wherein X⁵, R¹² and R¹³ are as defined above;
R³ is (C₁₋₆)alkyl or —C(R⁶)(R⁶)X⁶, wherein R⁶ is hydrogen or (C₁₋₆)alkyl and X⁶ is selected from —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵(O)R¹³, —X⁵NR¹²C(O)R¹³, —X⁵S(O)R¹³, —X⁵S(O)₂R¹³, —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹² wherein X⁵, R¹², R¹³ and R¹⁴ are as defined above;
R⁴ is selected from —X⁸NR¹²R¹², —X⁸NR¹²C(O)R¹², —X⁸NR¹²C(O)OR¹², —X⁸NR¹²C(O)NR¹²R¹², —X⁸NR¹²C(NR¹²)NR¹²R¹², —X⁸OR¹², —X⁸SR¹², —X⁵C(O)OR¹², —X⁵C(O)R¹², —X⁸OC(O)R¹², —X⁵C(O)NR¹²R¹², —X⁸S(O)₂NR¹²R¹², —X⁸NR¹²S(O)₂R¹², —X⁸P(O)(OR¹²)OR¹², —X⁸OP(O)(OR¹²)OR¹², —X⁵C(O)R¹³, —X⁸NR¹²C(O)R¹³, —X⁸S(O)R¹³, —X⁸S(O)₂R¹³, —R¹⁴, —X⁸OR¹⁴, —X⁸SR¹⁴, —X⁸S(O)R¹⁴, —X⁸S(O)₂R¹⁴, —X⁵C(O)R¹⁴, —X⁵C(O)OR¹⁴, —X⁸ OC(O)R¹⁴, —X⁸NR¹⁴R¹², —X⁸NR¹²C(O)R¹⁴, —X⁸NR¹²C(O)OR¹⁴, —X⁵C(O)NR¹⁴R¹², —X⁸S(O)₂NR¹⁴R¹², —X⁸NR¹²S(O)₂R¹⁴, —X⁸NR¹²C(O)NR¹⁴R¹² and —X⁸NR¹²C(NR¹²)NR¹⁴R¹² wherein X⁸ is (C₁₋₆)alkylene and X⁵, R¹², R¹³ and R¹⁴ are as defined above, with the proviso that when X³ is cyano and X² is —OR⁴, where R⁴ is defined as —R¹⁴, then R¹⁴ is (C₃₋₁₀)cycloalkyl(C₁₋₆)alkyl, hetero(C₃₋₁₀)cycloalkyl(C₁₋₃)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl;
R¹⁵ is (C₆₋₁₀)aryl, hetero(C₅₋₁₀)aryl, (C₉₋₁₀)bicycloaryl or hetero(C₈₋₁₀)bicycloaryl;
R¹⁷ is (C₁₋₆)alkyl, (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl, with the proviso that when X³ is cyano, then R¹⁷ is (C₁₋₆)alkyl, (C₃₋₁₀)cycloalkyl(C₁₋₆)alkyl, hetero(C₃₋₁₀)cycloalkyl(C₁₋₆)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl;
R¹⁸ is hydrogen, (C₁₋₆)alkyl, (C₃₋₁₀)cycloalkyl(C₀₋₆)alkyl, hetero(C₃₋₁₀)cycloalkyl(C₀₋₆)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl, with the proviso that when X³ is cyano, then R¹⁸ is (C₁₋₆)alkyl, (C₃₋₁₀)cycloalkyl(C₁₋₆)alkyl, hetero(C₃₋₁₀)cycloalkyl(C₁₋₆)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl; and
wherein within R³, R⁴, R¹⁵, R¹⁷ and R¹⁸ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹², R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵C(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹²; and within R³ and R⁴ any aliphatic moiety is unsubstituted or substituted further by 1 to 5 radicals independently selected from cyano, halo, nitro, —NR¹²R¹², —NR¹²C(O)R¹², —NR¹²C(O)OR¹², —NR¹²C(O)NR¹²R¹², —NR¹²C(NR¹²)NR¹²R¹², —OR¹², —SR¹², —C(O)OR¹², —C(O)R¹², —OC(O)R¹², —C(O)NR¹²R¹², —S(O)₂NR¹²R¹², —NR¹²S(O)₂R¹², —P(O)(OR¹²)OR¹², —OP(O)(OR¹²)OR¹², —NR¹²C(O)R¹³, —S(O)R¹³ and —S(O)₂R¹³; wherein X⁵, R¹², R¹³ and R¹⁴ are as described above, with the proviso that when X³ is cyano and X² is —OR⁴, where R⁴ is defined as —R¹⁴, or —NHR¹⁸, then any aromatic ring system present within R¹⁴ or R¹⁸ is not substituted further by halo, (C₃₋₁₀)cycloalkyl, hetero(C₃₋₁₀)cycloalkyl, (C₆₋₁₀)aryl, hetero(C₅₋₁₀)aryl, (C₉₋₁₀)bicycloary or hetero(C₈₋₁₀)bicycloaryl; with the proviso that only one bicyclic ring structure is present within R³, R⁴ or R¹⁵; and the N-oxide derivatives, prodrug derivatives, protected derivatives, individual isomers and mixtures of isomers thereof; and the pharmaceutically acceptable salts and solvates of such compounds and the N-oxide derivatives, prodrug derivatives, protected derivatives, individual isomers and mixtures of isomers thereof.

2. A compound of claim 1, which is of the following formula: 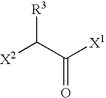
in which X² is hydrogen, fluoro, —OH, —OR⁴, —NHR¹⁵;
R³, R⁴, R¹⁵ and X¹ are the same as defined in claim 1.

3. A compound of claim 1 or claim 2 in which:
X¹ is —NHC(R¹)(R²)X³ or —NHCH(R¹⁹)C(O)R²⁰;
X² is hydrogen, fluoro, —OH, —OR⁴, —NHR¹⁵ or —NR¹⁷R¹⁸ and X⁷ is hydrogen or X² and X⁷ both represent fluoro;
X³ is cyano, —C(R⁷)(R⁸)R¹⁶, —C(R⁶)(OR⁶)₂, —CH₂C(O)R¹⁶, —CH═CHS(O)₂R⁵, —C(O)CF₂C(O)NR⁵R⁵, —C(O)C(O)NR⁵R⁶, —C(O)C(O)OR⁵, —C(O)CH₂OR⁵, —C(O)CH₂N(R⁶)SO₂R⁵ or —C(O)C(O)R⁵; wherein R⁵ is hydrogen, (C₁₋₄)alkyl, (C₃₋₁₀)cycloalkyl(C₀₋₆)alkyl, hetero(C₃₋₁₀)cycloalkyl(C₀₋₃)allyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl; R⁶ is hydrogen, hydroxy or (C₁₋₆)alkyl; or where X³ contains an —NR⁵R⁶ group, R⁵ and R⁶ together with the nitrogen atom to which they are both attached, form hetero(C₃₋₁₀)cycloalkyl, hetero(C₅₋₁₀)aryl or hetero(C₈₋₁₀)bicycloaryl; R⁷ is hydrogen or (C₁₋₄)alkyl and R⁸ is hydroxy or R⁷ and R⁸ together form oxo; R¹⁶ is hydrogen, —X⁴, —CF₃, —CF₂CF₂R⁹ or —N(R⁶)OR⁶; R⁹ is hydrogen, halo, (C₁₋₄)alkyl, (C₅₋₁₀)aryl(C₀₋₆)alkyl or (C₅₋₁₀)heteroaryl(C₀₋₆)alkyl, with the proviso that when X³ is cyano, then X² is hydrogen, fluoro, —OH, —OR⁴ or —NR¹⁷R¹⁸ and X⁷ is hydrogen or X² and X⁷ both represent fluoro;
X⁴ comprises a heteromonocyclic ring containing 4 to 7 ring member atoms or a fused heterobicyclic ring system containing 8 to 14 ring member atoms and any carbocyclic ketone, iminoketone or thioketone derivative thereof, with the proviso that when —X⁴ is other than a heteromonocyclic ring containing 5 ring member atoms, wherein no more than two of the ring member atoms comprising the ring are heteroatoms, then X² is fluoro, —OH, —OR⁴, —NHR¹⁵ or —NR¹⁷R¹⁸ and X⁷ is hydrogen or X² and X⁷ both represent fluoro;
wherein within R⁵, X³ or X⁴ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³ and —X⁵S(O)₂R¹³ and/or 1 radical selected from —R⁴, —X⁵OR¹⁴, —X⁵SR¹⁴, , —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵ is a bond or (C₁₋₆)alkylene;
R¹² at each occurrence independently is hydrogen, (C₁₋₆)alkyl or halo-substituted(C₁₋₆)alkyl;
R¹³ is (C₁₋₆)alkyl or halo-substituted(C₁₋₆)alkyl; and R¹⁴ is (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl;
R¹ is hydrogen or (C₁₋₆)alkyl and R² is selected from a group consisting of hydrogen, cyano, —X⁵NR¹²R¹², —X⁵NR¹²C(O)R¹², —X⁵NR¹²C(O)OR¹², —R¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵S(O)₂R¹³, —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵, R¹², R¹³ and R¹⁴ are as defined above; or R¹ and R² taken together with the carbon atom to which both R¹ and R² are attached form (C₃₋₈)cycloalkylene or (C₃₋₈)heterocycloalkylene; wherein within said R² any heteroaryl, aryl, cycloalkyl, heterocycloalkyl, cycloalkylene or heterocycloalkylene is unsubstituted or substituted with 1 to 3 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵S(O)₂R¹³ and —X⁵C(O)R¹³, wherein X⁵, R¹² and R¹³ are as defmed above;
R³ is (C₁₋₆)alkyl or —C(R⁶)(R⁶)X⁶, wherein R⁶ is hydrogen or (C₁₋₆)alkyl and X⁶ is selected from —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R^{12 , —X}⁵P(O)(OR¹²)OR¹², —X⁵OP(O)(OR¹²)OR¹², —X⁵C(O)R¹³, —X⁵NR¹²C(O)R¹³, —X⁵S(O)R¹³, —X⁵S(O)₂R¹³, —R¹⁴, —X⁵OR¹⁴, —X⁵SR¹⁴, —X⁵S(O)R¹⁴, —X⁵S(O)₂R¹⁴, —X⁵C(O)OR¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹² wherein X⁵, R¹², R¹³ and R¹⁴ are as defmed above;
R⁴ is selected from —X⁸NR¹²R¹², —X⁸NR¹²C(O)R¹², —X⁸NR¹²C(O)OR¹², —X⁸NR¹²C(O)NR¹²R¹², —X⁸NR¹²C(NR¹²)NR¹²R¹², —X⁸OR¹², —X⁸SR¹², —X⁵C(O)OR¹², —X⁵C(O)R¹², —X⁸OC(O)R¹², —X⁵C(O)NR¹²R¹², —X⁸S(O)₂NR¹²R¹², —X⁸NR¹²S(O)₂R¹², —X⁸P(O)(OR¹²)OR¹², —X⁸OP(O)(OR¹²)OR¹², —X⁵C(O)R¹³, —X⁸NR¹²C(O)R¹³, —X⁸S(O)R¹³, —X⁸S(O)₂R¹³, —R¹⁴, —X⁸OR¹⁴, —X⁸SR¹⁴, —X⁸S(O)R¹⁴, —X⁸S(O)₂R¹⁴, —X⁵C(O)R¹⁴, —X⁵C(O)OR¹⁴, —X⁸OC(O)R¹⁴, —X⁸NR¹⁴R¹², —X⁸NR¹²C(O)R¹⁴, —X⁸NR¹²C(O)OR¹⁴, —X⁵C(O)NR¹⁴R¹², —X⁸S(O)₂NR¹⁴R¹², —X⁸NR¹²S(O)₂R¹⁴, —X⁸NR¹²C(O)NR¹⁴R¹² and —X⁸NR¹²C(NR¹²)NR¹⁴R¹² wherein X⁸ is (C₁₋₆)alkylene and X⁵, R¹², R¹³ and R¹⁴ are as defmed above, with the proviso that when X³ is cyano and X² is —OR⁴, where R⁴ is defined as —R¹⁴, then R¹⁴ is (C₃₋₁₀)cycloalkyl(C₁₋₆)alkyl, hetero(C₃₋₁₀)cycloalkyl(C₁₋₃)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl;
R¹⁵ is (C₆₋₁₀)aryl, hetero(C₅₋₁₀)aryl, (C₉₋₁₀)bicycloaryl or hetero(C₈₋₁₀)bicycloaryl;
R¹⁷ is (C₁₋₆)alkyl, (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl, with the proviso that when X³ is cyano, then R¹⁷ is (C₁₋₆)alkyl, (C₃₋₁₀)cycloalkyl(C₁₋₆)alkyl, hetero(C₃₋₁₀)cycloalkyl(C₁₋₆)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl;
R¹⁸ is hydrogen, (C₁₋₆)alkyl, (C₃₋₁₀)cycloalkyl(C₀₋₆)alkyl, hetero(C₃₋₁₀)cycloalkyl(C₀₋₆)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl, with the proviso that when X³ is cyano, then R¹⁸ is (C₁₋₆)alkyl, (C₃₋₁₀)cycloalkyl(C₁₋₆)alkyl, hetero(C₃₋₁₀)cycloalkyl(C₁₋₆)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl; and
R¹⁹ and R²⁰ together with the atoms to which R¹⁹ and R²⁰ are attached form (C₄₋₈)heterocycloalkylene, wherein no more than one of the ring member atoms comprising the ring is a heteroatom selected from —NR²¹— or —O—, wherein the ring is unsubstituted or substituted with R², wherein R² is as defined above, and R²¹ is hydrogen, —C(O)OR¹², —C(O)R¹², —C(O)NR¹²R¹², —S(O)₂NR¹²R¹², —S(O)R¹³ and —S(O)₂R¹³, —S(O)R¹⁴, —S(O)₂R¹⁴, —C(O)R¹⁴, —C(O)OR¹⁴, —C(O)NR¹²R¹² and —S(O)₂NR¹⁴R¹², wherein R¹², R¹³ and R¹⁴ are as defined above;
wherein within R³, R⁴, R¹⁵, R¹⁷ and R¹⁸ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵C(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹²; and within R³ and R⁴ any aliphatic moiety is unsubstituted or substituted further by 1 to 5 radicals independently selected from cyano, halo, nitro, —NR¹²R¹², —NR¹²C(O)R¹², —NR¹²C(O)OR¹², —NR¹²C(O)NR¹²R¹², —NR¹²C(NR¹²)NR¹²R¹², —OR¹², —SR¹², —C(O)OR¹², —C(O)R¹², —OC(O)R¹², —C(O)NR¹²R¹², —S(O)₂NR¹²R¹², —NR¹²S(O)₂R¹², —P(O)(OR¹²)OR¹², —OP(O)(OR¹²)OR¹², —NR¹²C(O)R¹³, —S(O)R¹³ and —S(O)₂R¹³; wherein X⁵, R¹², R¹³ and R¹⁴ are as described above, with the proviso that when X³ is cyano and X² is —OR⁴, where R⁴ is defined as —R¹⁴, or —NHR¹⁸, then any aromatic ring system present within R¹⁴ or R¹⁸ is not substituted further by halo, (C₃₋₁₀)cycloalkyl, hetero(C₃₋₁₀)cycloalkyl, (C₆₋₁₀)aryl, hetero(C₅₋₁₀)aryl, (C₉₋₁₀)bicycloaryl or hetero(C₈₋₁₀)bicycloaryl; with the proviso that only one bicyclic ring structure is present within R³, R⁴ or R¹⁵; and the N-oxide derivatives, prodrug derivatives, protected derivatives, individual isomers and mixtures of isomers thereof; and the pharmaceutically acceptable salts and solvates of such compounds and the N-oxide derivatives, prodrug derivatives, protected derivatives, individual isomers and mixtures of isomers thereof.

4. The compound of claim 1 or claim 2 in which:
X¹ is —NHC(R¹)(R²)X³ or —NHCH(R¹⁹)C(O)R²⁰;
X² is hydrogen, fluoro, —OH, —OR⁴, —NHR¹⁵ or —NR¹⁷R¹⁸ and X⁷ is hydrogen or X² and X⁷ both represent fluoro;
X³ is —C(R⁷)(R⁸)R¹⁶, —C(R⁶)(OR⁶)₂, —CH₂C(O)R¹⁶, —CH═CHS(O)₂R⁵, —C(O)CF₂C(O)NR⁵R⁵, —C(O)C(O)NR⁵R⁶, —C(O)C(O)OR⁵, —(O)CH₂OR⁵, —C(O)CH₂N(R⁶)SO₂R⁵ or —C(O)C(O)R⁵; wherein R⁵ is hydrogen, (C₁₋₄)alkyl, (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl; R⁶ is hydrogen, hydroxy or (C₁₋₆)alkyl; or where X³ contains an —NR⁵R⁶ group, R⁵ and R⁶ together with the nitrogen atom to which they are both attached, form hetero(C₃₋₁₀)cycloalkyl, hetero(C₅₋₁₀)aryl or hetero(C₈₋₁₀)bicycloaryl; R⁷ is hydrogen or (C₁₋₄)alkyl and R⁸ is hydroxy or R⁷ and R⁸ together form oxo; R¹⁶ is hydrogen, —X⁴, —CF₃, —CF₂CF₂R⁹ or —N(R⁶)OR⁶; R⁹ is hydrogen, halo, (C₁₋₄)alkyl, (C₅₋₁₀)aryl(C₀₋₆)alkyl or (C₅₋₁₀)heteroaryl(C₀₋₆)alkyl;
X⁴ comprises a heteromonocyclic ring containing 4 to 7 ring member atoms or a fused heterobicyclic ring system containing 8 to 14 ring member atoms and any carbocyclic ketone, iminoketone or thioketone derivative thereof, with the proviso that when —X⁴ is other than a heteromonocyclic ring containing 5 ring member atoms, wherein no more than two of the ring member atoms comprising the ring are heteroatoms, then X² is fluoro, —OH, —OR⁴, —NHR¹⁵ or —NR¹⁷R¹⁸ and X⁷ is hydrogen or X² and X⁷ both represent fluoro;
wherein within R⁵, X³ or X⁴ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵ is a bond or (C₁₋₆)alkylene;
R¹² at each occurrence independently is hydrogen, (C₁₋₆)alkyl or halo-substituted(C₁₋₆)alkyl;
R¹³ is (C₁₋₆)alkyl or halo-substituted(C₁₋₆)alkyl; and R¹⁴ is (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl;
R¹ is hydrogen or (C₁₋₆)alkyl and R² is selected from a group consisting of hydrogen, cyano, —X⁵NR¹²R¹², —X⁵NR¹²C(O)R¹², —X⁵NR¹²C(O)OR¹², —R¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵S(O)₂R¹³, —R¹⁴, —X⁵OR₁₄, —X⁵SR¹⁴, —X⁵S(O)R¹⁴, —X⁵S(O)₂R¹⁴, —X⁵C(O)R¹⁴, —X⁵C(O)OR¹⁴, —X⁵C(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵, R¹², R¹³ and R¹⁴ are as defined above; or R¹ and R² taken together with the carbon atom to which both R¹ and R² are attached form (C₃₋₈)cycloalkylene or (C₃₋₈)heterocycloalkylene; wherein within said R² any heteroaryl, aryl, cycloalkyl, heterocycloalkyl, cycloalkylene or heterocycloalkylene is unsubstituted or substituted with 1 to 3 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵S(O)₂R¹³ and —X⁵C(O)R¹³, wherein X⁵, R¹² and R¹³ are as defined above;
R³ is (C₁₋₆)alkyl or —C(R⁶)(R⁶)X⁶, wherein R⁶ is hydrogen or (C₁₋₆)alkyl and X⁶ is selected from —X⁵NR¹²R¹², —X⁵NR¹²C(O)R¹², —X⁵NR¹²C(O)OR¹², —X⁵NR¹²R¹², —X⁵ NR¹²C(NR¹²)NR¹²R¹², —X⁵OR¹², —X⁵SR¹², —X⁵C(O)OR¹², —X⁵C(O)OR¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR ¹²)OR¹², —X⁵C(O)R¹³, —X⁵NR¹²C(O)R¹³, —X⁵S(O)R¹³, —X⁵S(O)₂R¹³, —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹² wherein X⁵, R¹², R¹³ and R¹⁴ are as defined above;
R⁴ is selected from —X⁸NR¹²R¹², —X⁸NR¹²C(O)R¹², —X⁸NR¹²C(O)R¹², —X⁸NR¹²C(O)OR¹², —X⁸NR¹²C(O)NR¹²R¹², —X⁸NR¹²C(NR¹²)NR¹²R¹², —X⁸OR¹², —X⁸SR¹², —X⁵C(O)OR¹², —X⁵C(O)R¹², —X⁸OC(O)R¹², —X⁵C(O)NR¹²R¹², —X⁸S(O)₂NR¹²R¹², —X⁸NR¹²S(O)₂R¹², —X⁸P(O)(OR¹²)OR¹², —X⁸OP(O)(OR¹²)OR¹², —X⁵C(O)R¹³, —X⁸NR¹²C(O)R¹³, —X⁸S(O)R¹³, —X⁸S(O)₂R¹³, —R¹⁴, —X⁸OR¹⁴, —X⁸S(O)R¹⁴, —X⁸S(O)₂R¹⁴, —X⁸S(O)₂R¹⁴, —X⁵C(O)R¹⁴, —X⁵C(O)OR¹⁴, —X⁸OC(O)R¹⁴, —X⁸NR¹⁴R¹², —X⁸NR¹²C(O)R¹⁴, —X⁸NR¹²C(O)OR¹⁴, —X⁵C(O)NR¹⁴R¹², —X⁸S(O)₂NR¹⁴R¹², —X⁸NR¹²S(O)₂R¹⁴, —X⁸NR^{12 C(O)NR}¹⁴R¹² and —X⁸NR¹²C(NR¹²)NR¹⁴R¹² wherein X⁸ is (C₁₋₆)alkylene and X⁵, R¹², R¹³ and R¹⁴ are as defined above;
R¹⁵ is (C₆₋₁₀)aryl, hetero(C₅₋₁₀)aryl, (C₉₋₁₀)bicycloaryl or hetero(C₈₋₁₀)bicycloaryl;
R¹⁷ is hydrogen, (C₁₋₆)alkyl, (C₃₋₁₀)cycloalkyl(C₀₋₆)alkyl, hetero(C₃₋₁₀)cycloalkyl(C₀₋₆)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl;
R¹⁸ is (C₁₋₆)alkyl, (C₃₋₁₀)cycloalkyl(C₀₋₆)alkyl, hetero(C₃₋₁₀)cycloalkyl(C₀₋₆)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl; and
R¹⁹ and R²⁰ together with the atoms to which R¹⁹ and R²⁰ are attached form (C₄₋₈)heterocycloalkylene, wherein no more than one of the ring member atoms comprising the ring is a heteroatom selected from —NR²¹— or —O—, wherein the ring is unsubstituted or substituted with R², wherein R² is as defined above, and R²¹ is hydrogen, —C(O)OR¹², —C(O)R¹², —C(O)NR¹²R¹², —S(O)₂NR¹²R¹², —S(O)R¹³ and —S(O)₂R¹³, —S(O)R¹⁴, —S(O)₂R¹⁴, —C(O)R¹⁴, —C(O)OR¹⁴, —C(O)NR¹²R¹² and —S(O)₂NR¹⁴R¹², wherein R¹², R¹³ and R¹⁴ are as defined above;
wherein within R³, R⁴, R¹⁵, R¹⁷ and R¹⁸ any alicyclic or aromatic ring system is unsubstituted or substituted further by 1 to 5 radicals independently selected from (C₁₋₆)alkyl, (C₁₋₆)allkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R^{12, —X}⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R^{13, —X}⁵S(O)R¹³, —X⁵C(O)R¹³ and —X⁵S(O)₂R¹³ and/or 1 radical selected from —R¹⁴, —X⁵OR¹⁴, —X⁵S(O)R¹⁴, —X⁵S(O)₂R¹⁴, X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹²; and within R³ and R⁴ any aliphatic moiety is unsubstituted or substituted further by 1 to 5 radicals independently selected from cyano, halo, nitro, —NR¹²R ¹², —NR¹²C(O)R¹², —NR¹²C(O)OR¹², —NR¹²C(O)NR¹²R ¹², —NR¹²C(NR¹²)NR¹²R¹², —OR¹², —SR¹², —C(O)OR¹², —C(O)R¹², —OC(O)R¹², —C(O)NR¹²R¹², —S(O)₂NR¹²R¹², —NR¹²S(O)₂R¹², —P(O)(OR¹²)OR¹², —OP(O)(OR¹²)OR¹², —NR¹²C(O)R¹³, —S(O)R¹³ and —S(O)₂R¹³; wherein X⁵, R¹², R¹³ and R¹⁴ are as described above; with the proviso that only one bicyclic ring structure is present within R³, R⁴ or R¹⁵; and the N-oxide derivatives, prodrug derivatives, protected derivatives, individual isomers and mixtures of isomers thereof; and the pharmaceutically acceptable salts and solvates of such compounds and the N-oxide derivatives, prodrug derivatives, protected derivatives, individual isomers and mixtures of isomers thereof.

5. A compound of claim 1 or claim 2 in which:
X¹ is —NHC(R¹)(R²)X³ or —NHCH(R¹⁹)C(O)R²⁰;
X² is hydrogen, fluoro, —OH, —OR⁴ or —NR¹⁷R¹⁸ and X⁷ is hydrogen or X² and X⁷ both represent fluoro;
X³ is cyano;
wherein within X³ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —XNR¹²R¹², —X⁵NR¹²C(O)R¹², —X⁵NR¹²C(O)OR²¹, —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵ is a bond or (C₁₋₆)alkylene; R¹² at each occurrence independently is hydrogen, (C₁₋₆)alkyl or halo-substituted(C₁₋₆)alkyl; R¹³ is (C₁₋₆)alkyl or halo-substituted(C₁₋₆)alkyl; and R¹⁴ is (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl;
R¹ is hydrogen or (C₁₋₆)alkyl and R² is selected from a group consisting of hydrogen, cyano, —X⁵NR¹²R¹², —X⁵NR¹²C(O)R¹², —X⁵NR¹²C(O)OR¹², —R¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵S(O)₂R¹³, —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)ORO¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵, R¹², R¹³ and R¹⁴ are as defined above; or R¹ and R² taken together with the carbon atom to which both R¹ and R² are attached form (C₃₋₈)cycloalkylene or (C₃₋₈)heterocycloalkylene; wherein within said R² any heteroaryl, aryl, cycloalkyl, heterocycloalkyl, cycloalkylene or heterocycloalkylene is unsubstituted or substituted with 1 to 3 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵S(O)₂R¹³ and —X⁵C(O)R¹³, wherein X⁵, R¹² and R¹³ are as defined above;
R³ is (C₁₋₆)alkyl or —C(R⁶)(R⁶)X⁶, wherein R⁶ is hydrogen or (C₁₋₆)alkyl and X⁶ is selected from —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵C(O)R¹³, —X⁵NR¹²C(O)R¹³, —X⁵S(O)R¹³, —X⁵S(O)₂R¹³, —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹² wherein X⁵, R¹², R¹³ and R¹⁴ are as defined above;
R⁴ is selected from —X⁸NR¹²R¹², —X⁸NR¹²C(O)R¹², —X⁸NR¹²C(O)OR¹², —X⁸NR¹²C(O)NR¹²R², —X⁸NR¹²C(NR¹²)NR¹²R¹², —X⁸OR¹², —X⁸SR¹², —X⁵C(O)OR¹², —X⁵C(O)R¹², —X⁸OC(O)R¹², —X⁵C(O)NR¹²R¹², —X⁸S(O)₂NR¹²R¹², —X⁸NR¹²S(O)₂R¹², —X⁸ P(O)(OR¹²)OR¹², —X⁸OP(O)(OR¹²)OR¹², —X⁵C(O)R¹³, —X⁸NR¹²C(O)R¹³, —X⁸S(O)R¹³, —X⁸ S(O)₂R¹³, —R¹⁴, —X⁸OR¹⁴, —X⁸SR¹⁴, —X⁸S(O)R¹⁴, —X⁸S(O)₂R¹⁴, —X⁵C(O)R¹⁴, —X⁵C(O)OR¹⁴, —X⁸OC(O)R¹⁴, —X⁸NR¹⁴R¹², —X⁸NR¹²C(O)R¹⁴, —X⁸NR¹²C(O)OR¹⁴, —X⁵C(O)NR¹⁴R¹², —X⁸S(O)₂NR¹⁴R¹², —X⁸NR¹²S(O)₂R¹⁴, —X⁸NR¹²C(O)NR¹⁴R¹² and —X⁸NR¹²C(NR¹²)NR¹⁴R¹² wherein X⁸ is (C₁₋₆)alkylene and X⁵, R¹², R¹³ and R¹⁴ are as defined above, with the proviso that when X³ is cyano and X² is —OR⁴, where R⁴ is defined as —R¹⁴, then R¹⁴ is (C₃₋₁₀)cycloalkyl(C₁₋₆)alkyl, hetero(C₃₋₁₀)cycloalkyl(C₁₋₃)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl;
R¹⁵ is (C₆₋₁₀)aryl, hetero(C₅₋₁₀)aryl, (C₉₋₁₀)bicycloaryl or hetero(C₈₋₁₀)bicycloaryl;
R¹⁷ is (C₁₋₆)alkyl, (C₃₋₁₀)cycloalkyl(C₁₋₆)alkyl, hetero(C₃₋₁₀)cycloalkyl(C₁₋₆)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl;
R¹⁸ is (C₁₋₆)alkyl, (C₃₋₁₀)cycloalkyl(C₁₋₆)alkyl, hetero(C₃₋₁₀)cycloalkyl(C₁₋₆)alkyl, (C₆₋₁₀)aryl(C₁₋₆)alkyl, hetero(C₅₋₁₀)aryl(C₁₋₆)alkyl, (C₉₋₁₀)bicycloaryl(C₁₋₆)alkyl or hetero(C₈₋₁₀)bicycloaryl(C₁₋₆)alkyl; and
R¹⁹ and R²⁰ together with the atoms to which R¹⁹ and R²⁰ are attached form (C₄₋₈)heterocycloalkylene, wherein no more than one of the ring member atoms comprising the ring is a heteroatom selected from —NR²¹— or —O—, wherein the ring is unsubstituted or substituted with R², wherein R² is as defined above, and R²¹ is hydrogen, —C(O)OR¹², —C(O)R¹², —C(O)NR¹²R¹², —S(O)₂NR¹²R¹², —S(O)R¹³ and —S(O)₂R¹³, —S(O)R¹⁴, —S(O)₂R¹⁴, —C(O)R¹⁴, —C(O)OR¹⁴, —C(O)NR¹²R¹² and —S(O)₂NR¹⁴R¹², wherein R¹², R¹³ and R¹⁴ are as defined above;
wherein within R³, R⁴, R¹⁵, R¹⁷ and R¹⁸ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵C(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹²; and within R³ and R⁴ any aliphatic moiety is unsubstituted or substituted further by 1 to 5 radicals independently selected from cyano, halo, nitro, —NR¹²R¹², —NR¹²C(O)R¹², —NR¹²C(O)OR¹², —NR¹²C(O)NR¹²R¹², —NR¹²C(NR¹² )NR¹²R¹², —OR¹², —SR¹², —C(O)OR¹², —C(O)R¹², —OC(O)R¹², —C(O)NR¹²R¹², —S(O)₂NR¹²R¹², —NR¹²S(O)₂R¹², —P(O)(OR¹²)OR¹², —OP(O)(OR¹²)OR¹², —NR¹²C(O)R¹³, —S(O)R¹³ and S(O)₂R¹³; wherein X⁵, R¹², R¹³ and R¹⁴ are as described above, with the proviso that when X² is —OR⁴, where R⁴ is defined as —R¹⁴, or —NHR¹⁸, then any aromatic ring system present within R¹⁴ or R¹⁸ is not substituted further by halo, (C₃₋₁₀)cycloalkyl, hetero(C₃₋₁₀)cycloalkyl, (C₆₋₁₀)aryl, hetero(C₅₋₁₀)aryl, (C₉₋₁₀)bicycloaryl or hetero(C₈₋₁₀)bicycloaryl; with the proviso that only one bicyclic ring structure is present within R³, R⁴ or R¹⁵; and the N-oxide derivatives, prodrug derivatives, protected derivatives, individual isomers and mixtures of isomers thereof; and the pharmaceutically acceptable salts and solvates of such compounds and the N-oxide derivatives, prodrug derivatives, protected derivatives, individual isomers and mixtures of isomers thereof.

6. A compound of claim 1 or 2 in which:
X¹ is —NHC(R¹)(R²)X³ or —NHCH(R¹⁹)C(O)R²⁰;
X² is —OH, —OC(O)NR¹²R¹² or —OC(O)R¹⁴, wherein R¹² and R¹⁴ are as defined below;
X³ is cyano, —C(R⁷)(R⁸)R¹⁶, —C(R⁶)(OR⁶)₂, —CH₂C(O)R¹⁶, —CH═CHS(O)₂R⁵, —C(O)CF₂C(O)NR⁵R⁵, —C(O)C(O)NR⁵R⁶, —C(O)C(O)OR⁵, —C(O)CH₂OR⁵, —C(O)CH₂N(R⁶)SO₂R⁵ or —C(O)C(O)R⁵; wherein R⁵ is hydrogen, (C₁₋₄)alkyl, (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl; R⁶ is hydrogen, hydroxy or (C₁₋₆)alkyl; or where X³ contains an —NR⁵R⁶ group, R⁵ and R⁶ together with the nitrogen atom to which they are both attached, form hetero(C₃₋₁₀)cycloalkyl, hetero(C₅₋₁₀)aryl or hetero(C₈₋₁₀)bicycloaryl; R⁷ is hydrogen or (C₁₋₄)alkyl and R⁸ is hydroxy or R⁷ and R⁸ together form oxo; R¹⁶ is hydrogen, —X⁴, —CF₃, —CF₂CF₂R⁹ or —N(R⁶)OR⁶; R⁹ is hydrogen, halo, (C₁₋₄)alkyl, (C₅₋₁₀)aryl(C₀₋₆)alkyl or (C₅₋₁₀)heteroaryl(C₀₋₆)alkyl;
X⁴ comprises a heteromonocyclic ring containing 4 to 7 ring member atoms or a fused heterobicyclic ring system containing 8 to 14 ring member atoms and any carbocyclic ketone, iminoketone or thioketone derivative thereof;
wherein within R⁵, X³ or X⁴ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵ is a bond or (C₁₋₆)alkylene;
R¹² at each occurrence independently is hydrogen, (C₁₋₆)alkyl or halo-substituted(C₁₋₆)alkyl;
R¹³ is (C₁₋₆)alkyl or halo-substituted(C₁₋₆)alkyl; and R¹⁴ is (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl;
R¹ is hydrogen or (C₁₋₆)alkyl and R² is selected from a group consisting of hydrogen, cyano, —X⁵NR¹²R¹², —X⁵NR¹²C(O)R¹², —X⁵NR¹²C(O)OR¹², —R¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵S(O)₂R¹³, —R¹⁴, —X⁵OR¹⁴, —X⁵SR¹⁴, —X⁵S(O)R¹⁴, —X⁵S(O)₂R¹⁴, —X⁵ C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵, R¹², R¹³ and R¹⁴ are as defined above; or R¹ and R² taken together with the carbon atom to which both R¹ and R² are attached form (C₃₋₈)cycloalkylene or (C₃₋₈)heterocycloalkylene; wherein within said R² any heteroaryl, aryl, cycloalkyl, heterocycloalkyl, cycloalkylene or heterocycloalkylene is unsubstituted or substituted with 1 to 3 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵S(O)₂R¹³ and —X⁵C(O)R¹³, wherein X⁵, R¹² and R¹³ are as defined above;
R³ is (C₁₋₆)alkyl or —C(R⁶)(R⁶)X⁶, wherein R⁶ is hydrogen or (C₁₋₆)alkyl and X⁶ is selected from —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵C(O)R¹³, —X⁵NR¹²C(O)R¹³, —X⁵S(O)R¹³, —X⁵S(O)₂R¹³, —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹² wherein X⁵, R¹², R¹³ and R¹⁴ are as defined above; and
R¹⁹ and R²⁰ together with the atoms to which R¹⁹ and R²⁰ are attached form (C₄₋₈)heterocycloalkylene, wherein no more than one of the ring member atoms comprising the ring is a heteroatom selected from —NR²¹— or —O—, wherein and the ring is unsubstituted or substituted with R², wherein R² is as defined above, and R²¹ is hydrogen, —C(O)OR¹², —C(O)R¹², —C(O)NR¹²R¹², —S(O)₂NR¹²R¹², —S(O)R¹³ and —S(O)₂R¹³, —S(O)R¹⁴, —S(O)₂R¹⁴, —C(O)R¹⁴, —C(O)OR¹⁴, —C(O)NR¹²R¹² and —S(O)₂NR¹⁴R¹², wherein R¹², R¹³ and R¹⁴ are as defined above;
wherein within R³, R⁴, R¹⁵, R¹⁷ and R¹⁸ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹², R¹², —X⁵OR¹², —X⁵SR¹², —X⁵C(O)OR¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵C(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹⁴R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹²; and within R³ and R⁴ any aliphatic moiety is unsubstituted or substituted further by 1 to 5 radicals independently selected from cyano, halo, nitro, —NR¹²R¹², —NR¹²C(O)R¹², —NR¹²C(O)OR¹², —NR¹²C(O)NR¹²R¹², —NR¹²C(NR¹²)NR¹²R¹², —OR¹², —SR¹², —C(O)OR¹², —C(O)R¹², —OC(O)R¹², —C(O)NR¹²R¹², —S(O)₂NR¹²R¹², —NR¹²S(O)₂R¹², —P(O)(OR¹²)OR¹², —OP(O)(OR¹²)OR¹², —NR¹²C(O)R¹³, —S(O)R¹³ and —S(O)₂R¹³; wherein X⁵, R¹², R¹³ and R¹⁴ are as described above; with proviso that only one bicyclic ring structure is present within R³, R⁴ or R¹⁵; and the N-oxide derivatives, prodrug derivatives, protected derivatives, individual isomers and mixtures of isomers thereof; and the pharmaceutically acceptable salts and solvates of such compounds and the N-oxide derivatives, prodrug derivatives, protected derivatives, individual isomers and mixtures of isomers thereof.

7. The compound of claim 1 or claim 2 in which:
X¹ is —NHC(R¹)(R²)C(O)C(O)NR⁵R⁶, wherein R⁵ is hydrogen, (C₁₋₄)alkyl, (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl and R⁶ is hydrogen, hydroxy or (C₁₋₆)alkyl or R⁵ and R⁶ together with the nitrogen atom to which they are both attached form hetero(C₃₋₁₀)cycloalkyl, hetero(C₅₋₁₀)aryl or hetero(C₈₋₁₀)bicycloaryl;
X² is hydrogen;
wherein within X¹ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵NR¹²S(O)₂R¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³ and —X⁵S(O)₂R¹³ and/or 1 radical selected from —R¹⁴, —X⁵OR¹⁴, —X⁵SR¹⁴, —X⁵S(O)R¹⁴, —X⁵S(O)₂R¹⁴, —X⁵C(O)R¹⁴, —X⁵C(O)OR¹⁴, —X⁵OC(O)R¹⁴, —X⁵NR¹⁴R¹², —X⁵NR¹²C(O)R¹⁴, —X⁵NR¹²C(O)OR¹⁴, —X⁵C(O)NR¹²R¹², —X⁵S(O)₂NR¹⁴R¹², —X⁵NR¹²S(O)₂R¹⁴, —X⁵NR¹²C(O)NR¹⁴R¹² and —X⁵NR¹²C(NR¹²)NR¹⁴R¹², wherein X⁵ is a bond or (C₁₋₆)alkylene; R¹² at each occurrence independently is hydrogen, (C₁₋₆)alkyl or halo-substituted(C₁₋₆)alkyl; R¹³ is (C₁₋₆)alkyl or halo-substituted(C₁₋₆)alkyl; and R¹⁴ is (C₃₋₁₀)cycloalkyl(C₀₋₆)alkyl, hetero(C₃₋₁₀)cycloalkyl(C₀₋₃)alkyl, (C₆₋₁₀)aryl(C₀₋₆)alkyl, hetero(C₅₋₁₀)aryl(C₀₋₆)alkyl, (C₉₋₁₀)bicycloaryl(C₀₋₆)alkyl or hetero(C₈₋₁₀)bicycloaryl(C₀₋₆)alkyl;
R¹ is hydrogen and R² is (C₁₋₆)alkyl; and
R³ is CH₂X⁶, wherein X⁶ is —X⁵NR¹²S(O)₂R¹² or —X⁵S(O)₂R¹⁴ wherein X⁵, R¹² and R¹⁴ are as defined above;
wherein within R³ any alicyclic or aromatic ring system is unsubstituted or substituted further by 1 to 5 radicals independently selected from (C₁₋₆)alkyl, (C₁₋₆)alkylidene, cyano, halo, halo-substituted(C₁₋₄)alkyl, nitro, —X⁵NR¹²R¹², —X⁵NR¹²C(O)R¹², —X⁵NR¹²C(O)OR¹², —X⁵NR¹²C(O)NR¹²R¹², —X⁵NR¹²C(NR¹²)NR¹²R¹², —X⁵OR¹², —X⁵SR¹², —X⁵NR¹²S(O)₂R¹², —X⁵C(O)R¹², —X⁵OC(O)R¹², —X⁵C(O)NR¹²R¹², —X⁵S(O)₂NR¹²R¹², —X⁵NR¹²S(O)₂R¹², —X⁵P(O)(OR¹²)OR¹², —X⁵OP(O)(OR¹²)OR¹², —X⁵NR¹²C(O)R¹³, —X⁵S(O)R¹³, —X⁵C(O)R¹³ and —X⁵S(O)₂R¹³ and within R³ any aliphatic moiety is unsubstituted or substituted further by 1 to 5 radicals independently selected from cyano, halo, nitro, —NR¹²R¹², —NR¹²C(O)R¹², —NR¹²C(O)OR¹², —NR¹²C(O)NR¹²R¹², —NR¹²C(NR¹²)NR¹²R¹², —OR¹², —SR¹², —C(O)OR¹², —C(O)R¹², —OC(O)R¹², —C(O)NR¹²R¹², —S(O)₂NR¹²R¹², —NR¹²S(O)₂R¹², —O(O)(OR¹²)OR¹², —OP(O)(OR¹²)OR¹², —NR¹²C(O)R¹³, —S(O)R¹³, —S(O)R¹³ and —S(O)₂R¹³; wherein X⁵, R¹², R¹³ and R¹⁴ are as described above; with the proviso that only one bicyclic ring structure is present within R³; and the N-oxide derivatives, prodrug derivatives, protected derivatives, individual isomers and mixtures of isomers thereof; and the pharmaceutically acceptable salts and solvates of such compounds and the N-oxide derivatives, prodrug derivatives, protected derivatives, individual isomers and mixtures of isomers thereof.

8. The compound of claim 3 in which:
X¹ is —NHC(R¹)(R²)X³ or —NHCH(R¹⁹)C(O)R²⁰, wherein R¹ is hydrogen or (C₁₋₆)alkyl and R² is hydrogen, (C₁₋₆)alkyl, —X⁵OR¹², —X⁵S(O)R¹³, —X⁵OR¹⁴, (C₆₋₁₀)aryl(C₀₋₆)alkyl or hetero(C₅₋₁₀)aryl(C₀₋₆)alkyl or R¹ and R² taken together with the carbon atom to which both R¹ and R² are attached form (C₃₋₆)cycloalkylene or (C₃₋₆)heterocycloalkylene, wherein within said R² any heteroaryl, aryl, cycloalkylene or heterocycloalkylene is unsubstituted or substituted with (C₁₋₆)alkyl or hydroxy, wherein X³ is cyano, —C(O)R¹⁶, —C(R⁶)(OR⁶)₂, 1 —CH═CHS(O)₂R⁵, —CH₂C(O)R¹⁶, —C(O)CF₂C(O)NR⁵R⁵, —C(O)C(O)NR⁵R⁶, —C(O)C(O)OR⁵, —C(O)CH₂OR⁵, —C(O)CH₂N(R⁶)SO₂R⁵ or —C(O)C(O)R⁵ and R¹⁹ and R²⁰ together with the atoms to which R¹⁹ and R²⁰ are attached form (C₄₋₈)heterocycloalkylene, wherein no more than one of the ring member atoms comprising the ring is a heteroatom selected from —NR²¹— or —O—, wherein the ring is unsubstituted or substituted with (C₁₋₆)alkyl or —X⁵C(O)OR¹² and R²¹ is hydrogen, (C₁₋₆)alkyl, —X⁵C(O)R¹², —X⁵C(O)OR¹², —R¹⁴, —X⁵C(O)R¹⁴ or —C(O)OR¹⁴;
X² is —OH or —OC(O)NR¹²R¹², wherein each R¹² independently represent hydrogen or (C₁₋₆)alkyl, wherein said alkyl is unsubstituted or substituted with hydroxy or methoxy, or X² is —OC(O)NHR¹⁴, wherein R¹⁴ is (C₃₋₁₀)cycloalkyl(C₀₋₆)alkyl or hetero(C₃₋₁₀)cycloalkyl(C₁₋₃)alkyl, or X² is —OC(O)R¹⁴, wherein R¹⁴ is —NR²²R²³ and R²² and R²³ together with the nitrogen atom to which both R²² and R²³ attached form a hetero(C₄₋₆)cycloalkyl ring, which ring may be unsubstituted or substituted with hydroxy; and
R³ is —CH₂X⁶; wherein X⁶ is is selected from —X⁵SR¹², —X⁵C(O)NR¹²R¹², —X⁵S(O)₂R¹³, —X⁵C(O)R¹³, —X⁵OR¹², —X⁵SR¹⁴, —X⁵R¹⁴, —X⁵S(O)₂R¹⁴, —X⁵C(O)R¹⁴, —X⁵C(O)NR¹⁴R¹²; and the N-oxide derivatives, prodrug derivatives, protected derivatives, individual isomers and mixtures of isomers thereof, and the pharmaceutically acceptable salts and solvates of such compounds and the N-oxide derivatives, prodrug derivatives, protected derivatives, individual isomers and mixtures of isomers thereof.

9. The compound of claim 8 in which:
X³ is cyano, —C(O)X⁴, —C(O)H, —C(O)N(CH₃)OCH₃, —CH(OCH₃)₂, —C(O)CF₃, —C(O)CF₂CF₃, —CH₂C(O)R¹⁶, (E)-2-benzenesulfonyl-vinyl, 2-dimethylcarbamoyl-2,2-difluoro-acetyl, 2-oxo-2-pyrrolidin-1-yl-acetyl, 2-morpholin-4-yl-2-oxo-acetyl, 2-oxo-2-piperazin-1-yl-acetyl, 2-(4-methanesulfonyl-piperazin-1-yl)-2-oxo-acetyl, 2-(1,1-dioxo-1□⁶-thiomorpholin-4-yl)-2-oxo-acetyl, dimethylaminooxalyl, tetrahydro-pyran-4-ylaminooxalyl, 2-morpholin-4-yl-ethylaminooxalyl, cyclopentyl-ethyl-aminooxalyl, pyridin-3-ylaminooxalyl, phenylaminooxalyl, 1-benzoyl-piperidin-4-ylaminooxalyl, 1-benzylcarbamoyl-methanoyl, 1-benzyloxy(oxalyl), 2-benzyloxy-acetyl, 2-benzenesulfonylamino-ethanoyl, 2-oxo,2-phenyl-ethanoyl, 3H-oxazole-2-carbonyl, 5-trifluoromethyl-oxazole-2-carbonyl, 3-trifluoromethyl-[1,2,4]oxadiazole-5-carbonyl, 2,2,3,3,3-pentafluoro-propionyl, hydroxyaminooxalyl, oxalyl, 2-(1,3-dihydro-isoindol-2-yl)-2-oxo-acetyl, benzothiazol-2-ylaminooxalyl, 2-oxo-ethyl, 2-oxazol-2-yl-2-oxo-ethyl or 2-benzooxazol-2-yl-2-oxo-ethyl;
X² is selected from —OH, dimethylcarbamoyloxy, morpholin-4-ylcarbonyloxy, piperidin-1-yl-carbonyloxy, pyrrolidin-1-yl-carbonyloxy, pyrimidin-2-ylamino, tetrahydro-pyran-4-ylamino, 1-methyl-piperidin-4-ylamino, N-(2-methoxyethyl)-N-(tetrahydro-pyran-4-yl)amino, isopropylamino and cyclohexylamino; 4-tert-butoxycarbonylpiperazin-1-ylcarbonyloxy, N-benzyl-carbamoyloxy, pyrrolidin-1-yl-carbonyloxy, N,N-dimethyl-carbamoyloxy, piperidin-1-yl-carbonyloxy, 4-methanesulfonyl-piperazin-1-yl-carbonyloxy, 4-ethoxycarbonylpiperazin-1-ylcarbonyloxy, N-cyclohexyl-carbamoyloxy, N-phenyl-carbamoyloxy, N-(5,6,7,8-tetrahydro-naphthalen-1-yl)-carbamoyloxy, N-butyl-N-methyl-carbamoyloxy, N-pyridin-3-yl-carbamoyloxy, N-isopropyl-carbamoyloxy, N-pyridin-4-yl-carbamoyloxy, N-cyanomethyl-N-methyl-carbamoyloxy, N,N-bis-(2-methoxy-ethyl)-carbamoyloxy, N-phenethyl-carbamoyloxy, piperazine-carbonyloxy, N-naphthalen-2-yl-carbamoyloxy, 4-benzyl-piperazine-1-carbamoyloxy, 4-(1-furan-2-yl-carbonyl)-piperazine-1-carbamoyloxy, thiomorpholin-4-yl-carbonyloxy, 1,1-dioxo-1λ⁶-thiomorpholin-4-yl)-carbonyloxy, bis-(2-methoxy-ethyl)-carbamoyloxy, morpholin-4-ylcarbonyloxy, 2-methoxyethylcarbamoyloxy, diethylcarbamoyloxy, pyrrolidin-1-ylcarbonyloxy, 2-hydroxyethylcarbamoyloxy, tetrahydro-furan-2-ylmethylcarbamoyloxy, cyclopropylcarbamoyloxy, tert-butylcarbamoyloxy, 3-hydroxy-pyrrolidin-1-yl-carbonyloxy and carbamoyloxy; and
R³ is thiophene-2-sulfonyl-methyl, 3-chloro-2-fluoro-phenyl-methane-sulfonyl-methyl, benzene-sulfonyl-methyl, phenyl-methane-sulfonyl-methyl, 2-(1,1-difluoro-methoxy)-phenyl-methane-sulfonyl-methyl, 2-benzene-sulfonyl-ethyl, 2-(pyridine-2-sulfonyl)-ethyl, 2-(pyridine-4-sulfonyl)-ethyl, 2-phenyl-methanesulfonyl-ethyl, oxy-pyridin-2-yl-methane-sulfonyl-methyl, prop-2-ene-1-sulfonyl-methyl, 4-methoxy-phenyl-methane-sulfonyl-methyl, p-tolyl-methane-sulfonyl-methyl, 4-chloro-phenyl-methane-sulfonyl-methyl, o-tolyl-methane-sulfonyl-methyl, 3,5-dimethyl-phenyl-methane-sulfonyl-methyl, 4-trifluoro-methyl-phenyl-methane-sulfonyl-methyl, 4-trifluoro-methoxy-phenyl-methane-sulfonyl-methyl, 2-bromo-phenyl-methane-sulfonyl-methyl, pyridin-2-yl-methane-sulfonyl-methyl, pyridin-3-yl-methane-sulfonyl-methyl, pyridin-4-yl-methane-sulfonyl-methyl, naphthalen-2-yl-methane-sulfonyl-methyl, 3-methyl-phenyl-methane-sulfonyl-methyl, 3-trifluoro-methyl-phenyl-methane-sulfonyl-methyl, 3-trifluoro-methoxy-phenyl-methane-sulfonyl-methyl, 4-fluoro-2-trifluoromethoxy-phenyl-methane-sulfonylmethyl, 2-fluoro-6-trifluoromethyl-phenylmethanesulfonylmethyl, 3-chloro-phenylmethanesulfonylmethyl, 2-fluoro-phenylmethanesulfonylmethyl, 2-trifluoro-phenylmethanesulfonylmethyl, 2-cyano-phenylmethanesulfonylmethyl, 4-tert-butyl-phenylmethanesulfonylmethyl, 2-fluoro-3-methyl-phenyl-methane-sulfonyl-methyl, 3-fluoro-phenylmethanesulfonylmethyl, 4-fluoro-phenylmethane-sulfonylmethyl, 2-chloro-phenylmethanesulfonylmethyl, 2,5-difluoro-phenylmethane-sulfonylmethyl, 2,6-difluoro-phenylmethanesulfonylmethyl, 2,5-dichloro-phenyl-methane-sulfonylmethyl, 3,4-dichloro-phenylmethanesulfonylmethyl, 2-(1,1-difluoro-methoxy)-phenyl-methanesulfonylmethyl, 2-cyano-phenyl-methane-sulfonyl-methyl, 3-cyano-phenylmethanesulfonylmethyl, 2-trifluoro-methoxy-phenyl-methane-sulfonylmethyl, 2,3-difluoro-phenylmethanesulfonylmethyl, 2,5-difluoro-phenyl-methanesulfonylmethyl, biphenyl-2-ylmethanesulfonylmethyl, cyclohexylmethyl, 3-fluoro-phenyl-methanesulfonylmethyl, 3,4-difluoro-phenyl-methanesulfonylmethyl, 2,4-difluoro-phenylmethanesulfonylmethyl, 2,4,6-trifluoro-phenylmethanesulfonylmethyl, 2,4,5-trifluoro-phenylmethanesulfonylmethyl, 2,3,4-trifluoro-phenylmethanesulfonylmethyl, 2,3,5-trifluoro-phenyl-methane-sulfonylmethyl, 2,5,6-trifluoro-phenylmethanesulfonylmethyl, 2-chloro-5-trifluoro-methylphenylmethanesulfonylmethyl, 2-methyl-propane-1-sulfonyl, 2-fluoro-3-trifluoro-methylphenylmethanesulfonylmethyl, 2-fluoro-4-trifluoro-methylphenylmethanesulfonylmethyl, 2-fluoro-5-trifluoro-methyl-phenyl-methane-sulfonyl-methyl, 4-fluoro-3-trifluoro-methylphenylmethanesulfonylmethyl, 2-methoxy-phenyl-methanesulfonylmethyl, 3,5-bis-trifluoromethyl-phenylmethanesulfonylmethyl, 4-difluoromethoxy-phenylmethanesulfonylmethyl, 2-difluoro-methoxy-phenyl-methanesulfonylmethyl, 3-difluoromethoxy-phenylmethanesulfonylmethyl, 2,6-dichloro-phenylmethanesulfonylmethyl, biphenyl-4-ylmethanesulfonylmethyl, 3,5-dimethyl-isoxazol-4-ylmethanesulfonylmethyl, 5-chloro-thien-2-yl-methane-sulfonylmethyl, 2-[4-(1,1-difluoro-methoxy)-benzenesulfonyl]-ethyl, 2-[2-(1,1-difluoro-methoxy)-benzenesulfonyl]-ethyl, 2-[3-(1,1-difluoro-methoxy)-benzenesulfonyl]-ethyl, 2-(4-trifluoromethoxy-benzenesulfonyl)-ethyl, 2-(3-trifluoromethoxy-benzenesulfonyl)-ethyl, 2-(2-trifluoro-methoxy-benzene-sulfonyl)-ethyl, (cyanomethyl-methyl-carbamoyl)-methyl, biphenyl-3-ylmethyl, 2-oxo-2-pyrrolidin-1-yl-ethyl, 2-benzenesulfonyl-ethyl, isobutylsulfanylmethyl, 2-phenylsulfanyl-ethyl, cyclohexylmethanesulfonylmethyl, 2-cyclohexyl-ethanesulfonyl, benzyl, naphthalen-2-yl, benzylsulfanylmethyl, 2-trifluoromethyl-benzylsulfanylmethyl, phenylsulfanyl-ethyl, cyclopropyl-methanesulfonylmethyl, 5-bromo-thien-2-ylmethyl, 3-phenyl-propyl, 2,2-difluoro-3-phenyl-propyl, 3,4,5-trimethoxy-phenylmethanesulfonylmethyl, 2,2-difluoro-3-thien-2-yl-propyl, cyclohexylethyl, cyclohexylmethyl, tert-butylmethyl, 1-methylcyclohexylmethyl, 1-methylcyclopentylmethyl, 2,2-difluoro-3-phenylpropyl, 2,2-dimethyl-3-phenylpropyl, 1-benzylcyclopropylmethyl, —X⁵S(O)₂R¹³ and —X⁵S(O)₂R¹⁴, wherein R¹³ is alkyl and R¹⁴ is phenyl which phenyl is unsubstituted or substituted; and the N-oxide derivatives, prodrug derivatives, protected derivatives, individual isomers and mixtures of isomers thereof, and the pharmaceutically acceptable salts and solvates of such compounds and the N-oxide derivatives, prodrug derivatives, protected derivatives, individual isomers and mixtures of isomers thereof.

10. A compound of claim 9 in which:
X³ is 1H-benzoimidazol-2-ylcarbonyl, pyrimidin-2-ylcarbonyl, benzooxazol-2-ylcarbonyl, benzothiazol-2-ylcarbonyl, pyridazin-3-ylcarbonyl, 3-phenyl-[1,2,4]oxadiazol-5-ylcarbonyl or 3-ethyl-[1,2,4]oxadiazol-5-ylcarbonyl, 2-oxo-2-pyrrolidin-1-yl-acetyl, 2-morpholin4-yl-2-oxo-acetyl, 2-oxo-2-piperazin-1-yl-acetyl, 2-(4-methanesulfonyl-piperazin-1-yl)-2-oxo-acetyl, 2-(1,1-dioxo-1□⁶-thiomorpholin-4-yl)-2-oxo-acetyl, dimethylaminooxalyl, tetrahydro-pyran-4-ylaminooxalyl, 2-morpholin-4-yl-ethylaminooxalyl, cyclopentyl-ethyl-aminooxalyl, pyridin-3-ylaminooxalyl, phenylaminooxalyl or 1-benzoyl-piperidin-4-ylaminooxalyl;
X² is selected from —OH, dimethylcarbamoyloxy, morpholin-4-ylcarbonyloxy, piperidin-1-yl-carbonyloxy, pyrrolidin-1-yl-carbonyloxy, pyrimidin-2-ylamino, tetrahydro-pyran-4-ylamino, 1-methyl-piperidin-4-ylamino, N-(2-methoxyethyl)-N-(tetrahydro-pyran-4-yl)amino, isopropylamino and cyclohexylamino;
R³ is cyclohexylethyl, cyclohexylmethyl, tert-butylmethyl, 1-methylcyclohexylmethyl, 1-methylcyclopentylmethyl, 2,2-difluoro-3-phenylpropyl, 2,2-dimethyl-3-phenylpropyl, 1-benzylcyclopropylmethyl, —X⁵S(O)₂R¹³ or —X⁵S(O)₂R¹⁴, wherein R¹³ is alkyl and R¹⁴ is phenyl which phenyl is unsubstituted or substituted; and the pharmaceutically acceptable salts and solvates of such compounds and the N-oxide derivatives, prodrug derivatives, protected derivatives, individual isomers and mixtures of isomers thereof.

11. The compound of claim 3 in which:
X¹ is —NHC(R¹)(R²)X³ or —NHCH(R¹⁹)C(O)R²⁰, wherein R¹ is hydrogen or (C₁₋₆)alkyl and R² is hydrogen, (C₁₋₆)alkyl, —X⁵OR¹², —X⁵S(O)R¹³, —X⁵OR¹⁴, (C₆₋₁₀)aryl(C₀₋₆)alkyl or hetero(C₅₋₁₀)aryl(C₀₋₆)alkyl or R¹ and R² taken together with the carbon atom to which both R¹ and R² are attached form (C₃₋₆)cycloalkylene or (C₃₋₆)heterocycloalkylene, wherein within said R² any heteroaryl, aryl, cycloalkylene or heterocycloalkylene is unsubstituted or substituted with (C₁₋₆)alkyl or hydroxy, wherein X³ is cyano, —C(O)R¹⁶, —C(R⁶)(OR⁶)₂, —CH═CHS(O)₂R⁵, —CH₂C(O)R¹⁶, —C(O)CF₂C(O)NR⁵R⁵, —C(O)C(O)NR⁵R⁶, —C(O)C(O)OR⁵, —C(O)CH₂OR⁵, —C(O)CH₂N(R⁶)SO₂R⁵ or —C(O)C(O)R⁵ and R¹⁹ and R²⁰ together with the atoms to which R¹⁹ and R²⁰ are attached form (C₄₋₈)heterocycloalkylene, wherein no more than one of the ring member atoms comprising the ring is a heteroatom selected from —NR²¹— or —O—, wherein the ring is unsubstituted or substituted with (C₁₋₆)alkyl or —X⁵C(O)OR¹² and R²¹ is hydrogen, (C₁₋₆)alkyl, —X⁵C(O)R¹², —X⁵C(O)OR¹², —R¹⁴, —X⁵C(O)R¹⁴ or —C(O)OR¹⁴;
X² is —NHR¹⁵, wherein R¹⁵ is (C₆₋₁₀)aryl, hetero(C₅₋₁₀)aryl, (C₉₋₁₀)bicycloaryl or hetero(C₈₋₁₀)bicycloaryl, or —NR¹⁷R¹⁸, wherein R¹⁷ is hetero(C₃₋₁₀)cycloalkyl and R¹⁸ is hydrogen or R¹⁷ and R¹⁸ independently are (C₆₋₁₀)aryl(C₁₋₆)alkyl or hetero(C₅₋₁₀)aryl(C₁₋₆)alkyl, wherein within R¹⁵, R¹⁷ and R¹⁸ any alicyclic or aromatic ring system is unsubstituted or substituted further by 1 to 5 radicals independently selected from (C₁₋₆)alkyl, cyano, halo, nitro, halo-substituted(C₁₋₄)alkyl, —X⁵OR¹², —X⁵C(O)OR¹², —X⁵C(O)R¹³, —X⁵C(O)NR¹²R¹², —X⁵NR¹²S(O)₂R¹² and/or 1 radical selected from —R¹⁴, —X⁵OR¹⁴ and —X⁵C(O)NR¹⁴R¹²; and
R³ is —CH₂X⁶; wherein X⁶ is is selected from —X⁵SR¹², —X⁵C(O)NR¹²R¹², —X⁵S(O)₂R¹³, —X⁵C(O)R¹³, —X⁵OR¹², —X⁵SR¹⁴, —X⁵R¹⁴, —X⁵S(O)₂R¹⁴, —X⁵C(O)R¹⁴, —X⁵C(O)NR¹⁴R¹²; and the N-oxide derivatives, prodrug derivatives, protected derivatives, individual isomers and mixtures of isomers thereof; and the pharmaceutically acceptable salts and solvates of such compounds and the N-oxide derivatives, prodrug derivatives, protected derivatives, individual isomers and mixtures of isomers thereof.

12. The compound of claim 11 in which:
X³ is cyano, —C(O)X⁴, —C(O)H, —C(O)N(CH₃)OCH₃, —CH(OCH₃)₂, —C(O)CF₃, —C(O)CF₂CF₃, —CH₂C(O)R¹⁶, (E)-2-benzenesulfonyl-vinyl, 2-dimethylcarbamoyl-2,2-difluoro-acetyl, 2-oxo-2-pyrrolidin-1-yl-acetyl, 2-morpholin-4-yl-2-oxo-acetyl, 2-oxo-2-piperazin-1-yl-acetyl, 2-(4-methanesulfonyl-piperazin-1-yl)-2-oxo-acetyl, 2-(1,1-dioxo-1□⁶-thiomorpholin-4-yl)-2-oxo-acetyl, dimethylaminooxalyl, tetrahydro-pyran-4-ylaminooxalyl, 2-morpholin-4-yl-ethylaminooxalyl, cyclopentyl-ethyl-aminooxalyl, pyridin-3-ylaminooxalyl, phenylaminooxalyl, 1-benzoyl-piperidin-4-ylaminooxalyl, 1-benzylcarbamoyl-methanoyl, 1-benzyloxy(oxalyl), 2-benzyloxy-acetyl, 2-benzenesulfonylamino-ethanoyl, 2-oxo-2-phenyl-ethanoyl, 3H-oxazole-2-carbonyl, 5-trifluoromethyl-oxazole-2-carbonyl, 3-trifluoromethyl-[1,2,4]oxadiazole-5-carbonyl, 2,2,3,3,3-pentafluoro-propionyl, hydroxyaminooxalyl, oxalyl, 2-(1,3-dihydro-isoindol-2-yl)-2-oxo-acetyl, benzothiazol-2-ylaminooxalyl, 2-oxo-ethyl, 2-oxazol-2-yl-2-oxo-ethyl or 2-benzooxazol-2-yl-2-oxo-ethyl;
X² is selected from 5-nitrothiazol-2-ylamino, 2-nitrophenylamino, pyrimidin-2-ylamino, tetrahydro-pyran-4-ylamino, N-(2-methoxyethyl)-N-(tetrahydro-pyran-4-yl)amino, 1-methyl-piperidin-4-ylamino, isopropylamino, di(thien-2-ylmethyl)amino or di(benzyl)amino; and
R³ is thiophene-2-sulfonyl-methyl, 3-chloro-2-fluoro-phenyl-methane-sulfonyl-methyl, benzene-sulfonyl-methyl, phenyl-methane-sulfonyl-methyl, 2-(1,1-difluoro-methoxy)-phenyl-methane-sulfonyl-methyl, 2-benzene-sulfonyl-ethyl, 2-(pyridine-2-sulfonyl)-ethyl, 2-(pyridine-4-sulfonyl)-ethyl, 2-phenyl-methanesulfonyl-ethyl, oxy-pyridin-2-yl-methane-sulfonyl-methyl, prop-2-ene-1-sulfonyl-methyl, 4-methoxy-phenyl-methane-sulfonyl-methyl, p-tolyl-methane-sulfonyl-methyl, 4-chloro-phenyl-methane-sulfonyl-methyl, o-tolyl-methane-sulfonyl-methyl, 3,5-dimethyl-phenyl-methane-sulfonyl-methyl, 4-trifluoro-methyl-phenyl-methane-sulfonyl-methyl, 4-trifluoro-methoxy-phenyl-methane-sulfonyl-methyl, 2-bromo-phenyl-methane-sulfonyl-methyl, pyridin-2-yl-methane-sulfonyl-methyl, pyridin-3-yl-methane-sulfonyl-methyl, pyridin-4-yl-methane-sulfonyl-methyl, naphthalen-2-yl-methane-sulfonyl-methyl, 3-methyl-phenyl-methane-sulfonyl-methyl, 3-trifluoro-methyl-phenyl-methane-sulfonyl-methyl, 3-trifluoro-methoxy-phenyl-methane-sulfonyl-methyl, 4-fluoro-2-trifluoromethoxy-phenyl-methane-sulfonylmethyl, 2-fluoro-6-trifluoromethyl-phenylmethanesulfonylmethyl, 3-chloro-phenylmethanesulfonylmethyl, 2-fluoro-phenylmethanesulfonylmethyl, 2-trifluoro-phenylmethanesulfonylmethyl, 2-cyano-phenylmethanesulfonylmethyl, 4-tert-butyl-phenylmethanesulfonylmethyl, 2-fluoro-3-methyl-phenyl-methane-sulfonyl-methyl, 3-fluoro-phenylmethanesulfonylmethyl, 4-fluoro-phenylmethane-sulfonylmethyl, 2-chloro-phenylmethanesulfonylmethyl, 2,5-difluoro-phenylmethane-sulfonylmethyl, 2,6-difluoro-phenylmethanesulfonylmethyl, 2,5-dichloro-phenyl-methane-sulfonylmethyl, 3,4-dichloro-phenylmethanesulfonylmethyl, 2-(1,1-difluoro-methoxy)-phenyl-methanesulfonylmethyl, 2-cyano-phenyl-methane-sulfonyl-methyl, 3-cyano-phenylmethanesulfonylmethyl, 2-trifluoro-methoxy-phenyl-methane-sulfonylmethyl, 2,3-difluoro-phenylmethanesulfonylmethyl, 2,5-difluoro-phenyl-methanesulfonylmethyl, biphenyl-2-ylmethanesulfonylmethyl, cyclohexylmethyl, 3-fluoro-phenyl-methanesulfonylmethyl, 3,4-difluoro-phenyl-methanesulfonylmethyl, 2,4-difluoro-phenylmethanesulfonylmethyl, 2,4,6-trifluoro-phenylmethanesulfonylmethyl, 2,4,5-trifluoro-phenylmethanesulfonylmethyl, 2,3,4-trifluoro-phenylmethanesulfonylmethyl, 2,3,5-trifluoro-phenyl-methane-sulfonylmethyl, 2,5,6-trifluoro-phenylmethanesulfonylmethyl, 2-chloro-5-trifluoro-methylphenylmethanesulfonylmethyl, 2-methyl-propane-1-sulfonyl, 2-fluoro-3-trifluoro-methylphenylmethanesulfonylmethyl, 2-fluoro-4-trifluoro-methylphenylmethanesulfonylmethyl, 2-fluoro-5-trifluoro-methyl-phenyl-methane-sulfonyl-methyl, 4-fluoro-3-trifluoro-methylphenylmethanesulfonylmethyl, 2-methoxy-phenyl-methanesulfonylmethyl, 3,5-bis-trifluoromethyl-phenylmethanesulfonylmethyl, 4-difluoromethoxy-phenylmethanesulfonylmethyl, 2-difluoro-methoxy-phenyl-methanesulfonylmethyl, 3-difluoromethoxy-phenylmethanesulfonylmethyl, 2,6-dichloro-phenylmethanesulfonylmethyl, biphenyl-4-ylmethanesulfonylmethyl, 3,5-dimethyl-isoxazol-4-ylmethanesulfonylmethyl, 5-chloro-thien-2-yl-methane-sulfonylmethyl, 2-[4-(1,1-difluoro-methoxy)-benzenesulfonyl]-ethyl, 2-[2-(1,1-difluoro-methoxy)-benzenesulfonyl]-ethyl, 2-[3-(1,1-difluoro-methoxy)-benzenesulfonyl]-ethyl, 2-(4-trifluoromethoxy-benzenesulfonyl)-ethyl, 2-(3-trifluoromethoxy-benzenesulfonyl)-ethyl, 2-(2-trifluoro-methoxy-benzene-sulfonyl)-ethyl, (cyanomethyl-methyl-carbamoyl)-methyl, biphenyl-3-ylmethyl, 2-oxo-2-pyrrolidin-1-yl-ethyl, 2-benzenesulfonyl-ethyl, isobutylsulfanylmethyl, 2-phenylsulfanyl-ethyl, cyclohexylmethanesulfonylmethyl, 2-cyclohexyl-ethanesulfonyl, benzyl, naphthalen-2-yl, benzylsulfanylmethyl, 2-trifluoromethyl-benzylsulfanylmethyl, phenylsulfanyl-ethyl, cyclopropyl-methanesulfonylmethyl, 5-bromo-thien-2-ylmethyl, 3-phenyl-propyl, 2,2-difluoro-3-phenyl-propyl, 3,4,5-trimethoxy-phenylmethanesulfonylmethyl, 2,2-difluoro-3-thien-2-yl-propyl, cyclohexylethyl, cyclohexylmethyl, tert-butylmethyl, 1-methylcyclohexylmethyl, 1-methylcyclopentylmethyl, 2,2-difluoro-3-phenylpropyl, 2,2-dimethyl-3-phenylpropyl, 1-benzylcyclopropylmethyl, —X⁵S(O)₂R¹³ and —X⁵S(O)₂R¹⁴, wherein R¹³ is alkyl and R¹⁴ is phenyl which phenyl is unsubstituted or substituted; and the N-oxide derivatives, prodrug derivatives, protected derivatives, individual isomers and mixtures of isomers thereof; and the pharmaceutically acceptable salts and solvates of such compounds and the N-oxide derivatives, prodrug derivatives, protected derivatives, individual isomers and mixtures of isomers thereof.

13. A compound of claim 12 in which:
X³ is 1H-benzoimidazol-2-ylcarbonyl, pyrimidin-2-ylcarbonyl, benzooxazol-2-ylcarbonyl, benzothiazol-2-ylcarbonyl, pyridazin-3-ylcarbonyl, 3-phenyl-[1,2,4]oxadiazol-5-ylcarbonyl or 3-ethyl-[1,2,4]oxadiazol-5-ylcarbonyl, 2-oxo-2-pyrrolidin-1-yl-acetyl, 2-morpholin-4-yl-2-oxo-acetyl, 2-oxo-2-piperazin-1-yl-acetyl, 2-(4-methanesulfonyl-piperazin-1-yl)-2-oxo-acetyl, 2-(1,1-dioxo-1□⁶-thiomorpholin-4-yl)-2-oxo-acetyl, dimethylaminooxalyl, tetrahydro-pyran-4-ylaminooxalyl, 2-morpholin-4-yl-ethylaminooxalyl, cyclopentyl-ethyl-aminooxalyl, pyridin-3-ylaminooxalyl, phenylaminooxalyl or 1-benzoyl-piperidin-4-ylaminooxalyl;
X² is selected from —OH, dimethylcarbamoyloxy, morpholin-4-ylcarbonyloxy, piperidin-1-yl-carbonyloxy, pyrrolidin-1-yl-carbonyloxy, pyrimidin-2-ylamino, tetrahydro-pyran-4-ylamino, 1-methyl-piperidin-4-ylamino, N-(2-methoxyethyl)-N-(tetrahydro-pyran-4-yl)amino, isopropylamino and cyclohexylamino;
R³ is cyclohexylethyl, cyclohexylmethyl, tert-butylmethyl, 1-methylcyclohexylmethyl, 1-methylcyclopentylmethyl, 2,2-difluoro-3-phenylpropyl, 2,2-dimethyl-3-phenylpropyl, 1-benzylcyclopropylmethyl, —X⁵S(O)₂R¹³ or —X⁵S(O)₂R¹⁴, wherein R¹³ is alkyl and R¹⁴ is phenyl which phenyl is unsubstituted or substituted; and the pharmaceutically acceptable salts and solvates of such compounds and the N-oxide derivatives, prodrug derivatives, protected derivatives, individual isomers and mixtures of isomers thereof.

14. A compound of claim 1 selected from the group consisting of:
(R)—N-cyanomethyl-2-hydroxy-3-phenylmethanesulfonyl-propionamide;
(R)—N-(1-cyano-1-thiophen-2-yl-methyl)-2-hydroxy-3-phenylmethanesulfonyl-propionamide;
(R)—N-(1-cyano-1-thiophen-2-yl-methyl)-3-[2-(1,1-difluoro-methoxy)-phenylmethanesulfonyl]-2-hydroxy-propionamide;
(R)—N-cyanomethyl-3-[2-(1,1-difluoro-methoxy)-phenylmethanesulfonyl]-2-hydroxy-propionamide;
morpholine-4-carboxylic acid (R)-1-(cyanomethyl-carbamoyl)-2-phenylmethanesulfonyl-ethyl ester;
morpholine-4-carboxylic acid (R)-1-(cyanomethyl-carbamoyl)-2-[2-(1,1-difluoro-methoxy)-phenylmethanesulfonyl]-ethyl ester;
(R)-(2-methoxy-ethyl)-carbamic acid 1-(cyanomethyl-carbamoyl)-2-phenylmethanesulfonyl-ethyl ester;
(S)-diethyl-carbamic acid 1-(cyanomethyl-carbamoyl)-2-cyclohexyl-ethyl ester;
(S)-pyrrolidine-1-carboxylic acid 1-(cyanomethyl-carbamoyl)-2-cyclohexyl-ethyl ester;
(S)-morpholine-4-carboxylic acid 1-(cyanomethyl-carbamoyl)-2-cyclohexyl-ethyl ester;
(S)-4-Ethyl-piperazine-1-carboxylic acid 1-(cyanomethyl-carbamoyl)-2-cyclohexyl-ethyl ester;
(S)-2-hydroxymethyl-pyrrolidine-1-carboxylic acid (S)-1-(cyanomethyl-carbamoyl)-2-cyclohexyl-ethyl ester;
(S)-(2,2,2-Trifluoro-ethyl)-carbamic acid 1-(cyanomethyl-carbamoyl)-.2-cyclohexyl-ethyl ester;
(S)-(2-hydroxyethyl)-carbamic acid 1-(cyanomethyl-carbamoyl)-2-cyclohexyl-ethyl ester;
(Tetrahydrofuran-2-ylmethyl)-carbamic acid (S)-1-(cyanomethyl-carbamoyl)-2-cyclohexyl-ethyl ester;
(S)-Azetidine-1-carboxylic acid 1-(cyanomethyl-carbamoyl)-2-cyclohexyl-ethyl ester;
(S)-cyclopropyl-carbamic acid 1-(cyanomethyl-carbamoyl)-2-cyclohexyl-ethyl ester;
(S)-piperidine-1-carboxylic acid 1-(cyanomethyl-carbamoyl)-2-cyclohexyl-ethyl ester;
(S)-(2-methoxy-ethyl)-carbamic acid 1-(cyanomethyl-carbamoyl)-2-cyclohexyl-ethyl ester;
(R)-3-hydroxy-pyrrolidine-1-carboxylic acid (S)-1-(cyanomethyl-carbamoyl)-2-cyclohexyl-ethyl ester;
(S)-3-hydroxy-pyrrolidine-1-carboxylic acid (S)-1-(cyanomethyl-carbamoyl)-2-cyclohexyl-propyl ester;
(S)-morpholine-4-carboxylic acid 1-(cyanomethyl-carbamoyl)-3-cyclohexyl-propyl ester;
morpholine-4-carboxylic acid (R)-1-[(S)-1-(1-benzooxazol-2-yl-methanoyl)-propylcarbamoyl]-2-phenylmethanesulfonyl-ethyl ester;
morpholine-4-carboxylic acid (R)-1-[(S)-1-(1-benzooxazol-2-yl-methanoyl)-propylcarbamoyl]-2-[2-(1,1-difluoro-methoxy)-phenylmethanesulfonyl]-ethyl ester;
morpholine-4-carboxylic acid (R)-1-[(S)-1-(1-benzothiazol-2-yl-methanoyl)-propylcarbamoyl]-2-[2-(1,1-difluoro-methoxy)-phenylmethanesulfonyl]-ethyl ester;
pyrrolidine-1-carboxylic acid (R)-1-[(S)-1-(1-benzooxazol-2-yl-methanoyl)-propylcarbamoyl]-2-phenylmethanesulfonyl-ethyl ester;
dimethyl-carbamic acid (R)-1-[(S)-1-(1-benzooxazol-2-yl-methanoyl)-propylcarbamoyl]-2-phenylmethanesulfonyl-ethyl ester;
morpholine-4-carboxylic acid (R)-1-[(S)-1-(1-benzylcarbamoyl-methanoyl)-propylcarbamoyl]-2-phenylmethanesulfonyl-ethyl ester;
morpholine-4-carboxylic acid (S)-1-[(S)-1-(oxazolo[4,5-b]pyridine-2-carbonyl)-propylcarbamoyl]-2-phenylmethanesulfonyl-ethyl ester;
morpholine-4-carboxylic acid (S)-1-[(S)-1-(5-ethyl-[1,3,4]oxadiazole-2-carbonyl)-propylcarbamoyl]-2-phenylmethanesulfonyl-ethyl ester;
(S)-2-{(R)-3-[2-(1,1-difluoro-methoxy)-phenylmethanesulfonyl]-2-hydroxy-propanoylamino}-N-methoxy-N-methyl-butyramide;
(R)-3-[2-(1,1-difluoro-methoxy)-phenylmethanesulfonyl]-N—((S)-1-formyl-propyl)-2-hydroxy-propionamide;
(R)—N—[(S)-1-(1-benzooxazol-2-yl-methanoyl)-propyl]-2-hydroxy-3-phenyl-methanesulfonyl-propionamide;
(S)-3-{3-[2-(1,1-difluoro-methoxy)-phenylmethanesulfonyl]-propanoylamino}-2-oxo-pentanoic acid benzylamide;
N—[(S)-1-(1-benzooxazol-2-yl-methanoyl)-propyl]-3-[2-(1,1-difluoro-methoxy)-phenylmethanesulfonyl]-propionamide;
N—[(S)-1-(1-benzooxazol-2-yl-methanoyl)-3-phenyl-propyl]-3-p-tolylmethanesulfonyl-propionamide;
3-(2-difluoromethoxy-phenylmethanesulfonyl)-N-(1-ethyl-2,3-dioxo-3-pyrrolidin-1-yl-propyl)-propionamide;
3-(2-difluoromethoxy-phenylmethanesulfonyl)-N-(1-ethyl-3-morpholin-4-yl-2,3-dioxo-propyl)-propionamide;
3-(2-difluoromethoxy-phenylmethanesulfonyl)-N-(1-ethyl-2,3-dioxo-3-piperazin-1-yl-propyl)-propionamide;
3-(2-difluoromethoxy-phenylmethanesulfonyl)-N-[3-(1,1-dioxo-116-thiomorpholin-4-yl)-1-ethyl-2,3-dioxo-propyl]-propionamide;
3-(2-difluoromethoxy-phenylmethanesulfonyl)-N-[1-ethyl-3-(4-methyl-sulfonyl-piperazin-1-yl)-2,3-dioxo-propyl]-propionamide;
3-[3-(2-difluoromethoxy-phenylmethanesulfonyl)-propionylamino]-2-oxo-pentanoic acid dimethylamide;
3-[3-(2-difluoromethoxy-phenylmethanesulfonyl)-propionylamino]-2-oxo-pentanoic acid cyclopentyl-ethyl-amide;
3-[3-(2-difluoromethoxy-phenylmethanesulfonyl)-propionylamino]-2-oxo-pentanoic acid phenylamide;
3-[3-(2-difluoromethoxy-phenylmethanesulfonyl)-propionylamino]-2-oxo-pentanoic acid pyridin-3-ylamide;
3-[3-(2-difluoromethoxy-phenylmethanesulfonyl)-propionylamino-]2-oxo-pentanoic acid (tetrahydro-pyran-4-yl)-amide;
3-[3-(2-difluoromethoxy-phenylmethanesulfonyl)-propionylamino]-2-oxo-pentanoic acid (1-benzoyl-piperidin-4-yl)-amide;
3-[3-(2-difluoromethoxy-phenylmethanesulfonyl)-propionylamino]-2-oxo-pentanoic acid (2-morpholin-4-yl-ethyl)-amide;
(R)—N—[(S)-1-(1-benzooxazol-2-yl-methanoyl)-propyl]-2-(2-nitro-phenylamino)-3-phenylmethanesulfonyl-propionamide;
N-[1-(benzooxazole-2-carbonyl)-propyl]-3-phenylmethanesulfonyl-2-(pyrimidin-2-ylamino)-propionamide.
(R)—N—[(S)-1-(1-benzooxazol-2-yl-methanoyl)-butyl]-2-(5-nitro-thiazol-2-ylamino)-3-phenylmethanesulfonyl-propionamide;
(2S) (4,4-difluoro-2-hydroxy-5-phenyl-pentanoic acid (1(S)-cyano-3-phenyl-propyl)-amide;
N-(1-(S)-cyano-3-phenyl-propyl)-2-(S)-(2-morpholin-4-yl-2-oxo-ethoxy)-4-phenyl-butyramide;
N-(1-(S)-cyano-3-phenyl-propyl)-2-(S)-fluoro-4-phenyl-butyramide;
N-(1-(S)-cyano-3-phenyl-propyl)-2,2-difluoro-4-phenyl-butyramide;
N-(1-(S)-cyano-3-phenyl-propyl)-2-(S)-hydroxy-4-phenyl-butyramide;
N-(1-(S)-cyano-3-phenyl-propyl)-2-(R)-hydroxy-4-phenyl-butyramide;
N-(1-(S)-cyano-3-phenyl-propyl)-2-(R)-methoxy-4-phenyl-butyramide;
2,2-difluoro-5-phenyl-pentanoic acid (1-cyano-cyclopropyl)-amide;
N-(1-(S)-cyano-3-phenyl-propyl)-4-phenyl-butyramide;
2,2-difluoro-5-phenyl-pentanoic acid ((S)-1-cyano-3-phenyl-propyl)-amide;
N-(4-cyano-1-ethyl-piperidin-4-yl)-3-cyclohexyl-propionamide;
N-(4-cyano-1-ethyl-piperidin-4-yl)-3-(2-difluoromethoxy-phenylmethanesulfonyl)-propionamide;
(S)-tert-butyl-carbamic acid 1-(cyanomethyl-carbamoyl)-2-cyclohexyl-ethyl ester;
(R)-carbamic acid 1-(cyanomethyl-carbamoyl)-2-(2-difluoromethoxy-phenylmethanesulfonyl)-ethyl ester;
(S)-carbamic acid 1-(cyanomethyl-carbamoyl)-2-cyclohexyl-ethyl ester;
(R)-morpholine-4-carboxylic acid 1-(1-cyano-cyclopropylcarbamoyl)-2-phenylmethanesulfonyl-ethyl ester;
(R)-morpholine-4-carboxylic acid 1-(4-cyano-tetrahydro-pyran-4-ylcarbamoyl)-2-phenylmethanesulfonyl-ethyl ester;
3-cyclohexyl-2-hydroxy-N-[1-(oxazolo[4,5-b]pyridine-2-carbonyl)-propyl]-propionamide;
(R)—N-[1-(benzothiazole-2-carbonyl)-butyl]-2-isopropylamino-3-phenylmethanesulfonyl-propionamide;
(R)—N-[1-(benzothiazole-2-carbonyl)-butyl]-3-phenylmethanesulfonyl-2-(tetrahydro-pyran-4-ylamino)-propionamide;
(R)—N-[1-(benzothiazole-2-carbonyl)-butyl]-2-dibenzylamino-3-phenylmethanesulfonyl-propionamide;
(R)—N-[1-(benzothiazole-2-carbonyl)-butyl]-2-dimethylamino-3-phenylmethanesulfonyl-propionamide;
(R)—N—[(S)-1-(benzoxazole-2-carbonyl)-butyl]-3-phenylmethanesulfonyl-2-(tetrahydro-pyran-4-ylamino)-propionamide;
(R)—N—[(S)-1-(benzoxazole-2-carbonyl)-butyl]-2-(1-methyl-piperidin-4-ylamino)-3-phenylmethanesulfonyl-propionamide;
(R)—N—[(S)-1-(benzoxazole-2-carbonyl)-butyl]-2-(bis-thiophen-2-ylmethyl-amino)-3-phenylmethanesulfonyl-propionamide;
(R)—N—[(S)-1-(benzoxazole-2-carbonyl)-butyl]-2-dibenzylamino-3-phenylmethanesulfonyl-propionamide;
(S)—N—[(S)-1-(benzoxazole-2-carbonyl)-butyl]-2-(tetrahydro-pyran-4-ylamino)-3-thiophen-2-yl-propionamide;
(S)—N—[(S)-1-(benzoxazole-2-carbonyl)-butyl]-2-isopropylamino-3-thiophen-2-yl-propionamide;
(R)—N-[1-(benzothiazole-2-carbonyl)-butyl]-3-phenylmethanesulfonyl-2-(tetrahydro-pyran-4-ylamino)-propionamide;
(R)—N—[(S)-1-(benzoxazole-2-carbonyl)-butyl]-3-phenylmethanesulfonyl-2-(tetrahydro-pyran-4-ylamino)-propionamide;
(R)—N—[(S)-1-(benzoxazole-2-carbonyl)-butyl]-2-isopropylamino-3-phenylmethanesulfonyl-propionamide;
(R)—N—[(S)-1-(benzoxazole-2-carbonyl)-butyl]-2-[(2-methoxy-ethyl)-(tetrahydro-pyran-4-yl)-amino]-3-phenylmethanesulfonyl-propionamide;
(R)—N—[(S)-1-(benzoxazole-2-carbonyl)-butyl]-2-cyclohexylamino-3-phenylmethanesulfonyl-propionamide;
(R)—N—[(S) 1-(benzoxazole-2-carbonyl)-butyl]-2-dimethylamino-3-phenylmethanesulfonyl-propionamide;
(1S)—N-[1-(benzooxazole-2-carbonyl)-butyl]-2-(S)-fluoro-4-phenyl-butyramide;
2,2-difluoro-5-phenyl-pentanoic acid [(S)-1-(benzoxazole-2-carbonyl)-butyl]-amide;
morpholine-4-carboxylic acid (S)-1-[(S)-1-(benzooxazole-2-carbonyl)-propylcarbamoyl]-2-cyclohexyl-ethyl ester;
morpholine-4-carboxylic acid (S)-2-cyclohexyl-1-[(S)-1-(oxazolo[4,5-b]pyridine-2-carbonyl)-propylcarbamoyl]-ethyl ester;
morpholine-4-carboxylic acid (S)-2-cyclohexyl-1-[(S)-1-(5-ethyl-[1,3,4]oxadiazole-2-carbonyl)-propylcarbamoyl]-ethyl ester;
morpholine-4-carboxylic acid (S)-2-cyclohexyl-1-[(S)-1-(5-phenyl-[1,3,4]oxadiazole-2-carbonyl)-propylcarbamoyl]-ethyl ester;
morpholine-4-carboxylic acid (S)-1-[(S)-1-(benzooxazole-2-carbonyl)-propylcarbamoyl]-3-cyclohexyl-propyl ester;
4-[4,4-dimethyl-2-(morpholine-4-carbonyloxy)-pentanoylamino]-3-oxo-azepane-1-carboxylic acid benzyl ester;
(R)—N—[(S)-1-(benzoxazole-2-carbonyl)-butyl]-3-cyclopropylmethanesulfonyl-2-(tetrahydro-pyran-4-ylamino)-propionamide;
(R)—N-[1-(benzoxazole-2-carbonyl)-butyl]-2-cyclohexylamino-3-cyclopropylmethanesulfonyl-propionamide;
(R)—N-[1-(benzoxazole-2-carbonyl)-butyl]-2-cycloheptylamino-3-cyclopropylmethanesulfonyl-propionamide;
(R)-3-phenylmethanesulfonyl-N—[(S)-3-phenyl-1-(thiazole-2-carbonyl)-propyl]-2-(tetrahydro-pyran-4-ylamino)-propionamide;
(R)—N—[(S)-1-(benzoxazole-2-carbonyl)-3-phenyl-propyl]-3-cyclopropylmethanesulfonyl-2-(tetrahydro-pyran-4-ylamino)-propionamide;
(R)-3-cyclopropylmethanesulfonyl-N-[1-(5-ethyl-1,2,4-oxadiazole-3-carbonyl)-propyl]-2-(tetrahydro-pyran-4-ylamino)-propionamide;
(R)-3-phenylmethanesulfonyl-N-[1-(3-phenyl-1,2,4-oxadiazole-5-carbonyl)-propyl]-2-(tetrahydro-pyran-4-ylamino)-propionamide;
(R)—N-[1-(3-cyclopropyl-1,2,4-oxadiazole-5-carbonyl)-propyl]-3-phenylmethanesulfonyl-2-(tetrahydro-pyran-4-ylamino)-propionamide;
{(R)-1-[1-(benzothiazol-2-yl-hydroxy-methyl)-butylcarbamoyl]-2-phenylmethanesulfonyl-ethyl}-carbamic acid tert-butyl ester;
{(R)-1-[(S)-1-(benzoxazol-2-yl-hydroxy-methyl)-butylcarbamoyl]-2-phenylmethanesulfonyl-ethyl}-carbamic acid tert-butyl ester;
{(S)-1-[(S)-1-(benzoxazol-2-yl-hydroxy-methyl)-butylcarbamoyl]-2-thiophen-2-yl-ethyl}-carbamic acid tert-butyl ester;
{(R)-1-[1-(benzothiazol-2-yl-hydroxy-methyl)-butylcarbamoyl]-2-phenylmethanesulfonyl-ethyl}-carbamic acid tert-butyl ester;
{(R)-1-[(S)-1-(benzoxazol-2-yl-hydroxy-methyl)-butylcarbamoyl]-2-phenylmethanesulfonyl-ethyl}-carbamic acid tert-butyl ester;
{(R)-1-[(S)-1-(benzoxazol-2-yl-hydroxy-methyl)-butylcarbamoyl]-2-cyclopropylmethanesulfonyl-ethyl}-carbamic acid tert-butyl ester;
(R)-1-{1-[hydroxy-(3-phenyl-1,2,4-oxadiazol-5-yl)-methyl]-propylcarbamoyl}-2-phenylmethanesulfonyl-ethyl)-carbamic acid tert-butyl ester;
((R)-2-cyclopropylmethanesulfonyl-1-{(S)-1-[(5-ethyl-1,2,4-oxadiazol-3-yl)-hydroxy-methyl]-propylcarbamoyl}-ethyl)-carbamic acid tert-butyl ester;
{(R)-1-[1-(benzoxazol-2-yl-hydroxy-methyl)-butylcarbamoyl]-2-phenylmethanesulfonyl-ethyl)}-carbamic acid tert-butyl ester;
{(R)-1-[(S)-1-(benzoxazol-2-yl-hydroxy-methyl)-3-phenyl-propylcarbamoyl]-2-cyclopropylmethanesulfonyl-ethyl}-carbamic acid tert-butyl ester;
{(R)-1-[(S)-1-(hydroxy-thiazol-2-yl-methyl)-3-phenyl-propylcarbamoyl]-2-phenylmethanesulfonyl-ethyl}-carbamic acid tert-butyl ester;
{(R)-1-[(S)-1-(benzoxazol-2-yl-hydroxy-methyl)-butylcarbamoyl]-2-cyclopropylmethanesulfonyl-ethyl}-carbamic acid tert-butyl ester;
(R)-1-{1-[hydroxy-(3-phenyl-1,2,4-oxadiazol-5-yl)-methyl]-propylcarbamoyl}-2-cyclopropylmethanesulfonyl-ethyl}-carbamic acid tert-butyl ester;
((R)-2-cyclopropylmethanesulfonyl-1-{(S)-1-[(5-ethyl-1,2,4-oxadiazol-3-yl)-hydroxy-methyl]-propylcarbamoyl}-ethyl)-carbamic acid tert-butyl ester;
{(R)-1-[1-(benzoxazol-2-yl-hydroxy-methyl)-butylcarbamoyl]-2-phenylmethanesulfonyl-ethyl}-carbamic acid tert-butyl ester;
{(R)-1-[(S)-1-(benzoxazol-2-yl-hydroxy-methyl)-3-phenyl-propylcarbamoyl]-2-cyclopropylmethanesulfonyl-ethyl}-carbamic acid tert-butyl ester;
{(R)-1-[(S)-1-(hydroxy-thiazol-2-yl-methyl)-3-phenyl-propylcarbamoyl]-2-phenylmethanesulfonyl-ethyl}-carbamic acid tert-butyl ester;
(R)-2-phenylmethanesulfonyl-1-{(S)-1-[(3-cyclopropyl-1,2,4-oxadiazol-5-yl)-hydroxy-methyl]-propylcarbamoyl}-ethyl)-carbamic acid tert-butyl ester;
(R)—N—[1-(Benzoxazole-2-carbonyl)-butyl]-2-[cyclopropylmethyl-(tetrahydro-pyran-4-ylmethyl)-amino]-3-phenylmethanesulfonyl-propionamide;
(R)—N-[1-(benzothiazol-2-yl-hydroxy-methyl)-butyl]-2-dibenzylamino-3-phenylmethanesulfonyl-propionamide;
(R)—N-[1-(benzothiazol-2-yl-hydroxy-methyl)-butyl]-3-phenylmethanesulfonyl-2-(tetrahydro-pyran4-ylamino)-propionamide;
(R)—N-[1-(benzothiazol-2-yl-hydroxy-methyl)-butyl]-2-isopropylamino-3-phenylmethanesulfonyl-propionamide;
(R)—N-[1-(benzothiazol-2-yl-hydroxy-methyl)-butyl]-2-dimethylamino-3-phenylmethanesulfonyl-propionamide;
(R)—N—[(S)-1-(benzoxazol-2-yl-hydroxy-methyl)-butyl]-3-phenylmethanesulfonyl-2-(tetrahydro-pyran-4-ylamino)-propionamide;
(R)—N—[(S)-1-(benzoxazol-2-yl-hydroxy-methyl)-butyl]-2-(1-methyl-piperidin-4-ylamino)-3-phenylmethanesulfonyl-propionamide;
(R)—N—[(S)-1-(benzoxazol-2-yl-hydroxy-methyl)-butyl]-2-(bis-thiophen-2-ylmethyl-amino)-3-phenylmethanesulfonyl-propionamide;
(R)—N—[(S)-1-(benzoxazol-2-yl-hydroxy-methyl)-butyl]-2-dibenzylamino-3-phenylmethanesulfonyl-propionamide;
(S)—N—[(S)-1-(benzoxazol-2-yl-hydroxy-methyl)-butyl]-2-(tetrahydro-pyran-4-ylamino)-3-thiophen-2-yl-propionamide;
S)—N—[(S)-1-(benzoxazol-2-yl-hydroxy-methyl)-butyl]-2-isopropylamino-3-thiophen-2-yl-propionamide;
(R)—N—[(S)-1-(benzoxazol-2-yl-hydroxy-methyl)-butyl]-2-isopropylamino-3-phenylmethanesulfonyl-propionamide;
(R)—N-[1-(benzothiazol-2-yl-hydroxy-methyl)-butyl]-3-phenylmethanesulfonyl-2-(tetrahydro-pyran-4-ylamino)-propionamide;
R)—N—[(S)-1-(benzoxazol-2-yl-hydroxy-methyl)-butyl]-3-phenylmethanesulfonyl-2-(tetrahydro-pyran-4-ylamino)-propionamide;
(R)—N—[(S)-1-(benzoxazol-2-yl-hydroxy-methyl)-butyl]-3-phenylmethanesulfonyl-2-(tetrahydro-pyran-4-ylamino)-propionamide;
(R)—N—[(S)-1-(benzoxazol-2-yl-hydroxy-methyl)-butyl]-2-[(2-methoxy-ethyl)-(tetrahydro-pyran-4-yl)-amino]-3-phenylmethanesulfonyl-propionamide;
(R)—N—[(S)-1-(benzoxazol-2-yl-hydroxy-methyl)-butyl]-2-cyclohexylamino-3-phenylmethanesulfonyl-propionamide;
(R)—N—[(S)-1-(benzoxazol-2-yl-hydroxy-methyl)-butyl]-2-dimethylamino-3-phenylmethanesulfonyl-propionamide;
N-cyanomethyl-3-cyclohexyl-propionamide;
N-cyanomethyl-3-(2-difluoromethoxy-phenylmethanesulfonyl)-propionamide;
3-(3-cyclohexyl-propionylamino)-2-oxo-5-phenyl-pentanoic acid thiazol-2-ylamide;
3-cyclohexyl-N-(1-formyl-3-phenyl-propyl)-propionamide;
3-(2-difluoromethoxy-phenylmethanesulfonyl)-N—[(S)-1-(5-ethyl-[1,3,4]oxadiazole-2-carbonyl)-propyl]-propionamide;
N—[(S)-1-(benzooxazole-2-carbonyl)-propyl]-2-(2-cyano-phenylamino)-3-cyclohexyl-propionamide;
N-Cyanomethyl-3-cyclohexyl-2-(4-methoxy-phenoxy)-propionamide;
2-benzyloxy-N-cyanomethyl-3-cyclohexyl-propionamide;
(R)—N—[(S)-1-(1-benzooxazol-2-yl-methanoyl)-butyl]-2-benzyloxy-3-phenylmethanesulfonyl-propionamide;
(R)—N—[(S)-1-(1-benzooxazol-2-yl-methanoyl)-propyl]-2-methoxymethoxy-3-phenylmethanesulfonyl-propionamide;
(S)—N—[(S)-1-(1-benzooxazol-2-yl-methanoyl)-butyl]-2-hydroxy-3-phenyl-propionamide;
(R)—N—[(S)-1-(1-benzooxazol-2-yl-methanoyl)-propyl]-3-phenylmethanesulfonyl-2-triisopropylsilanyloxy-propionamide;
(R)—N—[(S)-1-(1-benzothiazol-2-yl-methanoyl)-propyl]-2-hydroxy-3-phenylmethanesulfonyl-propionamide;
(R)-2-hydroxy-3-phenylmethanesulfonyl-N—[(S)-1-(1-pyridazin-3-yl-methanoyl)-butyl]-propionamide;
(S)-3-((R)-2-hydroxy-3-phenylmethanesulfonyl-propanoylamino)-2-oxo-pentanoic acid benzylamide;
(R)—N—[(S)-1-(1-benzooxazol-2-yl-methanoyl)-propyl]-3-[2-(1,1-difluoro-methoxy)-phenylmethanesulfonyl]-2-hydroxy-propionamide;
(R)—N—[(S)-1-(1-benzothiazol-2-yl-methanoyl)-propyl]-3-[2-(1,1-difluoro-methoxy)-phenylmethanesulfonyl]-2-hydroxy-propionamide;
(2R,5S)-2-[2-(1,1-difluoro-methoxy)-phenylmethanesulfonylmethyl]-6-ethoxy-5-ethyl-morpholin-3-one; and their corresponding N-oxides, and their prodrugs, and their protected derivatives, individual isomers and mixtures of isomers thereof; and the pharmaceutically acceptable salts and solvates (e.g. hydrates) of such compounds and their N-oxides and their prodrugs, and their protected derivatives, individual isomers and mixtures of isomers thereof.

15. A compound of claim 14 selected from the group consisting of:
(R)—N-cyanomethyl-2-hydroxy-3-phenylmethanesulfonyl-propionamide;
(R)—N-(1-cyano-1-thiophen-2-yl-methyl)-2-hydroxy-3-phenylmethanesulfonyl-propionamide;
(R)—N-(1-cyano-1-thiophen-2-yl-methyl)-3-[2-(1,1-difluoro-methoxy)-phenylmethanesulfonyl]-2-hydroxy-propionamide;
(R)—N-cyanomethyl-3-[2-(1,1-difluoro-methoxy)-phenylmethanesulfonyl]-2-hydroxy-propionamide;
morpholine-4-carboxylic acid (R)-1-(cyanomethyl-carbamoyl)-2-phenylmethanesulfonyl-ethyl ester;
morpholine-4-carboxylic acid (R)-1-(cyanomethyl-carbamoyl)-2-[2-(1,1-difluoro-methoxy)-phenylmethanesulfonyl]-ethyl ester;
(R)-(2-methoxy-ethyl)-carbamic acid 1-(cyanomethyl-carbamoyl)-2-phenylmethanesulfonyl-ethyl ester;
(S)-diethyl-carbamic acid 1-(cyanomethyl-carbamoyl)-2-cyclohexyl-ethyl ester;
(S)-pyrrolidine-1-carboxylic acid 1-(cyanomethyl-carbamoyl)-2-cyclohexyl-ethyl ester;
(S)-morpholine-4-carboxylic acid 1-(cyanomethyl-carbamoyl)-2-cyclohexyl-ethyl ester;
(S)-4-Ethyl-piperazine-1-carboxylic acid 1-(cyanomethyl-carbamoyl)-2-cyclohexyl-ethyl ester;
(S)-2-hydroxymethyl-pyrrolidine-1-carboxylic acid (S)-1-(cyanomethyl-carbamoyl)-2-cyclohexyl-ethyl ester;
(S)-(2,2,2-Trifluoro-ethyl)-carbamic acid 1-(cyanomethyl-carbamoyl)-2-cyclohexyl-ethyl ester;
(S)-(2-hydroxyethyl)-carbamic acid 1-(cyanomethyl-carbamoyl)-2-cyclohexyl-ethyl ester;
(Tetrahydrofuran-2-ylmethyl)-carbamic acid (S)-1-(cyanomethyl-carbamoyl)-2-cyclohexyl-ethyl ester;
(S)-Azetidine-1-carboxylic acid 1-(cyanomethyl-carbamoyl)-2-cyclohexyl-ethyl ester;
(S)-cyclopropyl-carbamic acid 1-(cyanomethyl-carbamoyl)-2-cyclohexyl-ethyl ester;
(S)-piperidine-1-carboxylic acid 1-(cyanomethyl-carbamoyl)-2-cyclohexyl-ethyl ester;
(S)-(2-methoxy-ethyl)-carbamic acid 1-(cyanomethyl-carbamoyl)-2-cyclohexyl-ethyl ester;
(R)-3-hydroxy-pyrrolidine-1-carboxylic acid (S)-1-(cyanomethyl-carbamoyl)-2-cyclohexyl-ethyl ester;
(S)-3-hydroxy-pyrrolidine-1-carboxylic acid (S)-1-(cyanomethyl-carbamoyl)-2-cyclohexyl-ethyl ester;
(S)-morpholine-4-carboxylic acid 1-(cyanomethyl-carbamoyl)-3-cyclohexyl-propyl ester;
morpholine4-carboxylic acid (R)-1-[(S)-1-(1-benzooxazol-2-yl-methanoyl)-propylcarbamoyl]-2-phenylmethanesulfonyl-ethyl ester;
morpholine-4-carboxylic acid (R)-1-[(S)-1-(1-benzooxazol-2-yl-methanoyl)-propylcarbamoyl]-2-[2-(1,1-difluoro-methoxy)-phenylmethanesulfonyl]-ethyl ester;
morpholine-4-carboxylic acid (R)-1-[(S)-1-(1-benzothiazol-2-yl-methanoyl)-propylcarbamoyl]-2-[2-(1,1-difluoro-methoxy)-phenylmethanesulfonyl]-ethyl ester;
pyrrolidine-1-carboxylic acid (R)-1-[(S)-1-(1-benzooxazol-2-yl-methanoyl)-propylcarbamoyl]-2-phenylmethanesulfonyl-ethyl ester;
dimethyl-carbamic acid (R)-1-[(S)-1-(1-benzooxazol-2-yl-methanoyl)-propylcarbamoyl]-2-phenylmethanesulfonyl-ethyl ester;
morpholine-4-carboxylic acid (R)-1-[(S)-1-(1-benzylcarbamoyl-methanoyl)-propylcarbamoyl]-2-phenylmethanesulfonyl-ethyl ester;
morpholine-4-carboxylic acid (S)-1-[(S)-1-(oxazolo[4,5-b]pyridine-2-carbonyl)-propylcarbamoyl]-2-phenylmethanesulfonyl-ethyl ester;
morpholine-4-carboxylic acid (S)-1-[(S)-1-(5-ethyl-[1,3,4]oxadiazole-2-carbonyl)-propylcarbamoyl]-2-phenylmethanesulfonyl-ethyl ester;
(S)-2-{(R)-3-[2-(1,1-difluoro-methoxy)-phenylmethanesulfonyl]-2-hydroxy-propanoylamino}-N-methoxy-N-methyl-butyramide;
(R)-3-[2-(1,1-difluoro-methoxy)-phenylmethanesulfonyl]-N—((S)-1-formyl-propyl)-2-hydroxy-propionamide;
(R)—N—[(S)-1-(1-benzooxazol-2-yl-methanoyl)-propyl-2-hydroxy-3-phenyl-methanesulfonyl-propionamide;
(S)-3-{3-[2-(1,1-difluoro-methoxy)-phenylmethanesulfonyl]-propanoylamino}-2-oxo-pentanoic acid benzylamide;
N—[(S)-1-(1-benzooxazol-2-yl-methanoyl)-propyl]-3-[2-(1,1-difluoro-methoxy)-phenylmethanesulfonyl]-propionamide;
N—[(S)-1-(1-benzooxazol-2-yl-methanoyl)-3-phenyl-propyl]-3-p-tolylmethanesulfonyl-propionamide;
3-(2-difluoromethoxy-phenylmethanesulfonyl)-N-(1-ethyl-2,3-dioxo-3-pyrrolidin-1-yl-propyl)-propionamide;
3-(2-difluoromethoxy-phenylmethanesulfonyl)-N-(1-ethyl-3-morpholin-4-yl-2,3-dioxo-propyl)-propionamide;
3-(2-difluoromethoxy-phenylmethanesulfonyl)-N-(1-ethyl-2,3-dioxo-3-piperazin-1-yl-propyl)-propionamide;
3-(2-difluoromethoxy-phenylmethanesulfonyl)-N-[3-(1,1-dioxo-116-thiomorpholin-4-yl)-1-ethyl-2,3-dioxo-propyl]-propionamide;
3-(2-difluoromethoxy-phenylmethanesulfonyl)-N-[1-ethyl-3-(4-methyl-sulfonyl-piperazin-1-yl)-2,3-dioxo-propyl]-propionamide;
3-[3-(2-difluoromethoxy-phenylmethanesulfonyl)-propionylamino]-2-oxo-pentanoic acid dimethylamide;
3-[3-(2-difluoromethoxy-phenylmethanesulfonyl)-propionylamino]-2-oxo-pentanoic acid cyclopentyl-ethyl-amide;
3-[3-(2-difluoromethoxy-phenylmethanesulfonyl)-propionylamino]-2-oxo-pentanoic acid phenylamide;
3-[3-(2-difluoromethoxy-phenylmethanesulfonyl)-propionylamino]-2-oxo-pentanoic acid pyridin-3-ylamide;
3-[3-(2-difluoromethoxy-phenylmethanesulfonyl)-propionylamino]-2-oxo-pentanoic acid (tetrahydro-pyran-4-yl)-amide;
3-[3-(2-difluoromethoxy-phenylmethanesulfonyl)-propionylamino]-2-oxo-pentanoic acid (1-benzoyl-piperidin-4-yl)-amide;
3-[3-(2-difluoromethoxy-phenylmethanesulfonyl)-propionylamino]-2-oxo-pentanoic acid (2-morpholin-4-yl-ethyl)-amide;
(R)—N—[(S)-1-(1-benzooxazol-2-yl-methanoyl)-propyl]-2-(2-nitro-phenylamino)-3-phenylmethanesulfonyl-propionamide;
N-[1-(benzooxazole-2-carbonyl)-propyl)-3-phenylmethanesulfonyl-2-(pyrimidin-2-ylamino)-propionamide.
(R)—N—[(S)-1-(1-benzooxazol-2-yl-methanoyl)-butyl]-2-(5-nitro-thiazol-2-ylamino)-3-phenylmethanesulfonyl-propionamide;
(2S) (4,4-difluoro-2-hydroxy-5-phenyl-pentanoic acid (1(S)-cyano-3-phenyl-propyl)-amide;
N-(1(S)-cyano-3-phenyl-propyl)-2-(S)-(2-morpholin-4-yl-2-oxo-ethoxy)-4-phenyl-butyramide;
N-(1-(S)-cyano-3-phenyl-propyl)-2-(S)-fluoro-4-phenyl-butyramide;
N-(1-(S)-cyano-3-phenyl-propyl)-2,2-difluoro-4-phenyl-butyramide;
N-(1-(S)-cyano-3-phenyl-propyl)-2-(S)-hydroxy-4-phenyl-butyramide;
N-(1-(S)-cyano-3-phenyl-propyl)-2-(R)-hydroxy-4-phenyl-butyramide;
N-(1-(S)-cyano-3-phenyl-propyl)-2-(R)-methoxy-4-phenyl-butyramide;
2,2-difluoro-5-phenyl-pentanoic acid (1-cyano-cyclopropyl)-amide;
N-(1-(S)-cyano-3-phenyl-propyl)-4-phenyl-butyramide;
2,2-difluoro-5-phenyl-pentanoic acid ((S)-1-cyano-3-phenyl-propyl)-amide;
N-(4-cyano-1-ethyl-piperidin-4-yl)-3-cyclohexyl-propionamide;
N-(4-cyano-1-ethyl-piperidin-4-yl)-3-(2-difluoromethoxy-phenylmethanesulfonyl)-propionamide;
(S)-tert-butyl-carbamic acid 1-(cyanomethyl-carbamoyl)-2-cyclohexyl-ethyl ester;
(R)-carbamic acid 1-(cyanomethyl-carbamoyl)-2-(2-difluoromethoxy-phenylmethanesulfonyl)-ethyl ester;
(S)-carbamic acid 1-(cyanomethyl-carbamoyl)-2-cyclohexyl-ethyl ester;
(R)-morpholine-4-carboxylic acid 1-(1-cyano-cyclopropylcarbamoyl)-2-phenylmethanesulfonyl-ethyl ester;
(R)-morpholine-4-carboxylic acid 1-(4-cyano-tetrahydro-pyran-4-ylcarbamoyl)-2-phenylmethanesulfonyl-ethyl ester;
3-cyclohexyl-2-hydroxy-N-[1-(oxazolo[4,5-b]pyridine-2-carbonyl)-propyl]-propionamide;
(R)—N-[1-(benzothiazole-2-carbonyl)-butyl]-2-isopropylamino-3-phenylmethanesulfonyl-propionamide;
(R)—N-[1-(benzothiazole-2-carbonyl)-butyl]-3-phenylmethanesulfonyl-2-(tetrahydro-pyran-4-ylamino)-propionamide;
(R)—N-[1-(benzothiazole-2-carbonyl)-butyl]-2-dibenzylamino-3-phenylmethanesulfonyl-propionamide;
(R)—N-[1-(benzothiazole-2-carbonyl)-butyl]-2-dimethylamino-3-phenylmethanesulfonyl-propionamide;
(R)—N—[(S)-1-(benzoxazole-2-carbonyl)-butyl]-3-phenylmethanesulfonyl-2-(tetrahydro-pyran-4-ylamino)-propionamide;
(R)—N—[(S)-1-(benzoxazole-2-carbonyl)-butyl]-2-(1-methyl-piperidin-4-ylamino)-3-phenylmethanesulfonyl-propionamide;
(R)—N—[(S)-1-(benzoxazole-2-carbonyl)-butyl]-2-(bis-thiophen-2-ylmethyl-amino)-3-phenylmethanesulfonyl-propionamide;
(R)—N—[(S)-1-(benzoxazole-2-carbonyl)-butyl]-2-dibenzylamino-3-phenylmethanesulfonyl-propionamide;
(S)—N—[(S)-1-(benzoxazole-2-carbonyl)-butyl]-2-(tetrahydro-pyran-4-ylamino)-3-thiophen-2-yl-propionamide;
(S)—N—[(S)-1-(benzoxazole-2-carbonyl)-butyl]-2-isopropylamino-3-thiophen-2-yl-propionamide;
(R)—N-[1-(benzothiazole-2-carbonyl)-butyl]-3-phenylmethanesulfonyl-2-(tetrahydro-pyran-4-ylamino)-propionamide;
(R)—N—[(S)-1-(benzoxazole-2-carbonyl)-butyl]-3-phenylmethanesulfonyl-2-(tetrahydro-pyran-4-ylamino)-propionamide;
(R)—N—[(S)-1-(benzoxazole-2-carbonyl)-butyl]-2-isopropylamino-3-phenylmethanesulfonyl-propionamide;
(R)—N—[(S)-1-(benzoxazole-2-carbonyl)-butyl]-2-[(2-methoxy-ethyl)-(tetrahydro-pyran-4-yl)-amino]-3-phenylmethanesulfonyl-propionamide;
(R)—N—[(S)-1-(benzoxazole-2-carbonyl)-butyl]-2-cyclohexylamino-3-phenylmethanesulfonyl-propionamide;
(R)—N—[(S)-1-(benzoxazole-2-carbonyl)-butyl]-2-dimethylamino-3-phenylmethanesulfonyl-propionamide;
(1S)—N-[1-(benzooxazole-2-carbonyl)-butyl]-2-(S)-fluoro-4-phenyl-butyramide;
2,2-difluoro-5-phenyl-pentanoic acid [(S)-1-(benzoxazole-2-carbonyl)-butyl]-amide;
morpholine-4-carboxylic acid (S)-1-[(S)-1-(benzooxazole-2-carbonyl)-propylcarbamoyl]-2-cyclohexyl-ethyl ester;
morpholine-4-carboxylic acid (S)-2-cyclohexyl-1-[(S)-1-(oxazolo[4,5-b]pyridine-2-carbonyl)-propylcarbamoyl]-ethyl ester;
morpholine-4-carboxylic acid (S)-2-cyclohexyl-1-[(S)-1-(5-ethyl-[1,3,4]oxadiazole-2-25 carbonyl)-propylcarbamoyl]-ethyl ester;
morpholine-4-carboxylic acid (S)-2-cyclohexyl-1-[(S)-1-(5-phenyl-[1,3,4]oxadiazole-2-carbonyl)-propylcarbamoyl]-ethyl ester;
morpholine-4-carboxylic acid (S)-1-[(S)-1-(benzooxazole-2-carbonyl)-propylcarbamoyl]-3-cyclohexyl-propyl ester;
4-[4,4-dimethyl-2-(morpholine-4-carbonyloxy)-pentanoylamino]-3-oxo-azepane-1-carboxylic acid benzyl ester;
(R)—N—[(S)-1-(benzoxazole-2-carbonyl)-butyl]-3-cyclopropylmethanesulfonyl-2-(tetrahydro-pyran-4-ylamino)-propionamide;
(R)—N-[1-(benzoxazole-2-carbonyl)-butyl]-2-cyclohexylamino-3-cyclopropylmethanesulfonyl-propionamide;
(R)—N-[1-(benzoxazole-2-carbonyl)-butyl]-2-cycloheptylamino-3-cyclopropylmethanesulfonyl-propionamide;
(R)-3-phenylmethanesulfonyl-N—[(S)-3-phenyl-1-(thiazole-2-carbonyl)-propyl]-2-(tetrahydro-pyran-4-ylamino)-propionamide;
(R)—N—[(S)-1-(benzoxazole-2-carbonyl)-3-phenyl-propyl]-3-cyclopropylmethanesulfonyl-2-(tetrahydro-pyran-4-ylamino)-propionamide;
(R)-3-cyclopropylmethanesulfonyl-N-[1-(5-ethyl-1,2,4-oxadiazole-3-carbonyl)-propyl]-2-(tetrahydro-pyran-4-ylamino)-propionamide;
(R)-3-phenylmethanesulfonyl-N-[1-(3-phenyl-1,2,4-oxadiazole-5-carbonyl)-propyl]-2-(tetrahydro-pyran-4-ylamino)-propionamide;
(R)—N-[1-(3-cyclopropyl-1,2,4-oxadiazole-5-carbonyl)-propyl]-3-phenylmethanesulfonyl-2-(tetrahydro-pyran-4-ylamino)-propionamide;
{(R)-1-[1-(benzothiazol-2-yl-hydroxy-methyl)-butylcarbamoyl]-2-phenylmethanesulfonyl-ethyl}-carbamic acid tert-butyl ester;
{(R)-1-[(S)-1-(benzoxazol-2-yl-hydroxy-methyl)-butylcarbamoyl]-2-phenylmethanesulfonyl-ethyl}-carbamic acid tert-butyl ester;
{(S)-1-[(S)-1-(benzoxazol-2-yl-hydroxy-methyl)-butylcarbamoyl]-2-thiophen-2-yl-ethyl}-carbamic acid tert-butyl ester;
{(R)-1-[1-(benzothiazol-2-yl-hydroxy-methyl)-butylcarbamoyl]-2-phenylmethanesulfonyl-ethyl}-carbamic acid tert-butyl ester;
{(R)-1-[(S)-1-(benzoxazol-2-yl-hydroxy-methyl)-butylcarbamoyl]-2-phenylmethanesulfonyl-ethyl}-carbamic acid tert-butyl ester;
{(R)-1-[(S)-1-(benzoxazol-2-yl-hydroxy-methyl)-butylcarbamoyl]-2-cyclopropylmethanesulfonyl-ethyl}-carbamic acid tert-butyl ester;
(R)-1-{1-[hydroxy-(3-phenyl-1,2,4-oxadiazol-5-yl)-methyl]-propylcarbamoyl}-2-phenylmethanesulfonyl-ethyl)-carbamic acid tert-butyl ester;
((R)-2-cyclopropylmethanesulfonyl-1-{(S)-1-[(5-ethyl-1,2,4-oxadiazol-3-yl)-hydroxy-methyl]-propylcarbamoyl}-ethyl)-carbamic acid tert-butyl ester;
{(R)-1-[1-(benzoxazol-2-yl-hydroxy-methyl)-butylcarbamoyl]-2-phenylmethanesulfonyl-ethyl}-carbamic acid tert-butyl ester;
{(R)-1-[(S)-1-(benzoxazol-2-yl-hydroxy-methyl)-3-phenyl-propylcarbamoyl]-2-cyclopropylmethanesulfonyl-ethyl}-carbamic acid tert-butyl ester;
{(R)-1-[(S)-1-(hydroxy-thiazol-2-yl-methyl)-3-phenyl-propylcarbamoyl]-2-phenylmethanesulfonyl-ethyl}-carbamic acid tert-butyl ester;
{(R)-1-[(S)-1-(benzoxazol-2-yl-hydroxy-methyl)-butylcarbamoyl]-2-cyclopropylmethanesulfonyl-ethyl}-carbamic acid tert-butyl ester;
(R)-1-{1-[hydroxy-(3-phenyl-1,2,4-oxadiazol-5-yl)-methyl]-propylcarbamoyl}-2-phenylmethanesulfonyl-ethyl)-carbamic acid tert-butyl ester;
((R)-2-cyclopropylmethanesulfonyl-1-{(S)-1-[(5-ethyl-1,2,4-oxadiazol-3-yl)-hydroxy-methyl]-propylcarbamoyl}-ethyl)-carbamic acid tert-butyl ester;
{(R)-1-[1-(benzoxazol-2-yl-hydroxy-methyl)-butylcarbamoyl]-2-phenylmethanesulfonyl-ethyl}-carbamic acid tert-butyl ester;
{(R)-1-[(S)-1-(benzoxazol-2-yl-hydroxy-methyl)-3-phenyl-propylcarbamoyl]-2-cyclopropylmethanesulfonyl-ethyl}-carbamic acid tert-butyl ester;
{(R)-1-[(S)-1-(hydroxy-thiazol-2-yl-methyl)-3-phenyl-propylcarbamoyl]-2-phenylmethanesulfonyl-ethyl}-carbamic acid tert-butyl ester;
(R)-2-phenylmethanesulfonyl-1-{(S)-1-[(3-cyclopropyl-1,2,4-oxadiazol-5-yl)-hydroxy-methyl]-propylcarbamoyl}-ethyl)-carbamic acid tert-butyl ester;
(R)—N-[1-(Benzoxazole-2-carbonyl)-butyl]-2-[cyclopropylmethyl-(tetrahydro-pyran-4-ylmethyl)-amino]-3-phenylmethanesulfonyl-propionamide;
(R)—N-[1-(benzothiazol-2-yl-hydroxy-methyl)-butyl]-2-dibenzylamino-3-phenylmethanesulfonyl-propionamide;
(R)—N-[1-(benzothiazol-2-yl-hydroxy-methyl)-butyl]-3-phenylmethanesulfonyl-2-(tetrahydro-pyran-4-ylamino)-propionamide;
(R)—N-[1-(benzothiazol-2-yl-hydroxy-methyl)-butyl]-2-isopropylamino-3-phenylmethanesulfonyl-propionamide;
(R)—N-[1-(benzothiazol-2-yl-hydroxy-methyl)-butyl]-2-dimethylamino-3-phenylmethanesulfonyl-propionamide;
(R)—N—[(S)-1-(benzoxazol-2-yl-hydroxy-methyl)-butyl]-3-phenylmethanesulfonyl-2-(tetrahydro-pyran-4-ylamino)-propionamide;
(R)—N—[(S)-1-(benzoxazol-2-yl-hydroxy-methyl)-butyl]-2-(1-methyl-piperidin4-ylamino)-3-phenylmethanesulfonyl-propionamide;
(R)—N—[(S)-1-(benzoxazol-2-yl-hydroxy-methyl)-butyl]-2-(bis-thiophen-2-ylmethyl-amino)-3-phenylmethanesulfonyl-propionamide;
(R)—N—[(S)-1-(benzoxazol-2-yl-hydroxy-methyl)-butyl]-2-dibenzylamino-3-phenylmethanesulfonyl-propionamide;
(S)—N—[(S)-1-(benzoxazol-2-yl-hydroxy-methyl)-butyl]-2-(tetrahydro-pyran-4-ylamino)-3-thiophen-2-yl-propionamide;
S)—N—[(S)-1-(benzoxazol-2-yl-hydroxy-methyl)-butyl]-2-isopropylamino-3-thiophen-2-yl-propionamide;
(R)—N—[(S)-1-(benzoxazol-2-yl-hydroxy-methyl)-butyl]-2-isopropylamino-3-phenylmethanesulfonyl-propionamide;
(R)—N-[1-(benzothiazol-2-yl-hydroxy-methyl)-butyl]-3-phenylmethanesulfonyl-2-(tetrahydro-pyran-4-ylamino)-propionamide;
R)—N—[(S)-1-(benzoxazol-2-yl-hydroxy-methyl)-butyl]-3-phenylmethanesulfonyl-2-(tetrahydro-pyran-4-ylamino)-propionamide;
(R)—N—[(S)-1-(benzoxazol-2-yl-hydroxy-methyl)-butyl]-3-phenylmethanesulfonyl-2-(tetrahydro-pyran-4-ylamino)-propionamide;
(R)—N—[(S)-1-(benzoxazol-2-yl-hydroxy-methyl)-butyl]-2-[(2-methoxy-ethyl)-(tetrahydro-pyran-4-yl)-amino]-3-phenylmethanesulfonyl-propionamide;
(R)—N—[(S)-1-(benzoxazol-2-yl-hydroxy-methyl)-butyl]-2-cyclohexylamino-3-phenylmethanesulfonyl-propionamide;
(R)—N—[(S)-1-(benzoxazol-2-yl-hydroxy-methyl)-butyl]-2-dimethylamino-3-phenylmethanesulfonyl-propionamide;
N-cyanomethyl-3-cyclohexyl-propionamide;
N-cyanomethyl-3-(2-difluoromethoxy-phenylmethanesulfonyl)-propionamide;
3-(3-cyclohexyl-propionylamino)-2-oxo-5-phenyl-pentanoic acid thiazol-2-ylamide;
3-cyclohexyl-N-(1-formyl-3-phenyl-propyl)-propionamide;
3-(2-difluoromethoxy-phenylmethanesulfonyl)-N—[(S)-1-(5-ethyl-[1,3,4]oxadiazole-2-carbonyl)-propyl]-propionamide;
N—[(S)-1-(benzooxazole-2-carbonyl)-propyl]-2-(2-cyano-phenylamino)-3-cyclohexyl-propionamide;
N-Cyanomethyl-3-cyclohexyl-2-(4-methoxy-phenoxy)-propionamide;
2-benzyloxy-N-cyanomethyl-3-cyclohexyl-propionamide;
(R)—N—[(S)-1-(1-benzooxazol-2-yl-methanoyl)-butyl]-2-benzyloxy-3-phenylmethanesulfonyl-propionamide;
(R)—N—[(S)-1-(1-benzooxazol-2-yl-methanoyl)-propyl]-2-methoxymethoxy-3-phenylmethanesulfonyl-propionamide;
(S)—N—[(S)-1-(1-benzooxazol-2-yl-methanoyl)-butyl]-2-hydroxy-3-phenyl-propionamide;
(R)—N—[(S)-1-(1-benzooxazol-2-yl-methanoyl)-propyl]-3-phenylmethanesulfonyl-2-triisopropylsilanyloxy-propionamide;
(R)—N—[(S)-1-(1-benzothiazol-2-yl-methanoyl)-propyl]-2-hydroxy-3-phenylmethanesulfonyl-propionamide;
(R)-2-hydroxy-3-phenylmethanesulfonyl-N—[(S)-1-(1-pyridazin-3-yl-methanoyl)-butyl]-propionamide;
(S)-3-((R)-2-hydroxy-3-phenylmethanesulfonyl-propanoylamino)-2-oxo-pentanoic acid benzylamide;
(R)—N—[(S)-1-(1-benzooxazol-2-yl-methanoyl)-propyl]-3-[2-(1,1-difluoro-methoxy)-phenylmethanesulfonyl]-2-hydroxy-propionamide;
(R)—N—[(S)-1-(1-benzothiazol-2-yl-methanoyl)-propyl]-3-[2-(1,1-difluoro-methoxy)-phenylmethanesulfonyl]-2-hydroxy-propionamide; and
(2R,5S)-2-[2-(1,1-difluoro-methoxy)-phenylmethanesulfonylmethyl]-6-ethoxy-5-ethyl-morpholin-3-one.

16. A compound of claim 15 selected from the group consisting of:
morpholine-4-carboxylic acid (R)-1-(cyanomethyl-carbamoyl)-2-[2-(1,1-difluoro-methoxy)-phenylmethanesulfonyl]-ethyl ester, (Compound 31);
morpholine-4-carboxylic acid (R)-1-[(S)-1-(1-benzooxazol-2-yl-methanoyl)-propylcarbamoyl]-2-phenylmethanesulfonyl-ethyl ester, (Compound 11);
morpholine-4-carboxylic acid (R)-1-[(S)-1-(1-benzooxazol-2-yl-methanoyl)-propylcarbamoyl]-2-[2-(1,1-difluoro-methoxy)-phenylmethanesulfonyl]-ethyl ester, (Compound 14);
morpholine-4-carboxylic acid (R)-1-[(S)-1-(1-benzothiazol-2-yl-methanoyl)-propylcarbamoyl]-2-[2-(1,1-difluoro-methoxy)-phenylmethanesulfonyl]-ethyl ester, (Compound 15);
pyrrolidine-1-carboxylic acid (R)-1-[(S)-1-(I-benzooxazol-2-yl-methanoyl)-propylcarbamoyl]-2-phenylmethanesulfonyl-ethyl ester, (Compound 19);
dimethyl-carbamic acid (R)-1-[(S)-1-(1-benzooxazol-2-yl-methanoyl)-propylcarbamoyl]-2-phenylmethanesulfonyl-ethyl ester, (Compound 20);
morpholine-4-carboxylic acid (R)-1-[(S)-1-(1-benzylcarbamoyl-methanoyl)-propylcarbamoyl]-2-phenylmethanesulfonyl-ethyl ester, (Compound 25);
morpholine-4-carboxylic acid (S)-1-[(S)-1-(oxazolo [4,5-b]pyridine-2-carbonyl)-propylcarbamoyl]-2-phenylmethanesulfonyl-ethyl ester;
morpholine-4-carboxylic acid (S)-1-[(S)-1-(5-ethyl-[1,3,4]oxadiazole-2-carbonyl)-propylcarbamoyl]-2-phenylmethanesulfonyl-ethyl ester;
(R)-3-[2-(1,1-difluoro-methoxy)-phenylmethanesulfonyl]-N—((S)-1-formyl-propyl)-2-hydroxy-propionamide;
(R)—N—[(S)-1-(1-benzooxazol-2-yl-methanoyl)-propyl]-2-hydroxy-3-phenyl-methanesulfonyl-propionamide;
(S)-3-{3-[2-(1, 1-difluoro-methoxy)-phenylmethanesulfonyl]-propanoylamino}-2-oxo-pentanoic acid benzylamide;
(R)—N—[(S)-1-(1-benzooxazol-2-yl-methanoyl)-propyl]-2-(2-nitro-phenylamino)-3-phenylmethanesulfonyl-propionamide;
(R)—N—[(S)-1-(1-benzooxazol-2-yl-methanoyl)-butyl]-2-(5-nitro-thiazol-2-ylamino)-3-phenylmethanesulfonyl-propionamide;
(R)—N—[(S)-1-(benzoxazole-2-carbonyl)-butyl]-3-phenylmethanesulfonyl-2-(tetrahydro-pyran-4-ylamino)-propionamide;
(R)—N—[(S)-1-(benzoxazole-2-carbonyl)-butyl]-2-isopropylamino-3-phenylmethanesulfonyl propionamide;
(R)—N—[(S)-1-(benzoxazole-2-carbonyl)-butyl]-2-[(2-methoxy-ethyl)-(tetrahydro-pyran-4-yl)-amino]-3-phenylmethanesulfonyl-propionamide;
(R)—N—[(S)-1-(benzoxazole-2-carbonyl)-butyl]-2-cyclohexylamino-3-phenylmethanesulfonyl-propionamide;
morpholine-4-carboxylic acid (S)-2-cyclohexyl-1-[(S)-1-(oxazolo[4,5-b]pyridine-2-carbonyl)-propylcarbamoyl]-ethyl ester;
(S)-3-((R)-2-hydroxy-3-phenylmethanesulfonyl-propanoylamino)-2-oxo-pentanoic acid benzylamide;
(R)—N—[(S)-1-(1-benzooxazol-2-yl-methanoyl)-propyl]-3-[2-(1,1-difluoro-methoxy)-2-hydroxy-propionamide.

17. A pharmaceutical composition comprising a therapeutically effective amount of a compound of claim 1 in combination with a pharmaceutically acceptable excipient.

18. A pharmaceutical composition comprising a therapeutically effective amount of a compound of claim 2 in combination with a pharmaceutically acceptable excipient.

19. A method for treating a disease in an animal in which inhibition of Cathepsin S can prevent, inhibit or ameliorate the pathology and/or symptomology of the disease, which method comprises administering to the animal a therapeutically effective amount of compound of claim 1 or claim 2.

20. The use of a compound of claim 1 or 2 in the manufacture of a medicament for treating a disease in an animal in which Cathepsin S activity contributes to the pathology and/or symptomology of the disease.
